(19) United States
(12) Patent Application Publication
(10) Pub. No.: US 2026/0224678 A1
(43) Pub. Date: Aug. 6, 2026

(54) EXPEDITED NEOANTIGEN VACCINES

(71) Applicant: IOGENETICS, LLC, Sun Prairie, WI (US)

(72) Inventors: Jane Homan, Sun Prairie, WI (US); Robert D. Bremel, Sun Prairie, WI (US)

(21) Appl. No.: 19/154,065

(22) PCT Filed: Feb. 8, 2024
(86) PCT No.: PCT/US2024/014987

§ 371 Date: Aug. 6, 2025

Related U.S. Application Data
(60) Provisional application No. 63/444,135, filed on Feb. 8, 2023.
(60) Provisional application No. 63/452,766, filed on Mar. 17, 2023.
(60) Provisional application No. 63/468,663, filed on May 24, 2023.

(51) Int. Cl. A61K 39/00; A61K 35/17; A61K 39/395; A61K 45/06; A61P 35/00; A61P 37/04; C07K 16/40; C12N 5/0783; G01N 33/575

(52) U.S. Cl. CPC A61K 39/0011; A61K 35/17; A61K 39/3955; A61K 45/06; A61P 35/00; A61P 37/04; C07K 16/40; C12N 5/0636; G01N 33/5758; A61K 2039/5154; C12N 2510/00

(57) ABSTRACT

The present invention provides methods for evaluation of potential tumor neoepitopes to assess the probability that they constitute immunogenic neoantigens in a cancer affected subject. The present invention provides vaccines comprising immunogenic neoepitopes for treatment of cancer in subjects in need thereof, optionally with the coadministration of cathepsin inhibitor.

### CROSS-REFERENCE TO RELATED APPLICATIONS

This application claims the benefit of U.S. Prov. Appl. 63/444,135 filed Feb. 8, 2023, U.S. Prov. Appl. 63/452,766, filed Mar. 17, 2023, and U.S. Prov. Appl. 63/468,663, filed May 24, 2023, each of which is incorporated by reference herein in their entirety.

### REFERENCE TO A SEQUENCE LISTING

The text of the computer readable sequence listing filed herewith, titled “IOGEN_41682_601_SequenceListing.xml”, created Feb. 8, 2024, having a file size of 2,275,766 bytes, is hereby incorporated by reference in its entirety.

### FIELD OF THE INVENTION

The present invention addresses methods that enable a rapid response to cancer diagnosis and tumor biopsy sequencing and which expedite personalized neoantigen vaccination. This is achieved by enabling rapid selection of T cell exposed amino acid motifs that have greatest potential for immunostimulation in the individual affected subject, followed by selection and design of peptides comprising these motifs, or the nucleic acid sequences that encode them. This enables the preparation of a library of neoantigens for common combinations of mutations and HLA alleles. The present invention also provides a modularized rapid assembly of peptides for inclusion in neoantigen vaccines.

### BACKGROUND OF THE INVENTION

Cancer immunotherapy functions by directing cytotoxic T cell responses to tumor cells. This depends on the recognition of tumor specific neoantigens in individual cancer patients (1). Considerable progress has been made in the development of neoantigen vaccines (2). Relatively few tumor mutations give rise to neoantigens which are effective immunogens (3) but the reasons for this have been poorly understood.

Tumors typically comprise many mutated proteins. Some mutations occur in known oncogenes or tumor suppressor gene products, identified as drivers of tumor progression, while others are in accompanying “passenger” gene products. Many common tumor-specific mutations have characteristics indicative of immune escape or evasion. Such evasion may arise because the mutant creates a very rare epitope that has no T cell precursor clone capable of responding. Evasion may also arise because the peptide encompassing the mutation is cleaved by an endopeptidase, including but not limited to a cathepsin, to preclude presentation to a T cell. In other instances, evasion of an effective CD8+ response may occur where no CD4+ helper response is elicited which enables the development of a mature CD8+ response and memory. In yet other instances, the peptide carrying the mutation may be bound by one or more of the affected subject's MHC in a register that preferentially hides the mutant amino acid in the MHC groove, thus avoiding T cell recognition of a tumor-specific neoepitope. Additionally, in many instances, the mutant-bearing peptide is not bound by any of the subject's MHC alleles and thus is not presented to T cells and so escapes detection. A further important mechanism of immune evasion is that a mutated gene product may not be expressed and is thus not subject to immune recognition.

Evaluating the common mutations in cancer driver gene products can identify many of these features which facilitate immune evasion and favor tumor progression. While neoepitope vaccines have shown promise, their efficacy and impact may be improved by focusing precisely on those epitopes which are most likely to actively engage a cognate T cell clone or clones to invoke an effective immune response, and which can also avoid exacerbating further immune downregulation in the tumor microenvironment.

Several of the characteristics of neoantigens in commonly mutated cancer gene products can be evaluated before they are identified in a biopsy to identify which are more or less likely to be immunogenic. These characteristics are independent of an affected subject's HLA genotype. They include determining if the potential T cell exposed amino acid motifs are rare or common relative to the number of counts of the corresponding pentamer amino acid motif counts in the normal human proteome and in other reference databases of amino acid motifs. The predicted cathepsin cleavage of the mutated peptides is also independent of HLA genotype. These features are in contrast to the more often considered MHC binding, which is dependent on a particular patient's HLA genotype. Prior evaluation and categorization of the characteristics of potential neoantigens can expedite the selection and preparation of a personalized neoepitope vaccine once a determination of which driver and passenger mutations are present in a subject is made by sequencing of a biopsy.

The optimum timing for applying a neoepitope vaccine to recruit effective T cell clones is as soon as possible after sequencing of a tumor biopsy is available. This enables the stimulation of tumor specific T cell clones, not only to act alone, but also to be further stimulated by other subsequent interventions such as checkpoint inhibitors, and to act in synergy with other immunomodulatory interventions. In light of the need for rapid response to cancer diagnosis, methods which can accelerate neoepitope vaccine design and synthesis are advantageous. This includes, but is not limited to, rapid down-selection of mutated peptides to key neoepitopes with highest probability of immunogenicity, the preparation of pre-designed peptides for common tumor mutations, and the preparation of ready-to-assemble peptide subcomponents.

What is need in the art is a rapid means to select, prepare and deliver vaccines which target these neoepitopes. Precision neoepitope vaccination can provide positive benefits to a cancer affected subject by recruiting tumor specific T cells. It can also enhance the efficacy of other immunotherapy interventions, while mitigating potential negative side effects.

### SUMMARY OF THE INVENTION

In some embodiments, the present invention provides methods for evaluation of potential tumor neoepitopes to assess the probability that they constitute immunogenic neoantigens in a subject with cancer.

The first criterion used in this evaluation is a comparison of the pentameric amino acid motifs in the potential T cell exposed motifs, which comprise a mutated amino acid in a mutated tumor protein, with the count of the same pentameric amino acid motifs in the normal human proteome and in other reference databases of proteins. Such reference databases are from proteins and proteomes which may have contributed to the establishment and shaping of the T cell repertoire. The purpose of this invention is to expedite evaluation of the probability that T cells cognate for the tumor specific mutation may be present in the T cell repertoire and capable of responding to a neoantigen vaccine and thus expedite the rapid design and synthesis of a neoantigen vaccine for administration to one or more subjects having cancer.

In one embodiment, in order to select peptides for inclusion in an immunological treatment of a subjects having cancer, or at risk of developing cancer, a biopsy of the tumor is first sequenced to provide the sequences of those proteins which are mutated in the tumor. From these, the mutations specific to the tumor are identified and the potential T cell exposed motifs that would comprise the mutated amino acids and expose them to the T cell receptor of a T cell are identified. The pentameric amino acid motifs that correspond to these T cell exposed motif are identified and the frequency of occurrence of each of these pentameric amino acid motifs is determined in a reference database of such motifs derived from a proteome of interest. The frequency of occurrence of the pentameric amino acid motif in that database is then applied as a selection criterion in order to select mutated peptides for synthesis, either directly as a peptide, or as a nucleotide sequence encoding the peptide. In preferred embodiments, the selected and synthesized peptides, or the nucleotides that encode them, are then incorporated into a neoantigen vaccine which is administered to the cancer-affected subject. In one embodiment of the invention, the reference database used to evaluate the frequency of pentameric amino acid motifs is the complete normal human proteome. In other embodiments, the reference database is compiled from the open reading frames encoding the proteins of a group of microorganisms. In particularly preferred embodiments, the microorganisms are those found in the gastrointestinal microbiome, however other microorganism groupings may be used, including but not limited to, pathogenic microorganisms or other environmental organisms. In yet further embodiments, the reference database applied in the determination of the frequency of each pentameric amino acid motif is a database comprised of the variable region sequences of human immunoglobulins. In some embodiments, the reference database is comprised of at least 1000 proteins, in other embodiments, the reference database comprises more than 10,000 proteins, and in a most preferred embodiment, the reference database is made up of more than 20,000 proteins. In some preferred instances, the evaluation of frequency is done by analysis of more than one reference database; as an example this may include the human proteome and the gastrointestinal microbiome databases.

Using the methods described above, neoantigen peptides are selected for synthesis and potentially administration to the cancer affected subject. In some embodiments, a criterion for inclusion of a particular peptide in this group of selected peptides is that the pentamer amino acid motif which would be exposed to a T cell receptor is present at least once in the reference database derived from the human proteome. In yet other embodiments, the pentamer amino acid motif is found to be present at least 5 times in this database. Conversely, in other embodiments, the selection is made to include peptides which are present not more than 10 times in the human proteome database. When evaluated relative to the database derived from the gastrointestinal microbiome, in some embodiments, the selected peptides are those which have at least one, and in other instances, at least five counts of the same pentamer amino acid motif in the database. In further preferred embodiments the count of the pentamer amino acid motifs in the gastrointestinal microbiome is at least 20.

The criteria described above based on frequency of occurrence of particular pentamer amino acid motifs are equally applicable to the selection of peptides to be presented by MHC I alleles, comprising a continuous amino acid pentamer motif, and those which are bound and presented by an MHC II allele having a discontinuous T cell exposed amino acid pentamer motif.

In yet other instances, the frequency of a pentamer amino acid motif matching a potential T cell epitope may be the basis for exclusion of the peptide from selection in order to avoid an increased risk of adverse epitope mimics. In these cases, the preferred embodiment is to exclude a pentamer motif of interest if it appears in the human proteome, and in other embodiments to exclude a motif which appears more than three times in the human proteome, more than twenty times in the human proteome, or more than fifty times in the human proteome.

The second criterion (which may be used alone or in conjunction with the first criterion described above or other criteria described herein) used to evaluate a potential tumor neoepitope is the probability of endopeptidase cleavage of the peptide bearing the mutation, thereby abrogating or altering its presentation to a T cell by binding in an MHC molecule. In some embodiments, the endopeptidase evaluated is a cathepsin. In some particularly preferred embodiments the cathepsin of interest is cathepsin L, S and/or B. Evaluation of the probability of cleavage may determine that this is unlikely, e.g., being less than a 0.5 or 0.8 probability and a determination may then be made to include the peptide in the selection. However, in other embodiments the probability of cleavage may be greater than 0.7 or greater than 0.9 and a determination may be made to exclude the peptide carrying the mutation from the selection included for synthesis and potential administration to the affected subject. In yet other embodiments, it is determined that it is more probable that an endopeptidase cleavage occurs and as a result creates a peptide with an entirely new epitope and T cell exposed motif which may be evaluated for selection.

In some potential tumor neoepitopes, a high probability of cathepsin cleavage has resulted in immune escape. For such mutated proteins the neoepitopes may be “rescued” as neoantigens by eliminating or reducing the cathepsin cleavage. In peptides where the probability of cleavage exceeds e.g., 0.5 or 0.8, or in further embodiments, where more than one cathepsin is anticipated to have a probability of cleavage of the peptide of over 0.8, the peptides are not selected for inclusion in a vaccine unless the cleavage can be mitigated. In some particularly preferred embodiments, the peptides with a high probability of cathepsin cleavage are derived from the Ras gene family including KRAS, NRAS and HRAS. In other embodiments, the highly cleaved potential neoepitope peptides may be from other proteins which have mutations in the tumor. However, for these easily cleaved neoepitopes, in preferred embodiments the neoepitope peptides, which may be encoded in a nucleic acid sequence, are co-administered to the subject along with an inhibitor of cathepsin. In some preferred embodiments, the inhibitor inhibits cathepsin B. In some embodiments, the cathepsin inhibitors are synthetic molecules and may be from the groups comprising nitrile derivatives, ketone derivatives, acryl hydrazine derivatives, vinyl sulfonate derivatives, epoxy succinic acids, surugamides, loxistatin derivatives, sulfonamide derivatives and betalactams. In other instances natural medicinal products, including but not limited to caffeic and chlorogenic acid containing plant products are a source of cathepsin inhibitors. In a particularly preferred embodiment, the cathepsin inhibitor is chosen from one of the cystatin family of proteins. Such a cystatin, or a polypeptide derived from it may be delivered as a protein or polypeptide or as a nucleic acid encoding the same.

In one embodiment described herein co-administration of a cathepsin inhibitor may be done contemporaneously with a neoantigen vaccine, and indeed may be comprised in the same formulation. Alternatively, a cathepsin inhibitor may be administered to the subject separately from the vaccine. The administration of the chosen cathepsin inhibitor may be accomplished by parenteral delivery or applied locally. Where a tumor is accessible, the cathepsin inhibitor may be applied topically, delivered to a mucosal surface, or provided by intratumoral application.

In another embodiment, an antibody to cathepsin is prepared as an inhibitor and applied either as a tetrameric complete immunoglobulin or by utilizing a sub-component such as a scFV comprising the variable regions of the antibody. Furthermore, antibodies targeting proteins upregulated and expressed on the tumor cell surface or the extracellular matrix thereof may be conjugated or fused to a cathepsin inhibitor for delivery to the tumor site. These are additional embodiments provided herein.

In some embodiments the cathepsin inhibitor is administered operably linked to a second molecule, as a genetic fusion or chemical conjugate. In some particular embodiments the second molecule is an antibody or portion thereof, in others it comprises a T cell receptor.

The third criterion (which may be used alone or in conjunction with the first and/or second criterion described above or other criteria described herein) is the probability that a peptide bearing a tumor specific mutation will be bound and presented to T cells by the affected subject's HLA alleles and that such binding preferentially occurs in a register that exposed the mutant amino acid to a cognate T cell. In some embodiments a peptide is considered for inclusion in the selection if, in addition to fulfilling one or more criteria noted above, it is predicted to bind with sufficient affinity to one or more of the subject's HLA alleles to be presented to a T cell. In preferred embodiments, this is an affinity which is in the top 25% of binding affinity to one or more of the subject's HLA alleles when the affinity of the mutated peptide is considered relative to (i.e. in competition with) other peptides present in the mutated protein. This approach may be applied to consider binding to alleles that are MHC I or MHC II alleles. In some embodiments, a peptide of interest may be determined to bind to multiple HLA alleles of interest, either within a particular subject or within a population of subjects at risk of developing cancer. In a further embodiment, a peptide bearing a T cell exposed motif that is considered desirable for selection based on the first two criteria may be modified to increase or decrease the predicted MHC binding. This may be accomplished by substituting one or more amino acids which are not in the T cell exposed positions so that a peptide is created which maintains the T cell exposed motif but differs from that present in the natural tumor sequence. In yet further preferred embodiments such amino acid substitution may be performed to create a peptide which is more suitable for manufacturing and/or formulation due to improved properties of solubility, stability or to reduce potential aggregation of the peptides.

The present invention guides the expeditious selection of peptides, or the nucleotides that encode them, from proteins which are mutated in tumors. In some embodiments, the proteins which are mutated are the products of oncogenes or tumor suppressor genes, including but not limited to those listed in Table 1. In some particular embodiments the peptide sequences are those of common mutations in driver genes shown as SEQ ID NOs: 1-420 in Table 2 and SEQ ID NOs: 841-1260 in Table 3. In some embodiments the selected peptides comprise the T cell exposed motifs show in these tables as SEQ ID NOs: 421-840 and SEQ ID NOs: 1261-1680. These T cell exposed motifs may then be synthesized as the naturally occurring peptides or as heteroclitic peptides with modified amino acids in the groove exposed positions as indicated above. In yet other embodiments the selected peptides are the product of mutations in passenger genes and are unique to a particular subject having cancer.

The present invention also provides methods for the inclusion of selected peptides, or nucleotide sequences that encode such peptides, in a pre-established library of neoantigens prepared in anticipation of administration to future cancer affected subjects. Such a library may provide the storage of multiple peptides, or nucleotide sequences that encode them, comprising T cell exposed motif sequences derived from 10 or more commonly tumor mutated proteins, or from 20 or 40 or more different commonly mutated proteins. In some embodiments, the library comprises peptides designed to present each of the T cell exposed motif of interest in peptides, each of which will bind to one of at least three common MHC I alleles; in preferred embodiments peptides are designed for presentation to each of five MHC I alleles of interest. In yet other embodiments, the peptides are longer and are designed to bind to similar numbers of MHC II alleles. In some embodiments, the design for binding to different MHC I and MHC II alleles is achieved by making heteroclitic peptides in which amino acids in the flanking groove exposed positions have been substituted to achieve the desired binding affinity. The library may embody peptides, or their encoding nucleic acids, derived from those listed in Tables 2 and 3 either as the naturally occurring mutated peptides shown therein, or as peptides which incorporate the T cell exposed motifs shown on these Tables. In some embodiments a selection of peptides, or the encoding nucleotides, drawn from the library may be supplemented by selected peptides designed to target the unique mutations of a specific subject's tumor.

Based on the methods and embodiments described above, the present invention provides for the design of a neoantigen vaccine for a subject affected by cancer. Such a vaccine may be administered as a group of selected peptides, or as a combination of nucleotide sequences, either RNA or DNA, encoding the selected peptides. In one embodiment, the neoantigen vaccine is delivered parenterally, including but not limited to intradermally, while in other embodiments the vaccine is delivered non-parenterally, including but not limited to orally. In particular embodiments, the formulation and administration of the vaccine may be as a coated tablet or incorporated into a lipid drug delivery system. Such a lipid drug delivery system may include any of the following formulations, or yet other formulations: lipid nanoparticles, emulsions, self-emulsifying drug delivery systems, nanocapsules and liposomes. In some formulations the vaccine is delivered with an adjuvant. While in most embodiments the selected peptides comprised in a neoantigen vaccine may be delivered directly to the affected subject, in other embodiments the vaccinal neoantigens are contacted in vitro with antigen-presenting cells drawn from the subject, including but not limited to dendritic cells, and these cells or T cells co-cultivated with them are administered to the subject. In situations where a tumor mutation generates one or more peptides which have a high probability of cleavage by cathepsin that would prevent the presentation of a neoantigen to a T cell, such neoantigen peptides may be included in the neoantigen vaccine and co-administered along with a cathepsin inhibitor. The cathepsin inhibitor may be comprised within the vaccine formulation for simultaneous administration, or delivered separately either parenterally or topically or to a mucosal surface. In some particular embodiments the cathepsin inhibitor accompanying the neoantigen vaccine may be provided intratumorally.

In another embodiment, the evaluation criteria described are used to select neoantigen peptides for the purpose of identifying neoepitope-cognate T cells and expanding their numbers for administration to an affected subject. In this embodiment, peptides are selected based on the various criteria laid out above and contacted in vitro with antigen presenting cells collected from the cancer affected subject. T cells also harvested from the affected subject are then cultured in contact with the antigen presenting cells and those clones which are stimulated to multiply are isolated and their numbers further expanded. In one embodiment, the T cells are then administered straight away as an autologous transfer, while in other preferred embodiments the T cell clones of interest are preserved for future use, typically cryopreserved. In some embodiments, the antigen presenting cells and T cells are harvested from the cancer-affected subject, but in yet other preferred embodiments the cells are harvested from an allele-matched donor. In this instance, the expanded T cell clones may be administered to as a non-autologous transfer or the expanded T cells may be stored for future use in the same or other affected subjects. The T cells harvested for expansion in this way may be derived by extraction from PBMCs in blood or may be extracted from a biopsy that comprises tumor infiltrating lymphocytes.

In yet further embodiments, T cells specific to T cell exposed motifs of interest that comprise a mutated amino acid are expanded by the steps laid out above and then the T cell receptors that engage the T cell exposed motifs are sequenced. In some preferred embodiments, this comprises both alpha and beta chain sequences of T cell receptors. In further embodiments, these sequences are then inserted into recipient cells to cause them to express the T cell receptors which specifically bind to the original mutated neoepitope of interest. In some embodiments, the recipient cell is a cell maintained in culture. In yet other embodiments, the recipient cell is an antigen-naïve T cell. The methods described herein for sequencing and transfer of the T cell receptor may, in preferred embodiments, be applied to provide cells bearing T cell receptors that are cognate for the peptides and T cell exposed motifs generated by the common mutations in driver gene products shown by their sequence ID numbers in Tables 2 and 3.

As a further method of expediting the preparation of a neoantigen vaccine, the present invention provides a strategy for accelerated synthesis of T cell stimulating and B cell stimulating neoantigen peptides through the preassembly of trimer amino acid building blocks. In one embodiment, the present invention provides methods for synthesizing trimer amino acid sequences and storing these in anticipation of identification of a selected neoantigen peptide. Once a group of neoantigen peptides has been selected by the criteria described above, the desired peptides are synthesized by combining the trimer sequences to provide neoantigen peptides for administration to an affected subject. In some embodiments, the resulting selected peptides are 9 amino acids to stimulate CD8+ responses; in other embodiments the resulting peptides are 15 amino acids long to stimulate CD4+ responses in the subject. In yet other embodiments, longer peptides may be assembled by combining trimer building blocks for use as linear B cell epitopes. To enable the assembly of 9mer and 15mer and longer peptides in any sequence selected for a particular subject an array, or collection, of trimers is prepared in advance and stored. In some embodiments the array may comprise all the 8000 possible trimer combinations of three amino acids; however in most embodiments a somewhat smaller array will enable assembly of the most frequently selected peptides needed. Hence in other embodiments an array of 4000 unique trimers, or 2000 unique trimers are prepared from which the longer desired peptides are assembled. The trimers may be assembled to provide peptides which comprise T cell epitopes or, in other embodiments, linear B cell epitopes. In some embodiments the assembled peptides are components of a neoantigen vaccine administered to a subject affected by cancer.

In some preferred embodiments, the present invention provides methods for selection of peptides for inclusion in a treatment for subjects affected by cancer, or at risk of being affected by cancer, comprising: Obtaining sequences of tumor proteins; Identifying amino acid mutations in the tumor proteins as compared to corresponding wild-type sequences of the protein in the subject or a reference human subject; Identifying pentamer amino acid motifs that correspond to T cell exposed motifs which comprise the identified amino acid mutations in the tumor proteins; Determining the frequency of occurrence of the pentamer amino acid motifs in a reference database of reference proteins; Selecting T cell exposed motifs comprising the mutant amino acids based on the frequency of occurrence of the pentamer amino acid motifs in the reference database; and Synthesizing one or more peptides that comprises each of the one or more selected T cell exposed motifs or nucleic acids encoding the one or more T cell exposed motifs. In some preferred embodiments, the methods further comprise incorporating the one or more synthetic peptide sequences, or the nucleic acid sequences encoding them, into a treatment formulation for administration to a subject.

In some preferred embodiments, the reference database of reference proteins comprises proteins of the human proteome. In some preferred embodiments, the reference database of reference proteins comprises proteins of microorganisms. In some preferred embodiments, the reference database of reference proteins comprises proteins of organisms of the gastrointestinal microbiome. In some preferred embodiments, the reference database of reference proteins comprises proteins of the human immunoglobulinome. In some preferred embodiments, the reference database of reference proteins comprises of more than 1,000 proteins. In some preferred embodiments, the reference database of reference proteins comprises more than 10,000 proteins. In some preferred embodiments, the reference database of reference proteins comprises more than 20,000 proteins.

In some preferred embodiments, the methods further comprise determining the frequency of the pentamer amino acid motifs in more than one reference database of reference proteins. In some preferred embodiments, the pentamer amino acid motifs that correspond to T cell exposed motifs in the tumor proteins are found at least once in the human proteome reference database. In some preferred embodiments, the pentamer amino acid motifs that correspond to T cell exposed motifs in the tumor proteins are found at least 5 times in the human proteome reference database. In some preferred embodiments, the pentamer amino acid motifs that correspond to T cell exposed motifs in the tumor proteins are found no more than 10 times in the human proteome reference database. In some preferred embodiments, the pentamer amino acid motifs that correspond to T cell exposed motifs in the tumor proteins are found at least once in the gastrointestinal reference database. In some preferred embodiments, the pentamer amino acid motifs that correspond to T cell exposed motifs in the tumor proteins occur at least 5 times in the gastrointestinal reference database. In some preferred embodiments, the pentamer amino acid motifs that correspond to T cell exposed motifs in the tumor proteins occur at least 20 times in a reference database of microbial proteins.

In some preferred embodiments, the T cell exposed motif is a motif exposed to a T cell by a peptide bound to an MHC I molecule. In some preferred embodiments, the T cell exposed motif is a motif exposed to a T cell by a peptide bound to an MHC II molecule.

In some preferred embodiments, determination of the frequency of occurrence of the pentamer amino acid motifs in a reference database of reference proteins informs a decision to exclude the peptide comprising a particular T cell exposed motif from a treatment formulation. In some preferred embodiments, the pentamer amino acid motif is absent from the human proteome database and the motif is excluded from a treatment formulation. In some preferred embodiments, the pentamer amino acid motif is present in less than 3 locations in the human proteome and the motif is excluded from a treatment formulation. In some preferred embodiments, the pentamer amino acid motif is present in more than 20 proteins in the human proteome and the motif is excluded from a treatment formulation. In some preferred embodiments, the pentamer amino acid motif is present in more than 50 proteins in the human proteome and the motif is excluded from a treatment formulation.

In some preferred embodiments, the methods further comprise: determining the predicted probability of cleavage by an endopeptidase of a peptide identified in the tumor protein as comprising an amino acid mutation, and selecting or excluding one or more peptides for synthesis based on the probability of cleavage of that peptide by a peptidase. In some preferred embodiments, the peptidase is a cathepsin. In some preferred embodiments, peptides are selected that have a probability less than 0.5 of cleavage. In some preferred embodiments, peptides are selected that have a probability less than 0.8 of cleavage. In some preferred embodiments, the probability of cleavage is greater than 0.7 and the peptide is excluded from a treatment formulation. In some preferred embodiments, the probability of cleavage is greater than 0.9 and the peptide is excluded from a treatment formulation. In some preferred embodiments, cleavage creates a novel T cell epitope and the novel T cell epitope is included in the treatment formulation. In those instances where the probability of cathepsin cleavage of a neoepitope peptide of interest is higher than 0.5 or higher than 0.8 or multiple cathepsins have a probability >0.8 of producing cleavage, a cathepsin inhibitor may be co-administered with the neoantigen vaccine. The cathepsin inhibitor may be administered as a component of the vaccine formulation or separately, and may be administered parenterally, topically, mucosally or intratumorally.

In some preferred embodiments, the methods further comprise selecting peptides that have a predicted probability of binding to one or more MHC alleles with an affinity in the top 25% as compared to all peptides in the protein from which it is derived. In some preferred embodiments, the MHC is an MHC I. In some preferred embodiments, the MHC is an MHC II. In some preferred embodiments, the binding is to at least three MHC alleles.

In some preferred embodiments, the methods further comprise for each selected T cell exposed motif, synthesizing a peptide of desired binding affinity for each of an array of MHCs of interest by selecting amino acids to comprise a groove exposed motif in the peptide, thereby synthesizing a peptide that is not naturally present in the tumor proteins of the subject or the reference human subject. In some preferred embodiments, the groove exposed motif amino acids are further selected based on a property or properties selected from one of more of solubility, stability, or reduced aggregation. In some preferred embodiments, the tumor protein is an oncogene or tumor suppressor gene product.

In some preferred embodiments, the tumor protein comprises a passenger gene mutation.

In some preferred embodiments, the tumor protein is selected from the group consisting of proteins corresponding to the gene identifiers listed in Table 1. In some preferred embodiments, the mutated peptide in the tumor protein is selected from the group consisting of SEQ ID NOs: 1-420 and SEQ ID NOs: 841-1260. In some preferred embodiments, the pentamer amino acid motif in the tumor protein that is mutated is selected from the group consisting of SEQ ID NOs: 421-840 and SEQ ID NOs: 1261-1680.

In some preferred embodiment, the mutated peptide is not a peptide from TP53. In some preferred embodiments, the mutated peptide is not a TP53 peptide having the following mutations: R17511, Y220C, G245D, G245S, R248L, R248Q, R248W, R249S, R273H, R273C, R273L, or R282W.

In some preferred embodiments, the one or more synthesized peptides, or nucleic acids encoding the peptides, have a maximum length of 60 amino acids or 180 nucleotides. In some preferred embodiments, the one or more synthesized peptides, or nucleic acids encoding the peptides, have a maximum length of 48 amino acids or 144 nucleotides. In some preferred embodiments, the one or more synthesized peptides, or nucleic acids encoding the peptides, have a maximum length of 36 amino acids or 108 nucleotides. In some preferred embodiments, the one or more synthesized peptides, or nucleic acids encoding the peptides, have a length of from 9 amino acids or 27 nucleotides to 48 amino acids or 144 nucleotides. In some preferred embodiments, the one or more synthesized peptides, or nucleic acids encoding the peptides, have a length of from 9 amino acids or 27 nucleotides to 36 amino acids or 108 nucleotides. In some preferred embodiments, the one or more synthesized peptides, or nucleic acids encoding the peptides, have a length of from 9 amino acids or 27 nucleotides to 18 amino acids or 54 nucleotides. In some preferred embodiments, the one or more synthesized peptides, or nucleic acids encoding the peptides, have a length of from 15 amino acids or 45 nucleotides to 48 amino acids or 144 nucleotides. In some preferred embodiments, the one or more synthesized peptides, or nucleic acids encoding the peptides, have a length of from 15 amino acids or 45 nucleotides to 36 amino acids or 108 nucleotides. In some preferred embodiments, the one or more synthesized peptides, or nucleic acids encoding the peptides, have a length of from 12 amino acids or 36 nucleotides to 38 amino acids or 114 nucleotides. In some preferred embodiments, the one or more synthesized peptides, or nucleic acids encoding the peptides, are any multiple of 3 amino acids.

In some preferred embodiments, the present invention provides methods of assembling a library of peptides for treatment of one or more subjects affected by or at risk of being affected by cancer comprising: Selecting peptides by application of the method as described above; and Synthesizing the peptides, or the nucleic acids encoding the peptides, and storing the peptides or nucleic acids.

In some preferred embodiments, the library of peptides comprises pentamer amino acid motifs comprising mutations from at least 10 different tumor proteins or 10 different mutations in the same tumor protein. In some preferred embodiments, the library of peptides comprises pentamer amino acid motifs comprising mutations from at least 20 different tumor proteins or 20 different mutations in the same tumor protein. In some preferred embodiments, the library of peptides comprises pentamer amino acid motifs comprises mutations from at least 40 different tumor proteins or 40 different mutations in the same tumor protein.

In some preferred embodiments, the library of peptides comprises peptides selected to bind at least 3 MHC I alleles. In some preferred embodiments, the library of peptides comprises peptides selected to bind at least 5 MHC I alleles. In some preferred embodiments, the library of peptides comprises peptides selected to bind at least 3 MHC II alleles. In some preferred embodiments, the library of peptides comprises peptides selected to bind at least 5 MHC II alleles. In some preferred embodiments, the library of peptides comprises one or more heteroclitic peptides.

In some preferred embodiments, the library of peptides comprises, consists essentially of, or consists of five or more of the peptides selected from the group consisting of SEQ ID NOs: 1-420 and SEQ ID NOs: 841-1260. In some preferred embodiments, the library of peptides comprises, consists essentially of, or consists of five or more of the T cell exposed motifs selected from the group consisting of SEQ ID NO.: NOs: 421-840 and SEQ ID NOs: 1261-1680. In some preferred embodiments, the library of peptides comprises, consists essentially of, or consists of five or more of the pentamer amino acid motifs selected from SEQ ID NOs: 421-840 and SEQ ID NOs: 1261-1680, with the proviso that the peptides are not one of sequences SEQ ID NOs: 1-420 or SEQ ID NOs: 841-1260. In some preferred embodiments, the library of peptides comprises, consists essentially of, or consists of five or more of the pentamer amino acid motifs selected from the sequences listed in Table 14. In some preferred embodiment, the mutated peptide is not a peptide from TP53. In some preferred embodiments, the mutated peptide is not a TP53 having the following mutations: R17511, Y220C, G245D, G245S, R248L, R248Q, R248W, R249S, R273H, R273C, R273L, or R282W. In some preferred embodiments, one or more peptides selected from the library are administered with additional peptides selected according to the methods described above to target mutations unique to a particular subject's tumor.

In some preferred embodiments, the present invention provides a library of nucleic acid sequences encoding the peptides described above or identified by a method described above.

In some preferred embodiments, the present invention provides a library of peptides, or nucleic acids encoding the peptides, for use in a treatment for one or more subjects affected by cancer, or at risk of being affected by cancer, comprising five or more of the peptides selected from the group consisting of SEQ ID NOs: 1-420 and SEQ ID NOs: 841-1260.

In some preferred embodiments, the present invention provides a library of peptides, or nucleic acids encoding the peptides, for use in a treatment for one or more subjects affected by cancer, or at risk of being affected by cancer, comprising five or more of the T cell exposed motifs selected from the group consisting of SEQ ID NOs: 421-840 and SEQ ID NOs: 1261-1680.

In some preferred embodiments, the present invention provides a library of peptides, or nucleic acids encoding the peptides, for use in a treatment for one or more subjects affected by cancer, or at risk of being affected by cancer, comprising five or more of the T cell exposed motifs selected from the group consisting of SEQ ID NOs: 421-840 and SEQ ID NOs: 1261-1680, with the proviso that the peptides are not SEQ ID NOs: 1-420 and SEQ ID NOs: 841-1260.

In some preferred embodiments, the present invention provides a library of peptides, or nucleic acids encoding the peptides, for use in a treatment for one or more subjects affected by cancer, or at risk of being affected by cancer, comprising five or more of the T cell exposed motifs selected from the group consisting of the sequences listed in Table 14.

In some preferred embodiments, the peptides in the library are from 9 to 60 amino acids in length.

In some preferred embodiments, the present invention provides a vaccine for a subject affected by cancer or at risk of being affected by cancer comprising peptides or nucleic acids encoding the peptides selected by one or more of the methods described above or identified above. In some preferred embodiments, the vaccine comprises peptides. In some preferred embodiments, the vaccine comprises nucleic acids encoding the selected peptides. In some preferred embodiments, the vaccine is formulated for parenteral delivery. In some preferred embodiments, the vaccine is formulated for non-parenteral delivery. In some preferred embodiments, the vaccine is formulated for oral delivery. In some preferred embodiments, the vaccine is formulated as a coated tablet. In some preferred embodiments, the vaccine is formulated for intradermal delivery. In some preferred embodiments, the vaccine is formulated as a lipid drug delivery system selected from the group consisting of lipid nanoparticles, emulsions, self-emulsifying drug delivery systems, nanocapsules and liposomes. In some preferred embodiments, the vaccine comprises an adjuvant. In some preferred embodiments, the vaccine comprises peptides or the nucleic acids encoding the peptides selected from a library of peptides designed prior to the diagnosis of cancer in a specific subject.

In some preferred embodiments, the present invention provides methods comprising: contacting antigen presenting cells collected from the subject ex vivo with a vaccine as described above, or peptides or nucleic acids encoding the peptides selected by one or more of the methods described above; and administering the cells to the subject.

In some preferred embodiments, the present invention provides a method for selecting one or more T cell clones comprising: Selecting and synthesizing a peptide, or a nucleic acid encoding the peptide, by a method as described above or identified above; Contacting the peptide in vitro with an antigen presenting cell harvested from a subject; Collecting T cells from that subject; Contacting the T cells with the antigen presenting cells thereby presenting the peptide of interest bound in the MHC of the antigen presenting cells to the T cells; Selecting T cells stimulated by contact with the peptide of interest presented by the antigen presenting cells; Culturing the T cells to provide expanded T cell clones; and Harvesting the expanded T cell clones.

In some preferred embodiments, the antigen presenting cell is a dendritic cell, a macrophage, or a B cell. In some preferred embodiments, T cells derived from the expanded T cell clones are administered autologously to the subject. In some preferred embodiments, T cells derived from the expanded T cell clones are administered to a non-autologous subject that shares one or more HLA alleles with the T cell source subject. In some preferred embodiments, T cells derived from the expanded T cell clone population are preserved for future use. In some preferred embodiments, T cells from the subject are harvested from PBMCs. In some preferred embodiments, T cells from the subject are harvested from tumor infiltrating lymphocytes.

In some preferred embodiments, the methods further comprise nucleotide sequencing the T cell receptors of representative T cells drawn from the expanded T cell clones. In some preferred embodiments, the alpha and beta chains of the receptors of selected T cells are sequenced and/or cloned.

In some preferred embodiments, the methods further comprise inserting the alpha and/or beta chain nucleotide sequences of the T cell receptors into a recipient cell. In some preferred embodiments, the recipient cell is a cell maintained in culture. In some preferred embodiments, the cell in culture is a mammalian cell, a bacterial cell, or a yeast cell. In some preferred embodiments, the mammalian cell is a recipient T cell. In some preferred embodiments, the recipient T cell is an antigen naïve T cell. In some preferred embodiments, the peptide for which the T cell receptor is specific comprises a T cell exposed motif selected from the group consisting of SEQ ID NOs: 421-840 and SEQ ID NOs: 1261-1680. In some preferred embodiments, the peptide comprises a T cell exposed motif selected from the group consisting of the T cell exposed motif sequences listed in Table 14. In some preferred embodiment, the mutated peptide is not a peptide from TP53. In some preferred embodiments, the mutated peptide is not a TP53 peptide having the following mutations: R17511, Y220C, G245D, G245S, R248L, R248Q, R248W, R249S, R273H, R273C, R273L, or R282W.

In some preferred embodiments, the present invention provides a T cell clone produced by a method as described above. In some preferred embodiments, the present invention provides a T cell receptor sequence produced by a method as described above. In some preferred embodiments, the present invention provides an engineered T cell comprising the T cell receptor sequence. In some preferred embodiments, the engineered T cell comprises a chimeric T cell receptor comprising the T cell receptor sequence.

The need to inhibit cathepsins to prevent their cleavage of certain mutated peptides leads to two further embodiments of the present invention. In one of these an antibody to cathepsin is prepared and a recombinant version of the antibody or a molecule comprising the variable regions of that antibody are provided as a means of reducing or neutralizing the activity of the cathepsin. In a second additional embodiment, an antibody targeting an epitope in a protein upregulated in the tumor cells or the extracellular matrix is provided as a fusion or conjugate to a cystatin or a subsequence of cystatin and provided to target the cystatin to the tumor cell. Examples of upregulated tumor proteins to which such targeting antibodies may be directed include, not only those mutated, but also unmutated proteins such as brevican, or MAGEA1 or NY-CSO.

In some preferred embodiments, the present invention provides methods of assembling an array of peptides for inclusion in a treatment for one or more subjects affected by cancer, or at risk of developing cancer, comprising: Synthesizing an array of trimer peptides; Selecting peptides for inclusion in a treatment regimen; Assembling desired peptides from the trimer peptides; and Administering the assembled peptides to the subject. In some preferred embodiments, the array comprises 8000 unique trimers. In some preferred embodiments, at least 7000 unique trimers. In some preferred embodiments, the array comprises at least 5000 unique trimers. In some preferred embodiments, the assembled peptides are 9 mers, or 15 mers. In some preferred embodiments, the assembled peptides are 12 mers to 36 mers. In some preferred embodiments, the assembled peptides are any multiple of 3 amino acids. In some preferred embodiments, the assembled peptides are selected according to the methods described above or identified above. In some preferred embodiments, the peptide comprises a T cell epitope. In some preferred embodiments, the peptide comprises a B cell epitope. In some preferred embodiments, the methods further comprise administering the assembled peptides as a vaccine to a subject in need thereof.

### DESCRIPTION OF THE FIGURES

FIG. 1: Linear B cell epitopes in Cathepsins B, L and S

Overview of MHC binding, B cell epitopes and topology. The X axis indicates the index position of sequential peptides with single amino acid displacement. The Y axis indicates predicted binding affinity of each peptide in standard deviation units for the protein. The red line shows the permuted average predicted MHC-IA and B (62 alleles) binding affinity of sequential 9-mer peptides with single amino acid displacement. The blue line shows the permuted average predicted MHC-II DRB allele (24 most common human alleles) binding affinity of sequential 15-mer peptides. Orange lines show the predicted probability of B-cell receptor binding for an amino acid centered in each sequential 9-mer peptide. Low numbers for MHC data represent high binding affinity, whereas low numbers equate to high B cell receptor contact probability. Ribbons (red: MHC-I, blue: MHC-II) indicate the 10% highest predicted MHC affinity binding. Orange ribbons indicate the top 25% predicted probability B-cell binding. Horizontal dotted lines demarcate the top 5% of binding affinity for the protein (red MHC I, blue MHC II).

FIG. 2: Pentamer motif frequencies in mutated oncogenes and suppressors, aligned at the mutant position.

Peptide 9mers adjacent to the mutant position in 123 oncogenes and suppressors were aligned with the mutant position set at 0. The Y axis shows the Z scale standardized frequency in the human proteome of the pentamer motifs corresponding to the potential TCEM at each position. Overall, the plot shows 679,210 pentamer motifs comprising each mutant amino acid in each possible position. The points are randomly jittered for visualization. The line shows the mean at each TCEM position and shows the downward shift in hPPF for those TCEM containing the mutant amino acid.

A few rare motifs occur outside the main pattern. These arise from 16 proteins in which the longest isoform was not the reference sequence, and in which there were single rare motifs at positions other than the mutant which then appears for each of multiple mutants (e.g. RUNX1 isoform Q01196_8 has 84 mutants and a single additional rare motif).

FIG. 3: Pentamer motif frequencies comprising the mutant amino acid in oncogene and suppressor gene products ranked by matching pentamer frequencies in the human proteome and GI microbiome.

The Y axis shows the count of pentamer motifs in the oncogene and suppressor gene product mutation dataset. Counts of pentamer positions which place the mutant amino acid in the MHC I GEM I or TCEM I positions are shown in red. Discontinuous pentamer positions which place the mutant amino acid in the MHC II GEM II or TCEM II positions are shown in blue. Wildtype homologues are shown in grey. In A and B the X axis is the Z scale standardized frequency of each pentamer motif in the human proteome (hPPF). The histogram bar on the far left of FIGS. 2A and 2B TCEM indicates motifs absent from the human proteome, the second bar is singletons, the third bar doubletons etc. In panels C and D the X axis is the Z scale standardized frequency of each pentamer motif in the GI microbiome (giPPF). This larger dataset more closely approaches a normal distribution, while still underlain by a Poisson distribution. k=Poisson mean, 7=fraction of zero counts.

FIG. 4: Comparison of featured of sequential peptide position in TP53 R175H and KRAS G12D Upper plot shows sequential 9mer peptides in TP53 wildtype and R175H tracking the change in GEM vs TCEM position, TCEM amino acids, hPPF, and giPPF and predicted binding for A*02:01 and A*24:01. The baseline hPFF in the wildtype is low, and in the mutant comprises multiple missing (hPPF=0).

Lower plot shows the same fields for KRAS G12D, where the baseline hPPF is high.

Column headings: Position: index amino acid position in protein; Position mutant relative: index amino acid relative to mutant position; pocket position indicated p1-p9 with TCEM shaded; A*02:01 and A*24:01 is the Z scale predicted binding affinity at every position for these alleles shown in standard deviation units (G) where blue shading indicates higher affinity.

FIG. 5: Motif frequency in passenger gene products in an example tumor.

This figure provides a replicate of Table 10. TCEM I and TCEM IIa provide the pentamer motifs which would expose the mutant amino acid to a T cell for each mutant gene shown in column 1. Pos is the index position in the mutated protein of the 9mer or 15mer peptide which encompasses the TCEM. hPPF I and hPPF II are the counts of the TCEM pentamers in the human proteome. gi PPF I and giPPF II are the counts in the GI microbiome reference database. The five columns on the right (A0201 etc) indicate the predicted binding affinity to each of the alleles shown in standard deviations below the mean for the parent protein.

FIG. 6: Predicted cathepsin cleavage of KRAS G12D.

Column headings: Position: index amino acid position in protein; Position mutant relative: index amino acid relative to mutant position; pocket position indicated p1-p9 with TCEM shaded; (pos)peptide shows where the predicted cleavage occurs at the ~ mark. hCAT_L, CAT_S, and CAT_B show the predicted probability of cleavage at the site indicated in the prior column by the respective cathepsins, where 1=100%.

FIG. 7: Predicted cathepsin cleavage of KRAS

All mutants of KRAS are overlaid and the predicted probability of three cathepsins are shown in the Y axis at each successive peptide position. Blue=cathepsin S, Red=cathepsin L and Green=cathepsin B. The black lines indicate common mutation points at G12-13, Q61 and between positions 110-117.

FIG. 8: Cathepsins are upregulated in tumors.

Y axis shows individual cathepsins and their RNA transcription in standardized FPKN (standardized fragments per kilobase per million). X axis at top shows representatives of biopsies of different types of cancer using TCGA type designations. It will be noted that cathepsin B and D are almost uniformly highly upregulated.

FIG. 9: Schematic diagram of potential cleavage site octomers

The potential CSOs are overlayed on potential 9mer peptides containing a mutation (represented as X). The are 8 octomers (spanning 8 potential cleavage dimers) for 9 mutant positions for a total of 72 octomers, but due to overlap, 16 are considered for each 9mer.

### DEFINITIONS

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif. Human proteome refers to all the proteins comprised in a human being. Multiple such sets of proteins have been sequenced and are accessible at the InterPro international repository (see world-wide web at ebi.ac.uk/interpro). Human proteome is also understood to include those proteins and antigens thereof which may be over-expressed in certain pathologies, or expressed in a different isoforms in certain pathologies. Hence, as used herein, tumor associated antigens are considered part of the human proteome. “Proteome” may also be used to describe a large compilation or collection of proteins, such as all the proteins in an immunoglobulin collection or a T cell receptor repertoire, or the proteins which comprise a collection such as the allergome, such that the collection is a proteome which may be subject to analysis. All the proteins in a bacteria or other microorganism are considered its proteome.

As used herein, the terms “protein,” “polypeptide,” and “peptide” refer to a molecule comprising amino acids joined via peptide bonds. In general “peptide” is used to refer to a sequence of 40 or less amino acids and “polypeptide” is used to refer to a sequence of greater than 40 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest. It may be applied to any protein to which further analysis is applied or the properties of which are tested or examined. Similarly, as used herein, “target protein” may be used to describe a protein of interest that is subject to further analysis.

As used herein “peptidase” refers to an enzyme which cleaves a protein or peptide. The term peptidase may be used interchangeably with protease, proteinases, oligopeptidases, and proteolytic enzymes. Peptidases may be endopeptidases (endoproteases), or exopeptidases (exoproteases). The the term peptidase would also include the proteasome which is a complex organelle containing different subunits each having a different type of characteristic scissile bond cleavage specificity. Similarly the term peptidase inhibitor may be used interchangeably with protease inhibitor or inhibitor of any of the other alternate terms for peptidase.

As used herein, the term “exopeptidase” refers to a peptidase that requires a free N-terminal amino group, C-terminal carboxyl group or both, and hydrolyses a bond not more than three residues from the terminus. The exopeptidases are further divided into aminopeptidases, carboxypeptidases, dipeptidyl-peptidases, peptidyl-dipeptidases, tripeptidyl-peptidases and dipeptidases.

As used herein, the term “endopeptidase” refers to a peptidase that hydrolyses internal, alpha-peptide bonds in a polypeptide chain, tending to act away from the N-terminus or C-terminus. Examples of endopeptidases are chymotrypsin, pepsin, papain and cathepsins. A very few endopeptidases act a fixed distance from one terminus of the substrate, an example being mitochondrial intermediate peptidase. Some endopeptidases act only on substrates smaller than proteins, and these are termed oligopeptidases. An example of an oligopeptidase is thimet oligopeptidase. Endopeptidases initiate the digestion of food proteins, generating new N- and C-termini that are substrates for the exopeptidases that complete the process. Endopeptidases also process proteins by limited proteolysis. Examples are the removal of signal peptides from secreted proteins (e.g. signal peptidase I) and the maturation of precursor proteins (e.g. enteropeptidase, furin, etc.). In the nomenclature of the Nomenclature Committee of the International Union of Biochemistry and Molecular Biology (NC-IUBMB) endopeptidases are allocated to sub-subclasses EC 3.4.21, EC 3.4.22, EC 3.4.23, EC 3.4.24 and EC 3.4.25 for serine-, cysteine-, aspartic-, metallo- and threonine-type endopeptidases, respectively. Endopeptidases of particular interest are the cathepsins, and especially cathepsin B, L and S known to be active in antigen presenting cells. Cathepsin B may function as an endo peptidase or an exopeptidase.

As used herein, the term “immunogen” refers to a molecule which stimulates a response from the adaptive immune system, which may include responses drawn from the group comprising an antibody response, a cytotoxic T cell response, a T helper response, and a T cell memory. An immunogen may stimulate an upregulation of the immune response with a resultant inflammatory response or may result in down regulation or immunosuppression. Thus the T-cell response may be a T regulatory response. An immunogen also may stimulate a B-cell response and lead to an increase in antibody titer. Another term used herein to describe a molecule or combination of molecules which stimulate an immune response is “antigen”.

As used herein, the term “native” (or wild type) when used in reference to a protein refers to proteins encoded by the genome of a cell, tissue, or organism, other than one manipulated to produce synthetic proteins.

As used herein the term “epitope” refers to a peptide sequence which elicits an immune response, from either T cells or B cells or antibody

As used herein, the term “B-cell epitope” refers to a polypeptide sequence that is recognized and bound by a B-cell receptor. A B-cell epitope may be a linear peptide or may comprise several discontinuous sequences which together are folded to form a structural epitope. Such component sequences which together make up a B-cell epitope are referred to herein as B-cell epitope sequences. Hence, a B-cell epitope may comprise one or more B-cell epitope sequences. Hence, a B cell epitope may comprise one or more B-cell epitope sequences. A linear B-cell epitope may comprise as few as 2-4 amino acids or more amino acids.

“B cell core peptides” or “core pentamer” when used herein refers to the central 5 amino acid peptide in a predicted B cell epitope sequence. The B cell epitope may be evaluated by predicting the binding of across a series of 9-mer windows, the core pentamer then is the central pentamer of the 9-mer window

As used herein, the term “predicted B-cell epitope” refers to a polypeptide sequence that is predicted to bind to a B-cell receptor by a computer program, for example, as described in PCT US2011/029192, PCT US2012/055038, US2014/014523, and PCT US2015/039969, each of which is incorporated herein by reference in its entirety, and in addition by Bepipred (Larsen, et al., Immunome Research 2:2, 2006) and others as referenced by Larsen et al (ibid) (Hopp T et al PNAS 78:3824-3828, 1981; Parker J et al, Biochem. 25:5425-5432, 1986). A predicted B-cell epitope may refer to the identification of B-cell epitope sequences forming part of a structural B-cell epitope or to a complete B-cell epitope.

As used herein, the term “T-cell epitope” refers to a polypeptide sequence which when bound to a major histocompatibility protein molecule provides a configuration recognized by a T-cell receptor. Typically, T-cell epitopes are presented bound to a MHC molecule on the surface of an antigen-presenting cell.

As used herein, the term “predicted T-cell epitope” refers to a polypeptide sequence that is predicted to bind to a major histocompatibility protein molecule by the neural network algorithms described herein, by other computerized methods, or as determined experimentally. As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells. The MHC is both polygenic (there are several MHC class I and MHC class II genes) and polyallelic or polymorphic (there are multiple alleles of each gene). The terms MHC-I, MHC-II, MHC-1 and MHC-2 are variously used herein to indicate these classes of molecules. Included are both classical and nonclassical MHC molecules. An MHC molecule is made up of multiple chains (alpha and beta chains) which associate to form a molecule. The MHC molecule contains a cleft or groove which forms a binding site for peptides. Peptides bound in the cleft or groove may then be presented to T-cell receptors. The term “MHC binding region” refers to the groove region of the MHC molecule where peptide binding occurs.

As used herein, a “MHC II binding groove” refers to the structure of an MHC molecule that binds to a peptide. The peptide that binds to the MHC II binding groove may be from about 11 amino acids to about 23 amino acids in length, but typically comprises a 15-mer. The amino acid positions in the peptide that binds to the groove are numbered based on a central core of 9 amino acids numbered 1-9, and positions outside the 9 amino acid core numbered as negative (N terminal) or positive (C terminal). Hence, in a 15mer the amino acid binding positions are numbered from −3 to +3 or as follows: −3, −2, −1, 1, 2, 3, 4, 5, 6, 7, 8, 9, +1, +2, +3.

As used herein, the term “haplotype” refers to the HLA alleles found on one chromosome and the proteins encoded thereby. Haplotype may also refer to the allele present at any one locus within the MHC. When referring to the HLA alleles on both chromosomes in a subject we refer to “HLA genotype”.

Each class of MHC-Is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein. HLA alleles are listed at hla.alleles.org/nomenclature/naming.html, which is incorporated herein by reference.

The MHCs exhibit extreme polymorphism: within the human population there are, at each genetic locus, a great number of haplotypes comprising distinct alleles—the IMGT/HLA database release (February 2010) lists 948 class I and 633 class II molecules, many of which are represented at high frequency (>1%). MHC alleles may differ by as many as 30-aa substitutions. Different polymorphic MHC alleles, of both class I and class II, have different peptide specificities: each allele encodes proteins that bind peptides exhibiting particular sequence patterns.

The naming of new HLA genes and allele sequences and their quality control is the responsibility of the WHO Nomenclature Committee for Factors of the HLA System, which first met in 1968, and laid down the criteria for successive meetings. This committee meets regularly to discuss issues of nomenclature and has published 19 major reports documenting firstly the HLA antigens and more recently the genes and alleles. The standardization of HLA antigenic specifications has been controlled by the exchange of typing reagents and cells in the International Histocompatibility Workshops. The IMGT/HLA Database collects both new and confirmatory sequences, which are then expertly analyzed and curated before been named by the Nomenclature Committee. The resulting sequences are then included in the tools and files made available from both the IMGT/HLA Database and at hla.alleles.org.

Each HLA allele name has a unique number corresponding to up to four sets of digits separated by colons. See e.g., hla.alleles.org/nomenclature/naming.html which provides a description of standard HLA nomenclature and Marsh et al., Nomenclature for Factors of the HLA System, 2010 Tissue Antigens 2010 75:291-455. HLA-DRB1*13:01 and HLA-DRB1*13:01:01:02 are examples of standard HLA nomenclature. The length of the allele designation is dependent on the sequence of the allele and that of its nearest relative. All alleles receive at least a four digit name, which corresponds to the first two sets of digits, longer names are only assigned when necessary.

The digits before the first colon describe the type, which often corresponds to the serological antigen carried by an allele, The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns or in the 5′ or 3′ untranslated regions that flank the exons and introns are distinguished by the use of the fourth set of digits. In addition to the unique allele number there are additional optional suffixes that may be added to an allele to indicate its expression status. Alleles that have been shown not to be expressed, ‘Null’ alleles have been given the suffix ‘N’. Those alleles which have been shown to be alternatively expressed may have the suffix ‘L’, ‘S’, ‘C’, ‘A’ or ‘Q’. The suffix ‘L’ is used to indicate an allele which has been shown to have ‘Low’ cell surface expression when compared to normal levels. The ‘S’ suffix is used to denote an allele specifying a protein which is expressed as a soluble ‘Secreted’ molecule but is not present on the cell surface. A ‘C’ suffix to indicate an allele product which is present in the ‘Cytoplasm’ but not on the cell surface. An ‘A’ suffix to indicate ‘Aberrant’ expression where there is some doubt as to whether a protein is expressed. A ‘Q’ suffix when the expression of an allele is ‘Questionable’ given that the mutation seen in the allele has previously been shown to affect normal expression levels.

In some instances, the HLA designations used herein may differ from the standard HLA nomenclature just described due to limitations in entering characters in the databases described herein. As an example, DRB1_0104, DRB1*0104, and DRB1-0104 are equivalent to the standard nomenclature of DRB1*01:04. In most instances, the asterisk is replaced with an underscore or dash and the semicolon between the two digit sets is omitted.

As used herein, the term “polypeptide sequence that binds to at least one major histocompatibility complex (MHC) binding region” refers to a polypeptide sequence that is recognized and bound by one or more particular MHC binding regions as predicted by the neural network algorithms described herein or as determined experimentally.

As used herein the terms “canonical” and “non-canonical” are used to refer to the orientation of an amino acid sequence. Canonical refers to an amino acid sequence presented or read in the N terminal to C terminal order; non-canonical is used to describe an amino acid sequence presented in the inverted or C terminal to N terminal order.

As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “affinity” refers to a measure of the strength of binding between two members of a binding pair, for example, an antibody and an epitope or an epitope and a MHC-I or II allele. K_{d} is the dissociation constant and has units of molarity. The affinity constant is the inverse of the dissociation constant. An affinity constant is sometimes used as a generic term to describe this chemical entity. It is a direct measure of the energy of binding. The natural logarithm of K is linearly related to the Gibbs free energy of binding through the equation ΔG₀=−RT LN(K) where R=gas constant and temperature is in degrees Kelvin. Affinity may be determined experimentally, for example by surface plasmon resonance (SPR) using commercially available Biacore SPR units (GE Healthcare) or in silico by methods such as those described herein in detail. Affinity may also be expressed as the ic50 or inhibitory concentration 50, that concentration at which 50% of the peptide is displaced. Likewise ln(ic50) refers to the natural log of the ic50.

The term “K_{off}”, as used herein, is intended to refer to the off rate constant, for example, for dissociation of an antibody from the antibody/antigen complex, or for dissociation of an epitope from an MHC molecule.

Binding affinity may also be expressed by the standard deviation from the mean binding found in the peptides making up a protein. Hence a binding affinity may be expressed as “−1σ” or <−1σ, where this refers to a binding affinity of 1 or more standard deviations below the mean. This is also commonly referred to as the Z-scale. A common mathematical transformation used in statistical analysis is a process called standardization wherein the distribution is transformed from its standard units to standard deviation units where the distribution has a mean of zero and a variance (and standard deviation) of 1. Because each protein comprises unique distributions for the different MHC alleles standardization of the affinity data to zero mean and unit variance provides a numerical scale where different alleles and different proteins can be compared. Analysis of a wide range of experimental results suggest that a criterion of standard deviation units can be used to discriminate between potential immunological responses and non-responses. An affinity of 1 standard deviation below the mean was found to be a useful threshold in this regard and thus approximately 15% (16.2% to be exact) of the peptides found in any protein will fall into this category.

The terms “specific binding” or “specifically binding” when used in reference to the interaction of an antibody and a protein or peptide or an epitope and an MHC allele means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc.

“Adjuvant” as used herein encompasses various adjuvants that are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, squalene, squalene emulsions, liposomes, imiquimod, keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

As used herein “immunoglobulin” means the distinct antibody molecule secreted by a clonal line of B cells; hence when the term “100 immunoglobulins” is used it conveys the distinct products of 100 different B-cell clones and their lineages.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “support vector machine” refers to a set of related supervised learning methods used for classification and regression. Given a set of training examples, each marked as belonging to one of two categories, an SVM training algorithm builds a model that predicts whether a new example falls into one category or the other.

As used herein, the term “classifier” when used in relation to statistical processes refers to processes such as neural nets and support vector machines.

As used herein “neural net”, which is used interchangeably with “neural network” and sometimes abbreviated as NN, refers to various configurations of classifiers used in machine learning, including multilayered perceptrons with one or more hidden layer, support vector machines and dynamic Bayesian networks. These methods share in common the ability to be trained, the quality of their training evaluated, and their ability to make either categorical classifications of non-numeric data or to generate equations for predictions of continuous numbers in a regression mode. Perceptron as used herein is a classifier which maps its input x to an output value which is a function of x, or a graphical representation thereof.

As used herein, the term “principal component analysis”, or as abbreviated “PCA”, refers to a mathematical process which reduces the dimensionality of a set of data (Wold, S., Sjorstrom, M., and Eriksson, L., Chemometrics and Intelligent Laboratory Systems 2001. 58: 109-130; Multivariate and Megavariate Data Analysis Basic Principles and Applications (Parts I&II) by L. Eriksson, E. Johansson, N. Kettaneh-Wold, and J. Trygg, 2006 2^{nd} Edit. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements. The application of PCA generates numerical coefficients (descriptors). The coefficients are effectively proxy variables whose numerical values are seen to be related to underlying physical properties of the molecules. A description of the application of PCA to generate descriptors of amino acids and by combination thereof peptides is provided in PCT US2011/029192 incorporated herein by reference in its entirety. Unlike neural nets PCA do not have any predictive capability. PCA is deductive not inductive.

As used herein, the term “vector” when used in relation to a computer algorithm or the present invention, refers to the mathematical properties of the amino acid sequence.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors. “Viral vector” as used herein includes but is not limited to adenoviral vectors, adeno-associated viral vectors, lentiviral vectors, retroviral vectors, poliovirus vectors, measles virus vectors, flavivirus vectors, poxvirus vectors, and other viral vectors which may be used to deliver a peptide or nucleic acid sequence to a host cell.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

A “subject” is an animal such as vertebrate, preferably a mammal such as a human, a bird, or a fish. Mammals are understood to include, but are not limited to, mice, simians, humans, bovines, sheep, cervids, equines, pigs, canines, felines etc.).

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

As used herein “Complementarity Determining Regions” (CDRs) are those parts of the immunoglobulin variable chains which determine how these molecules bind to their specific antigen. Each immunoglobulin variable region typically comprises three CDRs and these are the most highly variable regions of the molecule. T cell receptors also comprise similar CDRs and the term CDR may be applied to T cell receptors.

As used herein, the term “motif” refers to a characteristic sequence of amino acids forming a distinctive pattern.

The term “Groove Exposed Motif” (GEM) as used herein refers to a subset of amino acids within a peptide that binds to an MHC molecule; the GEM comprises those amino acids which are turned inward towards the groove formed by the MHC molecule and which play a significant role in determining the binding affinity. In the case of human MHC-I the GEM amino acids are typically (1,2,3,9). In the case of MHC-II molecules two formats of GEM are most common comprising amino acids (−3,2,−1,1,4,6,9,+1,+2,+3) and (−3,2,1,2,4,6,9,+1,+2,+3) based on a 15-mer peptide with a central core of 9 amino acids numbered 1-9 and positions outside the core numbered as negative (N terminal) or positive (C terminal).

“Immunoglobulin germline” is used herein to refer to the variable region sequences encoded in the inherited germline genes and which have not yet undergone any somatic hypermutation. Each individual carries and expresses multiple copies of germline genes for the variable regions of heavy and light chains. These undergo somatic hypermutation during affinity maturation. Information on the germline sequences of immunoglobulins is collated and referenced by www.imgt.org (4). “Germline family” as used herein refers to the 7 main gene groups, catalogued at IMGT, which share similarity in their sequences and which are further subdivided into subfamilies.

“Affinity maturation” is the molecular evolution that occurs during somatic hypermutation during which unique variable region sequences generated that are the best at targeting and neutralizing and antigen become clonally expanded and dominate the responding cell populations.

“Germline motif” as used herein describes the amino acid subsets that are found in germline immunoglobulins. Germline motifs comprise both GEM and TCEM motifs found in the variable regions of immunoglobulins which have not yet undergone somatic hypermutation.

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Illustrative examples of immunopathologies include, but are not limited to, B-cell lymphoma, T-cell lymphomas, Systemic Lupus Erythematosus (SLE), allergies, hypersensitivities, immunodeficiency syndromes, radiation exposure or chronic fatigue syndrome.

“pMHC” Is used to describe a complex of a peptide bound to an MHC molecule. In many instances a peptide bound to an MHC-I will be a 9-mer or 10-mer however other sizes of 7-11 amino acids may be thus bound. Similarly MHC-II molecules may form pMHC complexes with peptides of 15 amino acids or with peptides of other sizes from 11-23 amino acids. The term pMHC is thus understood to include any short peptide bound to a corresponding MHC.

“Somatic hypermutation” (SHM), as used herein refers to the process by which variability in the immunoglobulin variable region is generated during the proliferation of individual B-cells responding to an immune stimulus. SHM occurs in the complementarity determining regions.

“T-cell exposed motif” (abbreviated “TCEM”), as used herein, refers to the subset of amino acids in a peptide bound in a MHC molecule which are directed outwards and exposed to a T-cell binding to the pMHC complex. A T-cell binds to a complex molecular space-shape made up of the outer surface MHC of the particular HLA allele and the exposed amino acids of the peptide bound within the MHC. Hence any T-cell recognizes a space shape or receptor which is specific to the combination of HLA and peptide. The amino acids which comprise the TCEM in an MHC-I binding peptide typically comprise positions 4, 5, 6, 7, 8 of a 9-mer. The amino acids which comprise the TCEM in an MHC-11 binding peptide typically comprise 2, 3, 5, 7, 8 or −1, 3, 5, 7, 8 based on a 15-mer peptide with a central core of 9 amino acids numbered 1-9 and positions outside the core numbered as negative (N terminal) or positive (C terminal). As indicated under pMHC, the peptide bound to a MHC may be of other lengths and thus the numbering system here is considered a non-exclusive example of the instances of 9-mer and 15 mer peptides.

“Pentamer amino acid motif” or “pentameric amino acid motif” as used herein refers to a set of five amino acids arranged in the same configuration as a T cell exposed motif, but not necessarily bound in a MHC. Thus a pentamer amino acid motif may refer to a contiguous sequence of five amino acids in the format XXXXX, or to a discontinuous pentamer in the format XX~X~XX or X~X~~X~XX, where X is any amino acid. A T cell exposed motif is defined by its protrusion from an MHC and exposure to the T cell receptor when the underlying peptide is bound by a MHC molecule. A pentamer amino acid motif is the same pattern of amino acids occurring in a protein in the absence of any MHC binding. A pentamer amino acid motif only becomes a T cell exposed motif if the peptide in which it lies is appropriately cleaved out of a protein and the host's MHC alleles have the necessary affinity for binding that peptide to expose the pentamer motif.

As used herein “histotope” refers to the outward facing surface of the MHC molecules which surrounds the T cell exposed motif and in combination with the T cell exposed motif serves as the binding surface for the T cell receptor.

As used herein the T cell receptor refers to the molecules exposed on the surface of a T cell which engage the histotope of the MHC and the T cell exposed motif of a peptide bound in the MHC. The T cell receptor comprises two protein chains, known as the alpha and beta chain in 95% of human T cells and as the delta and gamma chains in the remaining 5% of human T cells. Each chain comprises a variable region and a constant region. Each variable region comprises three complementarity determining regions or CDRs

“Regulatory T-cell” or “Treg” as used herein, refers to a T-cell which has an immunosuppressive or down-regulatory function. Regulatory T-cells were formerly known as suppressor T-cells. Regulatory T-cells come in many forms but typically are characterized by expression CD4+, CD25, and Foxp3. Tregs are involved in shutting down immune responses after they have successfully eliminated invading organisms, and also in preventing immune responses to self-antigens or autoimmunity.

“uTOPE™ analysis” as used herein refers to the computer assisted processes for predicting binding of peptides to MHC and predicting cathepsin cleavage, described in PCT US2011/029192, PCT US2012/055038, and US2014/01452, each of which is incorporated herein by reference in its entirety.

“Framework region” as used herein refers to the amino acid sequences within an immunoglobulin variable region which do not undergo somatic hypermutation.

“Isotype” as used herein refers to the related proteins of particular gene family. Immunoglobulin isotype refers to the distinct forms of heavy and light chains in the immunoglobulins. In heavy chains there are five heavy chain isotypes (alpha, delta, gamma, epsilon, and mu, leading to the formation of IgA, IgD, IgG, IgE and IgM respectively) and light chains have two isotypes (kappa and lambda). Isotype when applied to immunoglobulins herein is used interchangeably with immunoglobulin “class”.

“Isoform” as used herein refers to different forms of a protein which differ in a small number of amino acids. The isoform may be a full length protein (i.e., by reference to a reference wild-type protein or isoform) or a modified form of a partial protein, i.e., be shorter in length than a reference wild-type protein or isoform. In accordance with the convention adopted by the Genome Data Commons, the isoform selected as the reference for numbering amino acid positions is the longest identified in Uniprot https://www.uniprot.org.

“Immunostimulation” as used herein refers to the signaling that leads to activation of an immune response, whether the immune response is characterized by a recruitment of cells or the release of cytokines which lead to suppression of the immune response. Thus, immunostimulation refers to both upregulation or down regulation.

“Up-regulation” as used herein refers to an immunostimulation which leads to cytokine release and cell recruitment tending to eliminate a non self or exogenous epitope. Such responses include recruitment of T cells, including effectors such as cytotoxic T cells, and inflammation. In an adverse reaction upregulation may be directed to a self-epitope.

“Down regulation” as used herein refers to an immunostimulation which leads to cytokine release that tends to dampen or eliminate a cell response. In some instances such elimination may include apoptosis of the responding T cells.

“Frequency class” or “frequency classification” as used herein is used to describe logarithmic based bins or subsets of amino acid motifs or cells. When applied to the counts of TCEM motifs found in a given dataset of peptides a logarithmic (log base 2) frequency categorization scheme was developed to describe the distribution of motifs in a dataset. As the cellular interactions between T-cells and antigen presenting cells displaying the motifs in MHC molecules on their surfaces are the ultimate result of the molecular interactions, using a log base 2 system implies that each adjacent frequency class would double or halve the cellular interactions with that motif. Thus, using such a frequency categorization scheme makes it possible to characterize subtle differences in motif usage as well as providing a comprehensible way of visualizing the cellular interaction dynamics with the different motifs. Hence a Frequency Class 2, or FC 2 means 1 in 4, a Frequency class 10 or FC 10 means 1 in 2¹⁰ or 1 in 1024. In other embodiments the frequency classification of the TCEM motif in the reference dataset is described by the quantile score of the TCEM in the reference dataset. Quantile scores are used, but is not limited to, applications where the reference dataset is the human proteome or a microbial proteome. “Frequency class” or “frequency classification” may also be applied to cellular clonotypic frequency where it refers to subgroups or bins defined by logarithmic based groupings, whether log base 2 or another selected log base.

“Frequency” as used herein in reference to the human proteome and microbial databases including the gastrointestinal microbiome reference database refers to the count of occurrences or count of a particular amino acid motif in that database or proteome.

“hPPF” as used herein refers to the human proteome pentamer frequency or the count of occurrences of a particular amino acid pentameric motif in the human proteome. “hPPF” I refers to the count of pentamers which are in the configuration presented by a TCEM I i.e. a contiguous pentamer like positions 4,5,6,7,8 within a 9 mer. “hPPF II” refers to the count of pentamers which are in the configuration presented by a TCEM II i.e. a discontinuous pentamer like positions 2,3,5,7,8 in a central core 9 mer of a 15mer. “giPPF I” and “giPPF II” refer to the corresponding pentameric amino acid motif counts within a representative gastrointestinal microbiome protein database.

A “rare TCEM” as used herein is one which is completely missing in the human proteome or present in up to only five instances in the human proteome. Similarly a TCEM may be rare with respect to the gastrointestinal microbiome reference database or other database if it is missing or only occurs five or less times.

“IGHV” as used herein is an abbreviation for immunoglobulin heavy chain variable regions.

“IGLV” as used herein is an abbreviation for immunoglobulin light chain variable regions.

“Adverse immune response” as used herein may refer to (a) the induction of immunosuppression when the appropriate response is an active immune response to eliminate a pathogen or tumor or (b) the induction of an upregulated active immune response to a self-antigen or (c) an excessive up-regulation unbalanced by any suppression, as may occur for instance in an allergic response.

“Clonotype” as used herein refers to the cell lineage arising from one unique cell. In the particular case of a B cell clonotype it refers to a clonal population of B cells that produces a unique sequence of IGV. The number of B cells that express that sequence varies from singletons to thousands in the repertoire of an individual. In the case of a T cell it refers to a cell lineage which expresses a particular TCR. A clonotype of cancer cells all arise from one cell and carry a particular mutation or mutations or the derivates thereof. The above are examples of clonotypes of cells and should not be considered limiting. “Clonal population” or “clonal line” may be used as a synonym for clonotype.

As used herein “epitope mimic” or “TCEM mimic” is used to describe a peptide which has an identical or overlapping TCEM, but may have a different GEM. Such a mimic occurring in one protein may induce an immune response directed towards another protein which carries the same TCEM motif. This may give rise to autoimmunity or inappropriate responses to the second protein.

“Cytokine” as used herein refers to a protein which is active in cell signaling and may include, among other examples, chemokines, interferons, interleukins, lymphokines, granulocyte colony-stimulating factor tumor necrosis factor and programmed death proteins.

“MHC subunit chain” as used herein refers to the alpha and beta subunits of MHC molecules. A MHC II molecule is made up of an alpha chain which is constant among each of the DR, DP, and DQ variants and a beta chain which varies by allele. The MHC I molecule is made up of a constant beta macroglobulin and a variable MHC A, B or C chain.

As used here in “virome” comprises the viruses present in a human subject, latently chronically or during acute infection, or a sub-set thereof made up of viruses of a particular taxonomic group or of the viruses located in a particular tissue or organ.

“Immunoglobulinome” as used herein refers to the total complement of immunoglobulins produced and carried by any one subject.

As used herein “allergome” refers to all proteins which may give rise to allergies. This includes proteins recorded in allergen datasets such as that represented on the world wide web at allergome.com, allergenonline.org, and comparedatabase.org, and allergen.com as well as included in Uniprot, Swiss-Prot, etc.

As used herein the term “repertoire” is used to describe a collection of molecules or cells making up a functional unit or whole. Thus, as one non limiting example, the entirely of the B cells or T cells in a subject comprise its repertoire of B cells or T cells. The entirety of all immunoglobulins expressed by the B cells are its immunoglobulinome or the repertoire of immunoglobulins. A collection of proteins or cell clonotypes which make up a tissue sample, an individual subject or a microorganism may be referred to as a repertoire.

As used herein “mutated amino acid” refers to the appearance of an amino acid in a protein that is the result of a nucleotide change, a missense mutation, or an insertion or deletion or fusion.

“Splice variant” as used herein refers to different proteins that are expressed from one gene as the result of inclusion or exclusion of particular exons of a gene in the final, processed messenger RNA produced from that gene or that is the result of cutting and re-annealing of RNA or DNA.

“T cell receptor” as used herein refers to the heterodimer (two proteins) located on the surface of a t cell that engage with the epitope peptide bound by an MHC molecule (pMHC). T cell receptor is abbreviated herein as TCR.

“TRAV” as used herein refers to the T cell receptor alpha variable region family or allele subgroups and “TRBV” refers to T cell receptor beta variable region family or allele subgroups as described in IMGT (on the world-wide web at imgt.org/IMGTrepertoire/Proteins/index.php#C, and imgt.org/IMGTrepertoire/Proteins/taballeles/human/TRA/TRAV/Hu_TRAVall.html.TRAV comprises at least 41 subgroups, with some having sub-subgroups. TRBV comprises at least 30 subgroups. Most combinations of alpha and beta variable region subgroups are encountered.

“hTRAV” refers to human TRAV. As used here in a “receptor bearing cell” is any cell which carries a ligand binding recognition motif on its surface. In some particular instances a receptor bearing cell is a B cell and its surface receptor comprises an immunoglobulin variable region, the immunoglobulin variable region comprising both heavy and light chains which make up the receptor. In other particular instances a receptor bearing cell may be a T cell which bears a receptor made up of both alpha and beta chains or both delta and gamma chains. Other examples of a receptor bearing cell include cells which carry other ligands such as, in one particular non limiting example, a programmed death protein of which there are multiple isoforms.

As used herein the term “bin” refers to a quantitative grouping and a “logarithmic bin” is used to describe a grouping according to the logarithm of the quantity.

As used herein “immunotherapy intervention” is used to describe any deliberate modification of the immune system including but not limited to through the administration of therapeutic drugs or biopharmaceuticals, radiation, T cell therapy, application of engineered T cells, which may include T cells linked to cytotoxic, chemotherapeutic or radiosensitive moieties, checkpoint inhibitor administration, cytokine or recombinant cytokine or cytokine enhancer, including but not limited to a IL-15 agonist, microbiome manipulation, vaccination, B or T cell depletion or ablation, or surgical intervention to remove any immune related tissues.

As used herein “immunomodulatory intervention” refers to any medical or nutritional treatment or prophylaxis administered with the intent of changing the immune response or the balance of immune responsive cells. Such an intervention may be delivered parenterally or orally or via inhalation. Such intervention may include, but is not limited to, a vaccine including both prophylactic and therapeutic vaccines, a biopharmaceutical, which may be from the group comprising an immunoglobulin or part thereof, a T cell stimulator, checkpoint inhibitor, or suppressor, an adjuvant, a cytokine, a cytotoxin, receptor binder, an enhancer of NK (natural killer) cells, an interleukin including but not limited to variants of IL15, superagonists, and a nutritional or dietary supplement. Immunomodulator interventions also includes protease inhibitors, including but not limited to inhibitors of cathepsins, and may include but are not limited to molecules from the group comprising nitrile derivatives, ketone derivatives, acryl hydrazine derivatives, vinyl sulfonate derivatives, epoxy succinic acids, surugamides, loxistatin derivatives, sulfonamide derivatives and betalactams, natural medicinal derivatives such as caffeic acid and chlorogenic acid. Additional cathepsin inhibitors are members of the cystatin family, including but not limited to, the stefins and cystatin C. The immunomodulatory intervention may also include radiation or chemotherapy to ablate a target group of cells. The impact on the immune response may be to stimulate or to down regulate.

“Checkpoint inhibitor” or “checkpoint blockade” as used herein refers to a type of drug that blocks certain proteins made by some types of immune system cells, such as T cells, and some cancer cells. These proteins help keep immune responses in check, limit the duration of T cell responses, and can prevent T cells from killing cancer cells. When these proteins are blocked, the “brakes” on the immune system are released and T cells are able to kill cancer cells better. Examples of checkpoint proteins found on T cells or cancer cells include, but are not limited to, PD-1/PD-L1 and CTLA-4/B7-1/B7-2 and LAG-3. Multiple check point inhibitors have been developed or are in development and include, but are not limited to, PD-1 inhibitors (e.g., Nivolumab, Pembrolizumab, Dostarlimab and Cemiplimab), PD-L1 inhibitors (e.g. Atezolizumab, Avelumab, Durvalumab), CTLA-4 inhibitors (e.g. Ipilimumab, Tremelimumab), and LAG-3 inhibitors (e.g. Retalimab).

As used herein the “cluster of differentiation” proteins refers to cell surface molecules providing targets for immunophenotyping of cells. The cluster of differentiation is also known as cluster of designation or classification determinant and may be abbreviated as CD. Examples of CD proteins include those listed at uniprot.org/docs/cdlist.

As used herein “microbiome” refers to the constellation of commensal microorganisms found within the human or other host body, inhabiting sites such as the gastrointestinal tract, skin, the urogenital tract, the oral cavity, the upper respiratory tract. While most frequently referring to bacteria, the microbiome also may include the viruses in these sites, referred to as the “virome,” or commensal fungi.

As used herein “tumor associated antigens” are antigens in proteins commonly upregulated in a tumor, or different types of tumor, but which are not mutated nor specific to that tumor and not differentiated form a wild type protein.

“Pattern” as used herein means a characteristic or consistent distribution of data points.

As used herein “presentome” refers to the multiplicity of peptides bound in MHC and simultaneously presented on the surface of antigen presenting cells. Mass spectroscopy detects some, but not all, peptides which are part of the presentome.

“Neoepitope” as used herein refers to a novel epitope amino acid motif or antigen created as the result of introduction of a mutation into an amino acid sequence. Thus, a neoepitope differentiates a wildtype protein from its mutant-bearing tumor protein homolog when such mutant is presented to T cells or B cells. A “neoantigen” is a neoepitope which elicits an immune response.

“Tumor specific antigen” or “tumor specific epitope” is used herein to designate an epitope or antigen that differentiates a mutated tumor protein from its unmutated wildtype homologue. Thus, a neoantigen or neoepitope is one type of tumor specific antigen.

As used herein “driver” mutations are those which arise early in tumorigenesis and are causally associated with the early steps of cell dysregulation. Driver mutations occur in oncogenes and tumor suppressor genes. Driver mutations are usually shared by all clonal offspring arising from the initial tumor cells and offer some additional fitness benefit to the clonal line within its microenvironment.

In contrast “passenger” is applied herein to mutations of genes and their products in a tumor which are not in oncogenes or tumor suppressor genes and which offer no particular benefit of fitness to the cell. Passengers may serve as biomarkers on tumor cells and may enable some immune evasion. Passenger mutations may differ at different time points in its development and among different parts of a tumor or among metastases. Any tumor may comprise any number of driver and passenger mutations. “Driver and passenger” are terms largely interchangeable with “trunk and branch” mutations.

“Oncogene” as used herein to describe a gene and gene product “oncoprotein” which have the capability to cause dysregulation of cell growth. Such dysregulation most often occurs when an oncogene is mutated.

“Tumor suppressor gene” as used herein refers to a gene and gene product that normally controls cell replication or nucleic acid replication or apoptosis. When mutated and such functions fail a mutated tumor suppressor may become a driver of tumor progression.

“Personal mutations” as used herein refers to mutations found in the tumor of a particular subject and not commonly shared with other affected subjects. In contrast “common mutations” are used to describe those mutations which occur in many tumors and many types of cancer. Illustrative examples are TP53 R175H, KRAS G12C, BRAF R640M.

“Bespoke peptides” or “bespoke vaccine” as used herein refers to a peptide or neoantigen or a combination of peptides, or nucleic acid encoding peptides, which are tailored or personalized specifically for an individual patient, taking into account that patient's HLA alleles and mutations.

“Heteroclitic” and “heteroclitic peptide” as used herein refers to a peptide in which amino acid substitutions have been made in the groove exposed motifs to alter the binding affinity to a particular HLA allele while maintaining the TCEM constant.

As used herein “TCGA” refers to The Cancer Genome Atlas (on the world-wide web at cancer.gov/about-nci/organization/ccg/research/structural-genomics/tcga.

As used herein a “polyhydrophobic amino acid” refers to a short chain of natural amino acids which are hydrophobic. Examples include, but are not limited to, leucines, isoleucines or tryptophans where these are assembled in multimers of 5-15 repeats of any one such amino acid. As a non-limiting example, a poly leucine comprising 8 leucines would be an example of a polyhydrophobic amino acid.

A “lipid core peptide system”, as used herein, refers to subunit vaccine comprising a lipoamino acid (LAA) moiety which allows the stimulation of immune activity. A combination of T cell stimulating epitopes or T and B cell stimulating epitopes are linked to a LAA. Multiple different constructs can be created with of different spatial orientation or LAA lengths (e.g. C12 2-amino-D,L-dodecanoic acid or C16, 2-amino-D,L-hexadecanoic acid). When dissolved in a standard phosphate buffer LCP particles form and the particles facilitate uptake by antigen presenting cells. Different LAA chain lengths lead to different particle sizes.

As used herein, the term “cleavage site octomer” refers to the 8 amino acids located four each side of the bond at which a peptidase cleaves an amino acid sequence. Cleavage site octomer is abbreviated as CSO. “Cathepsin cleavage site octomer” is used herein where the peptidase is a cathepsin.

“Cathepsin” as used herein may refer to any cathepsin encoded by the human genome including but not limited to cathepsins B, C, F, H, K, L, O, S, V, W, and X and whether they act as endopeptidases, carboxypeptidases or aminopeptidases.

As used herein “compounding pharmacy” has the meaning defined in sections 503A and 503B of the Federal Food, Drug, and Cosmetic Act

As used herein, a “BAM” file is a compressed binary version of a Sequence Alignment File “SAM” file wherein the all nucleotides are aligned to a reference genome. A “BAM slice” is a subset of the entire genome defined by genome coordinates. The HLA locus is located on Chromosome 6. In one particular instance a BAM slice is defined to contain just the HLA locus.

“Antigen presenting cell” (APC) as used herein refers to cells which are capable of presentation of peptides to T cells bound to MHC molecules. This includes but is not limited to the so called “professional” antigen presenting cells comprising but not limited to dendritic cells, B cells, and macrophages, and Langerhans cell, but also the so called non-professional antigen presenting cells which carry MHC molecules.

“PBMC” as used herein refers to peripheral blood mononuclear cells.

“Genome Data Commons” or GDC refers to the repository of cancer sequencing maintained by the National Cancer Institute. See the world wide web at gdc.cancer.gov.

“Multiplex” as used herein refers to a combination of peptides or nucleotides each of which provides a different epitope. Such combination may be delivered as individual epitopes in a single mixture or as a linked chain of epitopes, or the nucleotides that encode them, separates by appropriate spacer sequences.

### DESCRIPTION OF THE INVENTION

The present invention addresses methods for the rapid identification of the most effective potential immunogenic neoepitopes in commonly mutated tumor driver proteins and the most common mutations therein. It also enables methods to identify epitopes that are best excluded from a vaccine preparation as they are less likely to be presented to T cells in vivo. The present invention further enables exclusion of those epitopes which have a higher risk of eliciting an adverse off-target response.

The methods of neoepitope selection provided in the present invention address missense mutations, but similar approaches can be applied to other types of tumor specific mutations, including but not limited to, insertions, deletions, splice variants, and fusions.

The methods of neoepitope selection provided in the present invention are also applicable for rapid analysis of tumor-specific mutations which are unique to the individual subject, including passenger mutations and less common mutations in oncogenes and tumor suppressor gene products.

In addition to enabling expeditious selection of neoantigens for vaccine design, methods are also provided herein for use of selected neoantigens to select cognate T cell clones either for expansion and autologous or non-autologous transfer or for sequencing to introduce the TCR of interest into recipient cells.

The present invention further provides methods for expediting the assembly of neoepitope vaccinal peptides by advance preparation of an array or library of trimer amino acid building blocks for assembly to provide 9 mer or 15 mer neoantigen peptides, and also longer peptides if linear B cell epitopes are desired, thereby reducing the number of steps for neoantigen assembly and the corresponding quality control steps.

Recognition of tumor-specific neoepitopes by cytotoxic lymphocytes is the primary immunological mechanism for elimination of tumor cells (1). A fundamental premise is that for an effective tumor recognition response to occur, a mutation must generate an epitope that differs from the unmutated wildtype protein (5). Secondly, there must be one or more clones of T cells bearing receptors that bind to the mutant peptide:MHC complex (pMHC). Individual tumor-specific amino acid mutations create unique peptides which are potential targets for neoepitope vaccines (1, 6). However, very few mutations actually produce immunogenic neoantigens (3, 7). The present invention addresses methods to rapidly identify those tumor mutations which are most likely to be immunogenic and therefore actionable as targets of immune intervention. For commonly occurring oncogene and tumor suppressor gene product mutations such evaluation can be performed ahead of time and peptides or their encoding nucleic acids prepared and stored as a library in anticipation of a specific subject's diagnosis.

One criterion for selection of a particular neoepitope, enabled by the present invention, is to identify the pentameric amino acid motifs exposed to the T cell receptor (TCR) by a mutated peptide, when bound and presented by an MHC, and then to determine the count of the same pentamer amino acid motif in the normal human proteome, and/or in other reference databases including, but not limited to, a dataset of the proteins of organisms in a representative gastrointestinal (GI) microbiome, datasets of other microbial proteins, and a dataset of the variable regions of human immunoglobulins. This provides an indicator of whether the T cell exposed motif is rare or common and hence the likelihood of its encountering a cognate T cell in the subjects T cell repertoire.

T cell recognition of a tumor-specific mutation depends on presentation of short peptides bound in MHC molecules. Amino acids of the TCR engage the MHC histotope and the protruding amino acid side chains of the bound peptides (8, 9, 10). T cell recognition is highly polyclonal; the exposed amino acid motif of a bound peptide may be recognized by many cognate T cell clones with different alpha and beta subunits (11). The amino acids in a peptide whose side chain atoms interact with those within the MHC groove determine the binding affinity (12). These groove-facing amino acids in the so-called ‘anchor positions’ are hidden from the TCR. Only amino acid side chains in the in the non-anchor positions have atomic-level interactions with the TCR (9). Thus, for tumor-specific T cell recognition of a neoepitope, the mutant amino acids needs to be in a position exposed to the TCR and not hidden in the anchor positions (13, 14).

When a peptide is bound in an MHC groove, whether MHC I or MHC II, the exposed amino acids comprise a pentamer (9, 15, 16, 17). We refer to these exposed pentamers as the T cell exposed motif (TCEM) and the hidden residues in the anchor positions as groove-exposed motifs (GEM). In a 9mer peptide bound in an MHC I, the TCEM comprises amino acids p4, p5, p6, p7 and p8 (TCEM I). When a 15mer peptide is bound in an MHC II amino acids p2, p3, p5, p7, and p8 of the central 9mer are the dominant TCEM (TCEM II) (9, 16, 18, 19, 20, 21). As the amino acid combinations that engage the TCR may be continuous pentamers (MHC I) or discontinuous pentamers (MHC II), we refer to these herein as pentamer or pentameric amino acid motifs.

The total possible combinations of 20 amino acids as a pentamer is 205 or 3.2 million. We have previously shown that the human proteome only contains approximately 2.4 million of the possible 3.2 million unique pentamers for each MHC class (22). A dataset comprising the complete proteomes of 67 representative bacterial species found in the gastrointestinal microbiome (GI microbiome) was found to comprise 2.9 million of the possible pentamer motifs (22), partially overlapping those in the human proteome. Both CD8+ and CD4+ responses are needed for an effective tumor targeting response (23, 24, 25, 26, 27, 28).

Self-peptides in the human proteome are the basis of both positive and negative selection of naïve T cells during thymic processing of naïve CD4+ and CD8+ cells (29, 30, 31, 32, 33). The number of times any self-peptide is presented on thymocytes, and particularly presentation of the exposed amino acid pentamer motifs they comprise, plays a role in shaping the foundational T cell repertoire (34). If a given pentameric amino acid motif is not present in the human proteome, regardless of the MHC binding affinity or alleles, it cannot be presented in the thymus. In early post-natal life peptides derived from exogenous proteins, including peptides of the GI microbiome carried by antigen presenting cells, also contribute to positive selection of T cell clones (35, 36, 37, 38). The T cell repertoire is further shaped over a lifetime of exposure to peptides with recognized TCEM (39). Prior to puberty and early adulthood this expands the diversity of the repertoire. In later life immunosenescence leads to a progressive reduction in T cell repertoire diversity, in part due to exposure to chronic viral pathogens (40, 41, 42, 43, 44). T cells that recognize rare TCEM are both less likely to be presented during positive selection in the thymus, and also progressively less likely to be present in the T cell repertoire as it narrows in ageing, thereby handicapping a response to a rare epitope.

The T cell response to an exposed T cell exposed motif depends on there being a T cell in the subject's repertoire with a TCR that engages the T cell exposed motif with sufficient but not excessive affinity. For such a T cell to exist in the subject's T cell repertoire, the pentameric amino acid motif corresponding to the T cell exposed motif must have been previously encountered, either during thymic selection of naïve T cells based on the presence of such a pentameric motif in the subject's self-proteome or by presentation, directly or via a dendritic cell or other antigen presenting cell, of such a pentameric motif derived from an exogenous source. It has been demonstrated that peptides from the gastrointestinal microbiome play a role in generation of cognate T cell clones early in life (36). The GI microbiome is included as a recognized source of diverse T cell stimulation linked to cancer outcome (45, 46).

Conversely, the ability to mount an immune response to a neoepitope depends on that neoepitope peptide being intact and available for presentation by MHC binding. Prior cleavage of the peptide precludes such presentation. The present invention therefore also addresses the identification of potential neoepitopes which are cleaved by cathepsin.

In a third aspect of rapid identification of effective neoepitopes the present invention also addresses the affinity of MHC binding and the preferred binding register of a mutated peptide and how this determines whether a mutant amino acid is accessible to a T cell or hidden in the MHC binding groove.

Neoepitope vaccines have shown considerable promise in changing the course of cancer progression (47, 48, 49). Neoepitope vaccines may offer direct benefit in enabling a subject affected with cancer to mount an effective cytotoxic immune response that curtails tumor progression. Neoepitope vaccines may also enhance the efficacy of other immunomodulatory interventions such as checkpoint inhibitor drugs, or immune agonists such as IL15, or indeed the ability to mount an immune response to apoptotic cells created by radiation of chemotherapy. Prior vaccination with a neoepitope vaccine may also facilitate the identification and expansion of selected clones of T cells for autologous transfer back to the subject. However, it has also become apparent that very few neoepitopes are actually immunogenic and that considerable care and precision must be invested in selecting the optimum effective neoantigens suitable for each particular subject affected by cancer, or at risk of being affected by cancer, based on their particular tumor specific mutations and their HLA genotype. Many earlier approaches to neoepitope vaccination have failed to recognize the level of precision needed in selection of a vaccinal candidate and in particular the precision needed in identification of optimal T cell exposed motifs and the likelihood of there being cognate precursor T cell clones present which can be stimulated. The present invention addresses this problem.

### Rapid Response

When cancer is diagnosed, time is of the essence in planning and implementing a therapeutic intervention. Delay between sequencing of a biopsy and implementing a neoepitope vaccine represents time during which the tumor mutational landscape may be changing. Typically, a biopsy is obtained surgically shortly after diagnosis. This can provide the sequences of proteins mutated in the tumor, which may be compared to those in a normal tissue sample. By RNA sequencing, the expression profile of the mutated proteins is determined. The tumor normal DNA and RNA sequencing is the baseline input for selection of peptides, or their encoding nucleic acids, with which to formulate a neoepitope vaccine. Given the potential for a neoepitope vaccine to recruit relevant effector T cell clones that can be directly beneficial, and which can also synergize with other forms of intervention, rapid selection and assembly of peptides, or the nucleic acids that encode them, for inclusion in a vaccine for administration to the affected subject is highly desirable.

The present invention is directed to facilitating the rapid selection, design, synthesis and assembly of a neoepitope vaccine through multiple described embodiments. The methods and embodiments described herein are applicable to a wide variety of tumors, including both solid tumors and hematologic cancers.

In one embodiment, the present invention is directed to applying criteria for rapid down selection of potential neoepitopes, both in driver and in passenger genes, and in common and unique personal mutated tumor proteins. One important selective criterion is the frequency of occurrence of the pentamer motifs that comprise the mutated amino acids which is an indicator of the probability of whether a cognate precursor T cell clone or clones may be present and which may be stimulated. Comparing the frequency of a pentameric T cell exposed motif to the number of occurrences of that pentameric amino acid motif in the self-proteome also provides an indicator of the probability that such motifs, if used in a vaccine, may stimulate an unwanted off-target response. In a further embodiment, the probability of cathepsin cleavage is a further criterion considered in selection of a neoepitope. Another approach to down-selection, which is known to the art and more typically applied, is selection based on binding to particular HLA of interest. A secondary aspect of potential immune evasion, less recognized, that arises from determining the preferred binding positions of a mutant-bearing peptide is to determine if the mutant amino acid is actually exposed to recognition by a T cell receptor or hidden in a groove exposed position. A combination of these criteria may be applied to the unique array of mutant gene products found in any subject's tumor biopsy to identify actionable neoepitopes. To the extent that such criteria are applied, singly or in combination, to evaluate commonly occurring mutations in frequently identified driver genes and other mutated genes that give rise to amino acid mutations, a library of suitable neoepitopes can be established for the more common HLA alleles prior to their identification in a particular subject for rapid deployment when needed.

Neoepitope vaccination seeks to establish an effective cytotoxic and helper T cell response in vivo. Another approach is to expand T cell clones outside the subject for subsequent autologous administration and also for the engineering of T cells to carry an optimum T cell receptor. This may be accomplished for immediate administration to a particular subject or for establishment of a reserve of T cells to target common mutations in subjects of matching HLA. The present invention provides methods to rapidly define the optimal peptide to select T cell clones for this purpose.

While the primary focus of neoepitope vaccines is on T cell epitopes, it must not be overlooked that de novo B cell epitopes may also be created by mutations in tumors. B cell neoepitopes offer the opportunity to stimulate antibody responses and thus potentially antibody dependent cell mediated cytotoxicity. Furthermore, the creation by mutation of tumor specific B cell neoepitopes offers the possibility to assemble companion diagnostic antibody-based assays based on antibodies and diagnostic tests to identify common mutations. Single amino acid mutations may generate novel B cell epitopes and short peptides, even as few as 3-5 amino acids, may encode novel linear B cell epitopes

The embodiments described above to rapidly select optimal neoepitopes are agnostic of the mode by which a vaccine will be administered to the subject. A neoepitope vaccine may be administered as nucleic acids encoding peptides of interest (e.g., as mRNA or DNA vaccines), or as peptides. When peptide delivery is the method of choice for either vaccination or T cell expansion, rapid peptide synthesis is desirable. A further aspect of the methods for rapid response provided herein is therefore the establishment of a preassembled library of trimer peptides which can be assembled to more rapidly deliver neoepitope peptides. As neoepitope peptides are commonly delivered as 9 mers or 15 mers, the pre-positioning of a library or trimer subunits enables a speedier delivery of a peptide vaccine and facilitates quality control.

### Immune Evasion

Multiple modes of immune evasion have been described. Many mutated proteins are not expressed and so do not provide relevant neoepitopes (7). Peptides comprising tumor-specific mutations, but which are not bound by MHCs, leave the potential cognate T cells ignorant of their existence (50). MHC expression may be down-regulated, or absent, in tumor cells (51, 52). The tumor microenvironment may provide physical and immunosuppressive barriers to effective immunogenicity and surveillance (53, 54, 55, 56, 57). However, other modes of immune evasion are specific to the protein and the particular mutation, and indeed may be particular to the individual subject in which the tumor mutations arise. Biopsies of tumors enable the detection of those mutations that are the survivors of immune pressure and selection, sometimes known as immunoediting, often over years (58, 59, 60). The present invention addresses modes of immune evasion that provide a selective advantage within the context of the immune response of the affected subject. To be effective, a vaccine or a T cell clone expanded in response to an immunogen, must overcome immune evasion. Thus, selection of vaccinal immunogens which can overcome continued immune evasion is essential.

### T Cell Exposed Motif Frequency

A previously unrecognized mechanism of tumor immune evasion which we document here as being of critical importance is the creation, by the tumor mutation, of rare or uncommon epitopes. In particular, the pentamer T cell exposed motifs arising from a tumor mutation include an increased count of pentameric amino acid motifs that are very rarely encountered and for which an individual subject may not carry a cognate precursor T cell population that is readily stimulated. Such rare motifs include those comprising pentameric amino acid motifs that are absent or at very low frequency in the human proteome, and thus may not have participated in positive thymic selection as one mode of establishing a precursor clonal population. The pentameric amino acid motifs include continuous pentamers that correspond to the T cell exposed motifs exposed to the T cell receptor when a peptide is bound by an MHC I molecule (positions 4, 5, 6, 7, 8 of a 9mer peptide). They also include discontinuous pentamers exposed to a T cell receptor when a peptide is bound in an MHC II molecule (positions 2, 3, 5, 7, 8 of the central 9mer of a 15mer). In addition, corresponding pentameric T cell exposed motifs that are rarely encountered in the environment, including the microbial environment, are also likely to have a low probability of encountering a precursor T cell clone that may be stimulated. One source of the epitope diversity and frequency in the microbial environment is the gastrointestinal microbiome. Exposure to the varied epitopes in the microbiome contributes to the development and maintenance of the T cell repertoire starting early in life (36). We therefore have established a reference database comprising the pentameric amino acid motifs in the open reading frames of a representative gastrointestinal microbiome. This comprises both the continuous pentamers equivalent to the amino acids exposed in a pMHC I and the discontinuous pentamers equivalent to the amino acids exposed in a pMHC II. Another such reference database comprises the pentameric amino acid motifs in commonly occurring pathogens and routine vaccinations for the same. Yet another diverse source of T cell epitopes that may stimulate and give rise to T cell clonal populations is the immunoglobulinome, in particular comprising the variable regions of immunoglobulins (16, 22).

While neoantigens comprising T cell exposed motifs of medium and higher frequency in the human proteome may be more likely to have generated precursor T cell clones and respond to re-stimulation by a neoantigen vaccine, the higher frequency also comes with a higher risk of encountering an epitope mimic in the normal proteome which could have adverse consequences. Thus, in some preferred embodiments, a preferred peptide may be selected to have a T cell exposed motif with higher frequency of representation (count) in the human proteome and in other instances a lower frequency may be selected to avoid an epitope mimic. On the other hand, a higher count of a pentameric amino acid motif in the microbiome or in microbial pathogens may indicate a greater propensity to mount a T cell response following vaccination without associated risks of off-target responses.

### Peptidase Cleavage

A further critical mode of immune evasion is the cleavage, by a peptidase, of a potential tumor neoepitope peptide created by a tumor mutation, such that the tumor-specific peptide bearing the mutant amino acid is cleaved and rendered unavailable for binding in an MHC molecule and presentation to a T cell. If a neoepitope peptide is cleaved by a cathepsin, it is not available for binding by an MHC molecule and presentation to a T cell thus evading immune surveillance. Identifying peptides prone to be cleaved and not presented to T cells informs the decision of which neoepitopes to select for inclusion in a vaccine. In one embodiment the probability of cleavage of a potential tumor neoepitope peptide by a cathepsin is considered. Cathepsin cleavage patterns are unique to each protein and to each mutation, and thus must be evaluated for the mutants carried by each cancer patient. Furthermore, the impact of cathepsin cleavage may vary between different cell types that carry the mutation and different locations in tissues that affect temperature and pH optimal for particular peptidase activity.

We show here that for some neoepitopes cathepsin cleavage, including both the absence and presence of peptide cleavage, is an important factor in selection and management of neoantigens for inclusion in a cancer vaccine. Cathepsins of particular relevance include cathepsins L, S and B. Cathepsin B is of particular importance given its function as both an endopeptidase and exopeptidase and its secretion from cells and reported upregulation in tumors.

A desirable tumor specific neoantigen is one in which the peptide is not cleaved and so is presented to T cells by MHC I and MHC II binding, or at least has a lower probability of cleavage. However, where a neoepitope peptide bearing a mutant amino acid is shown to have a high probability of cleavage by a cathepsin, or by another peptidase, the application of that peptide, or the nucleic acids encoding it, in a neoantigen vaccine may be combined with the administration of a cathepsin inhibitor to increase the chance of presentation of an intact neoantigen. Such administration may include, but is not limited to, the contemporaneous administration of a cathepsin inhibitor drug systemically or locally, or in the event a neoantigen vaccine is administered intratumorally, it may be an integral part of the vaccine formulation.

While all tumor proteins exhibit a range of probability of cathepsin cleavage near mutant sites, there are some tumor driver proteins which stand out as extreme examples. The Ras oncogene products, KRAS, NRAS and HRAS are among these. Mutants of these proteins account for about 25% of all cancers (61). Of these KRAS is the oncogene mutated in 86% of RAS mutations. The KRAS, HRAS and NRAS proteins are completely aligned in their first 86 amino acids and very highly conserved thereafter. Over 90% of the mutations in KRAS and the other Ras occur in two hotspots, at amino acid G12 or G13, and at Q61.

T cell mediated control of KRAS mutation in a tumor has been demonstrated on at least one occasion (62), but is a rare occurrence and has not produced sustained control. While stimulation of T cell clones specific to G12 mutations have been demonstrated ex vivo their autologous transfer has not resulted in improved clinical response (63, 64). This is consistent with the cleavage of the neoepitopes at the tumor site, thus preventing epitope presentation. Another approach to engineer a high affinity T cell receptor to a G12 mutant has also not yet produced repeatable clinical results (65). Cell free DNA encoding the mutant genes has been detected in serum and used as a prognostic indicator (66), but the peptides which embody the mutant amino acids are not detected. Therefore, developing a method of stimulating immune control of the common mutations of the RAS gene products as tumor drivers remains, even after decades of research, to be major and urgent challenge in oncology.

Down-regulation or inhibition of cathepsin cleavage, and in preferred embodiments the down-regulation or inhibition of cathepsin B, is therefore an adjunct to immunization with KRAS (or NRAS, or HRAS) peptides comprising mutant amino acids which can facilitate an effective immune response to these important neoepitopes. In preferred embodiments the mutations are mutations at G12, G13 or Q61, but may also be applied to neoepitopes arising at other positions in these proteins.

While the application of cathepsin inhibitors is discussed here with reference to the Ras gene product mutations, KRAS HRAS and NRAS, it will be clear to those skilled in the art that where other mutations are observed to occur in peptides with a high predicted probability of cathepsin cleavage, or indeed cleavage by other peptidases, that the coadministration of a cathepsin inhibitor, cystatin or other peptidase inhibitor is an intervention which will enhance the stimulation of an effective tumor specific cytotoxic immune response.

Achieving an effective cytotoxic response when a neoepitope peptide is cleaved by cathepsin, or other peptidases, involves two components. First a neoepitope vaccine comprising the putative neoantigen has to successfully stimulate cognate T cell clones by antigen presenting cells at the site of vaccination. Secondly, the presentation of the neoepitope as a neoantigen in the tumor requires overcoming peptide cleavage in the tumor cells. Addressing these two components may call for multiple strategies, including either or both of systemic and local applications of cathepsin inhibitors. In some embodiments, therefore, cathepsin inhibitor(s) may be administered parenterally. In other embodiments, the cathepsin inhibitor(s) may be administered intratumorally or topically, where the tumor is accessible on the skin, or applied to an affected mucosal surface. In some embodiments, the inhibitor(s) maybe co-administered with the vaccine peptides or their encoding nucleic acid sequences. In other embodiments, the administration of the vaccine and the inhibitor(s) may be implemented independently and sequentially. In some particular embodiments, a cystatin protein or a sub-component polypeptide from cystatin may be encoded in a nucleic acid sequence for co-expression with the vaccine peptides in antigen presenting cells, or for intratumoral expression. The role of cathepsins and choice of cathepsin inhibitors is discussed further below.

### MHC Binding and Motif Positioning

A further mode of immune evasion, more broadly recognized by those skilled in the art, is the position and affinity of binding of a mutated protein to the subject's MHC I and MHC II molecules of each HLA allele. If binding of sufficient affinity does not occur, effective T cell engagement with that epitope will not occur. If the highest affinity of binding occurs to a peptide register that places the mutant amino acid in a groove exposed position, where it is hidden from the T cell receptor, there is no differentiation by a T cell between mutated and wildtype peptide. If MHC I binding occurs by one or more allele, but no overlapping or immediately adjacent MHC II binding occurs, the potential for a CD4+ response to support a fully matured CD8+ response is reduced. While peptide binding by a particular MHC I allele tends to be very sensitive to the specific register or position of the peptide, MHC II binding often occurs with high, or low, affinity across several consecutive peptides for many MHC II alleles. An optimal response to a tumor-specific neoantigen requires CD8+ and CD4+ cells (23, 24). Mutations occurring in regions of broad MHC II binding may have a greater propensity to elicit an immune response, and vice versa.

It follows that in selecting neoepitopes for inclusion in a cancer vaccine that care must be taken to avoid or minimize use of neoepitopes that have low potential to produce an effective response and to maximize inclusion of those neoepitopes which will elicit an effective cytotoxic response.

The criteria presented here provide an indicator for rapid identification of neoepitopes most likely to elicit a response. In preferred embodiments particular consideration is given to the frequency of the T cell exposed motif in the human proteome and in reference databases. Selection based on MHC binding alone is insufficient to optimize the immune response to neoepitopes.

### Common Tumor Specific Mutations

Mutations identified in tumors may be classified as “drivers” which are central to the process of tumorigenesis and tumor progression, either as active oncogenes or as tumor suppressor gene products which remove a regulator or gene repair function (67). Alternatively, tumor specific mutations may arise in “passenger” gene products which are secondarily or contemporaneously mutated. While present in most tumor cells, but in some cases only in some clonal lines or metastases of tumor cells, passengers do not play such an active role in driving progression. Both drivers and passengers may comprise potentially targetable neoepitopes as “markers” of the dysregulated cells to which T cell responses may be directed. Table 1 lists commonly recognized tumor driver genes.

TABLE 1
Oncogenes and Tumor suppressor genes analyzed

| Uniprot ID | Gene ID | Type | Uniprot ID | Gene ID | Type |
|---|---|---|---|---|---|
| P00519_2 | ABL1 | Oncogene | P46100 | ATRX | TSG |
| P31749 | AKT1 | Oncogene | O15169 | AXIN1 | TSG |
| Q9UM73 | ALK | Oncogene | P61769 | B2M | TSG |
| P10275 | AR | Oncogene | Q92560 | BAP1 | TSG |
| P10415 | BCL2 | Oncogene | Q6W2J9 | BCOR | TSG |
| A0A2R8Y8E0 | BRAF | Oncogene | P38398_7 | BRCA1 | TSG |
| Q9BXL7 | CARD11 | Oncogene | P51587 | BRCA2 | TSG |
| P22681 | CBL | Oncogene | Q9UM11 | CDH1 | TSG |
| Q9HC73 | CRLF2 | Oncogene | Q14790_9 | CASP8 | TSG |
| P07333 | CSF1R | Oncogene | Q6P1J9 | CDC73 | TSG |
| P35222 | CTNNB1 | Oncogene | P42771_4 | CDKN2A | TSG |
| P26358 | DNMT1 | Oncogene | P49715 | CEBPA | TSG |
| Q9Y6K1 | DNMT3A | Oncogene | I3L2J0 | CIC | TSG |
| P00533 | EGFR | Oncogene | Q92793 | CREBBP | TSG |
| P04626 | ERBB2 | Oncogene | Q9NQC7 | CYLD | TSG |
| Q15910_2 | EZH2 | Oncogene | Q9UER7 | DAXX | TSG |
| P21802_3 | FGFR2 | Oncogene | Q09472 | EP300 | TSG |
| P22607_3 | FGFR3 | Oncogene | Q969H0 | FBXW7 | TSG |
| P36888 | FLT3 | Oncogene | Q96AE4 | FUBP1 | TSG |
| P58012 | FOXL2 | Oncogene | P15976 | GATA1 | TSG |
| P23769 | GATA2 | Oncogene | P23771_2 | GATA3 | TSG |
| P29992 | GNA11 | Oncogene | P20823 | HNF1A | TSG |
| P50148 | GNAQ | Oncogene | P41229 | KDM5C | TSG |
| Q5JWF2 | GNAS | Oncogene | A0A087X0R0 | KDM6A | TSG |
| P84243 | H3-3A | Oncogene | Q8NEZ4 | KMT2C | TSG |
| P68431 | H3C2 | Oncogene | O14686 | KMT2D | TSG |
| P01112 | HRAS | Oncogene | Q13233 | MAP3K1 | TSG |
| Q75874 | IDH1 | Oncogene | O00255 | MEN1 | TSG |
| P48735 | IDH2 | Oncogene | P40692 | MLH1 | TSG |
| P23458 | JAK1 | Oncogene | P43246 | MSH2 | TSG |
| O60674 | JAK2 | Oncogene | P52701 | MSH6 | TSG |
| P52333 | JAK3 | Oncogene | O75376 | NCOR1 | TSG |
| P10721 | KIT | Oncogene | P21359 | NF1 | TSG |
| Q43474_1 | KLF4 | Oncogene | P35240 | NF2 | TSG |
| P01116 | KRAS | Oncogene | P46531 | NOTCH1 | TSG |
| Q02750 | MAP2K1 | Oncogene | Q04721 | NOTCH2 | TSG |
| Q93074 | MED12 | Oncogene | A0A712YQC0 | NPM1 | TSG |
| P08581 2 | MET | Oncogene | Q02548 | PAX5 | TSG |
| P40238 | MPL | Oncogene | Q86U86 | PBRM1 | TSG |
| Q99836 | MYD88 | Oncogene | A0AOD9SGE8 | PHF6 | TSG |
| Q16236 | NFE2L2 | Oncogene | P27986 | PIK3R1 | TSG |
| P01111 | NRAS | Oncogene | A0A3B3IU23 | PRDM1 | TSG |
| P16234 | PDGFRA | Oncogene | Q13635 | PTCH1 | TSG |
| P42336 | PIK3CA | Oncogene | P60484 | PTEN | TSG |
| P30153 | PPP2R1A | Oncogene | P06400 | RB1 | TSG |
| Q06124 | PTPN11 | Oncogene | Q68DV7 | RNF43 | TSG |
| P07949 | RET | Oncogene | Q01196_8 | RUNX1 | TSG |
| Q9Y6X0 | SETBP1 | Oncogene | Q9BYW2 | SETD2 | TSG |
| Q75533 | SF3B1 | Oncogene | Q15796 | SMAD2 | TSG |
| Q99835 | SMO | Oncogene | Q13485 | SMAD4 | TSG |
| Q43791 | SPOP | Oncogene | G5E975 | SMARCB1 | TSG |
| Q01130 | SRSF2 | Oncogene | P51532 | SMARCA4 | TSG |
| P16473 | TSHR | Oncogene | O15524 | SOCS1 | TSG |
| Q01081 | U2AF1 | Oncogene | P48436 | SOX9 | TSG |
| P36896_4 | ACVR1B | TSG | Q8N3U4_2 | STAG2 | TSG |
| Q5JTC6 | AMER1 | TSG | Q15831 | STK11 | TSG |
| P25054 | APC | TSG | Q6N021 | TET2 | TSG |
| Q14497 | ARID1A | TSG | P21580 | TNFAIP3 | TSG |
| Q8NFD5 | ARID1B | TSG | P04637 | TP53 | TSG |
| Q8NFD5_3 | ARID1B | TSG | Q6Q0C0 | TRAF7 | TSG |
| Q68CP9 | ARID2 | TSG | Q92574 | TSC1 | TSG |
| Q8IXJ9 | ASXL1 | TSG | P40337 | VHL | TSG |
| Q13315 | ATM | TSG | P19544_7 | WT1 | TSG |

The Uniprot Identifier shown is for the longest recorded isoform of each protein. TSG=tumor suppressor gene

While the array of tumor specific mutations present in any tumor biopsy will be unique to the individual affected subject, some mutations are much more commonly reported than others. Most of the most common mutations occur in driver gene products. Tables 2 and 3 lists the most commonly reported tumor missense mutations recorded in the Genome Data Commons (68). The common mutations are identified by their position in the longest isoform recorded in the UniProt repository at ftp.uniprot.org/pub/databases/uniprot/current_release/ (69). These Tables also identify the T cell exposed motifs that have the potential to expose the mutant amino acid and thus create a tumor specific T cell neoepitope.

Mutations in tumors take many forms. The most common are missense mutations in which a single non-synonymous amino acid substitution has occurred. But tumor specific mutations also comprise insertions and deletions (indels), splice variants, gene fusions, RNA fusions, and other unique configurations which, when present in an expressed protein and unique to the tumor, may affect immune evasion and also may provide a unique neoantigen target. The methods described herein are thus not limited to missense mutations, although these are the most commonly occurring tumor specific mutation. Similarly, the methods described herein are not particular to any one type of cancer but may be applied across the full range of solid and hematologic cancers.

The most common tumor mutations detected in many cancers exhibit one or more of the multiple modes of immune evasion as discussed in the examples below. Each of these has a bearing on the suitability of a particular peptide, or a nucleic acid encoding a peptide, for inclusion in a neoepitope vaccine.

### Vaccine Library

The occurrence of the common mutations in some driver gene products, such as those listed in Tables 2 and 3, provides the opportunity to assess the frequency of the pentameric amino acid motifs in their T cell exposed motif and the cathepsin cleavage probability prior to their detection in a particular patient and to identify preferred target T cell motifs most likely to lead to an effective immune response based on pentamer frequency in the reference databases and probability of cleavage. Those shown in Tables 2 and 3 are examples of such mutations and TCEM motifs, but the same process may be applied to other common driver gene product mutants and so these examples should not be considered liming. The remaining criterion, that of MHC binding and hence peptide presentation in a position that exposes the desired pentameric motif comprising the mutant amino acid(s) to a T cell receptor, then depends on the HLA genotype of the individual cancer affected subject. A library of peptides which satisfy the criteria for pentamer motif frequency, and hence the probable presence of T cell precursors, and the avoidance of cathepsin cleavage as well as MHC binding can be pre-assembled for the most common HLA alleles. This library of peptides enables rapid deployment as soon as biopsy sequencing and HLA determination is available.

TABLE 2
Peptides and T cell exposed motifs presented by MHC I to T cells from common mutations

| Mutant aa | | Mutant | | | | |
|---|---|---|---|---|---|---|
| position in | driver | aa | 9-mer | SEQ | | SEQ |
| 9mer | protein | change | mutated | ID NO.: | TCEM I | ID NO.: |
| p8 | AKT | E17K | GWLHKRGKY | 1 | ~~~HKRGK~ | 421 |
| p7 | AKT | E17K | WLHKRGKYI | 2 | ~~~KRGKY~ | 422 |
| p6 | AKT | E17K | LHKRGKYIK | 3 | ~~~RGKYI~ | 423 |
| p5 | AKT | E17K | HKRGKYIKT | 4 | ~~~GKYIK~ | 424 |
| p4 | AKT | E17K | KRGKYIKTW | 5 | ~~~KYIKT~ | 425 |
| p8 | ATM | R337C | GKYSSGFCN | 6 | ~~~SSGFC~ | 426 |
| p7 | ATM | R337C | KYSSGFCNI | 7 | ~~~SGFCN~ | 427 |
| p6 | ATM | R337C | YSSGFCNIA | 8 | ~~~GFCNI~ | 428 |
| p5 | ATM | R337C | SSGFCNIAV | 9 | ~~~FCNIA~ | 429 |
| p4 | ATM | R337C | SGFCNIAVK | 10 | ~~~CNIAV~ | 430 |
| p8 | BCOR | N1459S | EARRLIVSK | 11 | ~~~RLIVS~ | 431 |
| p7 | BCOR | N1459S | ARRLIVSKN | 12 | ~~~LIVSK~ | 432 |
| p6 | BCOR | N1459S | RRLIVSKNA | 13 | ~~~IVSKN~ | 433 |
| p5 | BCOR | N1459S | RLIVSKNAG | 14 | ~~~VSKNA~ | 434 |
| p4 | BCOR | N1459S | LIVSKNAGE | 15 | ~~~SKNAG~ | 435 |
| p8 | BRAF | V640E | GDFGLATEK | 16 | ~~~GLATE~ | 436 |
| p7 | BRAF | V640E | DFGLATEKS | 17 | ~~~LATEK~ | 437 |
| p6 | BRAF | V640E | FGLATEKSR | 18 | ~~~ATEKS~ | 438 |
| p5 | BRAF | V640E | GLATEKSRW | 19 | ~~~TEKSR~ | 439 |
| p4 | BRAF | V640E | LATEKSRWS | 20 | ~~~EKSRW~ | 440 |
| p8 | BRAF | V640M | GDFGLATMK | 21 | ~~~GLATM~ | 441 |
| p7 | BRAF | V640M | DFGLATMKS | 22 | ~~~LATMK~ | 442 |
| p6 | BRAF | V640M | FGLATMKSR | 23 | ~~~ATMKS~ | 443 |
| p5 | BRAF | V640M | GLATMKSRW | 24 | ~~~TMKSR~ | 444 |
| p4 | BRAF | V640M | LATMKSRWS | 25 | ~~~MKSRW~ | 445 |
| p8 | CDKN2A | H83Y | ATLTRPVYD | 26 | ~~~TRPVY~ | 446 |
| p7 | CDKN2A | H83Y | TLTRPVYDA | 27 | ~~~RPVYD~ | 447 |
| p6 | CDKN2A | H83Y | LTRPVYDAA | 28 | ~~~PVYDA~ | 448 |
| p5 | CDKN2A | H83Y | TRPVYDAAR | 29 | ~~~VYDAA~ | 449 |
| p4 | CDKN2A | H83Y | RPVYDAARE | 30 | ~~~YDAAR~ | 450 |
| p8 | CTNNB1 | S33C | QQQSYLDCG | 31 | ~~~SYLDC~ | 451 |
| p7 | CTNNB1 | S33C | QQSYLDCGI | 32 | ~~~YLDCG~ | 452 |
| p6 | CTNNB1 | S33C | QSYLDCGIH | 33 | ~~~LDCGI~ | 453 |
| p5 | CTNNB1 | S33C | SYLDCGIHS | 34 | ~~~DCGIH~ | 454 |
| p4 | CTNNB1 | S33C | YLDCGIHSG | 35 | ~~~CGIHS~ | 455 |
| p8 | CTNNB1 | S37C | YLDSGIHCG | 36 | ~~~SGIHC~ | 456 |
| p7 | CTNNB1 | S37C | LDSGIHCGA | 37 | ~~~GIHCG~ | 457 |
| p6 | CTNNB1 | S37C | DSGIHCGAT | 38 | ~~~IHCGA~ | 458 |
| p5 | CTNNB1 | S37C | SGIHCGATT | 39 | ~~~HCGAT~ | 459 |
| p4 | CTNNB1 | S37C | GIHCGATTT | 40 | ~~~CGATT~ | 460 |
| p8 | CTNNB1 | S37E | YLDSGIHFG | 41 | ~~~SGIHF~ | 461 |
| p7 | CTNNB1 | S37F | LDSGIHFGA | 42 | ~~~GIHFG~ | 462 |
| p6 | CTNNB1 | S37E | DSGIHFGAT | 43 | ~~~IHFGA~ | 463 |
| p5 | CTNNB1 | S37F | SGIHFGATT | 44 | ~~~HFGAT~ | 464 |
| p4 | CTNNB1 | S37F | GIHFGATTT | 45 | ~~~FGATT~ | 465 |
| p8 | CTNNB1 | S45P | GATTTAPPL | 46 | ~~~TTAPP~ | 466 |
| p7 | CTNNB1 | S45P | ATTTAPPLS | 47 | ~~~TAPPL~ | 467 |
| p6 | CTNNB1 | S45P | TTTAPPLSG | 48 | ~~~APPLS~ | 468 |
| p5 | CTNNB1 | S45P | TTAPPLSGK | 49 | ~~~PPLSG~ | 469 |
| p4 | CTNNB1 | S45P | TAPPLSGKG | 50 | ~~~PLSGK~ | 470 |
| p8 | CTNNB1 | T41A | GIHSGATAT | 51 | ~~~SGATA~ | 471 |
| p7 | CTNNB1 | T41A | IHSGATATA | 52 | ~~~GATAT~ | 472 |
| p6 | CTNNB1 | T41A | HSGATATAP | 53 | ~~~ATATA~ | 473 |
| p5 | CTNNB1 | T41A | SGATATAPS | 54 | ~~~TATAP~ | 474 |
| p4 | CTNNB1 | T41A | GATATAPSL | 55 | ~~~ATAPS~ | 475 |
| p8 | EGFR | A289V | EGKYSFGVT | 56 | ~~~YSFGV~ | 476 |
| p7 | EGFR | A289V | GKYSFGVTC | 57 | ~~~SFGVT~ | 477 |
| p6 | EGFR | A289V | KYSFGVTCV | 58 | ~~~FGVTC~ | 478 |
| p5 | EGFR | A289V | YSFGVTCVK | 59 | ~~~GVTCV~ | 479 |
| p4 | EGFR | A289V | SFGVTCVKK | 60 | ~~~VTCVK~ | 480 |
| p8 | EGFR | G598V | CVKTCPAVV | 61 | ~~~TCPAV~ | 481 |
| p7 | EGFR | G598V | VKTCPAVVM | 62 | ~~~CPAVV~ | 482 |
| p6 | EGFR | G598V | KTCPAVVMG | 63 | ~~~PAVVM~ | 483 |
| p5 | EGFR | G598V | TCPAVVMGE | 64 | ~~~AVVMG~ | 484 |
| p4 | EGFR | G598V | CPAVVMGEN | 65 | ~~~VVMGE~ | 485 |
| p8 | EGFR | L858R | VKITDFGRA | 66 | ~~~TDFGR~ | 486 |
| p7 | EGFR | L858R | KITDFGRAK | 67 | ~~~DEGRA~ | 487 |
| p6 | EGFR | L858R | ITDFGRAKL | 68 | ~~~FGRAK~ | 488 |
| ps | EGFR | L858R | TDFGRAKLL | 69 | ~~~GRAKL~ | 489 |
| p4 | EGFR | L858R | DFGRAKLLG | 70 | ~~~RAKLL~ | 490 |
| p8 | FBXW7 | R465C | YGHTSTVCC | 71 | ~~~TSTVC~ | 491 |
| p7 | FBXW7 | R465C | GHTSTVCCM | 72 | ~~~STVCC~ | 492 |
| p6 | FBXW7 | R465C | HTSTVCCMH | 73 | ~~~TVCCM~ | 493 |
| p5 | FBXW7 | R465C | TSTVCCMHL | 74 | ~~~VCCMH~ | 494 |
| p4 | FBXW7 | R465C | STVCCMHLH | 75 | ~~~CCMHL~ | 495 |
| p8 | FBXW7 | R465G | YGHTSTVGC | 76 | ~~~TSTVG~ | 496 |
| p7 | FBXW7 | R465G | GHTSTVGCM | 77 | ~~~STVGC~ | 497 |
| p6 | FBXW7 | R465G | HTSTVGCMH | 78 | ~~~TVGCM~ | 498 |
| p5 | FBXW7 | R465G | TSTVGCMHL | 79 | ~~~VGCMH~ | 499 |
| p4 | FBXW7 | R465G | STVGCMHLH | 80 | ~~~GCMHL~ | 500 |
| p8 | FBXW7 | R479Q | KRVVSGSQD | 81 | ~~~VSGSQ~ | 501 |
| p7 | FBXW7 | R479Q | RVVSGSQDA | 82 | ~~~SGSQD~ | 502 |
| p6 | FBXW7 | R479Q | VVSGSQDAT | 83 | ~~~GSQDA~ | 503 |
| p5 | FBXW7 | R479Q | VSGSQDATL | 84 | ~~~SQDAT~ | 504 |
| p4 | FBXW7 | R479Q | SGSQDATLR | 85 | ~~~QDATL~ | 505 |
| p8 | FBXW7 | R505C | MGHVAAVCC | 86 | ~~~VAAVC~ | 506 |
| p7 | FBXW7 | R505C | GHVAAVCCV | 87 | ~~~AAVCC~ | 507 |
| p6 | FBXW7 | R505C | HVAAVCCVQ | 88 | ~~~AVCCV~ | 508 |
| p5 | FBXW7 | R505C | VAAVCCVQY | 89 | ~~~VCCVQ~ | 509 |
| p4 | FBXW7 | R505C | AAVCCVQYD | 90 | ~~~CCVQY~ | 510 |
| p8 | FBXW7 | R505G | MGHVAAVGC | 91 | ~~~VAAVG~ | 511 |
| p7 | FBXW7 | R505G | GHVAAVGCV | 92 | ~~~AAVGC~ | 512 |
| p6 | FBXW7 | R505G | HVAAVGCVQ | 93 | ~~~AVGCV~ | 513 |
| p5 | FBXW7 | R505G | VAAVGCVQY | 94 | ~~~VGCVQ~ | 514 |
| p4 | FBXW7 | R505G | AAVGCVQYD | 95 | ~~~GCVQY~ | 515 |
| p8 | FGFR2 | S252W | HLDVVERWP | 96 | ~~~VVERW~ | 516 |
| p7 | FGFR2 | S252W | LDVVERWPH | 97 | ~~~VERWP~ | 517 |
| p6 | FGFR2 | S252W | DVVERWPHR | 98 | ~~~ERWPH~ | 518 |
| p3 | FGFR2 | S252W | VVERWPHRP | 99 | ~~~RWPHR~ | 519 |
| p4 | FGFR2 | S252W | VERWPHRPI | 100 | ~~~WPHRP~ | 520 |
| p8 | FGFR3 | S249C | TLDVLERCP | 101 | ~~~VLERC~ | 521 |
| p7 | FGFR3 | S249C | LDVLERCPH | 102 | ~~~LERCP~ | 522 |
| p6 | FGFR3 | S249C | DVLERCPHR | 103 | ~~~ERCPH~ | 523 |
| p5 | FGFR3 | S249C | VLERCPHRP | 104 | ~~~RCPHR~ | 524 |
| p4 | FGFR3 | S249C | LERCPHRPI | 105 | ~~~CPHRP~ | 525 |
| p8 | GNA | Q209L | RMVDVGGLR | 106 | ~~~DVGGL~ | 526 |
| p7 | GNA | Q209L | MVDVGGLRS | 107 | ~~~VGGLR~ | 527 |
| p6 | GNA | Q209L | VDVGGLRSE | 108 | ~~~GGLRS~ | 528 |
| p5 | GNA | Q209L | DVGGLRSER | 109 | ~~~GLRSE~ | 529 |
| p4 | GNA | Q209L | VGGLRSERR | 110 | ~~~LRSER~ | 530 |
| p8 | GNAQ | Q209L | RMVDVGGLR | 111 | ~~~DVGGL~ | 531 |
| p7 | GNAQ | Q209L | MVDVGGLRS | 112 | ~~~VGGLR~ | 532 |
| p6 | GNAQ | Q209L | VDVGGLRSE | 113 | ~~~GGLRS~ | 533 |
| p5 | GNAQ | Q209L | DVGGLRSER | 114 | ~~~GLRSE~ | 534 |
| p4 | GNAQ | Q209L | VGGLRSERR | 115 | ~~~LRSER~ | 535 |
| p8 | GNAQ | Q209P | RMVDVGGPR | 116 | ~~~DVGGP~ | 536 |
| p7 | GNAQ | Q209P | MVDVGGPRS | 117 | ~~~VGGPR~ | 537 |
| p6 | GNAQ | Q209P | VDVGGPRSE | 118 | ~~~GGPRS~ | 538 |
| p5 | GNAQ | Q209P | DVGGPRSER | 119 | ~~~GPRSE~ | 539 |
| p4 | GNAQ | Q209P | VGGPRSERR | 120 | ~~~PRSER~ | 540 |
| p8 | GNAS | R844C | DQDLLRCCV | 121 | ~~~LLRCC~ | 541 |
| p7 | GNAS | R844C | QDLLRCCVL | 122 | ~~~LRCCV~ | 542 |
| po | GNAS | R844C | DLLRCCVLT | 123 | ~~~RCCVL~ | 543 |
| p5 | GNAS | R844C | LLRCCVLTS | 124 | ~~~CCVLT~ | 544 |
| p4 | GNAS | R844C | LRCCVLTSG | 125 | ~~~CVLTS~ | 545 |
| p8 | HRAS | Q61R | DILDTAGRE | 126 | ~~~DTAGR~ | 546 |
| p7 | HRAS | Q61R | ILDTAGREE | 127 | ~~~TAGRE~ | 547 |
| p6 | HRAS | Q61R | LDTAGREEY | 128 | ~~~AGREE~ | 548 |
| ps | HRAS | 061R | DTAGREEYS | 129 | ~~~GREEY~ | 549 |
| p4 | HRAS | Q61R | TAGREEYSA | 130 | ~~~REEYS~ | 550 |
| p8 | KRAS | A146T | IPFIETSTK | 131 | ~~~IETST~ | 551 |
| p7 | KRAS | A146T | PFIETSTKT | 132 | ~~~ETSTK~ | 552 |
| p6 | KRAS | A146T | FIETSTKTR | 133 | ~~~TSTKT~ | 553 |
| p5 | KRAS | A146T | IETSTKTRQ | 134 | ~~~STKTR~ | 554 |
| p4 | KRAS | A146T | ETSTKTRQR | 135 | ~~~TKTRQ~ | 555 |
| p8 | KRAS | G12A | KLVVVGAAG | 136 | ~~~VVGAA~ | 556 |
| p7 | KRAS | G12A | LVVVGAAGV | 137 | ~~~VGAAG~ | 557 |
| p6 | KRAS | G12A | VVVGAAGVG | 138 | ~~~GAAGV~ | 558 |
| p5 | KRAS | G12A | VVGAAGVGK | 139 | ~~~AAGVG~ | 559 |
| p4 | KRAS | G12A | VGAAGVGKS | 140 | ~~~AGVGK~ | 560 |
| p8 | KRAS | G12C | KLVVVGACG | 141 | ~~~VVGAC~ | 561 |
| p7 | KRAS | G12C | LVVVGACGV | 142 | ~~~VGACG~ | 562 |
| p6 | KRAS | G12C | VVVGACGVG | 143 | ~~~GACGV~ | 563 |
| p5 | KRAS | G12C | VVGACGVGK | 144 | ~~~ACGVG~ | 564 |
| p4 | KRAS | G12C | VGACGVGKS | 145 | ~~~CGVGK~ | 565 |
| p8 | KRAS | G12D | KLVVVGADG | 146 | ~~~VVGAD~ | 566 |
| p7 | KRAS | G12D | LVVVGADGV | 147 | ~~~VGADG~ | 567 |
| p6 | KRAS | G12D | VVVGADGVG | 148 | ~~~GADGV~ | 568 |
| p5 | KRAS | G12D | VVGADGVGK | 149 | ~~~ADGVG~ | 569 |
| p4 | KRAS | G12D | VGADGVGKS | 150 | ~~~DGVGK~ | 570 |
| p8 | KRAS | G12R | KLVVVGARG | 151 | ~~~VVGAR~ | 571 |
| p7 | KRAS | G12R | LVVVGARGV | 152 | ~~~VGARG~ | 572 |
| p6 | KRAS | G12R | VVVGARGVG | 153 | ~~~GARGV~ | 573 |
| p5 | KRAS | G12R | VVGARGVGK | 154 | ~~~ARGVG~ | 574 |
| p4 | KRAS | G12R | VGARGVGKS | 155 | ~~~RGVGK~ | 575 |
| p8 | KRAS | G12S | KLVVVGASG | 156 | ~~~VVGAS~ | 576 |
| p7 | KRAS | G12S | LVVVGASGV | 157 | ~~~VGASG~ | 577 |
| p6 | KRAS | G12S | VVVGASGVG | 158 | ~~~GASGV~ | 578 |
| p5 | KRAS | G12S | VVGASGVGK | 159 | ~~~ASGVG~ | 579 |
| p4 | KRAS | G12S | VGASGVGKS | 160 | ~~~SGVGK~ | 580 |
| p8 | KRAS | G12V | KLVVVGAVG | 161 | ~~~VVGAV~ | 581 |
| p7 | KRAS | G12V | LVVVGAVGV | 162 | ~~~VGAVG~ | 582 |
| p6 | KRAS | G12V | VVVGAVGVG | 163 | ~~~GAVGV~ | 583 |
| p5 | KRAS | G12V | VVGAVGVGK | 164 | ~~~AVGVG~ | 584 |
| p4 | KRAS | G12V | VGAVGVGKS | 165 | ~~~VGVGK~ | 585 |
| p8 | KRAS | G13C | LVVVGAGCV | 166 | ~~~VGAGC~ | 586 |
| p7 | KRAS | G13C | VVVGAGCVG | 167 | ~~~GAGCV~ | 587 |
| po | KRAS | G13C | VVGAGCVGK | 168 | ~~~AGCVG~ | 588 |
| p5 | KRAS | G13C | VGAGCVGKS | 169 | ~~~GCVGK~ | 589 |
| p4 | KRAS | G13C | GAGCVGKSA | 170 | ~~~CVGKS~ | 590 |
| p8 | KRAS | G13D | LVVVGAGDV | 171 | ~~~VGAGD~ | 591 |
| p7 | KRAS | G13D | VVVGAGDVG | 172 | ~~~GAGDV~ | 592 |
| p6 | KRAS | G13D | VVGAGDVGK | 173 | ~~~AGDVG~ | 593 |
| p5 | KRAS | G13D | VGAGDVGKS | 174 | ~~~GDVGK~ | 594 |
| p4 | KRAS | G13D | GAGDVGKSA | 175 | ~~~DVGKS~ | 595 |
| p8 | KRAS | Q61H | DILDTAGHE | 176 | ~~~DTAGH~ | 596 |
| p7 | KRAS | Q61H | ILDTAGHEE | 177 | ~~~TAGHE~ | 597 |
| p6 | KRAS | Q61H | LDTAGHEEY | 178 | ~~~AGHEE~ | 598 |
| p5 | KRAS | 061H | DTAGHEEYS | 179 | ~~~GHEEY~ | 599 |
| p4 | KRAS | Q61H | TAGHEEYSA | 180 | ~~~HEEYS~ | 600 |
| p8 | KRAS | Q61H | DILDTAGHE | 181 | ~~~DTAGH~ | 601 |
| p7 | KRAS | Q61H | ILDTAGHEE | 182 | ~~~TAGHE~ | 602 |
| p6 | KRAS | Q61H | LDTAGHEEY | 183 | ~~~AGHEE~ | 603 |
| p5 | KRAS | Q61H | DTAGHEEYS | 184 | ~~~GHEEY~ | 604 |
| p4 | KRAS | Q61H | TAGHEEYSA | 185 | ~~~HEEYS~ | 605 |
| p8 | KRAS | Q61L | DILDTAGLE | 186 | ~~~DTAGL~ | 606 |
| p7 | KRAS | Q61L | ILDTAGLEE | 187 | ~~~TAGLE~ | 607 |
| p6 | KRAS | Q61L | LDTAGLEEY | 188 | ~~~AGLEE~ | 608 |
| p5 | KRAS | Q61L | DTAGLEEYS | 189 | ~~~GLEEY~ | 609 |
| p4 | KRAS | Q61L | TAGLEEYSA | 190 | ~~~LEEYS~ | 610 |
| p8 | NRAS | G12D | KLVVVGADG | 191 | ~~~VVGAD~ | 611 |
| p7 | NRAS | G12D | LVVVGADGV | 192 | ~~~VGADG~ | 612 |
| po | NRAS | G12D | VVVGADGVG | 193 | ~~~GADGV~ | 613 |
| p5 | NRAS | G12D | VVGADGVGK | 194 | ~~~ADGVG~ | 614 |
| p4 | NRAS | G12D | VGADGVGKS | 195 | ~~~DGVGK~ | 615 |
| p8 | NRAS | G13D | LVVVGAGDV | 196 | ~~~VGAGD~ | 616 |
| p7 | NRAS | G13D | VVVGAGDVG | 197 | ~~~GAGDV~ | 617 |
| p6 | NRAS | G13D | VVGAGDVGK | 198 | ~~~AGDVG~ | 618 |
| p5 | NRAS | G13D | VGAGDVGKS | 199 | ~~~GDVGK~ | 619 |
| p4 | NRAS | G13D | GAGDVGKSA | 200 | ~~~DVGKS~ | 620 |
| p8 | NRAS | G13R | LVVVGAGRV | 201 | ~~~VGAGR~ | 621 |
| p7 | NRAS | G13R | VVVGAGRVG | 202 | ~~~GAGRV~ | 622 |
| p6 | NRAS | G13R | VVGAGRVGK | 203 | ~~~AGRVG~ | 623 |
| p5 | NRAS | G13R | VGAGRVGKS | 204 | ~~~GRVGK~ | 624 |
| p4 | NRAS | G13R | GAGRVGKSA | 205 | ~~~RVGKS~ | 625 |
| p8 | NRAS | Q61K | DILDTAGKE | 206 | ~~~DTAGK~ | 626 |
| p7 | NRAS | Q61K | ILDTAGKEE | 207 | ~~~TAGKE~ | 627 |
| p6 | NRAS | Q61K | LDTAGKEEY | 208 | ~~~AGKEE~ | 628 |
| p5 | NRAS | Q61K | DTAGKEEYS | 209 | ~~~GKEEY~ | 629 |
| p4 | NRAS | Q61K | TAGKEEYSA | 210 | ~~~KEEYS~ | 630 |
| p8 | NRAS | Q61L | DILDTAGLE | 211 | ~~~DTAGL~ | 631 |
| p7 | NRAS | Q61L | ILDTAGLEE | 212 | ~~~TAGLE~ | 632 |
| p6 | NRAS | Q61L | LDTAGLEEY | 213 | ~~~AGLEE~ | 633 |
| p5 | NRAS | Q61L | DTAGLEEYS | 214 | ~~~GLEEY~ | 634 |
| p4 | NRAS | Q61L | TAGLEEYSA | 215 | ~~~LEEYS~ | 635 |
| p8 | PPP2R1A | P179R | NLCSDDTRM | 216 | ~~~SDDTR~ | 636 |
| p7 | PPP2R1A | P179R | LCSDDTRMV | 217 | ~~~DDTRM~ | 637 |
| p6 | PPP2R1A | P179R | CSDDTRMVR | 218 | ~~~DTRMV~ | 638 |
| p5 | PPP2R1A | P179R | SDDTRMVRR | 219 | ~~~TRMVR~ | 639 |
| p4 | PPP2R1A | P179R | DDTRMVRRA | 220 | ~~~RMVRR~ | 640 |
| p8 | PPP2R1A | R183W | DDTPMVRWA | 221 | ~~~PMVRW~ | 641 |
| p7 | PPP2R1A | R183W | DTPMVRWAA | 222 | ~~~MVRWA~ | 642 |
| p6 | PPP2R1A | R183W | TPMVRWAAA | 223 | ~~~VRWAA~ | 643 |
| po | PPP2R1A | R183W | PMVRWAAAS | 224 | ~~~RWAAA~ | 644 |
| p4 | PPP2R1A | R183W | MVRWAAASK | 225 | ~~~WAAAS~ | 645 |
| p8 | PTEN | R130G | HCKAGKGGT | 226 | ~~~AGKGG~ | 646 |
| p7 | PTEN | R130G | CKAGKGGTG | 227 | ~~~GKGGT~ | 647 |
| p6 | PTEN | R130G | KAGKGGTGV | 228 | ~~~KGGTG~ | 648 |
| p5 | PTEN | R130G | AGKGGTGVM | 229 | ~~~GGTGV~ | 649 |
| p4 | PTEN | R130G | GKGGTGVMI | 230 | ~~~GTGVM~ | 650 |
| p8 | PTEN | R130Q | HCKAGKGQT | 231 | ~~~AGKGQ~ | 651 |
| p7 | PTEN | R130Q | CKAGKGQTG | 232 | ~~~GKGQT~ | 652 |
| p6 | PTEN | R130Q | KAGKGQTGV | 233 | ~~~KGQTG~ | 653 |
| p5 | PTEN | R130Q | AGKGQTGVM | 234 | ~~~GQTGV~ | 654 |
| p4 | PTEN | R130Q | GKGQTGVMI | 235 | ~~~QTGVM~ | 655 |
| p8 | SMAD4 | R361H | VDPSGGDHF | 236 | ~~~SGGDH~ | 656 |
| p7 | SMAD4 | R361H | DPSGGDHFC | 237 | ~~~GGDHF~ | 657 |
| p6 | SMAD4 | R361H | PSGGDHFCL | 238 | ~~~GDHFC~ | 658 |
| p5 | SMAD4 | R361H | SGGDHFCLG | 239 | ~~~DHFCL~ | 659 |
| p4 | SMAD4 | R361H | GGDHFCLGQ | 240 | ~~~HFCLG~ | 660 |
| p8 | TP53 | C176F | MTEVVRRFP | 241 | ~~~VVRRF~ | 661 |
| p7 | TP53 | C176F | TEVVRRFPH | 242 | ~~~VRRFP~ | 662 |
| p6 | TP53 | C176F | EVVRRFPHH | 243 | ~~~RRFPH~ | 663 |
| p5 | TP53 | C176F | VVRRFPHHE | 244 | ~~~RFPHH~ | 664 |
| p4 | TP53 | C176F | VRRFPHHER | 245 | ~~~FPHHE~ | 665 |
| p8 | TP53 | C176Y | MTEVVRRYP | 246 | ~~~VVRRY~ | 666 |
| p7 | TP53 | C176Y | TEVVRRYPH | 247 | ~~~VRRYP~ | 667 |
| p6 | TP53 | C176Y | EVVRRYPHH | 248 | ~~~RRYPH~ | 668 |
| p5 | TP53 | C176Y | VVRRYPHHE | 249 | ~~~RYPHH~ | 669 |
| p4 | TP53 | C176Y | VRRYPHHER | 250 | ~~~YPHHE~ | 670 |
| p8 | TP53 | C238Y | TIHYNYMYN | 251 | ~~~YNYMY~ | 671 |
| p7 | TP53 | C238Y | IHYNYMYNS | 252 | ~~~NYMYN~ | 672 |
| p6 | TP53 | C238Y | HYNYMYNSS | 253 | ~~~YMYNS~ | 673 |
| p5 | TP53 | C238Y | YNYMYNSSC | 254 | ~~~MYNSS~ | 674 |
| p4 | TP53 | C238Y | NYMYNSSCM | 255 | ~~~YNSSC~ | 675 |
| p8 | TP53 | C275Y | NSFEVRVYA | 256 | ~~~EVRVY~ | 676 |
| p7 | TP53 | C275Y | SFEVRVYAC | 257 | ~~~VRVYA~ | 677 |
| p6 | TP53 | C275Y | FEVRVYACP | 258 | ~~~RVYAC~ | 678 |
| p5 | TP53 | C275Y | EVRVYACPG | 259 | ~~~VYACP~ | 679 |
| p4 | TP53 | C275Y | VRVYACPGR | 260 | ~~~YACPG~ | 680 |
| p8 | TP53 | E285K | PGRDRRTKE | 261 | ~~~DRRTK~ | 681 |
| p7 | TP53 | E285K | GRDRRTKEE | 262 | ~~~RRTKE~ | 682 |
| p6 | TP53 | E285K | RDRRTKEEN | 263 | ~~~RTKEE~ | 683 |
| p5 | TP53 | E285K | DRRTKEENL | 264 | ~~~TKEEN~ | 684 |
| p4 | TP53 | E285K | RRTKEENLR | 265 | ~~~KEENL~ | 685 |
| p8 | TP53 | E286K | GRDRRTEKE | 266 | ~~~RRTEK~ | 686 |
| p7 | TP53 | E286K | RDRRTEKEN | 267 | ~~~RTEKE~ | 687 |
| p6 | TP53 | E286K | DRRTEKENL | 268 | ~~~TEKEN~ | 688 |
| p5 | TP53 | E286K | RRTEKENLR | 269 | ~~~EKENL~ | 689 |
| p4 | TP53 | E286K | RTEKENLRK | 270 | ~~~KENLR~ | 690 |
| p8 | TP53 | G245D | CNSSCMGDM | 271 | ~~~SCMGD~ | 691 |
| p7 | TP53 | G245D | NSSCMGDMN | 272 | ~~~CMGDM~ | 692 |
| p6 | TP53 | G245D | SSCMGDMNR | 273 | ~~~MGDMN~ | 693 |
| p5 | TP53 | G245D | SCMGDMNRR | 274 | ~~~GDMNR~ | 694 |
| p4 | TP53 | G245D | CMGDMNRRP | 275 | ~~~DMNRR~ | 695 |
| p8 | TP53 | G245S | CNSSCMGSM | 276 | ~~~SCMGS~ | 696 |
| p7 | TP53 | G245S | NSSCMGSMN | 277 | ~~~CMGSM~ | 697 |
| p6 | TP53 | G245S | SSCMGSMNR | 278 | ~~~MGSMN~ | 698 |
| p5 | TP53 | G245S | SCMGSMNRR | 279 | ~~~GSMNR~ | 699 |
| p4 | TP53 | G245S | CMGSMNRRP | 280 | ~~~SMNRR~ | 700 |
| p8 | TP53 | G245V | CNSSCMGVM | 281 | ~~~SCMGV~ | 701 |
| p7 | TP53 | G245V | NSSCMGVMN | 282 | ~~~CMGVM~ | 702 |
| p6 | TP53 | G245V | SSCMGVMNR | 283 | ~~~MGVMN~ | 703 |
| p5 | TP53 | G245V | SCMGVMNRR | 284 | ~~~GVMNR~ | 704 |
| p4 | TP53 | G245V | CMGVMNRRP | 285 | ~~~VMNRR~ | 705 |
| p8 | TP53 | H179R | VVRRCPHRE | 286 | ~~~RCPHR~ | 706 |
| p7 | TP53 | H179R | VRRCPHRER | 287 | ~~~CPHRE~ | 707 |
| p6 | TP53 | H179R | RRCPHRERC | 288 | ~~~PHRER~ | 708 |
| p5 | TP53 | H179R | RCPHRERCS | 289 | ~~~HRERC~ | 709 |
| p4 | TP53 | H179R | CPHRERCSD | 290 | ~~~RERCS~ | 710 |
| p8 | TP53 | H179Y | VVRRCPHYE | 291 | ~~~RCPHY~ | 711 |
| p7 | TP53 | H179Y | VRRCPHYER | 292 | ~~~CPHYE~ | 712 |
| p6 | TP53 | H179Y | RRCPHYERC | 293 | ~~~PHYER~ | 713 |
| p5 | TP53 | H179Y | RCPHYERCS | 294 | ~~~HYERC~ | 714 |
| p4 | TP53 | H179Y | CPHYERCSD | 295 | ~~~YERCS~ | 715 |
| p8 | TP53 | H193R | DGLAPPQRL | 296 | ~~~APPQR~ | 716 |
| p7 | TP53 | H193R | GLAPPQRLI | 297 | ~~~PPQRL~ | 717 |
| p6 | TP53 | H193R | LAPPQRLIR | 298 | ~~~PQRLI~ | 718 |
| p5 | TP53 | H193R | APPQRLIRV | 299 | ~~~QRLIR~ | 719 |
| p4 | TP53 | H193R | PPQRLIRVE | 300 | ~~~RLIRV~ | 720 |
| p8 | TP53 | I195T | LAPPQHLTR | 301 | ~~~PQHLT~ | 721 |
| p7 | TP53 | I195T | APPQHLTRV | 302 | ~~~QHLTR~ | 722 |
| p6 | TP53 | I195T | PPQHLTRVE | 303 | ~~~HLTRV~ | 723 |
| p5 | TP53 | I195T | PQHLTRVEG | 304 | ~~~LTRVE~ | 724 |
| p4 | TP53 | I195T | QHLTRVEGN | 305 | ~~~TRVEG~ | 725 |
| p8 | TP53 | L194R | GLAPPQHRI | 306 | ~~~PPQHR~ | 726 |
| p7 | TP53 | L194R | LAPPQHRIR | 307 | ~~~PQHRI~ | 727 |
| p6 | TP53 | L194R | APPQHRIRV | 308 | ~~~QHRIR~ | 728 |
| p5 | TP53 | L194R | PPQHRIRVE | 309 | ~~~HRIRV~ | 729 |
| p4 | TP53 | L194R | PQHRIRVEG | 310 | ~~~RIRVE~ | 730 |
| p8 | TP53 | P151S | QLWVDSTSP | 311 | ~~~VDSTS~ | 731 |
| p7 | TP53 | P151S | LWVDSTSPP | 312 | ~~~DSTSP~ | 732 |
| p6 | TP53 | P151S | WVDSTSPPG | 313 | ~~~STSPP~ | 733 |
| p5 | TP53 | P151S | VDSTSPPGT | 314 | ~~~TSPPG~ | 734 |
| p4 | TP53 | P151S | DSTSPPGTR | 315 | ~~~SPPGT~ | 735 |
| p8 | TP53 | R158H | PPPGTRVHA | 316 | ~~~GTRVH~ | 736 |
| p7 | TP53 | R158H | PPGTRVHAM | 317 | ~~~TRVHA~ | 737 |
| p6 | TP53 | R158H | PGTRVHAMA | 318 | ~~~RVHAM~ | 738 |
| p5 | TP53 | R158H | GTRVHAMAI | 319 | ~~~VHAMA~ | 739 |
| p4 | TP53 | R158H | TRVHAMAIY | 320 | ~~~HAMAI~ | 740 |
| p8 | TP53 | R158L | PPPGTRVLA | 321 | ~~~GTRVL~ | 741 |
| p7 | TP53 | R158L | PPGTRVLAM | 322 | ~~~TRVLA~ | 742 |
| po | TP53 | R158L | PGTRVLAMA | 323 | ~~~RVLAM~ | 743 |
| p | TP53 | R158L | GTRVLAMAI | 324 | ~~~VLAMA~ | 744 |
| p4 | TP53 | R158L | TRVLAMAIY | 325 | ~~~LAMAI~ | 745 |
| p8 | TP53 | R175H | HMTEVVRHC | 326 | ~~~EVVRH~ | 746 |
| p7 | TP53 | R175H | MTEVVRHCP | 327 | ~~~VVRHC~ | 747 |
| p6 | TP53 | R175H | TEVVRHCPH | 328 | ~~~VRHCP~ | 748 |
| p5 | TP53 | R175H | EVVRHCPHH | 329 | ~~~RHCPH~ | 749 |
| p4 | TP53 | R175H | VVRHCPHHE | 330 | ~~~HCPHH~ | 750 |
| 08 | TP53 | R248Q | SCMGGMNQR | 331 | ~~~GGMNQ~ | 751 |
| p7 | TP53 | R248Q | CMGGMNQRP | 332 | ~~~GMNQR~ | 752 |
| p6 | TP53 | R248Q | MGGMNQRPI | 333 | ~~~MNQRP~ | 753 |
| p5 | TP53 | R248Q | GGMNQRPIL | 334 | ~~~NQRPI~ | 754 |
| p4 | TP53 | R248Q | GMNQRPILT | 335 | ~~~QRPIL~ | 755 |
| p8 | TP53 | R248W | SCMGGMNWR | 336 | ~~~GGMNW~ | 756 |
| p7 | TP53 | R248W | CMGGMNWRP | 337 | ~~~GMNWR~ | 757 |
| p6 | TP53 | R248W | MGGMNWRPI | 338 | ~~~MNWRP~ | 758 |
| ps | TP53 | R248W | GGMNWRPIL | 339 | ~~~NWRPI~ | 759 |
| p4 | TP53 | R248W | GMNWRPILT | 340 | ~~~WRPIL~ | 760 |
| p8 | TP53 | R249S | CMGGMNRSP | 341 | ~~~GMNRS~ | 761 |
| p7 | TP53 | R249S | MGGMNRSPI | 342 | ~~~MNRSP~ | 762 |
| p6 | TP53 | R249S | GGMNRSPIL | 343 | ~~~NRSPI~ | 763 |
| p5 | TP53 | R249S | GMNRSPILT | 344 | ~~~RSPIL~ | 764 |
| p4 | TP53 | R249S | MNRSPILTI | 345 | ~~~SPILT~ | 765 |
| p8 | TP53 | R249S | CMGGMNRSP | 346 | ~~~GMNRS~ | 766 |
| p7 | TP53 | R249S | MGGMNRSPI | 347 | ~~~MNRSP~ | 767 |
| p6 | TP53 | R249S | GGMNRSPIL | 348 | ~~~NRSPI~ | 768 |
| p5 | TP53 | R249S | GMNRSPILT | 349 | ~~~RSPIL~ | 769 |
| p4 | TP53 | R249S | MNRSPILTI | 350 | ~~~SPILT~ | 770 |
| p8 | TP53 | R273C | GRNSFEVCV | 351 | ~~~SFEVC~ | 771 |
| p7 | TP53 | R273C | RNSFEVCVC | 352 | ~~~FEVCV~ | 772 |
| p6 | TP53 | R273C | NSFEVCVCA | 353 | ~~~EVCVC~ | 773 |
| p5 | TP53 | R273C | SFEVCVCAC | 354 | ~~~VCVCA~ | 774 |
| p4 | TP53 | R273C | FEVCVCACP | 355 | ~~~CVCAC~ | 775 |
| p8 | TP53 | R273H | GRNSFEVHV | 356 | ~~~SFEVH~ | 776 |
| p7 | TP53 | R273H | RNSFEVHVC | 357 | ~~~FEVHV~ | 777 |
| p6 | TP53 | R273H | NSFEVHVCA | 358 | ~~~EVHVC~ | 778 |
| p5 | TP53 | R273H | SFEVHVCAC | 359 | ~~~VHVCA~ | 779 |
| p4 | TP53 | R273H | FEVHVCACP | 360 | ~~~HVCAC~ | 780 |
| p8 | TP53 | R273L | GRNSFEVLV | 361 | ~~~SFEVL~ | 781 |
| p7 | TP53 | R273L | RNSFEVLVC | 362 | ~~~FEVLV~ | 782 |
| p6 | TP53 | R273L | NSFEVLVCA | 363 | ~~~EVLVC~ | 783 |
| p5 | TP53 | R273L | SFEVLVCAC | 364 | ~~~VLVCA~ | 784 |
| p4 | TP53 | R273L | FEVLVCACP | 365 | ~~~LVCAC~ | 785 |
| p8 | TP53 | R280K | RVCACPGKD | 366 | ~~~ACPGK~ | 786 |
| p7 | TP53 | R280K | VCACPGKDR | 367 | ~~~CPGKD~ | 787 |
| p6 | TP53 | R280K | CACPGKDRR | 368 | ~~~PGKDR~ | 788 |
| p5 | TP53 | R280K | ACPGKDRRT | 369 | ~~~GKDRR~ | 789 |
| p4 | TP53 | R280K | CPGKDRRTE | 370 | ~~~KDRRT~ | 790 |
| p8 | TP53 | R280T | RVCACPGTD | 371 | ~~~ACPGT~ | 791 |
| p7 | TP53 | R280T | VCACPGTDR | 372 | ~~~CPGTD~ | 792 |
| p6 | TP53 | R280T | CACPGTDRR | 373 | ~~~PGTDR~ | 793 |
| p5 | TP53 | R280T | ACPGTDRRT | 374 | ~~~GTDRR~ | 794 |
| p4 | TP53 | R280T | CPGTDRRTE | 375 | ~~~TQRRT~ | 795 |
| p8 | TP53 | R282W | CACPGRDWR | 376 | ~~~PGRDW~ | 796 |
| p7 | TP53 | R282W | ACPGRDWRT | 377 | ~~~GRDWR~ | 797 |
| p6 | TP53 | R282W | CPGRDWRTE | 378 | ~~~RDWRT~ | 798 |
| p5 | TP53 | R282W | PGRDWRTEE | 379 | ~~~DWRTE~ | 799 |
| p4 | TP53 | R282W | GRDWRTEEE | 380 | ~~~WRTEE~ | 800 |
| p8 | TP53 | S241F | YNYMCNSFC | 381 | ~~~MCNSF~ | 801 |
| p7 | TP53 | S241F | NYMCNSFCM | 382 | ~~~CNSFC~ | 802 |
| p6 | TP53 | S241F | YMCNSFCMG | 383 | ~~~NSFCM~ | 803 |
| p5 | TP53 | S241F | MCNSFCMGG | 384 | ~~~SFCMG~ | 804 |
| p4 | TP53 | S241F | CNSFCMGGM | 385 | ~~~FCMGG~ | 805 |
| p8 | TP53 | V157F | TPPPGTRFR | 386 | ~~~PGTRF~ | 806 |
| p7 | TP53 | V157F | PPPGTRFRA | 387 | ~~~GTRFR~ | 807 |
| p6 | TP53 | V157F | PPGTRFRAM | 388 | ~~~TRFRA~ | 808 |
| p5 | TP53 | V157F | PGTRFRAMA | 389 | ~~~RFRAM~ | 809 |
| p4 | TP53 | V157F | GTRFRAMAI | 390 | ~~~FRAMA~ | 810 |
| p8 | TP53 | V173M | SQHMTEVMR | 391 | ~~~MTEVM~ | 811 |
| p7 | TP53 | V173M | QHMTEVMRR | 392 | ~~~TEVMR~ | 812 |
| p6 | TP53 | V173M | HMTEVMRRC | 393 | ~~~EVMRR~ | 813 |
| p5 | TP53 | V173M | MTEVMRRCP | 394 | ~~~VMRRC~ | 814 |
| p4 | TP53 | V173M | TEVMRRCPH | 395 | ~~~MRRCP~ | 815 |
| p8 | TP53 | V272M | LGRNSFEMR | 396 | ~~~NSFEM~ | 816 |
| p7 | TP53 | V272M | GRNSFEMRV | 397 | ~~~SFEMR~ | 817 |
| p6 | TP53 | V272M | RNSFEMRVC | 398 | ~~~FEMRV~ | 818 |
| p5 | TP53 | V272M | NSFEMRVCA | 399 | ~~~EMRVC~ | 819 |
| p4 | TP53 | V272M | SFEMRVCAC | 400 | ~~~MRVCA~ | 820 |
| p8 | TP53 | Y163C | RVRAMAICK | 401 | ~~~AMAIC~ | 821 |
| p7 | TP53 | Y163C | VRAMAICKQ | 402 | ~~~MAICK~ | 822 |
| po | TP53 | Y163C | RAMAICKQS | 403 | ~~~AICKQ~ | 823 |
| p5 | TP53 | Y163C | AMAICKQSQ | 404 | ~~~ICKQS~ | 824 |
| p4 | TP53 | Y163C | MAICKQSQH | 405 | ~~~CKQSQ~ | 825 |
| p8 | TP53 | Y205C | EGNLRVECL | 406 | ~~~LRVEC~ | 826 |
| p7 | TP53 | Y205C | GNLRVECLD | 407 | ~~~RVECL~ | 827 |
| p6 | TP53 | Y205C | NLRVECLDD | 408 | ~~~VECLD~ | 828 |
| p5 | TP53 | Y205C | LRVECLDDR | 409 | ~~~ECLDD~ | 829 |
| p4 | TP53 | Y205C | RVECLDDRN | 410 | ~~~CLDDR~ | 830 |
| p8 | TP53 | Y220C | RHSVVVPCE | 411 | ~~~VVVPC~ | 831 |
| p7 | TP53 | Y220C | HSVVVPCEP | 412 | ~~~VVPCE~ | 832 |
| p6 | TP53 | Y220C | SVVVPCEPP | 413 | ~~~VPCEP~ | 833 |
| p5 | TP53 | Y220C | VVVPCEPPE | 414 | ~~~PCEPP~ | 834 |
| p4 | TP53 | Y220C | VVPCEPPEV | 415 | ~~~CEPPE~ | 835 |
| p8 | TP53 | Y234C | SDCTTIHCN | 416 | ~~~TTIHC~ | 836 |
| p7 | TP53 | Y234C | DCTTIHCNY | 417 | ~~~TIHCN~ | 837 |
| p6 | TP53 | Y234C | CTTIHCNYM | 418 | ~~~IHCNY~ | 838 |
| p5 | TP53 | Y234C | TTIHCNYMC | 419 | ~~~HCNYM~ | 839 |
| p4 | TP53 | Y234C | TIHCNYMCN | 420 | ~~~CNYMC~ | 840 |

TABLE 3
Peptides and T cell exposed motifs presented by MHC II to
T cells from common mutations

| Mutant | | | | | | |
|---|---|---|---|---|---|---|
| aa | | Mutant | | SEQ | | SEQ |
| position | driver | aa | 15 mer mutated | ID | | ID |
| in 9 mer | protein | change | peptide | NO.: | TCEM II | NO.: |
| p8 | AKT | E17K | VKEGWLHKRGKYIKT | 841 | WL~K~GK | 1261 |
| p7 | AKT | E17K | KEGWLHKRGKYIKTW | 842 | LH~R~KY | 1262 |
| p5 | AKT | E17K | GWLHKRGKYIKTWRP | 843 | KR~K~IK | 1263 |
| p3 | AKT | E17K | LHKRGKYIKTWRPRY | 844 | GK~I~TW | 1264 |
| p2 | AKT | E17K | HKRGKYIKTWRPRYF | 845 | KY~K~WR | 1265 |
| p8 | ATM | R337C | GSRGKYSSGFCNIAV | 846 | KY~S~FC | 1266 |
| p7 | ATM | R337C | SRGKYSSGFCNIAVK | 847 | YS~G~CN | 1267 |
| p5 | ATM | R337C | GKYSSGFCNIAVKEN | 848 | SG~C~IA | 1268 |
| p3 | ATM | R337C | YSSGFCNIAVKENLI | 849 | FC~I~VK | 1269 |
| p2 | ATM | R337C | SSGFCNIAVKENLIE | 850 | CN~A~KE | 1270 |
| p8 | BCOR | N1459S | MPPEARRLIVSKNAG | 851 | AR~L~VS | 1271 |
| p7 | BCOR | N1459S | PPEARRLIVSKNAGE | 852 | RR~I~SK | 1272 |
| p5 | BCOR | N1459S | EARRLIVSKNAGETL | 853 | LI~S~NA | 1273 |
| p3 | BCOR | N1459S | RRLIVSKNAGETLLQ | 854 | VS~N~GE | 1274 |
| p2 | BCOR | N1459S | RLIVSKNAGETLLQR | 855 | SK~A~ET | 1275 |
| p8 | BRAF | V640E | VKIGDFGLATEKSRW | 856 | DF~L~TE | 1276 |
| p7 | BRAF | V640E | KIGDFGLATEKSRWS | 857 | FG~A~EK | 1277 |
| p5 | BRAF | V640E | GDFGLATEKSRWSGS | 858 | LA~E~SR | 1278 |
| p3 | BRAF | V640E | FGLATEKSRWSGSHQ | 859 | TE~S~WS | 1279 |
| p2 | BRAF | V640E | GLATEKSRWSGSHQF | 860 | EK~R~SG | 1280 |
| p8 | BRAF | V640M | VKIGDFGLATMKSRW | 861 | DF~L~TM | 1281 |
| p7 | BRAF | V640M | KIGDFGLATMKSRWS | 862 | FG~A~MK | 1282 |
| p5 | BRAF | V640M | GDFGLATMKSRWSGS | 863 | LA~M~SR | 1283 |
| p3 | BRAF | V640M | FGLATMKSRWSGSHQ | 864 | TM~S~WS | 1284 |
| p2 | BRAF | V640M | GLATMKSRWSGSHQF | 865 | MK~R~SG | 1285 |
| p8 | CDKN2A | H83Y | ADPATLTRPVYDAAR | 866 | TL~R~VY | 1286 |
| p7 | CDKN2A | H83Y | DPATLTRPVYDAARE | 867 | LT~P~YD | 1287 |
| p5 | CDKN2A | H83Y | ATLTRPVYDAAREGF | 868 | RP~Y~AA | 1288 |
| p3 | CDKN2A | H83Y | LTRPVYDAAREGFLD | 869 | VY~A~RE | 1289 |
| p2 | CDKN2A | H83Y | TRPVYDAAREGELDT | 870 | YD~A~EG | 1290 |
| p8 | CTNNB1 | S33C | SHWQQQSYLDCGIHS | 871 | QQ~Y~DC | 1291 |
| p7 | CTNNB1 | S33C | HWQQQSYLDCGIHSG | 872 | QS~L~CG | 1292 |
| p5 | CTNNB1 | S33C | QQQSYLDCGIHSGAT | 873 | YL~C~IH | 1293 |
| p3 | CTNNB1 | S33C | QSYLDCGIHSGATTT | 874 | DC~I~SG | 1294 |
| p2 | CTNNB1 | S33C | SYLDCGIHSGATTTA | 875 | CG~H~GA | 1295 |
| p8 | CTNNB1 | S37C | QQSYLDSGIHCGATT | 876 | LD~G~HC | 1296 |
| p7 | CTNNB1 | S37C | QSYLDSGIHCGATTT | 877 | DS~I~CG | 1297 |
| p5 | CTNNB1 | S37C | YLDSGIHCGATTTAP | 878 | GI~C~AT | 1298 |
| p3 | CTNNB1 | S37C | DSGIHCGATTTAPSL | 879 | HC~A~TT | 1299 |
| p2 | CTNNB1 | S37C | SGIHCGATTTAPSLS | 880 | CG~T~TA | 1300 |
| p8 | CTNNB1 | S37F | QQSYLDSGIHFGATT | 881 | LD~G~HF | 1301 |
| p7 | CTNNB1 | S37F | QSYLDSGIHFGATTT | 882 | DS~~~FG | 1302 |
| p5 | CTNNB1 | S37F | YLDSGIHFGATTTAP | 883 | GI~F~AT | 1303 |
| p3 | CTNNB1 | S37F | DSGIHFGATTTAPSL | 884 | HF~A~TT | 1304 |
| p2 | CTNNB1 | S37F | SGIHFGATTTAPSLS | 885 | FG~T~TA | 1305 |
| p8 | CTNNB1 | S45P | IHSGATTTAPPLSGK | 886 | AT~T~PP | 1306 |
| p7 | CTNNB1 | S45P | HSGATTTAPPLSGKG | 887 | TT~A~PL | 1307 |
| p5 | CTNNB1 | S45P | GATTTAPPLSGKGNP | 888 | TA~P~SG | 1308 |
| p3 | CTNNB1 | S45P | TTTAPPLSGKGNPEE | 889 | PP~S~KG | 1309 |
| p2 | CTNNB1 | S45P | TTAPPLSGKGNPEEE | 890 | PL~G~GN | 1310 |
| p8 | CTNNB1 | T41A | LDSGIHSGATATAPS | 891 | IH~G~TA | 1311 |
| p7 | CTNNB1 | T41A | DSGIHSGATATAPSL | 892 | HS~A~AT | 1312 |
| p5 | CTNNB1 | T41A | GIHSGATATAPSLSG | 893 | GA~A~AP | 1313 |
| p3 | CTNNB1 | T41A | HSGATATAPSLSGKG | 894 | TA~A~SL | 1314 |
| p2 | CTNNB1 | T41A | SGATATAPSLSGKGN | 895 | AT~P~LS | 1315 |
| p8 | EGFR | A289V | VNPEGKYSFGVTCVK | 896 | GK~S~GV | 1316 |
| p7 | EGFR | A289V | NPEGKYSFGVTCVKK | 897 | KY~F~VT | 1317 |
| p5 | EGFR | A289V | EGKYSFGVTCVKKCP | 898 | SF~V~CV | 1318 |
| p3 | EGFR | A289V | KYSFGVTCVKKCPRN | 899 | GV~C~KK | 1319 |
| p2 | EGFR | A289V | YSFGVTCVKKCPRNY | 900 | VT~V~KC | 1320 |
| p8 | EGFR | G598V | GPHCVKTCPAVVMGE | 901 | VK~C~AV | 1321 |
| p7 | EGFR | G598V | PHCVKTCPAVVMGEN | 902 | KT~P~VV | 1322 |
| p5 | EGFR | G598V | CVKTCPAVVMGENNT | 903 | CP~V~MG | 1323 |
| p3 | EGFR | G598V | KTCPAVVMGENNTLV | 904 | AV~M~EN | 1324 |
| p2 | EGFR | G598V | TCPAVVMGENNTLVW | 905 | VV~G~NN | 1325 |
| p8 | EGFR | L858R | PQHVKITDFGRAKLL | 906 | KI~D~GR | 1326 |
| p7 | EGFR | L858R | QHVKITDFGRAKLLG | 907 | IT~F~RA | 1327 |
| p5 | EGFR | L858R | VKITDFGRAKLLGAE | 908 | DF~R~KL | 1328 |
| p3 | EGFR | L858R | ITDFGRAKLLGAEEK | 909 | GR~K~LG | 1329 |
| p2 | EGFR | L858R | TDFGRAKLLGAEEKE | 910 | RA~L~GA | 1330 |
| p8 | FBXW7 | R465C | HTLYGHTSTVCCMHL | 911 | GH~S~VC | 1331 |
| p7 | FBXW7 | R465C | TLYGHTSTVCCMHLH | 912 | HT~T~CC | 1332 |
| p5 | FBXW7 | R465C | YGHTSTVCCMHLHEK | 913 | ST~C~MH | 1333 |
| p3 | FBXW7 | R465C | HTSTVCCMHLHEKRV | 914 | VC~M~LH | 1334 |
| p2 | FBXW7 | R465C | TSTVCCMHLHEKRVV | 915 | CC~H~HE | 1335 |
| p8 | FBXW7 | R465G | HTLYGHTSTVGCMHL | 916 | GH~S~VG | 1336 |
| p7 | FBXW7 | R465G | TLYGHTSTVGCMHLH | 917 | HT~T~GC | 1337 |
| p5 | FBXW7 | R465G | YGHTSTVGCMHLHEK | 918 | ST~G~MH | 1338 |
| p3 | FBXW7 | R465G | HTSTVGCMHLHEKRV | 919 | VG~M~LH | 1339 |
| p2 | FBXW7 | R465G | TSTVGCMHLHEKRVV | 920 | GC~H~HE | 1340 |
| p8 | FBXW7 | R479Q | LHEKRVVSGSQDATL | 921 | RV~S~SQ | 1341 |
| p7 | FBXW7 | R479Q | HEKRVVSGSQDATLR | 922 | VV~G~QD | 1342 |
| p5 | FBXW7 | R4790 | KRVVSGSQDATLRVW | 923 | SG~Q~AT | 1343 |
| p3 | FBXW7 | R479Q | VVSGSQDATLRVWDI | 924 | SQ~A~LR | 1344 |
| p2 | FBXW7 | R479Q | VSGSQDATLRVWDIE | 925 | QD~T~RV | 1345 |
| p8 | FBXW7 | R505C | HVLMGHVAAVCCVQY | 926 | GH~A~VC | 1346 |
| p7 | FBXW7 | R505C | VLMGHVAAVCCVQYD | 927 | HV~A~CC | 1345 |
| p5 | FBXW7 | R505C | MGHVAAVCCVQYDGR | 928 | AA~C~VQ | 1348 |
| p3 | FBXW7 | R505C | HVAAVCCVQYDGRRV | 929 | VC~V~YD | 1349 |
| p2 | FBXW7 | R505C | VAAVCCVQYDGRRVV | 930 | CC~Q~DG | 1350 |
| p8 | FBXW7 | R505G | HVLMGHVAAVGCVQY | 931 | GH~A~VG | 1351 |
| p7 | FBXW7 | R505G | VLMGHVAAVGCVQYD | 932 | HV~A~GC | 1352 |
| p5 | FBXW7 | R505G | MGHVAAVGCVQYDGR | 933 | AA~G~VQ | 1353 |
| p3 | FBXW7 | R505G | HVAAVGCVQYDGRRV | 934 | VG~V~YD | 1354 |
| p2 | FBXW7 | R505G | VAAVGCVQYDGRRVV | 935 | GC~Q~DG | 1355 |
| p8 | FGFR2 | S252W | HTYHLDVVERWPHRP | 936 | LD~V~RW | 1356 |
| p7 | FGFR2 | S252W | TYHLDVVERWPHRPI | 937 | DV~E~WP | 1357 |
| p5 | FGFR2 | S252W | HLDVVERWPHRPILQ | 938 | VE~W~HR | 1358 |
| p3 | FGFR2 | S252W | DVVERWPHRPILQAG | 939 | RW~H~PI | 1359 |
| p2 | FGFR2 | S252W | VVERWPHRPILQAGL | 940 | WP~R~IL | 1360 |
| p8 | FGFR3 | S249C | QTYTLDVLERCPHRP | 941 | LD~L~RC | 1361 |
| p7 | FGFR3 | S249C | TYTLDVLERCPHRPI | 942 | DV~E~CP | 1362 |
| p5 | FGFR3 | S249C | TLDVLERCPHRPILQ | 943 | LE~C~HR | 1363 |
| p3 | FGFR3 | S249C | DVLERCPHRPILQAG | 944 | RC~H~PI | 1364 |
| p2 | FGFR3 | S249C | VLERCPHRPILQAGL | 945 | CP~R~IL | 1365 |
| p8 | GNA | Q209L | IIFRMVDVGGLRSER | 946 | MV~V~GL | 1366 |
| p7 | GNA | Q209L | IFRMVDVGGLRSERR | 947 | VD~G~LR | 1367 |
| p5 | GNA | Q209L | RMVDVGGLRSERRKW | 948 | VG~L~SE | 1368 |
| p3 | GNA | Q209L | VDVGGLRSERRKWIH | 949 | GL~S~RR | 1369 |
| p2 | GNA | Q209L | DVGGLRSERRKWIHC | 950 | LR~E~RK | 1370 |
| p8 | GNAQ | Q209L | VIFRMVDVGGLRSER | 951 | MV~V~GL | 1371 |
| p7 | GNAQ | Q209L | IFRMVDVGGLRSERR | 952 | VD~G~LR | 1372 |
| p5 | GNAQ | Q209L | RMVDVGGLRSERRKW | 953 | VG~L~SE | 1373 |
| p3 | GNAQ | Q209L | VDVGGLRSERRKWIH | 954 | GL~S~RR | 1374 |
| p2 | GNAQ | Q209L | DVGGLRSERRKWIHC | 955 | LR~E~RK | 1375 |
| p8 | GNAQ | Q209P | VIFRMVDVGGPRSER | 956 | MV~V~GP | 1376 |
| p7 | GNAQ | Q209P | IFRMVDVGGPRSERR | 957 | VD~G~PR | 1377 |
| p5 | GNAQ | Q209P | RMVDVGGPRSERRKW | 958 | VG~P~SE | 1378 |
| p3 | GNAQ | Q209P | VDVGGPRSERRKWIH | 959 | GP~S~RR | 1379 |
| p2 | GNAQ | Q209P | DVGGPRSERRKWIHC | 960 | PR~E~RK | 1380 |
| p8 | GNAS | R844C | VPSDQDLLRCCVLTS | 961 | QD~L~CC | 1381 |
| p7 | GNAS | R844C | PSDQDLLRCCVLTSG | 962 | DL~R~CV | 1382 |
| p5 | GNAS | R844C | DQDLLRCCVLTSGIF | 963 | LR~C~LT | 1383 |
| p3 | GNAS | R844C | DLLRCCVLTSGIFET | 964 | CC~L~SG | 1384 |
| p2 | GNAS | R844C | LLRCCVLTSGIFETK | 965 | CV~T~GI | 1385 |
| p8 | HRAS | Q61R | CLLDILDTAGREEYS | 966 | IL~T~GR | 1386 |
| p7 | HRAS | Q61R | LLDILDTAGREEYSA | 967 | LD~A~RE | 1387 |
| p5 | HRAS | 061R | DILDTAGREEYSAMR | 968 | TA~R~EY | 1388 |
| p3 | HRAS | Q61R | LDTAGREEYSAMRDQ | 969 | GR~E~SA | 1389 |
| p2 | HRAS | 061R | DTAGREEYSAMRDQY | 970 | RE~Y~AM | 1390 |
| p8 | KRAS | A146T | SYGIPFIETSTKTRQ | 971 | PF~E~ST | 1391 |
| p7 | KRAS | A146T | YGIPFIETSTKTRQR | 972 | FI~T~TK | 1392 |
| p5 | KRAS | A146T | PFIETSTKTRQRVE | 973 | ET~T~TR | 1393 |
| p3 | KRAS | A146T | FIETSTKTRQRVEDA | 974 | ST~T~QR | 1394 |
| p2 | KRAS | A146T | IETSTKTRQRVEDAF | 975 | TK~R~RV | 1395 |
| p8 | KRAS | G12A | TEYKLVVVGAAGVGK | 976 | LV~V~AA | 1396 |
| p7 | KRAS | G12A | EYKLVVVGAAGVGKS | 977 | VV~G~AG | 1397 |
| p5 | KRAS | G12A | KLVVVGAAGVGKSAL | 978 | VG~A~VG | 1398 |
| p3 | KRAS | G12A | VVVGAAGVGKSALTI | 979 | AA~V~KS | 1399 |
| p2 | KRAS | G12A | VVGAAGVGKSALTIQ | 980 | AG~G~SA | 1400 |
| p8 | KRAS | G12C | TEYKLVVVGACGVGK | 981 | LV~V~AC | 1401 |
| p7 | KRAS | G12C | EYKLVVVGACGVGKS | 982 | VV~G~CG | 1402 |
| p5 | KRAS | G12C | KLVVVGACGVGKSAL | 983 | VG~C~VG | 1403 |
| p3 | KRAS | G12C | VVVGACGVGKSALTI | 984 | AC~V~KS | 1404 |
| p2 | KRAS | G12C | VVGACGVGKSALTIQ | 985 | CG~G~SA | 1405 |
| p8 | KRAS | G12D | TEYKLVVVGADGVGK | 986 | LV~V~AD | 1406 |
| p7 | KRAS | G12D | EYKLVVVGADGVGKS | 987 | VV~G~DG | 1407 |
| p5 | KRAS | G12D | KLVVVGADGVGKSAL | 988 | VG~D~VG | 1408 |
| p3 | KRAS | G12D | VVVGADGVGKSALTI | 989 | AD~V~KS | 1409 |
| p2 | KRAS | G12D | VVGADGVGKSALTIQ | 990 | DG~G~SA | 1410 |
| p8 | KRAS | G12R | TEYKLVVVGARGVGK | 991 | LV~V~AR | 1411 |
| p7 | KRAS | G12R | EYKLVVVGARGVGKS | 992 | VV~G~RG | 1412 |
| p5 | KRAS | G12R | KLVVVGARGVGKSAL | 993 | VG~R~VG | 1413 |
| p3 | KRAS | G12R | VVVGARGVGKSALTI | 994 | AR~V~KS | 1414 |
| p2 | KRAS | G12R | VVGARGVGKSALTIQ | 995 | RG~G~SA | 1415 |
| p8 | KRAS | G12S | TEYKLVVVGASGVGK | 996 | LV~V~AS | 1416 |
| p7 | KRAS | G12S | EYKLVVVGASGVGKS | 997 | VV~G~SG | 1417 |
| p5 | KRAS | G12S | KLVVVGASGVGKSAL | 998 | VG~S~VG | 1418 |
| p3 | KRAS | G12S | VVVGASGVGKSALTI | 999 | AS~V~KS | 1419 |
| p2 | KRAS | G12S | VVGASGVGKSALTIQ | 1000 | SG~G~SA | 1420 |
| p8 | KRAS | G12V | TEYKLVVVGAVGVGK | 1001 | LV~V~AV | 1421 |
| p7 | KRAS | G12V | EYKLVVVGAVGVGKS | 1002 | VV~G~VG | 1422 |
| p5 | KRAS | G12V | KLVVVGAVGVGKSAL | 1003 | VG~V~VG | 1423 |
| p3 | KRAS | G12V | VVVGAVGVGKSALTI | 1004 | AV~V~KS | 1424 |
| p2 | KRAS | G12V | VVGAVGVGKSALTIQ | 1005 | VG~G~SA | 1425 |
| p8 | KRAS | G13C | EYKLVVVGAGCVGKS | 1006 | VV~G~GC | 1426 |
| p7 | KRAS | G13C | YKLVVVGAGCVGKSA | 1007 | VV~A~CV | 1427 |
| p5 | KRAS | G13C | LVVVGAGCVGKSALT | 1008 | GA~C~GK | 1428 |
| p3 | KRAS | G13C | VVGAGCVGKSALTIQ | 1009 | GC-G~SA | 1429 |
| p2 | KRAS | G13C | VGAGCVGKSALTIQL | 1010 | CV~K~AL | 1430 |
| p8 | KRAS | G13D | EYKLVVVGAGDVGKS | 1011 | VV~G~GD | 1431 |
| p7 | KRAS | G13D | YKLVVVGAGDVGKSA | 1012 | VV~A~DV | 1432 |
| p5 | KRAS | G13D | LVVVGAGDVGKSALT | 1013 | GA~D~GK | 1433 |
| p3 | KRAS | G13D | VVGAGDVGKSALTIQ | 1014 | GD~G~SA | 1434 |
| p2 | KRAS | G13D | VGAGDVGKSALTIQL | 1015 | DV~K~AL | 1435 |
| p8 | KRAS | Q61H | CLLDILDTAGHEEYS | 1016 | IL~T~GH | 1436 |
| p7 | KRAS | Q61H | LLDILDTAGHEEYSA | 1017 | LD~A~HE | 1437 |
| p5 | KRAS | Q61H | DILDTAGHEEYSAMR | 1018 | TA~H~EY | 1438 |
| p3 | KRAS | 061H | LDTAGHEEYSAMRDQ | 1019 | GH~E~SA | 1439 |
| p2 | KRAS | Q61H | DTAGHEEYSAMRDQY | 1020 | HE~Y~AM | 1440 |
| p8 | KRAS | 061H | CLLDILDTAGHEEYS | 1021 | IL~T~GH | 1441 |
| p7 | KRAS | Q61H | LLDILDTAGHEEYSA | 1022 | LD~A~HE | 1442 |
| p5 | KRAS | Q61H | DILDTAGHEEYSAMR | 1023 | TA~H~EY | 1443 |
| p3 | KRAS | Q61H | LDTAGHEEYSAMRDQ | 1024 | GH~E~SA | 1444 |
| p2 | KRAS | Q61H | DTAGHEEYSAMRDQY | 1025 | HE~Y~AM | 1445 |
| p8 | KRAS | 061L | CLLDILDTAGLEEYS | 1026 | IL~T~GL | 1446 |
| p7 | KRAS | Q61L | LLDILDTAGLEEYSA | 1027 | LD~A~LE | 1447 |
| p5 | KRAS | 061L | DILDTAGLEEYSAMR | 1028 | TA~L~EY | 1448 |
| p3 | KRAS | Q61L | LDTAGLEEYSAMRDQ | 1029 | GL~E~SA | 1449 |
| p2 | KRAS | Q61L | DTAGLEEYSAMRDQY | 1030 | LE~Y~AM | 1450 |
| p8 | NRAS | G12D | TEYKLVVVGADGVGK | 1031 | LV~V~AD | 1451 |
| p7 | NRAS | G12D | EYKLVVVGADGVGKS | 1032 | VV~G~DG | 1452 |
| p5 | NRAS | G12D | KLVVVGADGVGKSAL | 1033 | VG~D~VG | 1453 |
| p3 | NRAS | G12D | VVVGADGVGKSALTI | 1034 | AD~V~KS | 1454 |
| p2 | NRAS | G12D | VVGADGVGKSALTIQ | 1035 | DG~G~SA | 1455 |
| p8 | NRAS | G13D | EYKLVVVGAGDVGKS | 1036 | VV~G~GD | 1456 |
| p7 | NRAS | G13D | YKLVVVGAGDVGKSA | 1037 | VV~A~DV | 1457 |
| p5 | NRAS | G13D | LVVVGAGDVGKSALT | 1038 | GA~D~GK | 1458 |
| p3 | NRAS | G13D | VVGAGDVGKSALTIQ | 1039 | GD~G~SA | 1459 |
| p2 | NRAS | G13D | VGAGDVGKSALTIQL | 1040 | DV~K~AL | 1460 |
| p8 | NRAS | G13R | EYKLVVVGAGRVGKS | 1041 | VV~G~GR | 1461 |
| p7 | NRAS | G13R | YKLVVVGAGRVGKSA | 1042 | VV~A~RV | 1462 |
| p5 | NRAS | G13R | LVVVGAGRVGKSALT | 1043 | GA~R~GK | 1463 |
| p3 | NRAS | G13R | VVGAGRVGKSALTIQ | 1044 | GR~G~SA | 1464 |
| p2 | NRAS | G13R | VGAGRVGKSALTIQL | 1045 | RV~K~AL | 1465 |
| p8 | NRAS | Q61K | CLLDILDTAGKEEYS | 1046 | IL~T~GK | 1466 |
| p7 | NRAS | Q61K | LLDILDTAGKEEYSA | 1047 | LD~A~KE | 1467 |
| p5 | NRAS | Q61K | DILDTAGKEEYSAMR | 1048 | TA~K~EY | 1468 |
| p3 | NRAS | Q61K | LDTAGKEEYSAMRDQ | 1049 | GK~E~SA | 1469 |
| p2 | NRAS | Q61K | DTAGKEEYSAMRDQY | 1050 | KE~Y~AM | 1470 |
| p8 | NRAS | 061L | CLLDILDTAGLEEYS | 1051 | IL~T~GL | 1471 |
| p7 | NRAS | Q61L | LLDILDTAGLEEYSA | 1052 | LD~A~LE | 1472 |
| p5 | NRAS | Q61L | DILDTAGLEEYSAMR | 1053 | TA~L~EY | 1473 |
| p3 | NRAS | Q61L | LDTAGLEEYSAMRDQ | 1054 | GL~E~SA | 1474 |
| p2 | NRAS | Q61L | DTAGLEEYSAMRDQY | 1055 | LE~Y~AM | 1475 |
| p8 | PPP2R1A | P179R | YFRNLCSDDTRMVRR | 1056 | LC~D~TR | 1476 |
| p7 | PPP2R1A | P179R | FRNLCSDDTRMVRRA | 1057 | CS~D~RM | 1477 |
| p5 | PPP2R1A | P179R | NLCSDDTRMVRRAAA | 1058 | DD~R~VR | 1478 |
| p3 | PPP2R1A | P179R | CSDDTRMVRRAAASK | 1059 | TR~V~RA | 1479 |
| p2 | PPP2R1A | P179R | SDDTRMVRRAAASKL | 1060 | RM~R~AA | 1480 |
| p8 | PPP2R1A | R183W | LCSDDTPMVRWAAAS | 1061 | DT~M~RW | 1481 |
| p7 | PPP2R1A | R183W | CSDDTPMVRWAAASK | 1062 | TP~V~WA | 1482 |
| p5 | PPP2R1A | R183W | DDTPMVRWAAASKLG | 1063 | MV~W~AA | 1483 |
| p3 | PPP2R1A | R183W | TPMVRWAAASKLGEF | 1064 | RW~A~SK | 1484 |
| p2 | PPP2R1A | R183W | PMVRWAAASKLGEFA | 1065 | WA~A~KL | 1485 |
| p8 | PTEN | R130G | AAIHCKAGKGGTGVM | 1066 | CK~G~GG | 1486 |
| p7 | PTEN | R130G | AIHCKAGKGGTGVMI | 1067 | KA~K~GT | 1487 |
| p5 | PTEN | R130G | HCKAGKGGTGVMICA | 1068 | GK~G~GV | 1488 |
| p3 | PTEN | R130G | KAGKGGTGVMICAYL | 1069 | GG~G~MI | 1489 |
| p2 | PTEN | R130G | AGKGGTGVMICAYLL | 1070 | GT~V~IC | 1490 |
| p8 | PTEN | R130Q | AAIHCKAGKGQTGVM | 1071 | CK~G~GQ | 1491 |
| p7 | PTEN | R130Q | AIHCKAGKGQTGVMI | 1072 | KA~K~QT | 1492 |
| p5 | PTEN | R130Q | HCKAGKGQTGVMICA | 1073 | GK~Q~GV | 1493 |
| p3 | PTEN | R130Q | KAGKGQTGVMICAYL | 1074 | GQ~G~MI | 1494 |
| p2 | PTEN | R130Q | AGKGQTGVMICAYLL | 1075 | QT~V~IC | 1495 |
| p8 | SMAD4 | R361H | DGYVDPSGGDHFCLG | 1076 | DP~G~DH | 1496 |
| p7 | SMAD4 | R361H | GYVDPSGGDHFCLGQ | 1077 | PS~G~HF | 1497 |
| p5 | SMAD4 | R361H | VDPSGGDHFCLGQLS | 1078 | GG~H~CL | 1498 |
| p3 | SMAD4 | R361H | PSGGDHFCLGQLSNV | 1079 | DH~C~GQ | 1499 |
| p2 | SMAD4 | R361H | SGGDHFCLGQLSNVH | 1080 | HF~L~QL | 1500 |
| p8 | TP53 | C176F | SQHMTEVVRRFPHHE | 1081 | TE~V~RF | 1501 |
| p7 | TP53 | C176F | QHMTEVVRRFPHHER | 1082 | EV~R~FP | 1502 |
| p5 | TP53 | C176F | MTEVVRRFPHHERCS | 1083 | VR~F~HH | 1503 |
| p3 | TP53 | C176F | EVVRRFPHHERCSDS | 1084 | RF~H~ER | 1504 |
| p2 | TP53 | C176F | VVRRFPHHERCSDSD | 1085 | FP~H~RC | 1505 |
| p8 | TP53 | C176Y | SQHMTEVVRRYPHHE | 1086 | TE~V~RY | 1506 |
| p7 | TP53 | C176Y | QHMTEVVRRYPHHER | 1087 | EV~R~YP | 1507 |
| p5 | TP53 | C176Y | MTEVVRRYPHHERCS | 1088 | VR~Y~HH | 1508 |
| p3 | TP53 | C176Y | EVVRRYPHHERCSDS | 1089 | RY~H~ER | 1509 |
| p2 | TP53 | C176Y | VVRRYPHHERCSDSD | 1090 | YP~H~RC | 1510 |
| p8 | TP53 | C238Y | DCTTIHYNYMYNSSC | 1091 | IH~N~MY | 1511 |
| p7 | TP53 | C238Y | CTTIHYNYMYNSSCM | 1092 | HY~Y~YN | 1512 |
| p5 | TP53 | C238Y | TIHYNYMYNSSCMGG | 1093 | NY~Y~SS | 1513 |
| p3 | TP53 | C238Y | HYNYMYNSSCMGGMN | 1094 | MY~S~CM | 1514 |
| p2 | TP53 | C238Y | YNYMYNSSCMGGMNR | 1095 | YN~S~MG | 1515 |
| p8 | TP53 | C275Y | LGRNSFEVRVYACPG | 1096 | SF~V~VY | 1516 |
| p7 | TP53 | C275Y | GRNSFEVRVYACPGR | 1097 | FE~R~YA | 1517 |
| p5 | TP53 | C275Y | NSFEVRVYACPGRDR | 1098 | VR~Y~CP | 1518 |
| p3 | TP53 | C275Y | FEVRVYACPGRDRRT | 1099 | VY~C~GR | 1519 |
| p2 | TP53 | C275Y | EVRVYACPGRDRRTE | 1100 | YA~P~RD | 1520 |
| p8 | TP53 | E285K | CACPGRDRRTKEENL | 1101 | GR~R~TK | 1521 |
| p7 | TP53 | E285K | ACPGRDRRTKEENLR | 1102 | RD~R~KE | 1522 |
| p5 | TP53 | E285K | PGRDRRTKEENLRKK | 1103 | RR~K~EN | 1523 |
| p3 | TP53 | E285K | RDRRTKEENLRKKGE | 1104 | TK~E~LR | 1524 |
| p2 | TP53 | E285K | DRRTKEENLRKKGEP | 1105 | KE~N~RK | 1525 |
| p8 | TP53 | E286K | ACPGRDRRTEKENLR | 1106 | RD~R~EK | 1526 |
| p7 | TP53 | E286K | CPGRDRRTEKENLRK | 1107 | DR~T~KE | 1527 |
| p5 | TP53 | E286K | GRDRRTEKENLRKKG | 1108 | RT~K~NL | 1528 |
| p3 | TP53 | E286K | DRRTEKENLRKKGEP | 1109 | EK~N~RK | 1529 |
| p2 | TP53 | E286K | RRTEKENLRKKGEPH | 1110 | KE~L~KK | 1530 |
| p8 | TP53 | G245D | NYMCNSSCMGDMNRR | 1111 | NS~C~GD | 1531 |
| p7 | TP53 | G245D | YMCNSSCMGDMNRRP | 1112 | SS~M~DM | 1532 |
| p5 | TP53 | G245D | CNSSCMGDMNRRPIL | 1113 | CM~D~NR | 1533 |
| p3 | TP53 | G245D | SSCMGDMNRRPILTI | 1114 | GD~N~RP | 1534 |
| p2 | TP53 | G245D | SCMGDMNRRPILTI | 1115 | DM~R~PI | 1535 |
| p8 | TP53 | G245S | NYMCNSSCMGSMNRR | 1116 | NS~C~GS | 1536 |
| p7 | TP53 | G245S | YMCNSSCMGSMNRRP | 1117 | SS~M~SM | 1537 |
| p5 | TP53 | G245S | CNSSCMGSMNRRPIL | 1118 | CM~S~NR | 1538 |
| p3 | TP53 | G245S | SSCMGSMNRRPILTI | 1119 | GS~N~RP | 1539 |
| p2 | TP53 | G245S | SCMGSMNRRPILTII | 1120 | SM~R~PI | 1540 |
| p8 | TP53 | G245V | NYMCNSSCMGVMNRR | 1121 | NS~C~GV | 1541 |
| p7 | TP53 | G245V | YMCNSSCMGVMNRRP | 1122 | SS~M~VM | 1542 |
| p5 | TP53 | G245V | CNSSCMGVMNRRPIL | 1123 | CM~V~NR | 1543 |
| p3 | TP53 | G245V | SSCMGVMNRRPILTI | 1124 | GV~N~RP | 1544 |
| p2 | TP53 | G245V | SCMGVMNRRPILTII | 1125 | VM~R~PI | 1545 |
| p8 | TP53 | H179R | MTEVVRRCPHRERCS | 1126 | VR~C~HR | 1546 |
| p7 | TP53 | H179R | TEVVRRCPHRERCSD | 1127 | RR~P~RE | 1547 |
| p5 | TP53 | H179R | VVRRCPHRERCSDSD | 1128 | CP~R~RC | 1548 |
| p3 | TP53 | H179R | RRCPHRERCSDSDGL | 1129 | HR~R~SD | 1549 |
| p2 | TP53 | H179R | RCPHRERCSDSDGLA | 1130 | RE~C~DS | 1550 |
| p8 | TP53 | H179Y | MTEVVRRCPHYERCS | 1131 | VR~C~HY | 1551 |
| p7 | TP53 | H179Y | TEVVRRCPHYERCSD | 1132 | RR~P~YE | 1552 |
| p5 | TP53 | H179Y | VVRRCPHYERCSDSD | 1133 | CP~Y~RC | 1553 |
| p3 | TP53 | H179Y | RRCPHYERCSDSDGL | 1134 | HY~R~SD | 1554 |
| p2 | TP53 | H179Y | RCPHYERCSDSDGLA | 1135 | YE~C~DS | 1555 |
| p8 | TP53 | H193R | SDSDGLAPPQRLIRV | 1136 | GL~P~QR | 1556 |
| p7 | TP53 | H193R | DSDGLAPPQRLIRVE | 1137 | LA~P~RL | 1557 |
| p5 | TP53 | H193R | DGLAPPQRLIRVEGN | 1138 | PP~R~IR | 1558 |
| p3 | TP53 | H193R | LAPPQRLIRVEGNLR | 1139 | QR~~~VE | 1559 |
| p2 | TP53 | H193R | APPQRLIRVEGNLRV | 1140 | RL~R~EG | 1560 |
| p8 | TP53 | I195T | SDGLAPPQHLTRVEG | 1141 | AP~Q~LT | 1561 |
| p7 | TP53 | I195T | DGLAPPQHLTRVEGN | 1142 | PP~H~TR | 1562 |
| p5 | TP53 | I195T | LAPPQHLTRVEGNLR | 1143 | QH~T~VE | 1563 |
| p3 | TP53 | I195T | PPQHLTRVEGNLRVE | 1144 | LT~V~GN | 1564 |
| p2 | TP53 | I195T | PQHLTRVEGNLRVEY | 1145 | TR~E~NL | 1565 |
| p8 | TP53 | L194R | DSDGLAPPQHRIRVE | 1146 | LA~P~HR | 1566 |
| p7 | TP53 | L194R | SDGLAPPQHRIRVEG | 1147 | AP~Q~RI | 1567 |
| p5 | TP53 | L194R | GLAPPQHRIRVEGNL | 1148 | PQ~R~RV | 1568 |
| p3 | TP53 | L194R | APPQHRIRVEGNLRV | 1149 | HR~R~EG | 1569 |
| p2 | TP53 | L194R | PPQHRIRVEGNLRVE | 1150 | RI~V~GN | 1570 |
| p8 | TP53 | P151S | CPVQLWVDSTSPPGT | 1151 | LW~D~TS | 1571 |
| p7 | TP53 | P151S | PVQLWVDSTSPPGTR | 1152 | WV~S~SP | 1572 |
| p5 | TP53 | P151S | QLWVDSTSPPGTRVR | 1153 | DS~S~PG | 1573 |
| p3 | TP53 | P151S | WVDSTSPPGTRVRAM | 1154 | TS~P~TR | 1574 |
| p2 | TP53 | P151S | VDSTSPPGTRVRAMA | 1155 | SP~G~RV | 1575 |
| p8 | TP53 | R158H | DSTPPPGTRVHAMAI | 1156 | PP~T~VH | 1576 |
| p7 | TP53 | R158H | STPPPGTRVHAMAIY | 1157 | PG~R~HA | 1577 |
| p5 | TP53 | R158H | PPPGTRVHAMAIYKQ | 1158 | TR~H~MA | 1578 |
| p3 | TP53 | R158H | PGTRVHAMAIYKQSQ | 1159 | VH~M~IY | 1579 |
| p2 | TP53 | R158H | GTRVHAMAIYKQSQH | 1160 | HA~A~YK | 1580 |
| p8 | TP53 | R158L | DSTPPPGTRVLAMAI | 1161 | PP~T~VL | 1581 |
| p7 | TP53 | R158L | STPPPGTRVLAMAIY | 1162 | PG~R~LA | 1582 |
| p5 | TP53 | R158L | PPPGTRVLAMAIYKQ | 1163 | TR~L~MA | 1583 |
| p3 | TP53 | R158L | PGTRVLAMAIYKQSQ | 1164 | VL~M~IY | 1584 |
| p2 | TP53 | R158L | GTRVLAMAIYKQSQH | 1165 | LA~A~YK | 1585 |
| p8 | TP53 | R175H | QSQHMTEVVRHCPHH | 1166 | MT~V~RH | 1586 |
| p7 | TP53 | R175H | SQHMTEVVRHCPHHE | 1167 | TE~V~HC | 1587 |
| p5 | TP53 | R175H | HMTEVVRHCPHHERC | 1168 | VV~H~PH | 1588 |
| p3 | TP53 | R175H | TEVVRHCPHHERCSD | 1169 | RH~P~HE | 1589 |
| p2 | TP53 | R175H | EVVRHCPHHERCSDS | 1170 | HC~H~ER | 1590 |
| p8 | TP53 | R248Q | CNSSCMGGMNQRPIL | 1171 | CM~G~NQ | 1591 |
| p7 | TP53 | R248Q | NSSCMGGMNQRPILT | 1172 | MG~M~QR | 1592 |
| p5 | TP53 | R248Q | SCMGGMNQRPILTII | 1173 | GM~Q~PI | 1593 |
| p3 | TP53 | R248Q | MGGMNQRPILTIITL | 1174 | NQ~P~LT | 1594 |
| p2 | TP53 | R248Q | GGMNQRPILTIITLE | 1175 | QR~~~TI | 1595 |
| p8 | TP53 | R248W | CNSSCMGGMNWRPIL | 1176 | CM~G~NW | 1596 |
| p7 | TP53 | R248W | NSSCMGGMNWRPILT | 1177 | MG~M~WR | 1597 |
| p5 | TP53 | R248W | SCMGGMNWRPILTII | 1178 | GM~W~PI | 1598 |
| p3 | TP53 | R248W | MGGMNWRPILTIITL | 1179 | NW~P~LT | 1599 |
| p2 | TP53 | R248W | GGMNWRPILTIITLE | 1180 | WR~I~TI | 1600 |
| p8 | TP53 | R249S | NSSCMGGMNRSPILT | 1181 | MG~M~RS | 1601 |
| p7 | TP53 | R249S | SSCMGGMNRSPILTI | 1182 | GG~N~SP | 1602 |
| p5 | TP53 | R249S | CMGGMNRSPILTIIT | 1183 | MN~S~IL | 1603 |
| p3 | TP53 | R249S | GGMNRSPILTIITLE | 1184 | RS~I~TI | 1604 |
| p2 | TP53 | R249S | GMNRSPILTIITLED | 1185 | SP~L~II | 1605 |
| p8 | TP53 | R249S | NSSCMGGMNRSPILT | 1186 | MG~M~RS | 1606 |
| p7 | TP53 | R249S | SSCMGGMNRSPILTI | 1187 | GG~N~SP | 1607 |
| p5 | TP53 | R249S | CMGGMNRSPILTIIT | 1188 | MN~S~IL | 1608 |
| p3 | TP53 | R249S | GGMNRSPILTIITLE | 1189 | RS~I~TI | 1609 |
| p2 | TP53 | R249S | GMNRSPILTIITLED | 1190 | SP~L~II | 1610 |
| p8 | TP53 | R273C | NLLGRNSFEVCVCAC | 1191 | RN~F~VC | 1611 |
| p7 | TP53 | R273C | LLGRNSFEVCVCACP | 1192 | NS~E~CV | 1612 |
| p5 | TP53 | R273C | GRNSFEVCVCACPGR | 1193 | FE~C~CA | 1613 |
| p3 | TP53 | R273C | NSFEVCVCACPGRDR | 1194 | VC~C~CP | 1614 |
| p2 | TP53 | R273C | SFEVCVCACPGRDRR | 1195 | CV~A~PG | 1615 |
| p8 | TP53 | R273H | NLLGRNSFEVHVCAC | 1196 | RN~F~VH | 1616 |
| p7 | TP53 | R273H | LLGRNSFEVHVCACP | 1197 | NS~E~HV | 1617 |
| p5 | TP53 | R273H | GRNSFEVHVCACPGR | 1198 | FE~H~CA | 1618 |
| p3 | TP53 | R273H | NSFEVHVCACPGRDR | 1199 | VH~C~CP | 1619 |
| p2 | TP53 | R273H | SFEVHVCACPGRDRR | 1200 | HV~A~PG | 1620 |
| p8 | TP53 | R273L | NLLGRNSFEVLVCAC | 1201 | RN~F~VL | 1621 |
| p7 | TP53 | R273L | LLGRNSFEVLVCACP | 1202 | NS~E~LV | 1622 |
| p5 | TP53 | R273L | GRNSFEVLVCACPGR | 1203 | FE~L~CA | 1623 |
| p3 | TP53 | R273L | NSFEVLVCACPGRDR | 1204 | VL~C~CP | 1624 |
| p2 | TP53 | R273L | SFEVLVCACPGRDRR | 1205 | LV~A~PG | 1625 |
| p8 | TP53 | R280K | FEVRVCACPGKDRRT | 1206 | VC~C~GK | 1626 |
| p7 | TP53 | R280K | EVRVCACPGKDRRTE | 1207 | CA~P~KD | 1627 |
| p5 | TP53 | R280K | RVCACPGKDRRTEEE | 1208 | CP~K~RR | 1628 |
| p3 | TP53 | R280K | CACPGKDRRTEEENL | 1209 | GK~R~TE | 1629 |
| p2 | TP53 | R280K | ACPGKDRRTEEENLR | 1210 | KD~R~EE | 1630 |
| p8 | TP53 | R280T | FEVRVCACPGTDRRT | 1211 | VC~C~GT | 1631 |
| p7 | TP53 | R280T | EVRVCACPGTDRRTE | 1212 | CA~P~TD | 1632 |
| p5 | TP53 | R280T | RVCACPGTDRRTEEE | 1213 | CP~T~RR | 1633 |
| p3 | TP53 | R280T | CACPGTDRRTEEENL | 1214 | GT~R~TE | 1634 |
| p2 | TP53 | R280T | ACPGTDRRTEEENLR | 1215 | TD~R~EE | 1635 |
| p8 | TP53 | R282W | VRVCACPGRDWRTEE | 1216 | AC~G~DW | 1636 |
| p7 | TP53 | R282W | RVCACPGRDWRTEEE | 1217 | CP~R~WR | 1637 |
| p5 | TP53 | R282W | CACPGRDWRTEEENL | 1218 | GR~W~TE | 1638 |
| p3 | TP53 | R282W | CPGRDWRTEEENLRK | 1219 | DW~T~EE | 1639 |
| p2 | TP53 | R282W | PGRDWRTEEENLRKK | 1220 | WR~E~EN | 1640 |
| p8 | TP53 | S241F | TIHYNYMCNSFCMGG | 1221 | NY~C~SF | 1641 |
| p7 | TP53 | S241F | IHYNYMCNSFCMGGM | 1222 | YM~N~FC | 1642 |
| p5 | TP53 | S241F | YNYMCNSFCMGGMNR | 1223 | CN~F~MG | 1643 |
| p3 | TP53 | S241F | YMCNSFCMGGMNRRP | 1224 | SF~M~GM | 1644 |
| p2 | TP53 | S241F | MCNSFCMGGMNRRPI | 1225 | FC~G~MN | 1645 |
| p8 | TP53 | V157F | VDSTPPPGTRFRAMA | 1226 | PP~G~RF | 1646 |
| p7 | TP53 | V157F | DSTPPPGTRFRAMAI | 1227 | PP~T~FR | 1647 |
| p5 | TP53 | V157F | TPPPGTRFRAMAIYK | 1228 | GT~F~AM | 1648 |
| p3 | TP53 | V157F | PPGTRFRAMAIYKQS | 1229 | RF~A~AI | 1649 |
| p2 | TP53 | V157F | PGTRFRAMAIYKQSQ | 1230 | FR~M~IY | 1650 |
| p8 | TP53 | V173M | YKQSQHMTEVMRRCP | 1231 | QH~T~VM | 1651 |
| p7 | TP53 | V173M | KQSQHMTEVMRRCPH | 1232 | HM~E~MR | 1652 |
| p5 | TP53 | V173M | SQHMTEVMRRCPHHE | 1233 | TE~M~RC | 1653 |
| p3 | TP53 | V173M | HMTEVMRRCPHHERC | 1234 | VM~R~PH | 1654 |
| p2 | TP53 | V173M | MTEVMRRCPHHERCS | 1235 | MR~C~HH | 1655 |
| p8 | TP53 | V272M | GNLLGRNSFEMRVCA | 1236 | GR~S~EM | 1656 |
| p7 | TP53 | V272M | NLLGRNSFEMRVCAC | 1237 | RN~F~MR | 1657 |
| p5 | TP53 | V272M | LGRNSFEMRVCACPG | 1238 | SF~M~VC | 1658 |
| p3 | TP53 | V272M | RNSFEMRVCACPGRD | 1239 | EM~V~AC | 1659 |
| p2 | TP53 | V272M | NSFEMRVCACPGRDR | 1240 | MR~C~CP | 1660 |
| p8 | TP53 | Y163C | PGTRVRAMAICKQSQ | 1241 | VR~M~IC | 1661 |
| p7 | TP53 | Y163C | GTRVRAMAICKQSQH | 1242 | RA~A~CK | 1662 |
| p5 | TP53 | Y163C | RVRAMAICKQSQHMT | 1243 | MA~C~QS | 1663 |
| p3 | TP53 | Y163C | RAMAICKQSQHMTEV | 1244 | IC~Q~QH | 1664 |
| p2 | TP53 | Y163C | AMAICKQSQHMTEVV | 1245 | CK~S~HM | 1665 |
| p8 | TP53 | Y205C | IRVEGNLRVECLDDR | 1246 | GN~R~EC | 1666 |
| p7 | TP53 | Y205C | RVEGNLRVECLDDRN | 1247 | NL~V~CL | 1667 |
| p5 | TP53 | Y205C | EGNLRVECLDDRNTF | 1248 | RV~C~DD | 1668 |
| p3 | TP53 | Y205C | NLRVECLDDRNTFRH | 1249 | EC~D~RN | 1669 |
| p2 | TP53 | Y205C | LRVECLDDRNTFRHS | 1250 | CL~D~NT | 1670 |
| p8 | TP53 | Y220C | NTFRHSVVVPCEPPE | 1251 | HS~V~PC | 1671 |
| p7 | TP53 | Y220C | TFRHSVVVPCEPPEV | 1252 | SV~V~CE | 1672 |
| p5 | TP53 | Y220C | RHSVVVPCEPPEVGS | 1253 | VV~C~PP | 1673 |
| p3 | TP53 | Y220C | SVVVPCEPPEVGSDC | 1254 | PC~P~EV | 1674 |
| p2 | TP53 | Y220C | VVVPCEPPEVGSDCT | 1255 | CE~P~VG | 1675 |
| p8 | TP53 | Y234C | EVGSDCTTIHCNYMC | 1256 | DC~T~HC | 1676 |
| p7 | TP53 | Y234C | VGSDCTTIHCNYMCN | 1257 | CT~I~CN | 1677 |
| p5 | TP53 | Y234C | SDCTTIHCNYMCNSS | 1258 | TI~C~YM | 1678 |
| p3 | TP53 | Y234C | CTTIHCNYMCNSSCM | 1259 | HC~Y~CN | 1679 |
| p2 | TP53 | Y234C | TTIHCNYMCNSSCMG | 1260 | CN~M~NS | 1680 |

### Addition of Peptides Targeting Further Mutations

Many of the considerations in neoepitope selection can be addressed prior to presentation of a clinical cancer case for those mutations that are known to occur commonly, and for a wide array of frequently occurring HLA alleles, thus allowing rapid deployment of a prepared vaccine. However, each individual subject will, in almost all instances, also carry personal mutations that it is not possible to anticipate in a pre-assembled library. Therefore, in some embodiments the present invention also provides methods to select peptides, or the nucleic acids encoding them, to target the personal mutations. Such personal mutations may occur at less common positions in a driver gene product or may be in a passenger gene product.

In some referred embodiments, a decision on selection of neoepitopes for inclusion in a personal vaccine of peptides, comprising the T cell exposed motifs of less common driver gene products and the passenger mutations in a particular tumor, is made following sequencing and comparison of tumor and normal sequences in the tumor. As is the case for the preassembled library, considerations in selection of peptides that expose the mutant amino acid in the T cell exposed motifs include, but are not limited to, RNA transcription and expression, T cell exposed motif frequency relative to reference databases of pentamer frequency, probability of cathepsin cleavage, and determination of the individual subject's HLA genotype and binding to MHC of the constituent alleles. The reference databases for evaluation of T cell exposed motif frequency may be drawn from the human proteome, the gastrointestinal microbiome, a database of other microorganisms or the human immunoglobulinome. These examples of suitable reference databases are not considered limiting. Thus, while preparation of a library of peptides responsive to common mutations expedites vaccination, the final composition of a vaccine is qualified by these individual aspects.

### T Cell Selection and Expansion

Embodiments of the methods disclosed herein also facilitate the selection and delivery of relevant expanded T cell populations by means of autologous transfer or as engineered naïve cells with tumor specific TCR sequences. Targeting of mutated proteins in tumor cells by T cells has been referred to as the ‘final common pathway’ of all immunotherapy (1). Ensuring that such targeting is completely tumor specific is a prerequisite to the transfer of T cells. In addition, enabling the rapid identification of such specific cells is highly desirable. The down-selection of epitopes using the methods described herein to evaluate T cell exposed motif pentamer frequency enables a focus on just those T cells which are more likely to have a clonal population of sufficient size in a patient's T cell repertoire in order to make their identification possible and to enable sufficient numbers to be harvested, expanded and targeted to the selected mutated T cell exposed motif presented in the tumor. This is then supplemented by prediction of MHC binding affinity and the probability of cathepsin cleavage. The pre-identification of the key T cell exposed motifs, and peptides that comprise these motifs, facilitates the capture, selection and expansion of T cells. Importantly, it also allows effective use of the limited numbers of T cells which can be harvested from blood or tumor biopsies, as the cells need to be exposed in vitro to only a limited number of selected potential cognate epitopes in the quest to identify T cell clones with tumor specificity and efficacy.

In some embodiments, T cells identified as responsive to the selected epitopes, and then multiplied in culture, may be transferred back to the subject as an autologous transfer. In other embodiments the TCR sequences in T cells from the expanded pools maybe determined and these sequences engineered into naïve T cells for clonal expansion and administration to the patient. In yet other embodiments the TCR sequences may be introduced into other cells, including but not limited to mammalian cells in culture for use in assays and research. These embodiments are examples and should not be considered limiting.

The ability to identify a priori the critical epitopes comprising T cell exposed motifs which are most likely to encounter a responsive precursor clonal population, based on their frequency of occurrence of the corresponding pentamer motifs in reference databases, may make it possible to treat more subjects. By applying the criteria of down-selection provided herein and identifying T cell clones specific to common mutations that have a desirable binding affinity for one or more common MHC alleles then it becomes possible to bank expanded T cells or T cells engineered with the TCR sequences for administration to other subjects affected by the common mutations.

The methods described in Example 7 are applicable to either peptides presented by MHC I or MHC II that comprise mutated T cell exposed motifs. Methods of culturing antigen presenting cells are well known to the art. The antigen presenting cells may comprise, but are not limited to, dendritic cells, macrophages, Langerhans cells, B-lymphocytes, and T-cells, and may include both professional and non-professional APCs. In most preferred embodiments dendritic cells are the antigen presenting cells pulsed with the selected epitopes for presentation to T cells. Sequencing of the TCR from the resultant cognate T cell population then enables delivery of a recombinant TCR sequence to a recipient cell. The recipient cell may be a cell in culture for use in research studies, or a recipient T cell thereby allowing establishment of a donor line for adoptive transfer that is specific to the mutation-allele combination. Many suitable gene transfer systems for recombinant expression are known to the art including, but not limited to, plasmids and vectors of viral origin, transfection and electroporation.

### Novel B Cell Epitopes

Tumor mutations also create novel B cell neoepitopes. In addition to directing T cells as CD4+ or CD8+ to a tumor specific epitope, it may also be advantageous to stimulate antibodies which can mediate tumor specific antibody mediated cellular cytotoxicity (ADCC) through recruitment of further T cells. Not all mutations will generate a B cell epitope, but approximately 25-30% of mutations will generate a qualitative and quantitative change in the B cell epitopes in mutated proteins. To the extent that these are surface displayed, or become exposed by tumor cell destruction by radiation or by apoptosis or necrosis, they are targetable tumor specific targets. A further method provided herein is therefore to identify peptides which are suitable antibody targets, i.e., B cell neoepitopes, and to allow the rapid synthesis of peptides that encompass the linear B cell epitope. In preferred embodiments such peptides may be extended to also comprise adjacent epitopes that will stimulate T helper cells. In some particular embodiments the antibodies thus generated may be utilized as the targeting ligands in a CAR T immunotherapy.

### Rapid Assembly

The present invention also speeds the synthesis of peptide vaccines by providing a method to pre-assemble trimer building blocks of peptides for assembly to 9mers and 15mers for inclusion in a vaccine. By preassembling a prefabricated library of trimer building blocks, the synthesis of 9 mer peptides is reduced from a 9 reaction process to a 2 reaction process, with a concomitant reduction in quality control and assurance steps. A 15 mer peptide synthesis is reduced from a 15 reaction process to a 4 step process. In some preferred instances the assembly from trimers may be reduced to a single step reaction.

In the case of T cell epitopes, peptides may be assembled from these trimer building blocks to match the naturally occurring 9mer or 15mer sequence found in the mutated tumor protein. In other embodiments the peptides may be designed to optimize binding and presentation by a particular HLA allele in a subject's genotype by substitution of the naturally occurring amino acids in the groove exposed positions with alternative amino acids selected to optimize binding to a desired affinity.

In instances where a peptide corresponding to a B cell epitope is constructed this may be longer than 15mer and may be designed to span both a B cell linear epitope and an adjacent MHC II binding T helper epitope. In such embodiments the peptide constructed from trimer building blocks may be longer than a 15 mer and may be up to 33 or 36 amino acids in length.

### Vaccine Delivery Systems

Many methods of vaccine delivery are known to the art. Neoepitope vaccines maybe delivered as nucleotide sequences, either DNA or RNA, which may be encoded in plasmids or vectors. In some embodiments, neoepitopes are delivered in a viral vector. Suitable nucleic acid vaccines may be designed, for example, as described of US patent publications US20200254086, US20220152178, US20180369419, and/or US20210268086, each of which is incorporated herein by reference in its entirety. Neoepitopes may also be delivered as peptide vaccines. Each neoepitope may be delivered singly or as a multiplex. Many different adjuvants may be used in conjunction with a neoepitope vaccine, including but not limited to, lipid A analogues (e.g., poly I:C), imidazoquinolines (e.g. imiquimod), CpG, saponins, C type lectin ligands, CD1d ligands 9 e.g. a-galactosylceramide), aluminum salts (e.g. aluminum hydroxide), emulsions (e.g. MF59), and many variants thereof. Many different routes of vaccination are also possible, including both parenteral and non-parenteral, including but not limited to intradermal, subcutaneous, intramuscular, intra tumoral, oral, enteric and other routes of administration known to the art. Formulation may be in any pharmaceutical carrier suitable for the mode of delivery.

The selection methods described herein to provide optimal T cell exposed motifs and MHC presentation thereof are not intended to be limited to a particular delivery method, route of administration, or formulation and may be embodied in any feasible delivery and formulation. Formulation and manufacture of peptide vaccines may be facilitated by selecting groove exposed motifs with consideration of their impact on the solubility, stability and propensity for aggregation of the peptides. See, PCT APPL. US/2021/062147, incorporated herein by reference in its entirety. An assessment of peptide solubility in aqueous solvents can be made by determining the polarity and the partition coefficient. To enhance stability by reducing potential oxidation, peptides are selected in which amino acids from the group comprising methionine, tryptophan, histidine, cystine and tyrosine are excluded in the groove exposed motif. To reduce deamidation, peptides are selected in which asparagine and glutamine are not present in the groove exposed motif. Exclusion of cysteine has the additional benefit in reducing cross linking between peptides by formation of disulfide bonds.

### Role of Cathepsins in Tumor Progression and Cathepsin Inhibition

Cathepsins, and especially cathepsin B, are often strongly upregulated and expressed in tumors and are recognized as biomarkers with prognostic value (66, 70, 71). Many roles have been ascribed to cathepsins in tumors, including proliferation, angiogenesis, invasion, metastasis, autophagy and apoptosis (72, 73, 74, 75, 76, 77). The role of the cysteine cathepsins in extracellular matrix degradation contributes to these effects (78). Cathepsin D may cleave the MHC II invariant chain (76). KRAS has been shown to stimulate cathepsin B secretion in colorectal cancer (79). Mice with cathepsin B deficiency show delay in progression of mammary carcinoma (80).

Nevertheless, the importance of cathepsins in cleavage of a tumor neoepitope peptide, precluding its presentation to T cells and enabling immune evasion, has heretofore not been recognized or considered as a factor in selecting and managing neoantigen vaccines for the RAS gene products or other tumor specific proteins.

Given the recognition of the broader roles of cathepsins identified in the tumor microenvironment and noted above, there is active interest in identifying means of modulating cathepsin activity (70, 81, 82, 83). A number of cathepsin inhibitors are known to the art. These include, but are not limited to, nitrile derivatives, ketone derivatives, acryl hydrazine, vinyl sulfonate derivatives, epoxy succinic acid, betalactams, surugamides, loxistatin derivatives, sulfonamide derivatives and many other products (84, 85, 86, 87, 88). Products with cathepsin inhibitory characteristics have been extensively reviewed and the properties of each discussed (70, 81, 84, 89, 90). In addition, some natural plant medicinal products, including but not limited to, caffeic acid and chlorogenic acid have been shown to be cathepsin inhibitors (91). Given the ongoing efforts to identify and characterize cathepsin inhibitors it is likely that additional cathepsin inhibitor compounds will be added to the list, which are included in those which may be used as an adjunct to vaccination with tumor peptides that otherwise would be cleaved by a cathepsin.

Regulation of cathepsin in vivo is effected by proteins of the cystatin family which inhibit cathepsins at pico and nanomolar levels (92). Disruption of cystatin expression has been associated with cancer progression. Type I cystatins (also known as stefins) are intracellular proteins of approximately 100 amino acids. Stefin A and B are closely associated with the inactivation of cathepsins B L and S. In contrast type II cystatins are extracellular and cystatin C is the type I cystatin most active against the cathepsins B, L and S, Sequences of these three cystatins are shown in Table 4.

TABLE 4
Sequences of cystatins most active in cathepsin inactivation

| | |
|---|---|
| SEQ ID | P01040ICYTA_HUMAN Cystatin-A |
| NO.: 2604 | MIPGGLSEAKPATPEIQEIVDKVKPQLEEKTNETYGKLEAVQYKTQVV |
| | AGTNYYIKVRAGDNKYMHLKVFKSLPGQNEDLVLTGYQVDKNKDDE |
| | LTGF |
| SEQ ID | P04080ICYTB_HUMAN Cystatin-B |
| NO.: 2605 | MMCGAPSATQPATAETQHIADQVRSQLEEKENKKFPVFKAVSFKSQV |
| | V |
| | AGTNYFIKVHVGDEDFVHLRVFQSLPHENKPLTLSNYQTNKAKHDELT |
| | YF |
| SEQ ID | P01034\|CYTC_HUMAN Cystatin-C |
| NO.: 2606 | MAGPLRAPLLLLAILAVALAVSPAAGSSPGKPPRLVGGPMDASVEEEG |
| | V |
| | RRALDFAVGEYNKASNDMYHSRALQVVRARKQIVAGVNYFLDVELG |
| | RT |
| | TCTKTQPNLDNCPFHDQPHLKRKAFCSFQIYAVPWQGTMTLSKSTCQD |
| | A |

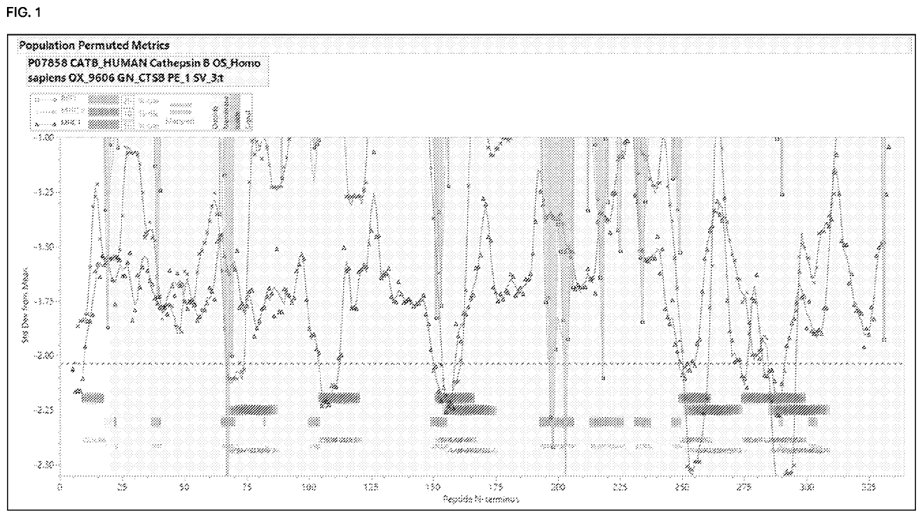

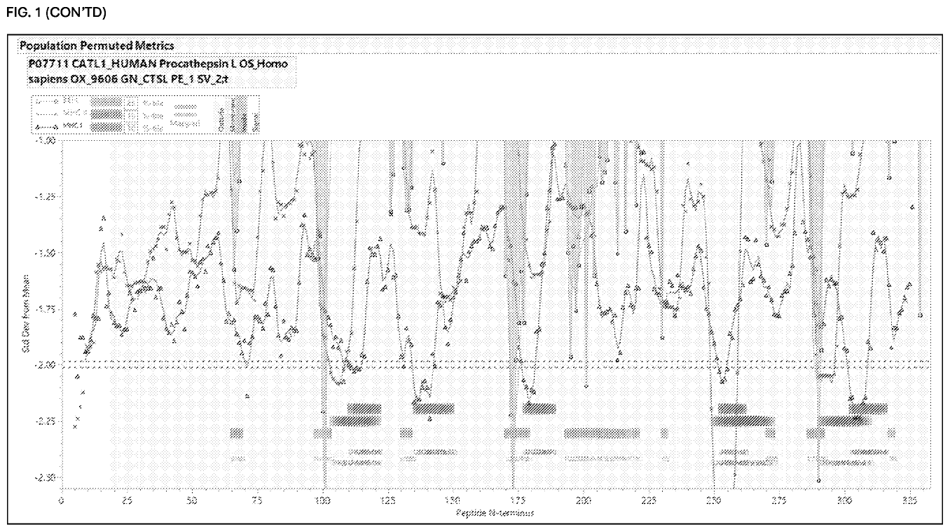

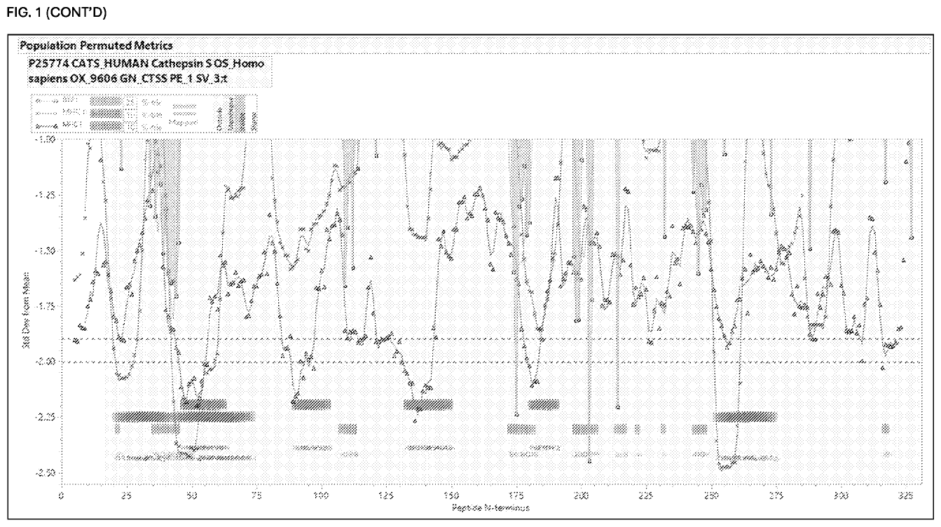

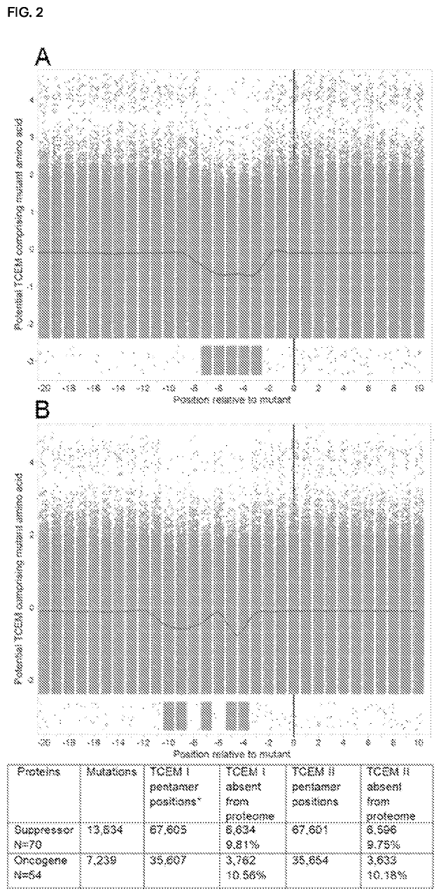

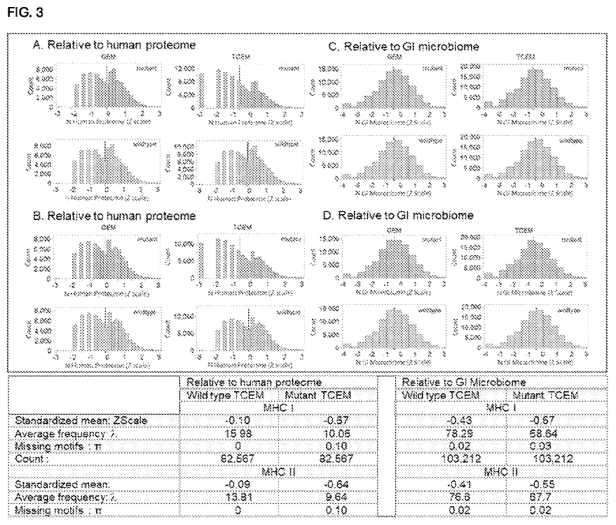

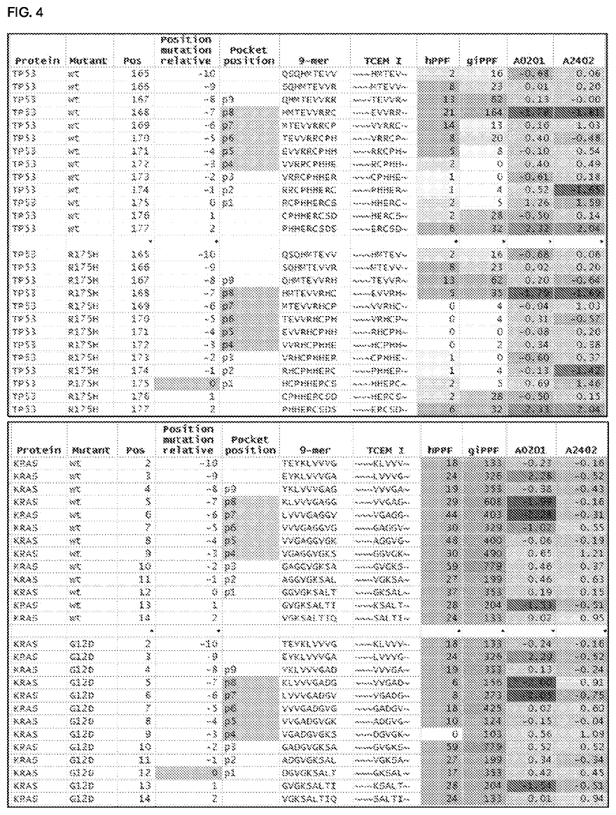

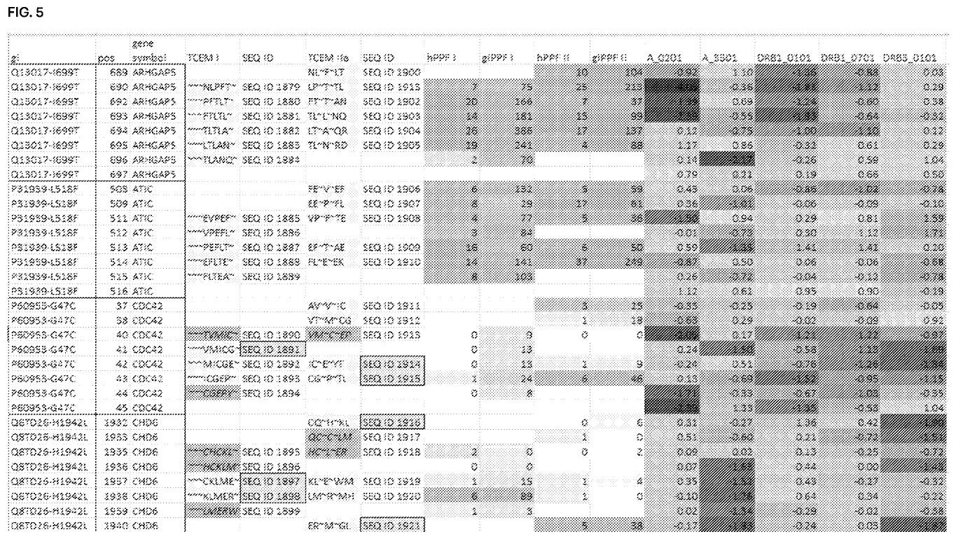

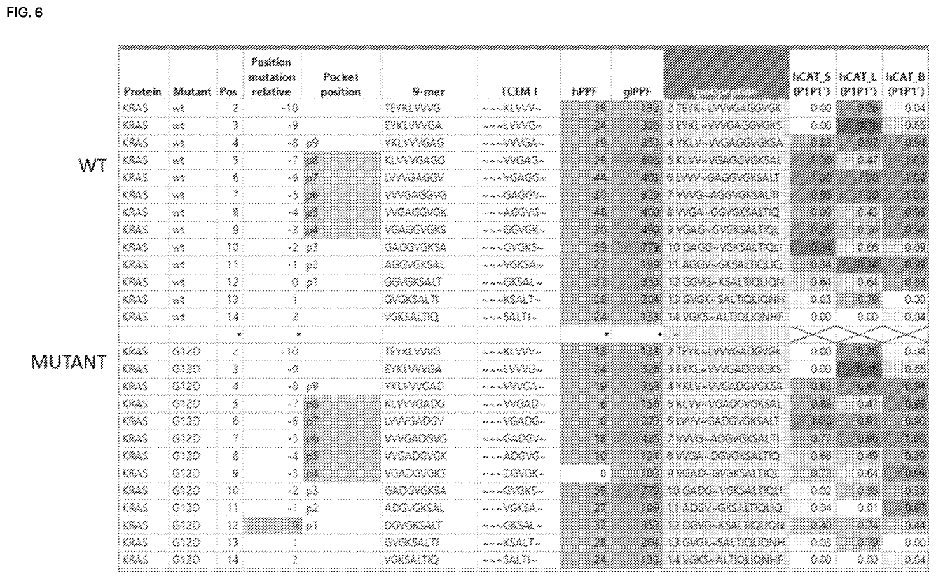

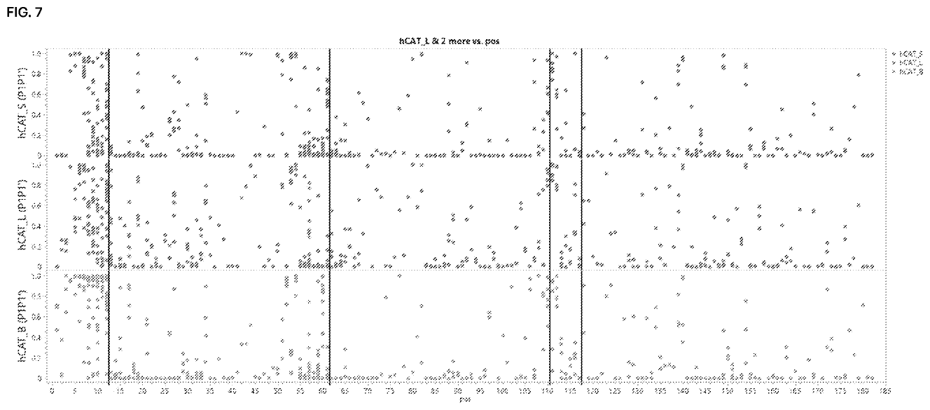

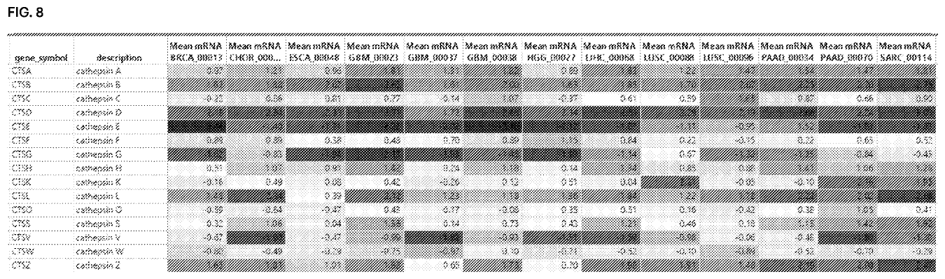

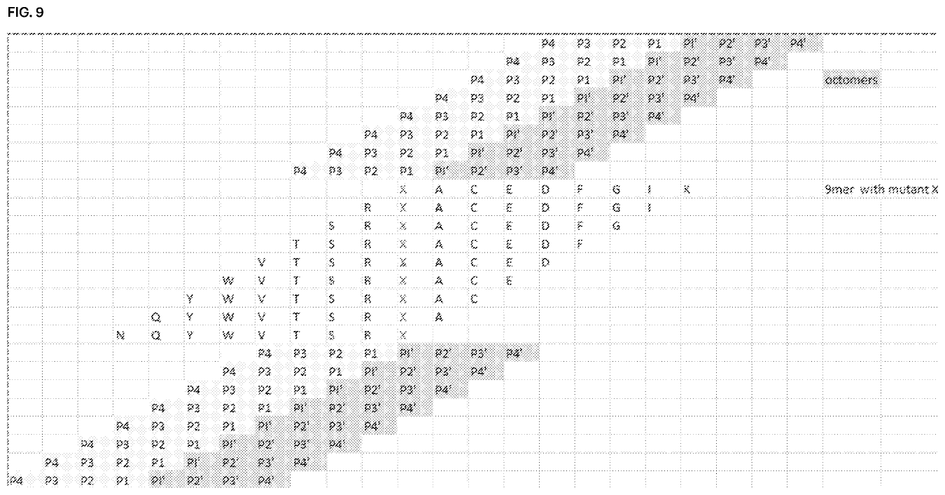

Given the relatively small size of the cystatins they may be produced recombinantly. Or encoded in a nucleic acid sequence for intracellular expression. Hence, a cystatin or an active subsequence therefrom, may be delivered to or expressed in an antigen presenting cell or a tumor cell if delivered as a component of a neoantigen vaccine formulation or delivered intratumorally. Cathepsins themselves have been shown to be antigenic and capable of generating antibody responses in the context of parasitic infections(93). Epitope mapping of human cathepsin L, B and S shown in FIG. 1 shows distinct and different linear B cell epitopes that would enable antibody targeting of cathepsin, either by standard tetrameric antibodies or subcomponents such as scFV. This is an approach which could enable neutralization of cathepsin or the targeting of other inhibitors to tumor cells with high upregulation of cathepsin. Given the relative lack of sequence conservation among these cathepsins a high degree of specificity would be expected. Sequences for cathepsin B, L and S are shown in Table 5 and sequences of the linear B cell epitopes in Table 6

TABLE 5
Sequences of Cathepsin B, L and S.

| | |
|---|---|
| SEQ | P07858 CATB_HUMAN Cathepsin B |
| ID | MWQLWASLCCLLVLANARSRPSFHPLSDELVNYVNKRNTTWQAGHNFYNVDMSYLKRLCG |
| NO.: | TFLGGPKPPQRVMFTEDLKLPASFDAREQWPQCPTIKEIRDQGSCGSCWAFGAVEAISDR |
| 2607 | ICIHTNAHVSVEVSAEDLLTCCGSMCGDGCNGGYPAEAWNFWTRKGLVSGGLYESHVGCR |
| | PYSIPPCEHHVNGSRPPCTGEGDTPKCSKICEPGYSPTYKQDKHYGYNSYSVSNSEKDIM |
| | AEIYKNGPVEGAFSVYSDFLLYKSGVYQHVTGEMMGGHAIRILGWGVENGTPYWLVANSW |
| | NTDWGDNGFFKILRGQDHCGIESEVVAGIPRTDQYWEKI |
| SEQ | P07711CATL1_HUMAN Procathepsin L |
| ID | MNPTLILAAFCLGIASATLTFDHSLEAQWTKWKAMHNRLYGMNEEGWRRAVWEKNMKMIE |
| NO.: | LHNQEYREGKHSFTMAMNAFGDMTSEEFRQVMNGFQNRKPRKGKVFQEPLFYEAPRSVDW |
| 2608 | REKGYVTPVKNQGQCGSCWAFSATGALEGQMFRKTGRLISLSEQNLVDCSGPQGNEGCNG |
| | GLMDYAFQYVQDNGGLDSEESYPYEATEESCKYNPKYSVANDTGFVDIPKQEKALMKAVA |
| | TVGPISVAIDAGHESFLFYKEGIYFEPDCSSEDMDHGVLVVGYGFESTESDNNKYWLVKN |
| | SWGEEWGMGGYVKMAKDRRNHCGIASAASYPTV |
| SEQ | P25774 CATS_HUMAN Cathepsin S |
| ID | MKRLVCVLLVCSSAVAQLHKDPTLDHHWHLWKKTYGKQYKEKNEEAVRRLIWEKNLKFVM |
| NO.: | LHNLEHSMGMHSYDLGMNHLGDMTSEEVMSLMSSLRVPSQWQRNITYKSNPNRILPDSVD |
| 2609 | WREKGCVTEVKYQGSCGACWAFSAVGALEAQLKLKTGKLVSLSAQNLVDCSTEKYGNKGC |
| | NGGFMTTAFQYIIDNKGIDSDASYPYKAMDQKCQYDSKYRAATCSKYTELPYGREDVLKE |
| | AVANKGPVSVGVDARHPSFFLYRSGVYYEPSCTQNVNHGVLVVGYGDLNGKEYWLVKNSW |
| | GHNFGEEGYIRMARNKGNHCGIASFPSYPEI |

TABLE 6
B cell epitopes in cathepsins

| | | |
|---|---|---|
| Cathepsin B | NARSRPSFHPLSDEL | SEQ ID NO.: 2610 |
| | KRNTTWQAGHNFY | SEQ ID NO.: 2611 |
| | LGGPKPPQRVMFTED | SEQ ID NO.: 2612 |
| | IRDQGSCGSCWAF | SEQ ID NO.: 2613 |
| | DGCNGGYPAEAWNFW | SEQ ID NO.: 2614 |
| | VNGSRPPCTGEGDTPKCS | SEQ ID NO.: 2615 |
| | KICEPG | |
| | EPGYSPTYKQDKHYGYN | SEQ ID NO.: 2616 |
| | YSVSNSEKDIMAEIY | SEQ ID NO.: 2617 |
| Cathepsin L | NQEYREGKHSFTMA | SEQ ID NO.: 2618 |
| | FQNRKPRKGKVFQEPLF | SEQ ID NO.: 2619 |
| | KNQGQCGSCWAF | SEQ ID NO.: 2620 |
| | DCSGPQGNEGCNGGLMDYA | SEQ ID NO.: 2621 |
| | QDNGGLDSEESYPYEATEE | SEQ ID NO.: 2622 |
| | SCKYN | |
| | CSSEDMDHGVLVV | SEQ ID NO.: 2623 |
| | GFESTESDNNKYWLVKN | SEQ ID NO.: 2624 |
| Cathepsin S | TYGKQYKEKNEEAVRRLIWE | SEQ ID NO.: 2625 |
| | YKSNPNRILPDSVDW | SEQ ID NO.: 2626 |
| | CSTEKYGNKGCNGGFMTTA | SEQ ID NO.: 2627 |
| | NKGIDSDASYPYKAMDQK | SEQ ID NO.: 2628 |
| | VANKGPVSVGVDARH | SEQ ID NO.: 2629 |

Cathepsin inhibitors may be quite specific as to which cathepsin they inhibit (83), for example a cathepsin inhibitor being specifically selected to target only cathepsin C (U.S. Pat. No. 10,238,633B2) and another specific to cathepsin S (See www.opnme.com) while showing selection against cathepsin B. In a preferred embodiment of the present invention an inhibitor effective against cathepsin B is desirable.

In a further embodiment an antibody directed to a tumor upregulated protein, including for instance, as a non-limiting examples, brevican, EGFR, or a tumor associated antigen such as CEA or MAGEA1, and used to target a cathepsin inhibitor fused to or conjugated to that antibody to a particular tumor site. The antibody in this instance may be a standard tetrameric immunoglobulin or a sub-component such as a scFV.

In yet another embodiment a cathepsin inhibitor may be delivered intratumorally or in the case of tumors accessible in a dermal or mucosal surface administered topically or to the mucosa. While such a cathepsin inhibitor may include one of those listed above for systemic use, in some embodiments it includes a cystatin delivered as a protein or as a nucleic acid sequence encoding a cystatin.

A number of cathepsin inhibitors are known to the art and useful in the present invention. These include, but are not limited to, peptidylaldehydes, aziridinyl peptides and epoxysuccinyl peptides, nitrile derivatives, ketone derivatives, acryl hydrazine, vinyl sulfonate derivatives, epoxy succinic acid, betalactams, surugamides, loxistatin derivatives, sulfonamide derivatives, flavonoids, and many other products (84, 85, 86, 87, 88, 89). Products with cathepsin inhibitory characteristics have been extensively reviewed and the properties of each discussed (70, 81, 84, 89, 90). In addition, some natural plant medicinal products, including but not limited to, caffeic acid and chlorogenic acid have been shown to be cathepsin inhibitors (91). Useful cathepsin inhibitors are also described in the following United States patents and patent publications, each of which is incorporated by reference herein in its entirety: U.S. Pat. Nos. 8,748,649, 8,680,152, 8,518,874, 8,450,373, 8,431,733B2, U.S. Pat. Nos. 8,367,732, 8,324,417, 8,211,897, 8,163,735, 8,143,448, 8,106,059, 8,013,186, 8,013,183, 7,893,112, 7,893,093, 7,781,487, 7,737,300, 7,696,250, 7,662,849, 7,608,592, 7,547,701, 7,488,848, US20150191459, US20140256698, US20140221478, US20140018421, US20120329837, US20120282267, US20120190714, US20110281879, US20110172310, US20110046406, US20100305331, US20100266537, US20090312571, US20090270415, US20090234127, US20090233909, US20090203629, US20090170909, US20090023781, US20080293819, US20080214676, US20080161254, US20070287699. Given the ongoing efforts to identify and characterize cathepsin inhibitors it is likely that additional cathepsin inhibitor compounds will be added, so this list is not considered limiting.

In light of the differential sites of expression of cathepsin B and other cathepsins, the inhibition of cathepsin B is most desired. Natural peptidic cathepsin B inhibitors comprise 3 groups: aldehydes, aziridinyl peptides and epoxysuccinyl peptides. The first two groups include miraziridine and tokaramide A isolated from a marine sponge and leupeptin and YM-51084 peptides isolated from Streptomyces ((94) (89) (83) Among the epoxysuccinyl peptides the best recognized is E64 originally isolated from Aspergillus japonicus. Many derivatives of E64 have been evaluated, the best studied being E64d (also known as loxistatin and aloxistatin) which has improved cell permeability and adsorption. Loxistatin is an epoxysuccinyl peptide and is the best studied cathepsin B inhibitor, and along with various derivatives thereof has been evaluated in humans for its potential effect in muscular dystrophy, demonstrating its safety and enabling pharmacokinetic studies (95). Some further derivatives of E64d show improved selectivity for cathepsin B. Some derivates have shown further selectivity for Cathepsin B including CA074. The relative activity of these are reviewed in Frlan (89)(incorporated herein by reference in its entirety.

In some preferred embodiments, the cathepsin inhibitor is an irreversible covalent inhibitor (e.g., aloxistatin). In some preferred embodiments, the cathepsin inhibitor is a reversible cathepsin inhibitor. In some preferred embodiments, the cathepsin inhibitor is a non-covalent inhibitor.

Suitable cathepsin inhibitors include, but are not limited to, the following compounds described in Siklos et al., Acta Pharmaceutical Sinica B (2015) 5(6):506-519.

Epoxysuccinate cysteine protease inhibitors (e.g., compounds 1 to 12 and derivatives and salts thereof; aloxistatin is E-64d).

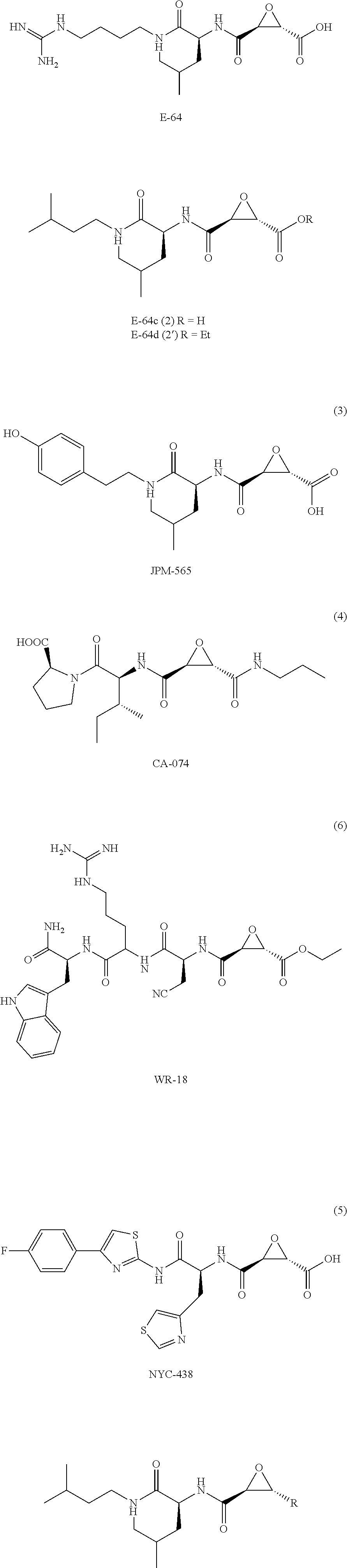

R=COOH (2), CONHOH (7), CONH₂ (8), COCH_{Z}(9), COOEt (10), CH₂OH (11), H (12)

Aziridine and β-lactone cysteine protease inhibitors (e.g., compounds 13-15 and derivatives and salts thereof).

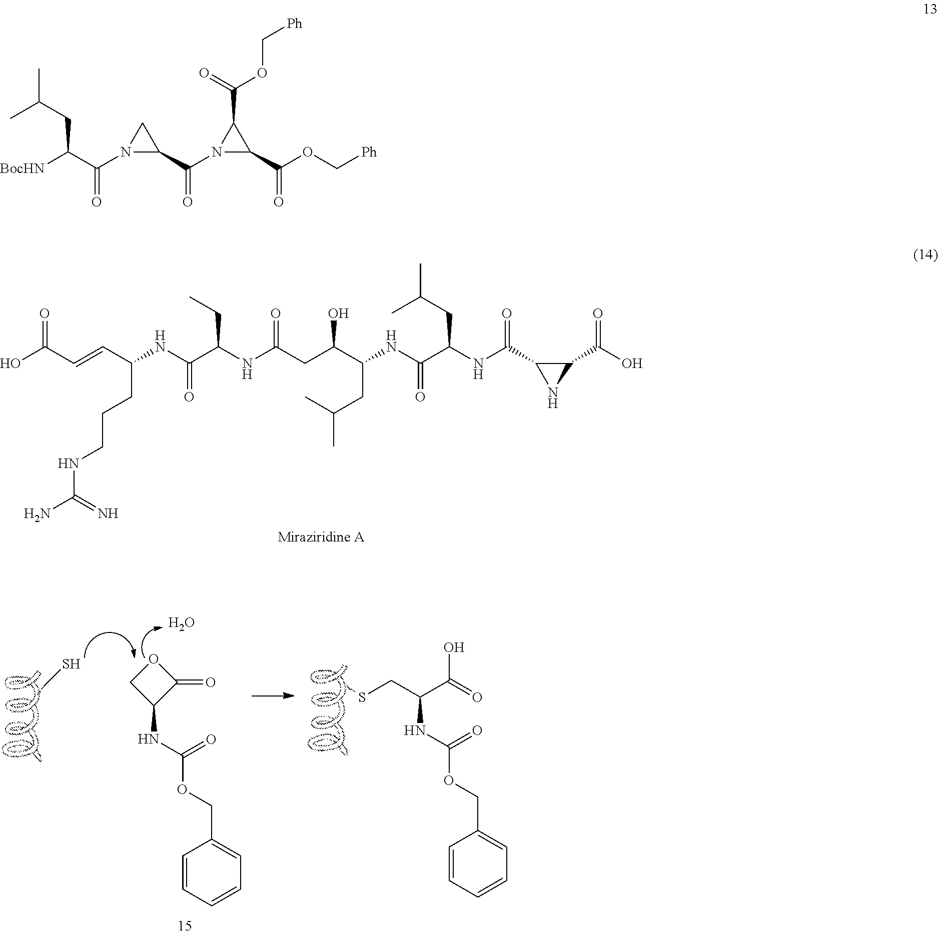

Michael acceptor warheads in cysteine protease inhibitors (e.g., compounds 16 to 20 and derivatives and salts thereof).

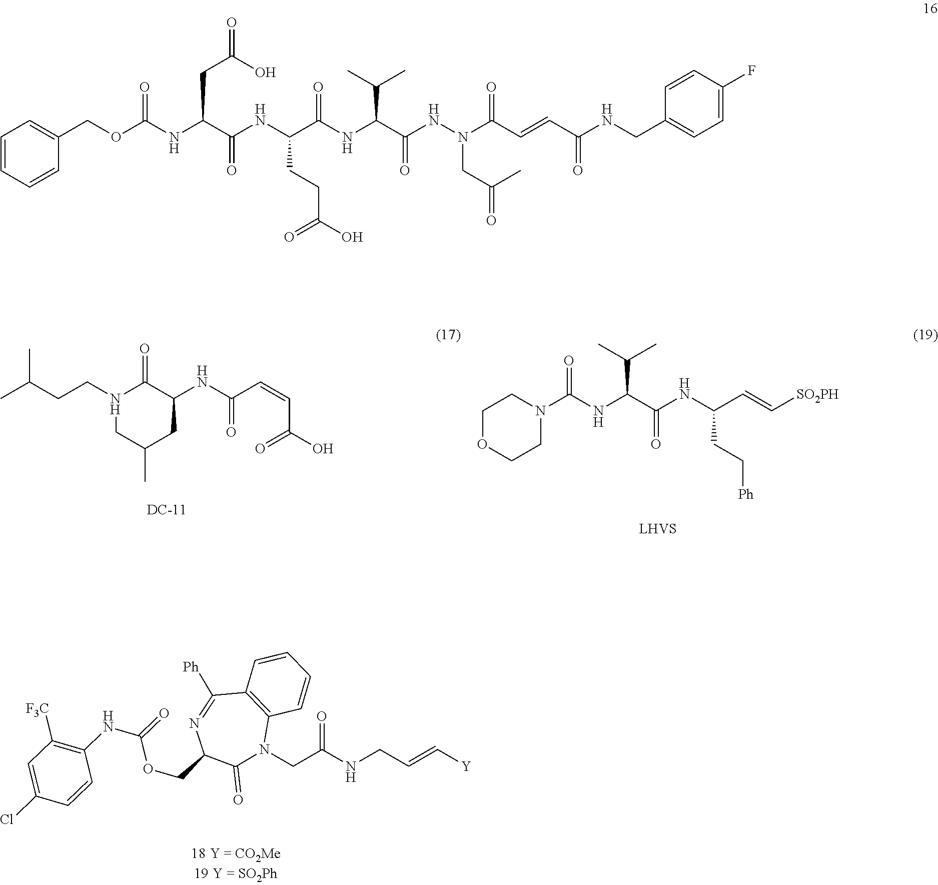

Diazomethyl, acyloxy and other ketone cysteine protease inhibitors (e.g., compounds 21 to 28 and derivatives and salts thereof).

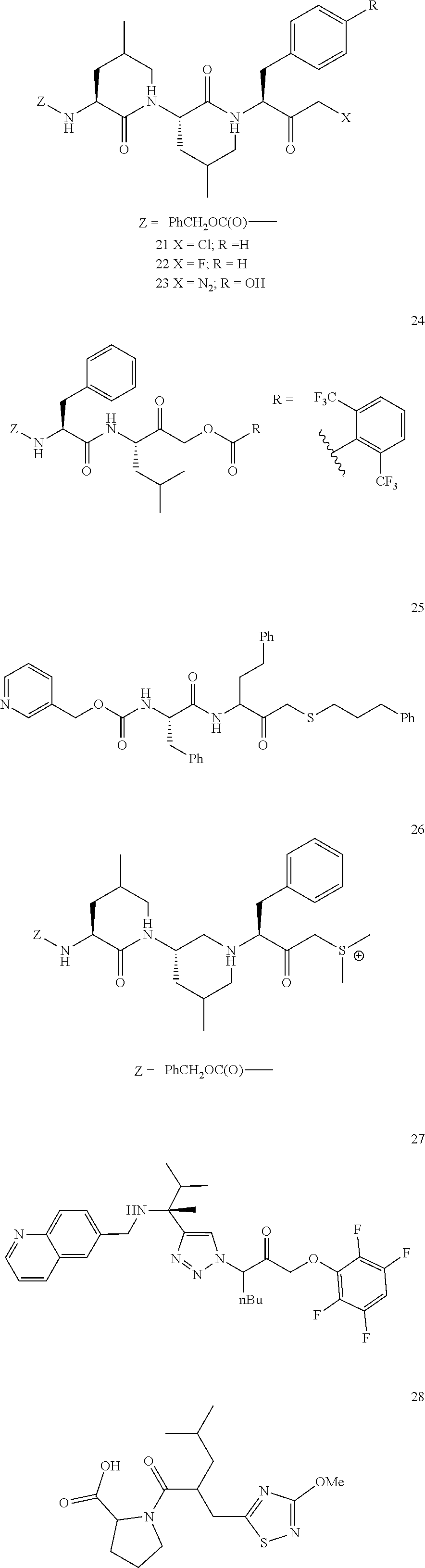

Aldehyde and cyclopropenone inhibitors (e.g., compounds 29-35 and derivatives and salts thereof)

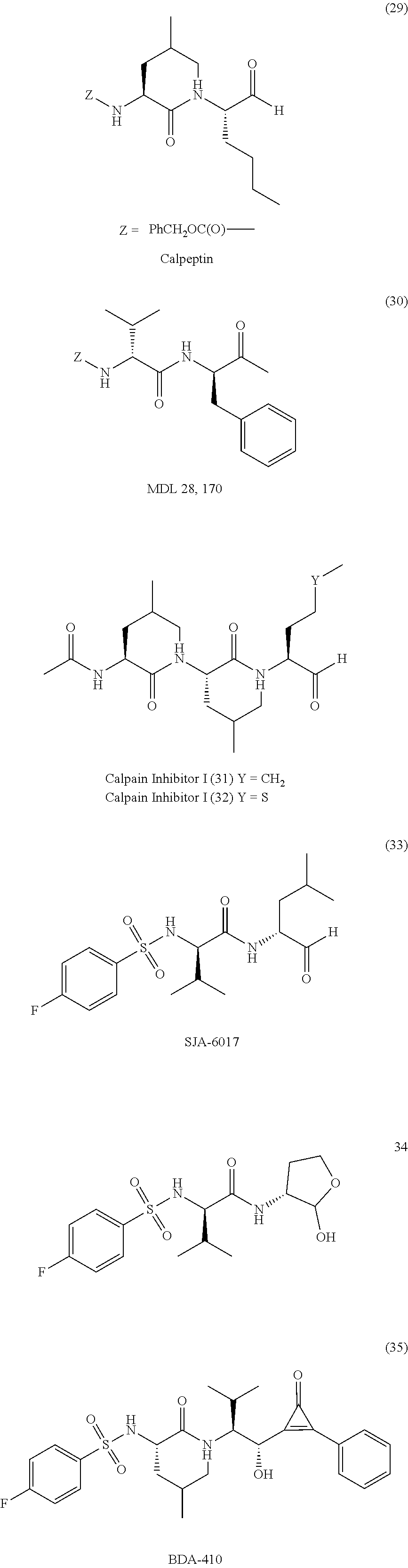

Ketoamide and ketoheterocycle cysteine protease inhibitors (e.g., compounds 36-45 and derivatives and salts thereof.)

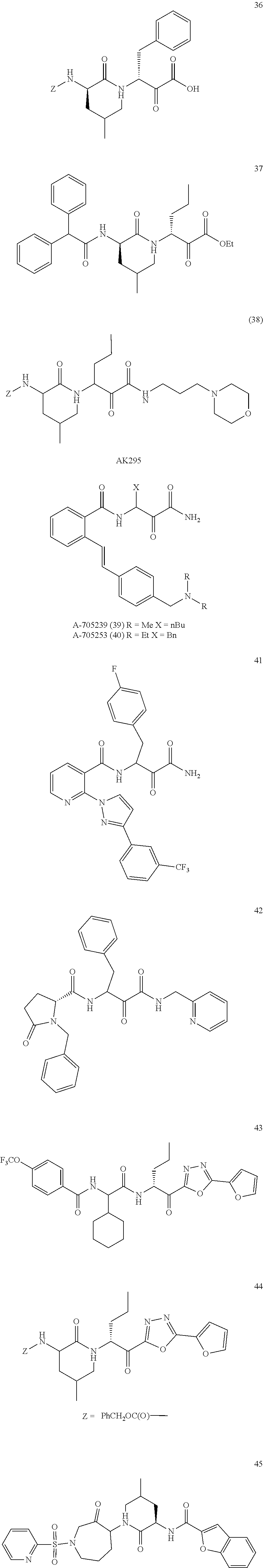

Nitrile and carbodiimide inhibitors (e.g., compounds 45 to 55 and derivatives and salts thereof).

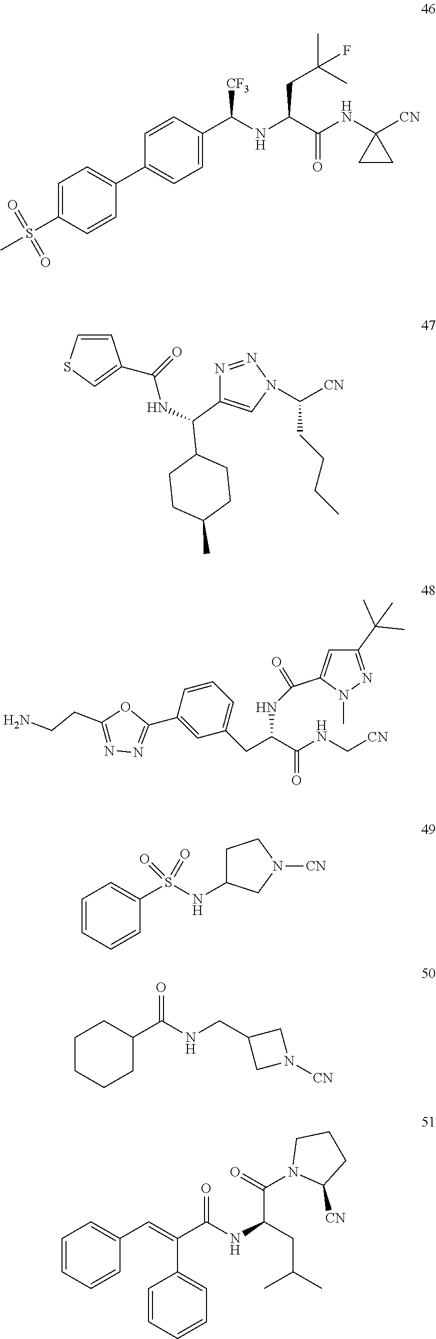

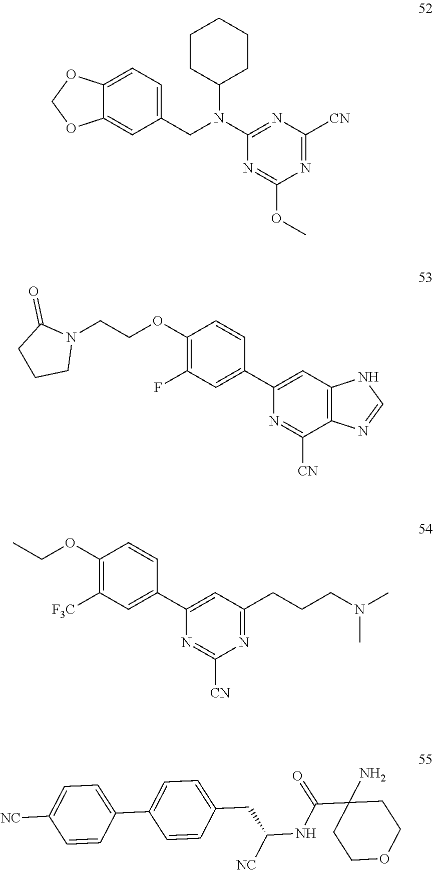 [SMILES: COc1nc(C#N)nc(N(Cc2ccc3c(c2)OCO3)C2CCCCC2)n1 ; N#Cc1nc(-c2ccc(OCCN3CCCC3=O)c(F)c2)cc2[nH]cnc12 ; CCOc1ccc(-c2cc(CCCN(C)C)nc(C#N)n2)cc1C(F)(F)F ; N#Cc1ccc(-c2ccc(C[C@@H](C#N)NC(=O)C3(N)CCOCC3)cc2)cc1]

Allosteric inhibitors (e.g., compounds 56 to 59 and derivatives and salts thereof).

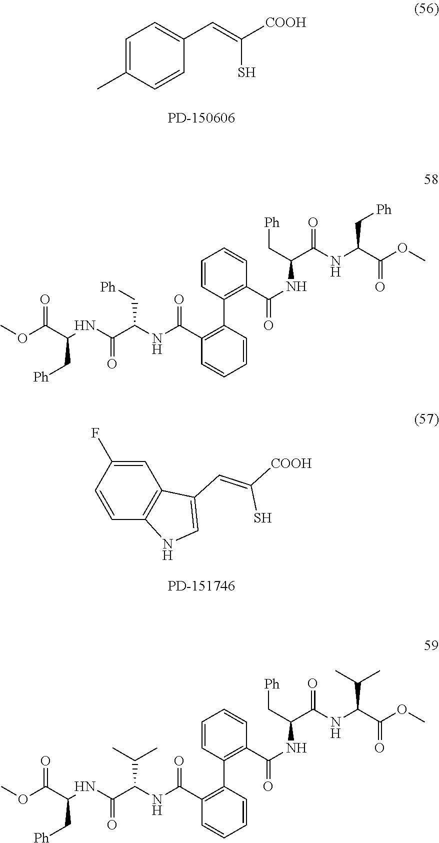

Non-peptidic natural compounds include astaxanthin and various flavonoids including amentoflavone, methylamentoflavone and dimethylamentoflavone. Additional groups of irreversible cathepsin B inhibitors include aziridines, 1,2,4-thiadiazoles, acycloxymethylketones, beta lactams, and organotellurium compounds. Reversible cathepsin B inhibitors include members of the groups of aldehydes, ketones, cyclopropenones and cyclometallated compounds and nitriles (89).

### EXAMPLES

### Example 1: Frequency Distributions of Pentameric Amino Acid Motifs Corresponding to Potential T Cell Exposed Motif

The T cell exposed motif for an MHC I bound peptide comprise amino acids 4,5,6,7,8 of a 9mer peptide. The T cell exposed motif for an MHC II bound peptide most often comprise amino acids 2,3,5,7,8 of the central 9mer peptide of a 15mer peptide or amino acids −1,3,5,7,8 relative to the central 9mer of the 15mer. Presentation of pentameric motifs in these configurations in the thymus and from exogenous sources is key to the establishment of the T cell repertoire.

The T cell response to an exposed T cell exposed motif depends on there being a T cell in the subject's repertoire with a TCR that engages the T cell exposed motif with sufficient but not excessive affinity. For such a T cell to exist in the subjects T cell repertoire, the pentameric amino acid motif corresponding to the T cell exposed motif must have been previously encountered, either during thymic selection of naïve T cells based on the presence of such a pentameric motif in the subject's self-proteome or by presentation, directly or via a dendritic cell or other antigen presenting cell, of such a pentameric motif derived from an exogenous source. It has been demonstrated that peptides from the gastrointestinal microbiome play a role in generation of cognate T cell clones early in life (36).

The frequency of occurrence of each pentamer amino acid motif in the configurations indicated above in a reference protein database can be determined. Relevant reference databases include, but is not limited to, the human proteome, proteins of a representative set of bacterial of the gastrointestinal microbiome, the variable regions of the human immunoglobulinome, common pathogens and vaccinal antigens and other collections of exogenous proteins to which a subject may be exposed. Determination of the frequency of pentamer motifs has been previously described (16, 22). See also PCT APPL. US/2015/039969, incorporated herein by reference in its entirety. Not all of the pentamer motifs of either configuration are found in the normal human proteome. A different subset is absent from the proteins of a representative gastrointestinal microbiome. We show here how comparing the T cell exposed motif created by a tumor mutation to such pentamer amino acid motif frequencies is an important and useful criterion in selecting the most desired peptides for neoepitope vaccine inclusion and for avoiding the least desired peptides.

As shown in FIG. 2 when the frequencies of the pentamers comprising the T cell exposed motifs in mutated oncogene and tumor suppressor proteins are determined relative to the counts of the same pentameric amino acid motifs in the normal human proteome, it is noted that tumor mutations produce pentameric T cell exposed motifs that are less common overall in the normal proteome than the corresponding pentamer motifs in their unmutated wildtype counterparts. In about 10% of cases a tumor mutation generates a pentameric amino acid motif which is entirely absent from the normal human proteome. FIG. 3 shows that this trend to less common pentamer motifs is confined to the T cell exposed motif positions of the mutant peptide and has no impact on pentamers which position the mutated amino acid outside the T cell exposed motif, including in the groove exposed motifs.

In FIGS. 2 and 3 each protein in the human proteome is represented by its longest isoform. The human proteome reference database derived from Hg38 comprises 20,916 proteins which collectively comprise 2,347,798 unique pentameric amino acid motifs in the configuration for presentation by MHC I and 2,384,149 unique motifs in the configuration for presentation by MHC II. The terms human proteome pentamer frequency (hPPF) refers to the count of occurrences of a particular amino acid pentameric motif in the human proteome and MHC I and MHC II configurations are styled as PPFI and PPF II.

The gastrointestinal microbiome reference database comprises by all open reading frames in the genomes of 67 bacterial species in 35 genera assembled from the NIH Human Microbiome Project Reference Genomes database (www.hmpdacc.org/HMRGD) (22, 96). This database comprises over 211,404 proteins comprising 109 million sequential 15 mer peptides. These contain 2,906,376 unique PPF I motifs and 2,921,480 PPF II motifs. The gastrointestinal microbiome database used here is an illustrative reference database made up of a typical combination of bacterial species. However, this should not be considered limiting as the gastrointestinal microbiome may vary between individuals and over time.

Individual wild type proteins may differ considerably in the baseline frequency of each sequential pentameric amino acid motif that they comprise. Some, including TP53 comprise a high percentage of uncommon pentameric motifs and mutations frequently generate tumor specific T cell exposed pentamer motifs that have no match among the pentameric amino acid motifs in the normal human proteome and are rare or absent in the reference microbiome dataset. In other proteins, which have a higher frequency in the wildtype protein, mutation produces a mutant T cell exposed motif corresponding to a lower count of the same pentameric amino acid motifs in the human proteome, but not necessarily completely absent from it. In yet others, with KRAS being an example, there is a high frequency of representation of the mutated pentamers elsewhere in the proteome although substantially fewer than is the case in the wildtype. FIG. 4 compares pentameric amino acid motif frequency in two common mutations, TP53 R175H and KRAS G12D. KRAS has a notably reduced motif frequency in the example shown but additional factors enter into immune evasion.

### Example 2: Peptide Selection Based on T Cell Exposed Motif Frequency

Absence of a particular pentameric amino acid motif from the human proteome precludes its presentation to uncommitted T cells in the thymus during positive selection, thus reducing the chance of establishment of a precursor T cell clone (29). This is compensated in early life by the exposure of naïve T cells, via dendritic cells, to T cell exposed motifs from environmental sources including but not limited to the gastrointestinal microbiome (36). In the figures shown herewith, in addition to frequencies in the human proteome, we have applied frequency analysis of pentamers in a representative gastrointestinal microbiome as an example of such non-human exposure to pentamer diversity. A low count in both databases is indicative of a likely low count of precursor T cell clones that could be responsive to the T cell exposed motif. Conversely, a low count in the human proteome is also indicative of a lower probability of adverse epitope mimic motifs in the proteome. A high count of pentamers in either reference database indicates that there is a better chance of cognate T cells in the repertoire. A high count in the human proteome may presage a higher chance of epitope mimics with adverse outcomes. Pentamers in the human proteome that match T cell exposed motifs, thus having the potential to be epitope mimics, have to be considered in the context of binding to each subject's HLA alleles. Only when MHC binding and presentation of that pentameric amino acid motif occurs in the proteome context does it lead to T cell presentation and hence potential mimics. Hence peptides of interest should be evaluated in the context of the subject's HLA to determine the risk of such mimics.

A single missense mutation results in 5 potential unique T cell exposed motifs for MHC I presentation and similarly 5 for MHC II presentation. Each differs in their relative frequency of representation (count) in the proteome and gastrointestinal microbiome reference databases.

Table 7 provides examples from 4 common mutations in known driver gene products. As they are provided as examples it will be understood that they are not considered limiting. It shows the variation in frequency of representation (count of occurrences) in the human and gastrointestinal microbiome databases by each position and sequential pentamer T cell exposed motif. This ranges from zero to several hundred counts. The most preferred T cell exposed motifs based on their frequency are indicated for each example. It will be evident to those skilled in the art, and from discussion herein, that the only peptide positions of relevance are those in which the mutant amino acid is exposed to the T cell receptor and not hidden in the groove exposed motif. These positions are indicated as T cell exposed (TCEM) or groove exposed (GEM) facets.

Table 8 shows the naturally occurring peptides and their predicted binding affinity for an individual subject of one exemplar HLA genotype (A0101, A3301, B1501, B3501, C0501, C1203 and DRB1_0301, DRB1_0701. This table may be aligned with Table 8 based on the index positions of the peptides. Binding affinity for each allele is shown in standard deviation units below the mean for the protein, based on the concept that binding is competitive. A value of −1 SD approximates the Kd of binding and is indicative of binding sufficient to ensure presentation. While absolute binding will vary by protein and allele this corresponds to approximately 2000 nM. Binding affinity in excess of −2.75 SD units (approximately 5 nM) is indicative of binding of such high affinity that it may lead towards T cell exhaustion.

For some of the peptides, MHC binding affinity is so low that it is unlikely that the peptide would be bound in the natural tumor setting. In yet others binding affinity is lower than optimal such that it would be presented in the natural setting, but that T cell clones responsive to the T cell exposed motifs may be better stimulated by a heteroclitic peptide comprising the T cell exposed motif of interest but with amino acid residues substituted by alternate amino acids in the groove exposed motifs to enhance binding for a particular allele. Examples are shown for the EGFR G598V mutation in Table 9.

Four representative mutation examples are shown of the proteins in Tables 7 and 8. These illustrations are provided as non-limiting examples and it will be further understood that different motifs may be selected for individuals of other HLA genotype for these specific mutations. Furthermore, this is a non-limiting illustration of the selection in exemplar driver gene products, but the same approach may be equally applied to other driver gene products, other mutations in driver gene products and mutations in passenger gene products.

TABLE 7

| | | SEQ | | SEQ | | | | | | | |
|---|---|---|---|---|---|---|---|---|---|---|---|
| Protein | | ID | | ID | | | Index | | | | giPPF |
| mutation | TCEM I | NO.: | TCEM II | NO.: | facet I | facet II | position | hPPF I | giPPF I | hPPF II | II |
| | ~~~DGPHC~ | 1729 | GP~C~KT | 1790 | | | 584 | 2 | 0 | 8 | 18 |
| EGFR- | ~~~GPHCV~ | 1730 | PH~V~TC | 1791 | | | 585 | 3 | 0 | 1 | 0 |
| G598V | | | | | | | | | | | |
| EGFR- | ~~~PHCVK~ | 1731 | HC~K~CP | 1792 | | | 586 | 1 | 3 | 3 | 5 |
| G598V | | | | | | | | | | | |
| EGFR- | ~~~HCVKT~ | 1732 | CV~T~PA | 1793 | | GEM_II | 587 | 2 | 7 | 5 | 25 |
| G598V | | | | | | | | | | | |
| EGFR- | ~~~CVKTC~ | 1733 | VK~C~AV | 1794 | | TCEM_II* | 588 | 5 | 47 | 5 | 55 |
| G598V | | | | | | | | | | | |
| EGFR- | ~~~VKTCP~ | 1734 | KT~P~VV | 1795 | | TCEM_II* | 589 | 1 | 51 | 8 | 100 |
| G598V | | | | | | | | | | | |
| EGFR- | ~~~KTCPA~ | 1735 | TC~A~VM | 1796 | GEM_I | GEM_II | 590 | 2 | 10 | 2 | 2 |
| G598V | | | | | | | | | | | |
| EGFR- | ~~~TCPAV~ | 1736 | CP~V~MG | 1797 | TCEM_I* | TCEM_II | 591 | 4 | 14 | 1 | 2 |
| G598V | | | | | | | | | | | |
| EGFR- | ~~~CPAVV~ | 1737 | PA~V~GE | 1798 | TCEM_I* | GEM_II | 592 | 5 | 16 | 20 | 154 |
| G598V | | | | | | | | | | | |
| EGFR- | ~~~PAVVM~ | 1738 | AV~M~EN | 1799 | TCEM_I* | TCEM_II* | 593 | 10 | 77 | 3 | 30 |
| G598V | | | | | | | | | | | |
| EGFR- | ~~~AVVMG~ | 1739 | VV~G~NN | 1800 | TCEM_I* | TCEM_II* | 594 | 14 | 135 | 6 | 86 |
| G598V | | | | | | | | | | | |
| EGFR- | ~~~VVMGE~ | 1740 | VM~E~NT | 1801 | TCEM_I | GEM_II | 595 | 1 | 68 | 2 | 32 |
| G598V | | | | | | | | | | | |
| EGFR- | ~~~VMGEN~ | 1741 | MG~N~TL | 1802 | GEM_I | | 596 | 1 | 63 | 6 | 33 |
| G598V | | | | | | | | | | | |
| EGFR- | ~~~MGENN~ | 1742 | GE~N~LV | 1803 | GEM_I | | 597 | 2 | 23 | 11 | 79 |
| G598V | | | | | | | | | | | |
| EGFR- | ~~~GENNT~ | 1743 | EN~T~VW | 1804 | GEM_I | | 598 | 3 | 40 | 3 | 39 |
| G598V | | | | | | | | | | | |
| — | | | | | | | | | | | |
| FBXW7- | ~~~IHTLY~ | 1744 | HT~Y~HT | 1805 | | | 451 | 4 | 20 | 1 | 6 |
| R465C | | | | | | | | | | | |
| FBXW7- | ~~~HTLYG~ | 1745 | TL~G~TS | 1806 | | | 452 | 3 | 33 | 19 | 158 |
| R465C | | | | | | | | | | | |
| FBXW7- | ~~~TLYGH~ | 1746 | LY~H~ST | 1807 | | | 453 | 4 | 21 | 5 | 40 |
| R465C | | | | | | | | | | | |
| FBXW7- | ~~~LYGHT~ | 1747 | YG~T~TV | 1808 | | GEM_II | 454 | 8 | 26 | 8 | 59 |
| R465C | | | | | | | | | | | |
| FBXW7- | ~~~YGHTS~ | 1748 | GH~S~VC | 1809 | | TCEM_II* | 455 | 5 | 8 | 2 | 10 |
| R465C | | | | | | | | | | | |
| FBXW7- | ~~~GHTST~ | 1749 | HT~T~CC | 1810 | | TCEM_II | 456 | 6 | 41 | 0 | 0 |
| R465C | | | | | | | | | | | |
| FBXW7- | ~~~HTSTV~ | 1750 | TS~V~CM | 1811 | GEM_I | GEM_II | 457 | 12 | 28 | 4 | 5 |
| R465C | | | | | | | | | | | |
| FBXW7- | ~~~TSTVC~ | 1751 | ST~C~MH | 1812 | TCEM_I* | TCEM_II | 458 | 1 | 8 | 0 | 0 |
| R465C | | | | | | | | | | | |
| FBXW7- | ~~~STVCC~ | 1752 | TV~C~HL | 1813 | TCEM_I | GEM_II | 459 | 1 | 4 | 4 | 16 |
| R465C | | | | | | | | | | | |
| FBXW7- | ~~~TVCCM~ | 1753 | VC~M~LH | 1814 | TCEM_I | TCEM_II* | 460 | 1 | 0 | 1 | 18 |
| R465C | | | | | | | | | | | |
| FBXW7- | ~~~VCCMH~ | 1754 | CC~H~HE | 1815 | TCEM_I | TCEM_II | 461 | 0 | 0 | 0 | 2 |
| R465C | | | | | | | | | | | |
| FBXW7- | ~~~CCMHL~ | 1755 | CM~L~EK | 1816 | TCEM_I | GEM_II | 462 | 0 | 2 | 2 | 27 |
| R465C | | | | | | | | | | | |
| FBXW7- | ~~~CMHLH~ | 1756 | MH~H~KR | 1817 | GEM_I | | 463 | 1 | 0 | 3 | 4 |
| R465C | | | | | | | | | | | |
| FBXW7- | ~~~MHLHE~ | 1757 | HL~E~RV | 1818 | GEM_I | | 464 | 1 | 10 | 9 | 16 |
| R465C | | | | | | | | | | | |
| FBXW7- | ~~~HLHEK~ | 1758 | LH~K~VV | 1819 | GEM_I | | 465 | 4 | 40 | 10 | 35 |
| R465C | | | | | | | | | | | |
| — | | | | | | | | | | | |
| FGFR2- | ~~~NHTYH~ | 1759 | HT~H~DV | 1820 | | | 238 | 3 | 14 | 2 | 13 |
| S252W | | | | | | | | | | | |
| FGFR2- | ~~~HTYHL~ | 1760 | TY~L~VV | 1821 | | | 239 | 3 | 8 | 15 | 60 |
| S252W | | | | | | | | | | | |
| FGFR2- | ~~~TYHLD~ | 1761 | YH~D~VE | 1822 | | | 240 | 3 | 34 | 3 | 17 |
| S252W | | | | | | | | | | | |
| FGFR2- | ~~~YHLDV~ | 1762 | HL~V~ER | 1823 | | GEM_II | 241 | 5 | 62 | 16 | 39 |
| S252W | | | | | | | | | | | |
| FGFR2- | ~~~HLDVV~ | 1763 | LD~V~RW | 1824 | | TCEM_II* | 242 | 16 | 110 | 2 | 36 |
| S252W | | | | | | | | | | | |
| FGFR2- | ~~~LDVVE~ | 1764 | DV~E~WP | 1825 | | TCEM_II | 243 | 21 | 205 | 0 | 3 |
| S252W | | | | | | | | | | | |
| FGFR2- | ~~~DVVER~ | 1765 | VV~R~PH | 1826 | GEM_I | GEM_II | 244 | 16 | 90 | 4 | 14 |
| S252W | | | | | | | | | | | |
| FGFR2- | ~~~VVERW~ | 1766 | VE~W~HR | 1827 | TCEM_I | TCEM_II | 245 | 3 | 19 | 1 | 4 |
| S252W | | | | | | | | | | | |
| FGFR2- | ~~~VERWP~ | 1767 | ER~P~RP | 1828 | TCEM_I | GEM_II | 246 | 0 | 14 | 19 | 34 |
| S252W | | | | | | | | | | | |
| FGFR2- | ~~~ERWPH~ | 1768 | RW~H~PI | 1829 | TCEM_I | TCEM_II | 247 | 0 | 6 | 0 | 4 |
| S252W | | | | | | | | | | | |
| FGFR2- | ~~~RWPHR~ | 1769 | WP~R~IL | 1830 | TCEM_I | TCEM_II* | 248 | 1 | 2 | 0 | 20 |
| S252W | | | | | | | | | | | |
| FGFR2- | ~~~WPHRP~ | 1770 | PH~P~LQ | 1831 | TCEM_I | GEM_II | 249 | 0 | 0 | 13 | 4 |
| S252W | | | | | | | | | | | |
| FGFR2- | ~~~PHRPI~ | 1771 | HR~I~QA | 1832 | GEM_I | | 250 | 8 | 5 | 11 | 25 |
| S252W | | | | | | | | | | | |
| FGFR2- | ~~~HRPIL~ | 1772 | RP~L~AG | 1833 | GEM_I | | 251 | 6 | 38 | 18 | 100 |
| S252W | | | | | | | | | | | |
| FGFR2- | ~~~RPILQ~ | 1773 | PI~Q~GL | 1834 | GEM_I | | 252 | 15 | 25 | 11 | 38 |
| S252W | | | | | | | | | | | |
| — | | | | | | | | | | | |
| TP53-C176Y | ~~~QSQHM~ | 1774 | SQ~M~EV | 1835 | | | 162 | 4 | 10 | 5 | 14 |
| TP53-C176Y | ~~~SQHMT~ | 1775 | QH~T~VV | 1836 | | | 163 | 3 | 12 | 3 | 26 |
| TP53-C176Y | ~~~QHMTE~ | 1776 | HM~E~VR | 1837 | | | 164 | 3 | 2 | 3 | 16 |
| TP53-C176Y | ~~~HMTEV~ | 1777 | MT~V~RR | 1838 | | GEM_II | 165 | 2 | 16 | 5 | 12 |
| TP53-C176Y | ~~~MTEVV~ | 1778 | TE~V~RY | 1839 | | TCEM_II* | 166 | 8 | 23 | 2 | 40 |
| TP53-C176Y | ~~~TEVVR~ | 1779 | EV~R~YP | 1840 | | TCEM_II* | 167 | 13 | 62 | 5 | 46 |
| TP53-C176Y | ~~~EVVRR~ | 1780 | VV~R~PH | 1841 | GEM_I | GEM_II | 168 | 21 | 164 | 4 | 14 |
| TP53-C176Y | ~~~VVRRY~ | 1781 | VR~Y~HH | 1842 | TCEM_I* | TCEM_II | 169 | 3 | 44 | 2 | 10 |
| TP53-C176Y | ~~~VRRYP~ | 1782 | RR~P~HE | 1843 | TCEM_I* | GEM_IIa | 170 | 0 | 62 | 5 | 11 |
| TP53-C176Y | ~~~RRYPH~ | 1783 | RY~H~ER | 1844 | TCEM_I* | TCEM_II* | 171 | 4 | 19 | 1 | 48 |
| TP53-C176Y | ~~~RYPHH~ | 1784 | YP~H~RC | 1845 | TCEM_I | TCEM_II | 172 | 0 | 4 | 1 | 0 |
| TP53-C176Y | ~~~YPHHE~ | 1785 | PH~E~CS | 1846 | TCEM_I | GEM_IIa | 173 | 0 | 2 | 3 | 0 |
| TP53-C176Y | ~~~PHHER~ | 1786 | HH~R~SD | 1847 | GEM_I | | 174 | 1 | 4 | 2 | 4 |
| TP53-C176Y | ~~~HHERC~ | 1787 | HE~C~DS | 1848 | GEM_I | | 175 | 2 | 5 | 1 | 4 |
| TP53-C176Y | ~~~HERCS~ | 1788 | ER~S~SD | 1849 | GEM_I | | 176 | 2 | 28 | 12 | 45 |

hPPF I(human proteome pentamer frequency)indicates the count of the TCEM I pentamer in the human proteome (longest isoforms);
hPPF II indicates the count of the TCEM II discontinuous pentamer in the human proteome. The giPPF (gastrointestinal proteome pentamer frequency) shows the corresponding counts in the representative gastrointestinal proteome database. Preferred peptides comprise the TCEM I or TCEM II that are asterisked.

TABLE 8

| | | | SEQ | | SEQ | | SEQ | | SEQ |
|---|---|---|---|---|---|---|---|---|---|
| Protein | posi- | | ID | | ID | | ID | | ID |
| mutation | tion | TCEM I | NO.: | TCEM II | NO.: | 9-mer | NO.: | 15-mer | NO.: |
| EGFR- | 588 | | | VK~C~AV | 1794 | | | GPHCVKTCPAVVMGE | 1856 |
| G598V | | | | | | | | | |
| EGFR- | 593 | ~~~PAVVM~ | 1738 | AV~M~EN | 1799 | KTCPAVVMG | 1850 | KTCPAVVMGENNTLV | 1857 |
| G598V | | | | | | | | | |
| EGFR- | 594 | ~~~AVVMG~ | 1739 | VV~G~NN | 1800 | TCPAVVMGE | 1851 | TCPAVVMGENNTLVW | 1858 |
| G598V | | | | | | | | | |
| - | | | | | | | | | |
| TP53- | 166 | | | TE~V~RY | 1839 | | | SQHMTEVVRRYPHHE | 1859 |
| C176Y | | | | | | | | | |
| TP53- | 167 | | | EV~R~YP | 1840 | | | QHMTEVVRRYPHHER | 1860 |
| C176Y | | | | | | | | | |
| TP53- | 169 | ~~~VVRRY~ | 1781 | | | MTEVVRRYP | 1852 | | |
| C176Y | | | | | | | | | |
| TP53- | 171 | ~~~RRYPH~ | 1783 | RY~H~ER | 1844 | EVVRRYPHH | 1853 | EVVRRYPHHERCSDS | 1861 |
| C176Y | | | | | | | | | |
| - | | | | | | | | | |
| FGFR2- | 242 | | | LD~V~RW | 1824 | | | HTYHLDVVERWPHRP | 1862 |
| S252W | | | | | | | | | |
| FGFR2- | 245 | ~~~VVERW~ | 1766 | VE~W~HR | 1827 | HLDVVERWP | 1854 | HLDVVERWPHRPILQ | 1863 |
| S252W | | | | | | | | | |
| - | | | | | | | | | |
| FBXW7- | 455 | | | GH~S~VC | 1809 | | | HTLYGHTSTVCCMHL | 1864 |
| R465C | | | | | | | | | |
| FBXW7- | 458 | ~~~TSTVC~ | 1751 | | | YGHTSTVCC | 1855 | | |
| R465C | | | | | | | | | |
| FBXW7- | 460 | | | VC~M~LH | 1814 | | | HTSTVCCMHLHEKRV | 1865 |
| R465C | | | | | | | | | |

| posi- | A_ | A_ | B_1501 | B_ | C_ | C_ | DRB1_ | DRB1_ | hPPF | giPPF | hPPF | giPPF |
|---|---|---|---|---|---|---|---|---|---|---|---|---|
| tion | 0101 | 3301 | | 3501 | 0501 | 1203 | 0301 | 0701 | I | I | II | II |
| 588 | | | | | | | −0.28 | −0.58 | | | 5 | 55 |
| 593 | −0.56 | 1.74 | −0.80 | −0.50 | −0.45 | −0.91 | −0.80 | −0.82 | 10 | 77 | 3 | 30 |
| 594 | 0.49 | −0.71 | −0.74 | 0.47 | 0.15 | −1.45 | −0.95 | −0.55 | 14 | 135 | 6 | 86 |
| 166 | | | | | | | −2.63 | 0.07 | | | 2 | 40 |
| 167 | | | | | | | −1.73 | 0.58 | | | 5 | 46 |
| 169 | −1.96 | −0.56 | 1.65 | −1.22 | −1.25 | 0.14 | | | 3 | 44 | | |
| 171 | 0.65 | −1.30 | 0.06 | −1.09 | −1.08 | 0.67 | −0.59 | 1.00 | 4 | 19 | 1 | 48 |
| 242 | | | | | | | −3.32 | −0.97 | | | 2 | 36 |
| 245 | −1.63 | −1.23 | 0.18 | −0.21 | −0.84 | 0.76 | −1.33 | −0.37 | 3 | 19 | 1 | 4 |
| 455 | | | | | | | 0.18 | −1.16 | | | 2 | 10 |
| 458 | −0.66 | −0.20 | −1.52 | −1.70 | −2.68 | −1.98 | | | 1 | 8 | | |
| 460 | | | | | | | −1.66 | 0.32 | | | 1 | 18 |

Binding affinity for each allele is shown in standard deviation units below the mean for the protein

TABLE 9

| | | | | | | | natural | heteroclitic |
|---|---|---|---|---|---|---|---|---|
| | | | | | | | peptide | peptide |
| gene_ | | Heteroclitic | SEQ ID | | SEQ ID | | binding | binding |
| symbol | pos | peptide | NO.: | TCEM core | NO.: | Allele | A0101 | A0101 |
| EGFR- | 594 | KVGAVVMGR | 1866 | ~~~AVVMG~ | 1739 | A_0301 | −0.73 | −2.00 |
| G598V | | | | | | | | |
| EGFR- | 594 | HAEAVVMGY | 1867 | ~~~AVVMG~ | 1739 | B_1501 | −0.74 | −2.00 |
| G598V | | | | | | | | |
| EGFR- | 594 | KKFAVVMGD | 1868 | ~~~AVVMG~ | 1739 | C 1203 | −1.45 | −2.07 |
| G598V | | | | | | | | |
| EGFR- | 593 | TTEPAVVMA | 1869 | ~~~PAVVM~ | 1738 | A_0101 | −0.56 | −2.01 |
| G598V | | | | | | | | |
| EGFR- | 593 | ESEPAVVMF | 1870 | ~~~PAVVM~ | 1738 | B_1501 | −0.80 | −2.03 |
| G598V | | | | | | | | |
| EGFR- | 593 | EADPAVVMM | 187 | ~~~PAVVM~ | 1738 | C_0501 | −0.45 | −2.08 |
| G598V | | | | | | | | |
| EGFR- | 593 | YARPAVVME | 1872 | ~~~PAVVM~ | 1738 | C_1203 | −0.91 | −2.07 |
| G598V | | | | | | | | |
| gene_ | | Heteroclitic | | | | | nDRB1_ | DRB1_ |
| symbol | pos | peptide | | TCEM core | | Allele | 0701 | 0701 |
| EGFR- | 588 | KAVAVKTCPAVLEVV | 1873 | VK~C~AV | 1794 | DRB1_ | −0.58 | −2.02 |
| G598V | | | | | | 0701 | | |
| EGFR- | 588 | QGRYVKTCPAVAYHI | 1874 | VK~C~AV | 1794 | DRB1_ | −0.58 | −2.01 |
| G598V | | | | | | 0701 | | |
| EGFR- | 594 | AKYAVVMGENNRRPY | 1875 | VV~G~NN | 1800 | DRB1_ | −0.95 | −2.02 |
| G598V | | | | | | 0301 | | |
| EGFR- | 594 | PAVFVVMGENNRYAE | 1876 | VV~G~NN | 1800 | DRB1_ | −0.95 | −2.01 |
| G598V | | | | | | 0301 | | |
| EGFR- | 594 | EKRFVVMGENNLVEL | 1877 | VV~G~NN | 1800 | DRB1_ | −0.55 | −2.02 |
| G598V | | | | | | 0701 | | |
| EGFR- | 594 | LTQFVVMGENNALKL | 1878 | VV~G~NN | 1800 | DRB1_ | −0.55 | −1.99 |
| G598V | | | | | | 0701 | | |

Binding is shown in standard deviation units below the mean (z scale). While absolute predicted binding varies between proteins and alleles a score of −2 approximates to 200 nM and a shift from −0.73 to −2.04 represents an approximately 100 fold increase in binding

As noted above, T cell exposed motif frequency is also a criterion for selection of vaccinal peptides from mutated passenger gene products. Table 10—(also replicated as FIG. 5) provides an example for a particular individual cancer-affected subject showing the selection of the most advantageous peptides based on frequency of occurrence in reference databases of pentamer counts and the subject's HLA alleles. When the pentamers in tumor specific T cell exposed motifs that expose a mutated amino acid are absent or low frequency in the human proteome, particular attention is paid to the frequency count in databases of exogenous proteins. In the table we show application of the representative gastrointestinal microbiome dataset. Application of this database should not be considered limiting and other reference sets likely to be indicative of prior epitope exposure may be applied. In this example the top two passenger mutations (in genes ARHGAP5 and ATIC) have T cell exposed motifs which are common. However, the lower two mutations (in CDC42 and CHD6) are poorly represented in the human and gastrointestinal microbiome reference datasets. Selected peptides for these two mutated proteins (margins boxed in FIG. 5) that best fulfil the criteria of frequency and also binding to this subject's known HLA alleles include SEQ ID NO.:s. 1891, 1897, 1898, 1914, 1915, 1916, 1921. In another embodiment a subject affected with a R132H mutation of IDH1 was vaccinated multiple times with two 9 mer peptides both exposing the mutant histidine. One peptide carried T cell exposed motif ~~~IGHHA~ (SEQ ID NO.: 1924) and the other carrying ~~~GHHAY~ SEQ ID NO.: 1925 and a 15 mer peptide comprising KP~I~GH SEQ ID NO.: 1927. A positive Elispot response was detected to SEQ ID NO.: 1924 and 1927 but not SEQ ID NO.: 1925. The is consistent with the frequencies in the human proteome and gastrointestinal microbiome of the corresponding pentamers as shown in Table 11, where it is seen that the pentamer of SEQ 1925 is very rare in both the human proteome and the gastrointestinal microbiome.

TABLE 10
Passenger mutation examples from one subject

| gi | pos | gene symbol | TCEM I | SEQ ID NO.: | TCEM IIa | SEQ ID NO.: | hPPF I | giPPF I | hPPF II | giPPF II | A 0201 | A 3301 | DRB1 0101 | DRB1 0701 | DRB3 0101 |
|---|---|---|---|---|---|---|---|---|---|---|---|---|---|---|---|
| Q13017- | 689 | ARHGAP5 | | | NL~F~LT | 1900 | | | 10 | 104 | −0.92 | 1.10 | −1.36 | −0.88 | 0.03 |
| I699T | | | | | | | | | | | | | | | |
| Q13017- | 690 | ARHGAP5 | ~~~NLPFT~ | 1879 | LP~T~TL | 1901 | 7 | 75 | 25 | 213 | −4.05 | −0.36 | −1.83 | −1.12 | 0.29 |
| I699T | | | | | | | | | | | | | | | |
| Q13017- | 692 | ARHGAP5 | ~~~PFTLT~ | 1880 | FT~T~AN | 1902 | 20 | 166 | 7 | 37 | −1.99 | 0.69 | −1.24 | −0.60 | 0.38 |
| I699T | | | | | | | | | | | | | | | |
| Q13017- | 693 | ARHGAP5 | ~~~FTLTL~ | 1881 | TL~L~NQ | 1903 | 14 | 181 | 15 | 99 | −2.39 | −0.55 | −1.93 | −0.64 | −0.32 |
| I699T | | | | | | | | | | | | | | | |
| Q13017- | 694 | ARHGAP5 | ~~~TLTLA~ | 1882 | LT~A~QR | 1904 | 26 | 386 | 17 | 137 | 0.12 | −0.75 | −1.00 | −1.10 | 0.12 |
| I699T | | | | | | | | | | | | | | | |
| Q13017- | 695 | ARHGAP5 | ~~~LTLAN~ | 1883 | TL~N~RD | 1905 | 19 | 241 | 4 | 88 | 1.17 | 0.86 | −0.32 | 0.61 | 0.29 |
| I699T | | | | | | | | | | | | | | | |
| Q13017- | 696 | ARHGAP5 | ~~~TLANQ~ | 1884 | | | 2 | 70 | | | 0.14 | −2.17 | −0.26 | 0.59 | 1.04 |
| I699T | | | | | | | | | | | | | | | |
| Q13017- | 697 | ARHGAP5 | | | | | | | | | 0.79 | 0.21 | 0.19 | 0.66 | 0.50 |
| I699T | | | | | | | | | | | | | | | |
| P31939- | 508 | ATIC | | | FE~V~EF | 1906 | 6 | 132 | 5 | 59 | 0.43 | 0.06 | −0.86 | −1.02 | −0.78 |
| L518F | | | | | | | | | | | | | | | |
| P31939- | 509 | ATIC | | | EE~P~FL | 1907 | 8 | 29 | 17 | 61 | 0.36 | −1.01 | −0.06 | −0.09 | −0.10 |
| L518F | | | | | | | | | | | | | | | |
| P31939- | 511 | ATIC | ~~~EVPEF~ | 1885 | VP~F~TE | 1908 | 4 | 77 | 5 | 36 | −1.50 | 0.94 | 0.29 | 0.81 | 1.59 |
| L518F | | | | | | | | | | | | | | | |
| P31939- | 512 | ATIC | ~~~VPEFL~ | 1886 | | | 3 | 84 | | | −0.01 | −0.73 | 0.30 | 1.12 | 1.71 |
| L518F | | | | | | | | | | | | | | | |
| P31939- | 513 | ATIC | ~~~PEFLT~ | 1887 | EF~T~AE | 1909 | 16 | 60 | 6 | 50 | 0.59 | −1.35 | 1.41 | 1.41 | 0.20 |
| L518F | | | | | | | | | | | | | | | |
| P31939- | 514 | ATIC | ~~~EFLTE~ | 1888 | FL~E~EK | 1910 | 14 | 141 | 37 | 249 | −0.87 | 0.50 | 0.06 | −0.06 | −0.68 |
| L518F | | | | | | | | | | | | | | | |
| P31939- | 515 | ATIC | ~~~FLTEA~ | 1889 | | | 8 | 103 | | | 0.26 | −0.72 | −0.04 | −0.12 | −0.78 |
| L518F | | | | | | | | | | | | | | | |
| P31939- | 516 | ATIC | | | | | | | | | 1.12 | 0.61 | 0.95 | 0.90 | −0.19 |
| L518F | | | | | | | | | | | | | | | |
| P60953- | 37 | CDC42 | | | AV~V~IC | 1911 | | | 3 | 25 | −0.33 | −0.25 | −0.19 | −0.64 | −0.05 |
| G47C | | | | | | | | | | | | | | | |
| P60953- | 38 | CDC42 | | | VT~M~CG | 1912 | | | 1 | 18 | −0.63 | 0.29 | −0.02 | −0.09 | 0.92 |
| G47C | | | | | | | | | | | | | | | |
| P60953- | 40 | CDC42 | ~~~TVMIC~ | 1890 | VM~C~EP | 1913 | 0 | 9 | 0 | 0 | −2.05 | 0.17 | −1.21 | −1.22 | −0.97 |
| G47C | | | | | | | | | | | | | | | |
| P60953- | 41 | CDC42 | ~~~VMICG~ | 1891 | | | 0 | 13 | | | 0.24 | −1.50 | −0.58 | −1.13 | −1.69 |
| G47C | | | | | | | | | | | | | | | |
| P60953- | 42 | CDC42 | ~~~MICGE~ | 1892 | IC~E~YT | 1914 | 0 | 13 | 1 | 9 | −0.24 | 0.51 | −0.76 | −1.26 | −1.84 |
| G47C | | | | | | | | | | | | | | | |
| P60953- | 43 | CDC42 | ~~~ICGEP~ | 1893 | CG~P~TL | 1915 | 1 | 24 | 6 | 46 | 0.13 | −0.69 | −1.52 | −0.95 | −1.15 |
| G47C | | | | | | | | | | | | | | | |
| P60953- | 44 | CDC42 | ~~~CGEPY~ | 1894 | | | 0 | 8 | | | −1.71 | −0.33 | −0.67 | −1.03 | −0.35 |
| G47C | | | | | | | | | | | | | | | |
| P60953- | 45 | CDC42 | | | | | | | | | −2.39 | 1.33 | −1.35 | −0.53 | 1.04 |
| G47C | | | | | | | | | | | | | | | |
| Q8TD26- | 1932 | CHD6 | | | CQ~H~KL | 1916 | | | 0 | 6 | 0.31 | −0.27 | 1.36 | 0.42 | −1.90 |
| H1942L | | | | | | | | | | | | | | | |
| Q8TD26- | 1933 | CHD6 | | | QC~C~LM | 1917 | | | 1 | 0 | 0.51 | −0.60 | 0.21 | −0.72 | −1.51 |
| H1942L | | | | | | | | | | | | | | | |
| Q8TD26- | 1935 | CHD6 | ~~~CHCKL~ | 1895 | HC~L~ER | 1918 | 2 | 0 | 0 | 2 | 0.09 | 0.02 | 0.13 | −0.25 | −0.72 |
| H1942L | | | | | | | | | | | | | | | |
| Q8TD26- | 1936 | CHD6 | ~~~HCKLM~ | 1896 | | | 0 | 0 | | | 0.07 | −1.63 | 0.44 | 0.00 | −1.43 |
| H1942L | | | | | | | | | | | | | | | |
| Q8TD26- | 1937 | CHD6 | ~~~CKLME~ | 1897 | KL~E~WM | 1919 | 1 | 15 | 1 | 4 | 0.35 | −1.52 | 0.43 | −0.27 | −0.32 |
| H1942L | | | | | | | | | | | | | | | |
| Q8TD26- | 1938 | CHD6 | ~~~KLMER~ | 1898 | LM~R~MH | 1920 | 6 | 89 | 1 | 0 | −0.10 | −1.76 | 0.64 | 0.34 | −0.22 |
| H1942L | | | | | | | | | | | | | | | |
| Q8TD26- | 1939 | CHD6 | ~~~LMERW~ | 1899 | | | 1 | 3 | | | 0.02 | −1.34 | −0.29 | −0.02 | −0.38 |
| H1942L | | | | | | | | | | | | | | | |
| Q8TD26- | 1940 | CHD6 | | | ER~M~GL | 1921 | | | 5 | 38 | −0.17 | −1.83 | −0.24 | 0.03 | −1.87 |
| H1942L | | | | | | | | | | | | | | | |

TABLE 11

| | index | | SEQ ID | | SEQ ID | hPPF | giPPF | hPPF | giPPF | |
|---|---|---|---|---|---|---|---|---|---|---|
| gi | pos | TCEM I | NO.: | TCEM IIa | NO.: | I | I | Iia | Iia | Elispot |
| O75874- | 122 | | | KP~I~GH | 1927 | | | 3 | 6 | positive |
| R132H | | | | | | | | | | |
| O75874- | 123 | | | PI~I~HH | 1928 | | | 0 | 13 | |
| R132H | | | | | | | | | | |
| O75874- | 125 | ~~~IIIGH~ | 1922 | II~H~AY | 1929 | 2 | 109 | 3 | 30 | |
| R132H | | | | | | | | | | |
| O75874- | 126 | ~~~IIGHH~ | 1923 | | | 1 | 18 | 2 | 64 | |
| R132H | | | | | | | | | | |
| O75874- | 127 | ~~~IGHHA~ | 1924 | GH~A~GD | 1930 | 1 | 8 | 6 | 53 | positive |
| R132H | | | | | | | | | | |
| O75874- | 128 | ~~~GHHAY~ | 1925 | HH~Y~DQ | 1931 | 3 | 0 | 1 | 2 | negative |
| R132H | | | | | | | | | | |
| O75874- | 129 | ~~~HHAYG~ | 1926 | | | 1 | 6 | 3 | 14 | |
| R132H | | | | | | | | | | |

### Example 3: Cathepsin Cleavage May Impact Immune Evasion

Peptidase cleavage, and in particular cathepsin cleavage, may sever a peptide that otherwise would be bound in an MHC to present a T cell motif that comprises a tumor specific mutation to a T cell. A peptide that is cleaved is thus removed from recognition by T cells and will evade CD8+ and/or CD4+ immune responses. The profile of expression of different cathepsins varies between cell types (97). Different cathepsins are more active at particular temperatures and ph. Thus, the impact of cathepsin cleavage may differ between tissues and tumors. Cathepsin cleavage sites can be determinant in generating short peptides which become available for MHC binding, as well as cleavage that precludes availability for MHC binding of certain peptides (27). The role of such cleavage may differ between peptides binding MHC I molecules and their tightly constrained grooves and the more open ended MHC II which are more tolerant of peptides of different lengths. Methods for predicting the cleavage patterns of cathepsin have been previously described (see PCT APPL. US/2014/041525, incorporated herein by reference in its entirety) and validated in clinical settings (98).

The inventors have previously developed predictive algorithms for determining the probability for cathepsin B, L and S cleavage (27, 98) (See e.g., U.S. Pat. No. 11,069,427 incorporated herein by reference in its entirety). Briefly, these algorithms were developed using the following steps. Multiple chemical and physical properties reported amino acids were used to derive sets of principal components to provide proxies for each amino acid that encompass their variables. Drawing on large sets of experimentally determined peptide cleavage events (99, 100) for each cathepsin B, L or S, sets of octomer peptides which were cleaved and sets which were uncleaved were used to train a classifier and generate an ensemble of predictive equations to predict cleavage or non-cleavage. These were further refined by bagging (boot strap aggregation) repetition of multiple random subsets of the experimental dataset. To generate a cathepsin cleavage probability profile of a protein of interest (in the present instance of a mutated tumor protein) each sequential octomer in the protein of interest is subjected to multiple repetitions of the ensemble of predictive equations which in effect vote as a Condorcet jury (101) on whether the octomer is cleaved at its central dimer or not. The output is characterized as a probability score of 0-100% for cleavage of each possible dimer in the 9mer that constitutes a potential neoepitope bound by a MHC I, or the central 9mer of a 15mer bound by a MHC II. The probability of a mutated neoepitope being cleaved may be expressed according to the individual dimer cleavage probability or as the aggregate of cleavage probabilities across the neoepitope peptide.

An MHC class I 9mer is comprises eight potential scissile bonds. Cleavage of any these bonds will result in a loss of exposure of the TCEM within that 9mer to a T cell. Thus, a quantitative scoring metric for each TCEM pentamer is a summation of the number of scissile bonds in the peptide that are predicted to be cleaved by the enzyme with a probability of cleavage greater than a threshold. Thus, in preferred embodiments, the probability of cleavage by a cathepsin of each octomer centered on a potential scissile bond (i.e., four amino acids on either side) in any 9mer peptides that comprise the identified amino acid mutations in the tumor protein is determined. FIG. 9 provides a schematic depiction of how the octomers overlap with each of the 9mers that comprise a mutant amino acid (depicted as “X”). In practice a threshold of 0.8 (80%) is used and a maximum score of 8 occurs when all bonds have a high probability of being cleaved.

$$Score = {\sum }_{i = 1}^{8} ( cleavage prob scissle bond ( i ) \geq threshold )$$

By applying these predictive algorithms it is possible to draw a cathepsin profile for any protein showing the probability of cleavage at any amino acid dimer of interest in the protein, by cathepsin L, S or B and to derive a cleavage probability score for each 9mer that may play a role in tumor mutation recognition. When applied to a mutated tumor protein of interest this allows a prediction of whether a peptide comprising a mutant amino acid is likely to be excised as a peptide of suitable size for MHC binding and presentation and exposure of the mutant amino acid to the TCR, while in a further embodiment it predicts whether the peptide comprising the mutant amino acid is destroyed or retained intact to allow presentation.

Cleavage by peptidases is therefore a factor which can affect the probability that a particular peptide comprising a tumor specific mutation may be available for MHC binding. While in silico predictions can provide a probability of cleavage, rather than determine an absolute frequency, it is a further factor in selection of a preferred neoepitope. Table 12 shows comparative cathepsin cleavage probabilities in a group of peptides comprising common mutations of interest. In the interests of space, this example is illustrative, but should not be considered limiting as the same method can be applied to any mutated tumor protein of interest, including but not limited to those listed in Tables 1 and 2 and to any passenger mutation.

In Table 12 we show examples where a range of probabilities of cathepsin cleavage offers the opportunity to select away from peptides which may be cleaved and in contrast to select those peptides, and within them the TCEM comprising mutant amino acids, which have a lower probability of being eliminated by cathepsin cleavage. The peptides that are retained intact are desirable as neoantigen targets. It will be understood by those skilled in the art that a similar evaluation of probability of cleavage may be carried out for any mutated protein of interest and that the table provides representative examples

TABLE 12
Exemplars of the probability of cathepsin cleavage in a mutated tumor peptide.

| Mutation ID | facet I | facet II | peptide mut | CatS | CatL | CatB | SEQ ID NO.: |
|---|---|---|---|---|---|---|---|
| CDKN2A-H83Y-G > A | | TCEM_IIa | ADPATLTRPVYDAAR | 0.00 | 0.00 | 0.00 | 1681 |
| CDKN2A-H83Y-G > A | | TCEM_IIa | DPATLTRPVYDAARE | 0.00 | 0.11 | 0.01 | 1682 |
| CDKN2A-H83Y-G > A | GEM_I | GEM_IIa | PATLTRPVYDAAREG | 0.00 | 0.00 | 0.00 | 1683 |
| CDKN2A-H83Y-G > A | TCEM_I | TCEM_IIa | ATLTRPVYDAAREGF | 0.83 | 0.92 | 0.00 | 1684 |
| CDKN2A-H83Y-G > A | TCEM_I | GEM_IIa | TLTRPVYDAAREGFL | 0.00 | 0.06 | 0.00 | 1685 |
| CDKN2A-H83Y-G > A | TCEM_I | TCEM_IIa | LTRPVYDAAREGFLD | 0.34 | 0.58 | 0.01 | 1686 |
| CDKN2A-H83Y-G > A | TCEM_I | TCEM_IIa | TRPVYDAAREGELDT | 0.02 | 0.00 | 0.13 | 1687 |
| CDKN2A-H83Y-G > A | TCEM_I | GEM_IIa | RPVYDAAREGELDTL | 0.27 | 0.75 | 0.88 | 1688 |
| CTNNB1-S33C-C > G | | TCEM_IIa | SHWQQQSYLDCGIHS | 0.06 | 0.16 | 0.00 | 1689 |
| CTNNB1-S33C-C > G | | TCEM_IIa | HWQQQSYLDCGIHSG | 0.05 | 0.13 | 0.07 | 1690 |
| CTNNB1-S3C-C > G | GEM_I | GEM_IIa | WQQQSYLDCGIHSGA | 0.00 | 0.00 | 0.00 | 1691 |
| CTNNB1-S33C-C > G | TCEM_I | TCEM_IIa | QQQSYLDCGIHSGAT | 0.00 | 0.00 | 0.00 | 1692 |
| CTNNB1-S33C-C > G | TCEM_I | GEM_IIa | QQSYLDCGIHSGATT | 0.00 | 0.21 | 0.02 | 1693 |
| CTNNB1-S33C-C > G | TCEM_I | TCEM_IIa | QSYLDCGIHSGATTT | 0.82 | 0.87 | 0.58 | 1694 |
| CTNNB1-S33C-C > G | TCEM_I | TCEM_IIa | SYLDCGIHSGATTTA | 0.98 | 0.68 | 0.2 | 1695 |
| CTNNB1-S33C-C > G | TCEM_I | GEM_IIa | YLDCGIHSGATTTAP | 0.02 | 0.15 | 0.60 | 1696 |
| FGFR2-S252W-G > C | | TCEM_IIa | HTYHLDVVERWPHRP | 0.00 | 0.19 | 0.62 | 1697 |
| FGFR2-S252W-G > C | | TCEM_IIa | TYHLDVVERWPHRPI | 0.26 | 0.02 | 0.91 | 1698 |
| FGFR2-S252W-G > C | GEM_I | GEM_IIa | YHLDVVERWPHRPIL | 0.27 | 0.61 | 0.06 | 1699 |
| FGFR2-S252W-G > C | TCEM_I | TCEM_IIa | HLDVVERWPHRPILQ | 0.04 | 0.20 | 0.00 | 1700 |
| FGFR2-S252W-G > C | TCEM_I | GEM_IIa | LDVVERWPHRPILQA | 0.05 | 0.31 | 0.00 | 1701 |
| FGFR2-S252W-G > C | TCEM_I | TCEM_IIa | DVVERWPHRPILQAG | 0.01 | 0.24 | 0.00 | 1702 |
| FGFR2-S252W-G > C | TCEM_I | TCEM_IIa | VVERWPHRPILQAGL | 0.05 | 0.00 | 0.00 | 1703 |
| FGFR2-S252W-G > C | TCEM_I | GEM_IIa | VERWPHRPILQAGLP | 0.02 | 0.18 | 0.05 | 1704 |
| FGFR3-S249C-C > G | | TCEM_IIa | QTYTLDVLERCPHRP | 0.00 | 0.45 | 0.01 | 1705 |
| FGFR3-S249C-C > G | | TCEM_IIa | TYTLDVLERCPHRPI | 0.04 | 0.07 | 0.47 | 1706 |
| FGFR3-S249C-C > G | GEM_I | GEM_IIa | YTLDVLERCPHRPIL | 0.88 | 0.26 | 0.02 | 1707 |
| FGFR3-S249C-C > G | TCEM_I | TCEM_IIa | TLDVLERCPHRPILQ | 0.00 | 0.00 | 0.00 | 1708 |
| FGFR3-S249C-C > G | TCEM_I | GEM_IIa | LDVLERCPHRPILQA | 0.00 | 0.14 | 0.00 | 1709 |
| FGFR3-S249C-C > G | TCEM_I | TCEM_IIa | DVLERCPHRPILQAG | 0.29 | 0.43 | 0.00 | 1710 |
| FGFR3-S249C-C > G | TCEM_I | TCEM_IIa | VLERCPHRPILQAGL | 0.00 | 0.00 | 0.00 | 1711 |
| FGFR3-S249C-C > G | TCEM_I | GEM_IIa | LERCPHRPILQAGLP | 0.00 | 0.37 | 0.00 | 1712 |

### Example 4: Impact of Cathepsin Cleavage on Ras Gene Products

While cathepsin cleavage probability is a consideration in selection of any potential neoantigen, it is of particular concern and consideration for mutations of Ras gene products, including but not limited to KRAS, HRAS, and NRAS. Mutations of the Ras genes, and primarily KRAS, contribute to a large proportion of cancers, in some estimates as many as 25% of all cancers (61). Of these KRAS comprises over 86% of Ras mutations. The KRAS, HRAS and NRAS proteins are completely aligned in their first 86 amino acids and very highly conserved thereafter. Over 90% of the mutations in KRAS and the other Ras occur in two hotspots, at amino acid G12 or G13 and at Q61. As we show in FIG. 4, these are positions with T cell exposed motifs that have a very high degree of representation in the human proteome and in the representative gastrointestinal microbiome. Predicted MHC binding of those peptides that comprise the commonly mutated sites in KRAS includes some peptides where binding is moderately high for common alleles including A 0201 and other A02 alleles, but lower for some other A alleles including A2402. Similarly, about half of the DRB1 alleles evaluated show binding to one or more TCEM positions with moderate to high affinity. Binding is therefore not a limitation. However, as shown in FIGS. 6 and 7 when the predicted probability of cathepsin B, L and S cleavage is examined relative to the dominant mutant positions, it is found to be very high, in excess of 0.8 for many positions and cathepsins, and 1.0 (100%) in multiple positions that would result in cleavage of the peptide that carries the mutant amino acid in the otherwise T cell exposed position. This is particularly evident for cathepsin B which has a probability of cleavage of >0.9 in most positions spanning the mutations at G12 and G13 and in at least two positions comprising mutations of Q61. This distribution of predicted cathepsin cleavage is also shown in Table 13, together with the positions at which such cleavage is predicted to occur.

TABLE 13
Cathepsin cleavage sites and probabilities in KRAS peptides

| | | | | | 9 mer | | SEQ ID | | | |
|---|---|---|---|---|---|---|---|---|---|---|
| | | | | | SEQ | | NO.: | | | |
| | | facet | facet | 9-mer | ID | | Cut | CAT_ | CAT_ | CAT_ |
| mutant | pos | 1 | 2 | peptide | NO.: | Peptide cutsite | site | S | L | B |
| G12A | 1 | | GEM2 | MTEYKLVVV | 2080 | 1 | 2342 | 0.00 | 0.00 | 0.47 |
| | | | | | | MTEY~KLVVVGAAGVG | | | | |
| G12A | 2 | | TCEM2 | TEYKLVVVG | 2081 | 2 | 2343 | 0.00 | 0.26 | 0.04 |
| | | | | | | TEYK~LVVVGAAGVGK | | | | |
| G12A | 3 | | TCEM2 | EYKLVVVGA | 2082 | 3 | 2344 | 0.00 | 0.16 | 0.65 |
| | | | | | | EYKL~VVVGAAGVGKS | | | | |
| G12A | 4 | GEM1 | GEM2 | YKLVVVGAA | 2083 | 4 | 2345 | 0.83 | 0.97 | 0.94 |
| | | | | | | YKLV~VVGAAGVGKSA | | | | |
| G12A | 5 | TCEM | TCEM2 | KLVVVGAAG | 2084 | 5 | 2346 | 1.00 | 0.66 | 1.00 |
| | | 1 | | | | KLVV~VGAAGVGKSAL | | | | |
| G12A | 6 | TCEM | GEM2 | LVVVGAAGV | 2085 | 6 | 2347 | 1.00 | 1.00 | 1.00 |
| | | 1 | | | | LVVV~GAAGVGKSALT | | | | |
| G12A | 7 | TCEM | TCEM2 | VVVGAAGVG | 2086 | 7 | 2348 | 0.95 | 1.00 | 1.00 |
| | | 1 | | | | VVVG~AAGVGKSALTI | | | | |
| G12A | 8 | TCEM | TCEM2 | VVGAAGVGK | 208 | 8 | 2349 | 0.42 | 0.13 | 0.99 |
| | | 1 | | | | VVGA~AGVGKSALTIQ | | | | |
| G12A | 9 | TCEM | GEM2 | VGAAGVGKS | 2088 | 9 | 2350 | 0.19 | 0.20 | 0.99 |
| | | 1 | | | | VGAA~GVGKSALTIQL | | | | |
| G12A | 10 | GEM1 | | GAAGVGKSA | 2089 | 10 | 2351 | 0.31 | 0.59 | 0.77 |
| | | | | | | GAAG~VGKSALTIQLI | | | | |
| G12A | 11 | GEM1 | | AAGVGKSAL | 2090 | 11 | 2352 | 0.12 | 0.08 | 0.99 |
| | | | | | | AAGV~GKSALTIQLIQ | | | | |
| G12A | 12 | GEM1 | | AGVGKSALT | 2091 | 12 | 2353 | 0.70 | 0.68 | 0.82 |
| | | | | | | AGVG~KSALTIQLIQN | | | | |
| G12A | 13 | | | GVGKSALTI | 2092 | 13 | 2354 | 0.03 | 0.79 | 0.00 |
| | | | | | | GVGK~SALTIQLIQNH | | | | |
| G12A | 14 | | | VGKSALTIQ | 2093 | 14 | 2355 | 0.00 | 0.00 | 0.04 |
| | | | | | | VGKS~ALTIQLIQNHF | | | | |
| G12A | 15 | | | GKSALTIQL | 2094 | 15 | 2356 | 0.00 | 0.02 | 0.28 |
| | | | | | | GKSA~LTIQLIQNHFV | | | | |
| G12C | 1 | | GEM2 | MTEYKLVVV | 2095 | 1 | 2357 | 0.00 | 0.00 | 0.47 |
| | | | | | | MTEY~KLVVVGACGVG | | | | |
| G12C | 2 | | TCEM2 | TEYKLVVVG | 2096 | 2 | 2358 | 0.00 | 0.26 | 0.04 |
| | | | | | | TEYK~LVVVGACGVGK | | | | |
| G12C | 3 | | TCEM2 | EYKLVVVGA | 2097 | 3 | 2359 | 0.00 | 0.16 | 0.65 |
| | | | | | | EYKL~VVVGACGVGKS | | | | |
| G12C | 4 | GEM1 | GEM2 | YKLVVVGAC | 2098 | 4 | 2360 | 0.83 | 0.97 | 0.94 |
| | | | | | | YKLV~VVGACGVGKSA | | | | |
| G12C | 5 | TCEM | TCEM2 | KLVVVGACG | 209 | 5 | 2361 | 0.99 | 0.34 | 1.00 |
| | | 1 | | | | KLVV~VGACGVGKSAL | | | | |
| G12C | 6 | TCEM | GEM2 | LVVVGACGV | 2100 | 6 | 2362 | 1.00 | 0.98 | 0.9 |
| | | 1 | | | | LVVV~GACGVGKSALT | | | | |
| G12C | 7 | TCEM | TCEM2 | VVVGACGVG | 2101 | 7 | 2363 | 0.79 | 0.83 | 0.98 |
| | | 1 | | | | VVVG~ACGVGKSALTI | | | | |
| G12C | 8 | TCEM | TCEM2 | VVGACGVGK | 2102 | 8 | 2364 | 0.96 | 0.95 | 0.98 |
| | | 1 | | | | VVGA~CGVGKSALTIQ | | | | |
| G12C | 9 | TCEM | GEM2 | VGACGVGKS | 2103 | 9 | 2365 | 0.23 | 0.22 | 1.00 |
| | | 1 | | | | VGAC~GVGKSALTIQL | | | | |
| G12C | 10 | GEM1 | | GACGVGKSA | 2104 | 10 | 2366 | 0.49 | 0.79 | 0.97 |
| | | | | | | GACG~VGKSALTIQLI | | | | |
| G12C | 11 | GEM1 | | ACGVGKSAL | 2105 | 11 | 2367 | 1.00 | 0.57 | 0.99 |
| | | | | | | ACGV~GKSALTIQLIQ | | | | |
| G12C | 12 | GEM1 | | CGVGKSALT | 2106 | 12 | 2368 | 0.27 | 0.94 | 0.74 |
| | | | | | | CGVG~KSALTIQLIQN | | | | |
| G12C | 13 | | | GVGKSALTI | 2107 | 13 | 2369 | 0.03 | 0.79 | 0.00 |
| | | | | | | GVGK~SALTIQLIQNH | | | | |
| G12C | 14 | | | VGKSALTIQ | 2108 | 14 | 2370 | 0.00 | 0.00 | 0.04 |
| | | | | | | VGKS~ALTIQLIQNHF | | | | |
| G12C | 15 | | | GKSALTIQL | 2109 | 15 | 2371 | 0.00 | 0.02 | 0.28 |
| | | | | | | GKSA~LTIQLIQNHFV | | | | |
| G12D | 1 | | GEM2 | MTEYKLVVV | 2110 | 1 | 2372 | 0.00 | 0.00 | 0.47 |
| | | | | | | MTEY~KLVVVGADGVG | | | | |
| G12D | 2 | | TCEM2 | TEYKLVVVG | 2111 | 2 | 2373 | 0.00 | 0.26 | 0.04 |
| | | | | | | TEYK~LVVVGADGVGK | | | | |
| G12D | 3 | | TCEM2 | EYKLVVVGA | 2112 | 3 | 2374 | 0.00 | 0.16 | 0.65 |
| | | | | | | EYKL~VVVGADGVGKS | | | | |
| G12D | 4 | GEM1 | GEM2 | YKLVVVGAD | 2113 | 4 | 2375 | 0.83 | 0.97 | 0.94 |
| | | | | | | YKLV~VVGADGVGKSA | | | | |
| G12D | 5 | TCEM | TCEM2 | KLVVVGADG | 2114 | 5 | 2376 | 0.88 | 0.47 | 0.99 |
| | | 1 | | | | KLVV~VGADGVGKSAL | | | | |
| G12D | 6 | TCEM | GEM2 | LVVVGADGV | 2115 | 6 | 2377 | 1.00 | 0.91 | 0.90 |
| | | 1 | | | | LVVV~GADGVGKSALT | | | | |
| G12D | 7 | TCEM | TCEM2 | VVVGADGVG | 2116 | 7 | 2378 | 0.77 | 0.96 | 1.00 |
| | | 1 | | | | VVVG~ADGVGKSALTI | | | | |
| G12D | 8 | TCEM | TCEM2 | VVGADGVGK | 2117 | 8 | 2379 | 0.66 | 0.49 | 0.29 |
| | | 1 | | | | VVGA~DGVGKSALTIQ | | | | |
| G12D | 9 | TCEM | GEM2 | VGADGVGKS | 2118 | 9 | 2380 | 0.72 | 0.64 | 0.99 |
| | | 1 | | | | VGAD~GVGKSALTIQL | | | | |
| G12D | 10 | GEM1 | | GADGVGKSA | 2119 | 10 | 2381 | 0.02 | 0.38 | 0.35 |
| | | | | | | GADG~VGKSALTIQLI | | | | |
| G12D | 11 | GEM1 | | ADGVGKSAL | 2120 | 11 | 2382 | 0.04 | 0.01 | 0.97 |
| | | | | | | ADGV~GKSALTIQLIQ | | | | |
| G12D | 12 | GEM1 | | DGVGKSALT | 2121 | 12 | 2383 | 0.40 | 0.74 | 0.44 |
| | | | | | | DGVG~KSALTIQLIQN | | | | |
| G12D | 13 | | | GVGKSALTI | 2122 | 13 | 2384 | 0.03 | 0.79 | 0.00 |
| | | | | | | GVGK~SALTIQLIQNH | | | | |
| G12D | 14 | | | VGKSALTIQ | 2123 | 14 | 2385 | 0.00 | 0.00 | 0.04 |
| | | | | | | VGKS~ALTIQLIQNHF | | | | |
| G12D | 15 | | | GKSALTIQL | 2124 | 15 | 2386 | 0.00 | 0.02 | 0.28 |
| | | | | | | GKSA~LTIQLIQNHFV | | | | |
| G12R | 1 | | GEM2 | MTEYKLVVV | 2125 | 1 | 2387 | 0.00 | 0.00 | 0.47 |
| | | | | | | MTEY~KLVVVGARGVG | | | | |
| G12R | 2 | | TCEM2 | TEYKLVVVG | 2126 | 2 | 2388 | 0.00 | 0.26 | 0.04 |
| | | | | | | TEYK~LVVVGARGVGK | | | | |
| G12R | 3 | | TCEM2 | EYKLVVVGA | 2127 | 3 | 2389 | 0.00 | 0.16 | 0.65 |
| | | | | | | EYKL~VVVGARGVGKS | | | | |
| G12R | 4 | GEM1 | GEM2 | YKLVVVGAR | 2128 | 4 | 2390 | 0.83 | 0.97 | 0.94 |
| | | | | | | YKLV~VVGARGVGKSA | | | | |
| G12R | 5 | TCEM | TCEM2 | KLVVVGARG | 2129 | 5 | 2391 | 0.88 | 0.48 | 0.82 |
| | | 1 | | | | KLVV~VGARGVGKSAL | | | | |
| G12R | 6 | TCEM | GEM2 | LVVVGARGV | 2130 | 6 | 2392 | 1.00 | 0.81 | 0.55 |
| | | 1 | | | | LVVV~GARGVGKSALT | | | | |
| G12R | 7 | TCEM | TCEM2 | VVVGARGVG | 2131 | 7 | 2393 | 0.41 | 0.38 | 0.89 |
| | | 1 | | | | VVVG~ARGVGKSALTI | | | | |
| G12R | 8 | TCEM | TCEM2 | VVGARGVGK | 2132 | 8 | 2394 | 0.92 | 0.64 | 0.00 |
| | | 1 | | | | VVGA~RGVGKSALTIQ | | | | |
| G12R | 9 | TCEM | GEM2 | VGARGVGKS | 2133 | 9 | 2395 | 0.04 | 0.67 | 0.02 |
| | | 1 | | | | VGAR~GVGKSALTIQL | | | | |
| G12R | 10 | GEM1 | | GARGVGKSA | 2134 | 10 | 2396 | 0.04 | 0.20 | 0.19 |
| | | | | | | GARG~VGKSALTIQLI | | | | |
| G12R | 11 | GEM1 | | ARGVGKSAL | 2135 | 11 | 2397 | 0.02 | 0.01 | 0.89 |
| | | | | | | ARGV~GKSALTIQLIQ | | | | |
| G12R | 12 | GEM1 | | RGVGKSALT | 2136 | 12 | 2398 | 0.36 | 0.77 | 0.04 |
| | | | | | | RGVG~KSALTIQLIQN | | | | |
| G12R | 13 | | | GVGKSALTI | 2137 | 13 | 2399 | 0.03 | 0.79 | 0.00 |
| | | | | | | GVGK~SALTIQLIQNH | | | | |
| G12R | 14 | | | VGKSALTIQ | 2138 | 14 | 2400 | 0.00 | 0.00 | 0.04 |
| | | | | | | VGKS~ALTIQLIQNHF | | | | |
| G12R | 15 | | | GKSALTIQL | 2139 | 15 | 2401 | 0.00 | 0.02 | 0.28 |
| | | | | | | GKSA~LTIQLIQNHFV | | | | |
| G12S | 1 | | GEM2 | MTEYKLVVV | 2140 | 1 | 2402 | 0.00 | 0.00 | 0.47 |
| | | | | | | MTEY~KLVVVGASGVG | | | | |
| G12S | 2 | | TCEM2 | TEYKLVVVG | 2141 | 2 | 2403 | 0.00 | 0.26 | 0.04 |
| | | | | | | TEYK~LVVVGASGVGK | | | | |
| G12S | 3 | | TCEM2 | EYKLVVVGA | 2142 | 3 | 2404 | 0.00 | 0.16 | 0.65 |
| | | | | | | EYKL~VVVGASGVGKS | | | | |
| G12S | 4 | GEM1 | GEM2 | YKLVVVGAS | 2143 | 4 | 2405 | 0.83 | 0.97 | 0.94 |
| | | | | | | YKLV~VVGASGVGKSA | | | | |
| G12S | 5 | TCEM | TCEM2 | KLVVVGASG | 2144 | 5 | 2406 | 1.00 | 0.76 | 1.00 |
| | | 1 | | | | KLVV~VGASGVGKSAL | | | | |
| G12S | 6 | TCEM | GEM2 | LVVVGASGV | 2145 | 6 | 2407 | 1.00 | 0.99 | 1.00 |
| | | 1 | | | | LVVV~GASGVGKSALT | | | | |
| G12S | 7 | TCEM | TCEM2 | VVVGASGVG | 2146 | 7 | 2408 | 0.94 | 0.99 | 1.00 |
| | | 1 | | | | VVVG~ASGVGKSALTI | | | | |
| G12S | 8 | TCEM | TCEM2 | VVGASGVGK | 2147 | 8 | 2409 | 0.34 | 0.41 | 0.76 |
| | | 1 | | | | VVGA~SGVGKSALTIQ | | | | |
| G12S | 9 | TCEM | GEM2 | VGASGVGKS | 2148 | 9 | 2410 | 0.19 | 0.96 | 0.97 |
| | | 1 | | | | VGAS~GVGKSALTIQL | | | | |
| G12S | 10 | GEM1 | | GASGVGKSA | 2149 | 10 | 2411 | 0.06 | 0.54 | 0.52 |
| | | | | | | GASG~VGKSALTIQLI | | | | |
| G12S | 11 | GEM1 | | ASGVGKSAL | 2150 | 11 | 2412 | 0.09 | 0.05 | 0.98 |
| | | | | | | ASGV~GKSALTIQLIQ | | | | |
| G12S | 12 | GEM1 | | SGVGKSALT | 2151 | 12 | 2413 | 0.65 | 0.84 | 0.67 |
| | | | | | | SGVG~KSALTIQLIQN | | | | |
| G12S | 13 | | | GVGKSALTI | 2152 | 13 | 2414 | 0.03 | 0.79 | 0.00 |
| | | | | | | GVGK~SALTIQLIQNH | | | | |
| G12S | 14 | | | VGKSALTIQ | 2153 | 14 | 2415 | 0.00 | 0.00 | 0.04 |
| | | | | | | VGKS~ALTIQLIQNHF | | | | |
| G12S | 15 | | | GKSALTIQL | 2154 | 15 | 2416 | 0.00 | 0.02 | 0.28 |
| | | | | | | GKSA~LTIQLIQNHFV | | | | |
| G12V | 1 | | GEM2 | MTEYKLVVV | 2155 | 1 | 2417 | 0.00 | 0.00 | 0.47 |
| | | | | | | MTEY~KLVVVGAVGVG | | | | |
| G12V | 2 | | TCEM2 | TEYKLVVVG | 2156 | 2 | 2418 | 0.00 | 0.26 | 0.04 |
| | | | | | | TEYK~LVVVGAVGVGK | | | | |
| G12V | 3 | | TCEM2 | EYKLVVVGA | 2157 | 3 | 2419 | 0.00 | 0.16 | 0.65 |
| | | | | | | EYKL~VVVGAVGVGKS | | | | |
| G12V | 4 | GEM1 | GEM2 | YKLVVVGAV | 2158 | 4 | 2420 | 0.83 | 0.97 | 0.94 |
| | | | | | | YKLV~VVGAVGVGKSA | | | | |
| G12V | 5 | TCEM | TCEM2 | KLVVVGAVG | 2159 | 5 | 2421 | 1.00 | 0.39 | 1.00 |
| | | 1 | | | | KLVV~VGAVGVGKSAL | | | | |
| G12V | 6 | TCEM | GEM2 | LVVVGAVGV | 2160 | 6 | 2422 | 1.00 | 0.99 | 0.99 |
| | | 1 | | | | LVVV~GAVGVGKSALT | | | | |
| G12V | 7 | TCEM | TCEM2 | VVVGAVGVG | 2161 | 7 | 2423 | 0.81 | 0.94 | 1.00 |
| | | 1 | | | | VVVG~AVGVGKSALTI | | | | |
| G12V | 8 | TCEM | TCEM2 | VVGAVGVGK | 2162 | 8 | 2424 | 0.08 | 0.62 | 0.96 |
| | | 1 | | | | VVGA~VGVGKSALTIQ | | | | |
| G12V | 9 | TCEM | GEM2 | VGAVGVGKS | 2163 | 9 | 2425 | 0.11 | 0.25 | 1.00 |
| | | 1 | | | | VGAV~GVGKSALTIQL | | | | |
| G12V | 10 | GEM1 | | GAVGVGKSA | 2164 | 10 | 2426 | 0.95 | 0.66 | 0.95 |
| | | | | | | GAVG~VGKSALTIQLI | | | | |
| G12V | 11 | GEM1 | | AVGVGKSAL | 2165 | 11 | 2427 | 0.87 | 0.48 | 1.00 |
| | | | | | | AVGV~GKSALTIQLIQ | | | | |
| G12V | 12 | GEM1 | | VGVGKSALT | 2166 | 12 | 2428 | 0.48 | 0.37 | 0.58 |
| | | | | | | VGVG~KSALTIQLIQN | | | | |
| G12V | 13 | | | GVGKSALTI | 2167 | 13 | 2429 | 0.03 | 0.79 | 0.00 |
| | | | | | | GVGK~SALTIQLIQNH | | | | |
| G12V | 14 | | | VGKSALTIQ | 2168 | 14 | 2430 | 0.00 | 0.00 | 0.04 |
| | | | | | | VGKS~ALTIQLIQNHF | | | | |
| G12V | 15 | | | GKSALTIQL | 2169 | 1 | 2431 | 0.00 | 0.02 | 0.28 |
| | | | | | | GKSA~LTIQLIQNHFV | | | | |
| G13C | 1 | | | MTEYKLVVV | 2170 | 1 | 2432 | 0.00 | 0.00 | 0.47 |
| | | | | | | MTEY~KLVVVGAGCVG | | | | |
| G13C | 2 | | GEM2 | TEYKLVVVG | 2171 | 2 | 2433 | 0.00 | 0.26 | 0.04 |
| | | | | | | TEYK~LVVVGAGCVGK | | | | |
| G13C | 3 | | TCEM2 | EYKLVVVGA | 2172 | 3 | 2434 | 0.00 | 0.16 | 0.65 |
| | | | | | | EYKL~VVVGAGCVGKS | | | | |
| G13C | 4 | | TCEM2 | YKLVVVGAG | 2173 | 4 | 2435 | 0.83 | 0.97 | 0.94 |
| | | | | | | YKLV~VVGAGCVGKSA | | | | |
| G13C | 5 | GEM1 | GEM2 | KLVVVGAGC | 2174 | 5 | 2436 | 1.00 | 0.47 | 1.00 |
| | | | | | | KLVV~VGAGCVGKSAL | | | | |
| G13C | 6 | TCEM | TCEM2 | LVVVGAGCV | 2175 | 6 | 2437 | 0.99 | 0.47 | 1.00 |
| | | 1 | | | | LVVV~GAGCVGKSALT | | | | |
| G13C | 7 | TCEM | GEM2 | VVVGAGCVG | 2176 | 7 | 2438 | 0.79 | 0.67 | 0.86 |
| | | 1 | | | | VVVG~AGCVGKSALTI | | | | |
| G13C | 8 | TCEM | TCEM2 | VVGAGCVGK | 2177 | 8 | 2439 | 0.04 | 0.59 | 0.69 |
| | | 1 | | | | VVGA~GCVGKSALTIQ | | | | |
| G13C | 9 | TCEM | TCEM2 | VGAGCVGKS | 2178 | 9 | 2440 | 0.91 | 0.85 | 0.59 |
| | | 1 | | | | VGAG~CVGKSALTIQL | | | | |
| G13C | 10 | TCEM | GEM2 | GAGCVGKSA | 2179 | 10 | 2441 | 0.54 | 0.04 | 1.00 |
| | | 1 | | | | GAGC~VGKSALTIQLI | | | | |
| G13C | 11 | GEM1 | | AGCVGKSAL | 2180 | 11 | 2442 | 1.00 | 0.42 | 0.85 |
| | | | | | | AGCV~GKSALTIQLIQ | | | | |
| G13C | 12 | GEM1 | | GCVGKSALT | 2181 | 12 | 2443 | 1.00 | 0.98 | 1.00 |
| | | | | | | GCVG~KSALTIQLIQN | | | | |
| G13C | 13 | GEM1 | | CVGKSALTI | 2182 | 13 | 2444 | 0.07 | 0.11 | 0.00 |
| | | | | | | CVGK~SALTIQLIQNH | | | | |
| G13C | 14 | | | VGKSALTIQ | 2183 | 14 | 2445 | 0.00 | 0.00 | 0.04 |
| | | | | | | VGKS~ALTIQLIQNHF | | | | |
| G13C | 15 | | | GKSALTIQL | 2184 | 15 | 2446 | 0.00 | 0.02 | 0.28 |
| | | | | | | GKSA~LTIQLIQNHFV | | | | |
| G13C | 16 | | | KSALTIQLI | 2185 | 16 | 2447 | 0.00 | 0.01 | 0.00 |
| | | | | | | KSAL~TIQLIQNHFVD | | | | |
| G13D | 1 | | | MTEYKLVVV | 2186 | 1 | 2448 | 0.00 | 0.00 | 0.47 |
| | | | | | | MTEY~KLVVVGAGDVG | | | | |
| G13D | 2 | | GEM2 | TEYKLVVVG | 2187 | 2 | 2449 | 0.00 | 0.26 | 0.04 |
| | | | | | | TEYK~LVVVGAGDVGK | | | | |
| G13D | 3 | | TCEM2 | EYKLVVVGA | 2188 | 3 | 2450 | 0.00 | 0.16 | 0.65 |
| | | | | | | EYKL~VVVGAGDVGKS | | | | |
| G13D | 4 | | TCEM2 | YKLVVVGAG | 2189 | 4 | 2451 | 0.83 | 0.97 | 0.94 |
| | | | | | | YKLV~VVGAGDVGKSA | | | | |
| G13D | 5 | GEM1 | GEM2 | KLVVVGAGD | 2190 | 5 | 2452 | 1.00 | 0.47 | 1.00 |
| | | | | | | KLVV~VGAGDVGKSAL | | | | |
| G13D | 6 | TCEM | TCEM2 | LVVVGAGDV | 2191 | 6 | 2453 | 1.00 | 1.00 | 1.00 |
| | | 1 | | | | LVVV~GAGDVGKSALT | | | | |
| G13D | 7 | TCEM | GEM2 | VVVGAGDVG | 2192 | 7 | 2454 | 0.92 | 1.00 | 0.98 |
| | | 1 | | | | VVVG~AGDVGKSALTI | | | | |
| G13D | 8 | TCEM | TCEM2 | VVGAGDVGK | 2193 | 8 | 2455 | 0.01 | 0.32 | 0.50 |
| | | 1 | | | | VVGA~GDVGKSALTIQ | | | | |
| G13D | 9 | TCEM | TCEM2 | VGAGDVGKS | 2194 | 9 | 2456 | 0.61 | 0.65 | 0.96 |
| | | 1 | | | | VGAG~DVGKSALTIQL | | | | |
| G13D | 10 | TCEM | GEM2 | GAGDVGKSA | 2195 | 10 | 2457 | 0.03 | 0.13 | 0.50 |
| | | 1 | | | | GAGD~VGKSALTIQLI | | | | |
| G13D | 11 | GEM1 | | AGDVGKSAL | 2196 | 11 | 2458 | 0.03 | 0.02 | 0.79 |
| | | | | | | AGDV~GKSALTIQLIQ | | | | |
| G13D | 12 | GEM1 | | GDVGKSALT | 2197 | 12 | 2459 | 0.37 | 0.30 | 0.97 |
| | | | | | | GDVG~KSALTIQLQN | | | | |
| G13D | 13 | GEM1 | | DVGKSALTI | 2198 | 13 | 2460 | 0.02 | 0.23 | 0.00 |
| | | | | | | DVGK~SALTIQLIQNH | | | | |
| G13D | 14 | | | VGKSALTIQ | 2199 | 14 | 2461 | 0.00 | 0.00 | 0.04 |
| | | | | | | VGKS~ALTIQLIQNHF | | | | |
| G13D | 15 | | | GKSALTIQL | 2200 | 15 | 2462 | 0.00 | 0.02 | 0.28 |
| | | | | | | GKSA~LTIQLIQNHFV | | | | |
| G13D | 16 | | | KSALTIQLI | 2201 | 16 | 2463 | 0.00 | 0.01 | 0.00 |
| | | | | | | KSAL~TIQLIQNHFVD | | | | |
| G13R | 1 | | | MTEYKLVVV | 2202 | 1 | 2464 | 0.00 | 0.00 | 0.47 |
| | | | | | | MTEY~KLVVVGAGRVG | | | | |
| G13R | 2 | | GEM2 | TEYKLVVVG | 2203 | 2 | 2465 | 0.00 | 0.26 | 0.04 |
| | | | | | | TEYK~LVVVGAGRVGK | | | | |
| G13R | 3 | | TCEM2 | EYKLVVVGA | 2204 | 3 | 2466 | 0.00 | 0.16 | 0.65 |
| | | | | | | EYKL~VVVGAGRVGKS | | | | |
| G13R | 4 | | TCEM2 | YKLVVVGAG | 2205 | 4 | 2467 | 0.83 | 0.97 | 0.94 |
| | | | | | | YKLV~VVGAGRVGKSA | | | | |
| G13R | 5 | GEM1 | GEM2 | KLVVVGAGR | 2206 | 5 | 2468 | 1.00 | 0.47 | 1.00 |
| | | | | | | KLVV~VGAGRVGKSAL | | | | |
| G13R | 6 | TCEM | TCEM2 | LVVVGAGRV | 2207 | 6 | 2469 | 1.00 | 0.95 | 1.00 |
| | | 1 | | | | LVVV~GAGRVGKSALT | | | | |
| G13R | 7 | TCEM | GEM2 | VVVGAGRVG | 2208 | 7 | 2470 | 0.87 | 0.99 | 0.16 |
| | | 1 | | | | VVVG~AGRVGKSALTI | | | | |
| G13R | 8 | TCEM | TCEM2 | VVGAGRVGK | 2209 | 8 | 2471 | 0.00 | 0.42 | 0.15 |
| | | 1 | | | | VVGA~GRVGKSALTIQ | | | | |
| G13R | 9 | TCEM | TCEM2 | VGAGRVGKS | 2210 | 9 | 2472 | 0.14 | 0.74 | 0.05 |
| | | 1 | | | | VGAG~RVGKSALTIQL | | | | |
| G13R | 10 | TCEM | GEM2 | GAGRVGKSA | 2211 | 10 | 2473 | 0.00 | 0.08 | 0.06 |
| | | 1 | | | | GAGR~VGKSALTIQLI | | | | |
| G13R | 11 | GEM1 | | AGRVGKSAL | 2212 | 11 | 2474 | 0.04 | 0.28 | 0.94 |
| | | | | | | AGRV~GKSALTIQLIQ | | | | |
| G13R | 12 | GEM1 | | GRVGKSALT | 2213 | 12 | 2475 | 0.16 | 0.07 | 0.98 |
| | | | | | | GRVG~KSALTIQLIQN | | | | |
| G13R | 13 | GEM1 | | RVGKSALTI | 2214 | 13 | 2476 | 0.03 | 0.07 | 0.00 |
| | | | | | | RVGK~SALTIQLIQNH | | | | |
| G13R | 14 | | | VGKSALTIQ | 2215 | 14 | 2477 | 0.00 | 0.00 | 0.04 |
| | | | | | | VGKS~ALTIQLIQNHF | | | | |
| G13R | 15 | | | GKSALTIQL | 2216 | 15 | 2478 | 0.00 | 0.02 | 0.28 |
| | | | | | | GKSA~LTIQLIQNHFV | | | | |
| G13R | 16 | | | KSALTIQLI | 2217 | 16 | 2479 | 0.00 | 0.01 | 0.00 |
| | | | | | | KSAL~TIQLIQNHFVD | | | | |
| G13V | 1 | | | MTEYKLVVV | 2218 | 1 | 2480 | 0.00 | 0.00 | 0.4 |
| | | | | | | MTEY~KLVVVGAGVVG | | | | |
| G13V | 2 | | GEM2 | TEYKLVVVG | 2219 | 2 | 2481 | 0.00 | 0.26 | 0.04 |
| | | | | | | TEYK~LVVVGAGVVGK | | | | |
| G13V | 3 | | TCEM2 | EYKLVVVGA | 2220 | 3 | 2482 | 0.00 | 0.16 | 0.65 |
| | | | | | | EYKL~VVVGAGVVGKS | | | | |
| G13V | 4 | | TCEM2 | YKLVVVGAG | 2221 | 4 | 2483 | 0.83 | 0.97 | 0.94 |
| | | | | | | YKLV~VVGAGVVGKSA | | | | |
| G13V | 5 | GEM1 | GEM2 | KLVVVGAGV | 2222 | 5 | 2484 | 1.00 | 0.47 | 1.00 |
| | | | | | | KLVV~VGAGVVGKSAL | | | | |
| G13V | 6 | TCEM | TCEM2 | LVVVGAGVV | 2223 | 6 | 2485 | 1.00 | 0.93 | 1.00 |
| | | 1 | | | | LVVV~GAGVVGKSALT | | | | |
| G13V | 7 | TCEM | GEM2 | VVVGAGVVG | 2224 | 7 | 2486 | 0.92 | 0.94 | 1.00 |
| | | 1 | | | | VVVG~AGVVGKSALTI | | | | |
| G13V | 8 | TCEM | TCEM2 | VVGAGVVGK | 2225 | 8 | 2487 | 0.02 | 0.64 | 0.91 |
| | | 1 | | | | VVGA~GVVGKSALTIQ | | | | |
| G13V | 9 | TCEM | TCEM2 | VGAGVVGKS | 2226 | 9 | 2488 | 0.74 | 0.33 | 1.00 |
| | | 1 | | | | VGAG~VVGKSALTIQL | | | | |
| G13V | 10 | TCEM | GEM2 | GAGVVGKSA | 2227 | 10 | 2489 | 0.46 | 0.61 | 0.89 |
| | | 1 | | | | GAGV~VGKSALTIQLI | | | | |
| G13V | 11 | GEM1 | | AGVVGKSAL | 2228 | 11 | 2490 | 1.00 | 0.86 | 0.99 |
| | | | | | | AGVV~GKSALTIQLIQ | | | | |
| G13V | 12 | GEM1 | | GVVGKSALT | 2229 | 12 | 2491 | 0.53 | 0.81 | 0.99 |
| | | | | | | GVVG~KSALTIQLIQN | | | | |
| G13V | 13 | GEM1 | | VVGKSALTI | 2230 | 13 | 2492 | 0.03 | 0.43 | 0.00 |
| | | | | | | VVGK~SALTIQLIQNH | | | | |
| G13V | 14 | | | VGKSALTIQ | 2231 | 14 | 2493 | 0.00 | 0.00 | 0.04 |
| | | | | | | VGKS~ALTIQLIQNHF | | | | |
| G13V | 15 | | | GKSALTIQL | 2232 | 15 | 2494 | 0.00 | 0.02 | 0.28 |
| | | | | | | GKSA~LTIQLIQNHFV | | | | |
| G13V | 16 | | | KSALTIQLI | 2233 | 16 | 2495 | 0.00 | 0.01 | 0.00 |
| | | | | | | KSAL~TIQLIQNHFVD | | | | |
| Q61E | 47 | | | DGETCLLDI | 2234 | 47 | 2496 | 0.28 | 0.00 | 0.04 |
| | | | | | | DGET~CLLDILDTAGE | | | | |
| Q61E | 48 | | | GETCLLDIL | 2235 | 48 | 2497 | 0.00 | 0.06 | 0.00 |
| | | | | | | GETC~LLDILDTAGEE | | | | |
| Q61E | 49 | | | ETCLLDILD | 2236 | 49 | 2498 | 0.00 | 0.00 | 0.10 |
| | | | | | | ETCL~LDILDTAGEEE | | | | |
| Q61E | 50 | | GEM2 | TCLLDILDT | 2237 | 50 | 2499 | 0.99 | 0.15 | 0.09 |
| | | | | | | TCLL~DILDTAGEEEY | | | | |
| Q61E | 51 | | TCEM2 | CLLDILDTA | 2238 | 51 | 2500 | 0.79 | 0.78 | 0.37 |
| | | | | | | CLLD~ILDTAGEEEYS | | | | |
| Q61E | 52 | | TCEM2 | LLDILDTAG | 2239 | 52 | 2501 | 0.00 | 0.00 | 0.98 |
| | | | | | | LLDI~LDTAGEEEYSA | | | | |
| Q61E | 53 | GEM1 | GEM2 | LDILDTAGE | 2240 | 53 | 2502 | 0.96 | 0.95 | 0.27 |
| | | | | | | LDIL~DTAGEEEYSAM | | | | |
| Q61E | 54 | TCEM | TCEM2 | DILDTAGEE | 2241 | 54 | 2503 | 1.00 | 1.00 | 1.00 |
| | | 1 | | | | DILD~TAGEEEYSAMR | | | | |
| Q61E | 55 | TCEM | GEM2 | ILDTAGEEE | 2242 | 55 | 2504 | 0.00 | 0.01 | 0.03 |
| | | 1 | | | | ILDT~AGEEEYSAMRD | | | | |
| Q61E | 56 | TCEM | TCEM2 | LDTAGEEEY | 2243 | 56 | 2505 | 0.02 | 0.03 | 0.00 |
| | | 1 | | | | LDTA~GEEEYSAMRDQ | | | | |
| Q61E | 57 | TCEM | TCEM2 | DTAGEEEYS | 2244 | 57 | 2506 | 0.22 | 0.36 | 0.00 |
| | | 1 | | | | DTAG~EEEYSAMRDQY | | | | |
| Q61E | 58 | TCEM | GEM2 | TAGEEEYSA | 2245 | 58 | 2507 | 0.10 | 0.01 | 0.00 |
| | | 1 | | | | TAGE~EEYSAMRDQYM | | | | |
| Q61E | 59 | GEM1 | | AGEEEYSAM | 2246 | 59 | 2508 | 0.00 | 0.00 | 0.22 |
| | | | | | | AGEE~EYSAMRDQYMR | | | | |
| Q61E | 60 | GEM1 | | GEEEYSAMR | 2247 | 60 | 2509 | 0.01 | 0.00 | 0.75 |
| | | | | | | GEEE~YSAMRDQYMRT | | | | |
| Q61E | 61 | GEM1 | | EEEYSAMRD | 2248 | 61 | 2510 | 0.73 | 0.00 | 0.00 |
| | | | | | | EEEY~SAMRDQYMRTG | | | | |
| Q61E | 62 | | | EEYSAMRDQ | 2249 | 62 | 2511 | 0.05 | 0.10 | 0.00 |
| | | | | | | EEYS-AMRDQYMRTGE | | | | |
| Q61E | 63 | | | EYSAMRDQY | 2250 | 63 | 2512 | 0.02 | 0.01 | 0.00 |
| | | | | | | EYSA~MRDQYMRTGEG | | | | |
| Q61E | 64 | | | YSAMRDQYM | 225 | 64 | 251 | 0.00 | 0.01 | 0.00 |
| | | | | | | YSAM~RDQYMRTGEGF | | | | |
| Q61H | 47 | | | DGETCLLDI | 2252 | 47 | 2514 | 0.28 | 0.00 | 0.04 |
| | | | | | | DGET~CLLDILDTAGH | | | | |
| Q61H | 48 | | | GETCLLDIL | 2253 | 48 | 2515 | 0.00 | 0.06 | 0.00 |
| | | | | | | GETC~LLDILDTAGHE | | | | |
| Q61H | 49 | | | ETCLLDILD | 2254 | 49 | 2516 | 0.00 | 0.00 | 0.10 |
| | | | | | | ETCL~LDILDTAGHEE | | | | |
| Q61H | 50 | | GEM2 | TCLLDILDT | 2255 | 50 | 2517 | 0.99 | 0.15 | 0.09 |
| | | | | | | TCLL~DILDTAGHEEY | | | | |
| Q61H | 51 | | TCEM2 | CLLDILDTA | 2256 | 51 | 2518 | 0.79 | 0.78 | 0.37 |
| | | | | | | CLLD~ILDTAGHEEYS | | | | |
| Q61H | 52 | | TCEM2 | LLDILDTAG | 2257 | 52 | 2519 | 0.00 | 0.00 | 0.98 |
| | | | | | | LLDI~LDTAGHEEYSA | | | | |
| Q61H | 53 | GEM1 | GEM2 | LDILDTAGH | 2258 | 53 | 2520 | 0.96 | 0.95 | 0.27 |
| | | | | | | LDIL~DTAGHEEYSAM | | | | |
| Q61H | 54 | TCEM | TCEM2 | DILDTAGHE | 2259 | 54 | 2521 | 1.00 | 0.99 | 1.00 |
| | | 1 | | | | DILD~TAGHEEYSAMR | | | | |
| Q61H | 55 | TCEM | GEM2 | ILDTAGHEE | 2260 | 55 | 2522 | 0.00 | 0.01 | 0.00 |
| | | 1 | | | | ILDT~AGHEEYSAMRD | | | | |
| Q61H | 56 | TCEM | TCEM2 | LDTAGHEEY | 2261 | 56 | 2523 | 0.01 | 0.01 | 0.00 |
| | | 1 | | | | LDTA~GHEEYSAMRDQ | | | | |
| Q61H | 57 | TCEM | TCEM2 | DTAGHEEYS | 2262 | 57 | 2524 | 0.03 | 0.01 | 0.00 |
| | | 1 | | | | DTAG~HEEYSAMRDQY | | | | |
| Q61H | 58 | TCEM | GEM2 | TAGHEEYSA | 2263 | 58 | 2525 | 0.00 | 0.00 | 0.00 |
| | | 1 | | | | TAGH~EEYSAMRDQYM | | | | |
| Q61H | 59 | GEM1 | | AGHEEYSAM | 2264 | 59 | 2526 | 0.03 | 0.00 | 0.24 |
| | | | | | | AGHE~EYSAMRDQYMR | | | | |
| Q61H | 60 | GEM1 | | GHEEYSAMR | 2265 | 60 | 2527 | 0.03 | 0.00 | 0.84 |
| | | | | | | GHEE~YSAMRDQYMRT | | | | |
| Q61H | 61 | GEM1 | | HEEYSAMRD | 2266 | 61 | 2528 | 0.75 | 0.00 | 0.00 |
| | | | | | | HEEY~SAMRDQYMRTG | | | | |
| Q61H | 62 | | | EEYSAMRDQ | 2267 | 62 | 2529 | 0.05 | 0.10 | 0.00 |
| | | | | | | EEYS-AMRDQYMRTGE | | | | |
| Q61H | 63 | | | EYSAMRDQY | 2268 | 63 | 2530 | 0.02 | 0.01 | 0.00 |
| | | | | | | EYSA~MRDQYMRTGEG | | | | |
| Q61H | 64 | | | YSAMRDQYM | 2269 | 64 | 2531 | 0.00 | 0.01 | 0.00 |
| | | | | | | YSAM~RDQYMRTGEGF | | | | |
| Q61K | 47 | | | DGETCLLDI | 2270 | 47 | 2532 | 0.28 | 0.00 | 0.04 |
| | | | | | | DGET~CLLDILDTAGK | | | | |
| Q61K | 48 | | | GETCLLDIL | 2271 | 48 | 2533 | 0.00 | 0.06 | 0.00 |
| | | | | | | GETC~LLDILDTAGKE | | | | |
| Q61K | 49 | | | ETCLLDILD | 2272 | 49 | 2534 | 0.00 | 0.00 | 0.10 |
| | | | | | | ETCL~LDILDTAGKEE | | | | |
| Q61K | 50 | | GEM2 | TCLLDILDT | 2273 | 50 | 2535 | 0.99 | 0.15 | 0.09 |
| | | | | | | TCLL~DILDTAGKEEY | | | | |
| Q61K | 51 | | TCEM2 | CLLDILDTA | 2274 | 51 | 2536 | 0.79 | 0.78 | 0.37 |
| | | | | | | CLLD~ILDTAGKEEYS | | | | |
| Q61K | 52 | | TCEM2 | LLDILDTAG | 2275 | 52 | 2537 | 0.00 | 0.00 | 0.98 |
| | | | | | | LLDI~LDTAGKEEYSA | | | | |
| Q61K | 53 | GEM1 | GEM2 | LDILDTAGK | 2276 | 53 | 2538 | 0.96 | 0.95 | 0.27 |
| | | | | | | LDIL~DTAGKEEYSAM | | | | |
| Q61K | 54 | TCEM | TCEM2 | DILDTAGKE | 2277 | 54 | 2539 | 1.00 | 0.99 | 1.00 |
| | | 1 | | | | DILD~TAGKEEYSAMR | | | | |
| Q61K | 55 | TCEM | GEM2 | ILDTAGKEE | 2278 | 55 | 2540 | 0.03 | 0.02 | 0.02 |
| | | 1 | | | | ILDT~AGKEEYSAMRD | | | | |
| Q61K | 56 | TCEM | TCEM2 | LDTAGKEEY | 2279 | 56 | 2541 | 0.07 | 0.01 | 0.00 |
| | | 1 | | | | LDTA~GKEEYSAMRDQ | | | | |
| Q61K | 57 | TCEM | TCEM2 | DTAGKEEYS | 2280 | 57 | 2542 | 0.03 | 0.00 | 0.11 |
| | | 1 | | | | DTAG~KEEYSAMRDQY | | | | |
| Q61K | 58 | TCEM | GEM2 | TAGKEEYSA | 2281 | 58 | 2543 | 0.01 | 0.00 | |
| | | 1 | | | | TAGK~EEYSAMRDQYM | | | | |
| Q61K | 59 | GEM1 | | AGKEEYSAM | 2282 | 59 | 2544 | 0.00 | 0.00 | 0.00 |
| | | | | | | AGKE~EYSAMRDQYMR | | | | |
| Q61K | 60 | GEM1 | | GKEEYSAMR | 2283 | 60 | 2545 | 0.08 | 0.00 | 0.90 |
| | | | | | | GKEE~YSAMRDQYMRT | | | | |
| Q61K | 61 | GEM1 | | KEEYSAMRD | 2284 | 61 | 2546 | 0.74 | 0.30 | 0.04 |
| | | | | | | KEEY~SAMRDQYMRTG | | | | |
| Q61K | 62 | | | EEYSAMRDQ | 2285 | 62 | 2547 | 0.05 | 0.10 | 0.00 |
| | | | | | | EEYS-AMRDQYMRTGE | | | | |
| Q61K | 63 | | | EYSAMRDQY | 2286 | 63 | 2548 | 0.02 | 0.01 | 0.00 |
| | | | | | | EYSA~MRDQYMRTGEG | | | | |
| Q61K | 64 | | | YSAMRDQYM | 2287 | 64 | 2549 | 0.00 | 0.01 | 0.00 |
| | | | | | | YSAM~RDQYMRTGEGF | | | | |
| Q61L | 47 | | | DGETCLLDI | 2288 | 47 | 2550 | 0.28 | 0.00 | 0.04 |
| | | | | | | DGET~CLLDILDTAGL | | | | |
| Q61L | 48 | | | GETCLLDIL | 2289 | 48 | 2551 | 0.00 | 0.06 | 0.00 |
| | | | | | | GETC~LLDILDTAGLE | | | | |
| Q61L | 49 | | | ETCLLDILD | 2290 | 49 | 2552 | 0.00 | 0.00 | 0.10 |
| | | | | | | ETCL~LDILDTAGLEE | | | | |
| Q61L | 50 | | GEM2 | TCLLDILDT | 2291 | 50 | 2553 | 0.99 | 0.15 | 0.09 |
| | | | | | | TCLL~DILDTAGLEEY | | | | |
| Q61L | 51 | | TCEM2 | CLLDILDTA | 2292 | 51 | 2554 | 0.79 | 0.78 | 0.37 |
| | | | | | | CLLD~ILDTAGLEEYS | | | | |
| Q61L | 52 | | TCEM2 | LLDILDTAG | 2293 | 52 | 2555 | 0.00 | 0.00 | 0.98 |
| | | | | | | LLDI~LDTAGLEEYSA | | | | |
| Q61L | 53 | GEM1 | GEM2 | LDILDTAGL | 2294 | 53 | 2556 | 0.96 | 0.95 | 0.27 |
| | | | | | | LDIL~DTAGLEEYSAM | | | | |
| Q61L | 54 | TCEM | TCEM2 | DILDTAGLE | 2295 | 54 | 2557 | 0.98 | 0.99 | 1.00 |
| | | 1 | | | | DILD~TAGLEEYSAMR | | | | |
| Q61L | 55 | TCEM | GEM2 | ILDTAGLEE | 2296 | 55 | 2558 | 0.01 | 0.01 | 0.03 |
| | | 1 | | | | ILDT~AGLEEYSAMRD | | | | |
| Q61L | 56 | TCEM | TCEM2 | LDTAGLEEY | 229 | 56 | 2559 | 0.01 | 0.00 | 0.01 |
| | | 1 | | | | LDTA~GLEEYSAMRDQ | | | | |
| Q61L | 57 | TCEM | TCEM2 | DTAGLEEYS | 2298 | 57 | 2560 | 0.01 | 0.00 | 0.30 |
| | | 1 | | | | DTAG~LEEYSAMRDQY | | | | |
| Q61L | 58 | TCEM | GEM2 | TAGLEEYSA | 2299 | 58 | 2561 | 0.00 | 0.00 | 0.00 |
| | | 1 | | | | TAGL~EEYSAMRDQYM | | | | |
| Q61L | 59 | GEM1 | | AGLEEYSAM | 2300 | 59 | 2562 | 0.85 | 0.70 | 0.27 |
| | | | | | | AGLE~EYSAMRDQYMR | | | | |
| Q61L | 60 | GEM1 | | GLEEYSAMR | 2301 | 60 | 2563 | 0.11 | 0.01 | 1.00 |
| | | | | | | GLEE~YSAMRDQYMRT | | | | |
| Q61L | 61 | GEM1 | | LEEYSAMRD | 2302 | 61 | 2564 | 0.25 | 0.00 | 0.00 |
| | | | | | | LEEY~SAMRDQYMRTG | | | | |
| Q61L | 62 | | | EEYSAMRDQ | 2303 | 62 | 2565 | 0.05 | 0.10 | 0.00 |
| | | | | | | EEYS~AMRDQYMRTGE | | | | |
| Q61L | 63 | | | EYSAMRDQY | 2304 | 63 | 2566 | 0.02 | 0.01 | 0.00 |
| | | | | | | EYSA~MRDQYMRTGEG | | | | |
| Q61L | 64 | | | YSAMRDQYM | 2305 | 64 | 2567 | 0.00 | 0.01 | 0.00 |
| | | | | | | YSAM~RDQYMRTGEGF | | | | |
| Q61P | 47 | | | DGETCLLDI | 2306 | 47 | 2568 | 0.28 | 0.00 | 0.04 |
| | | | | | | DGET~CLLDILDTAGP | | | | |
| Q61P | 48 | | | GETCLLDIL | 2307 | 48 | 2569 | 0.00 | 0.06 | 0.00 |
| | | | | | | GETC~LLDILDTAGPE | | | | |
| Q61P | 49 | | | ETCLLDILD | 2308 | 49 | 2570 | 0.00 | 0.00 | 0.10 |
| | | | | | | ETCL~LDILDTAGPEE | | | | |
| Q61P | 50 | | GEM2 | TCLLDILDT | 2309 | 50 | 2571 | 0.99 | 0.15 | 0.09 |
| | | | | | | TCLL~DILDTAGPEEY | | | | |
| Q61P | 51 | | TCEM2 | CLLDILDTA | 2310 | 51 | 2572 | 0.79 | 0.78 | 0.37 |
| | | | | | | CLLD~ILDTAGPEEYS | | | | |
| Q61P | 52 | | TCEM2 | LLDILDTAG | 2311 | 52 | 2573 | 0.00 | 0.00 | 0.98 |
| | | | | | | LLDI~LDTAGPEEYSA | | | | |
| Q61F | 53 | GEM1 | GEM2 | LDILDTAGP | 2312 | 53 | 2574 | 0.96 | 0.95 | 0.27 |
| | | | | | | LDIL~DTAGPEEYSAM | | | | |
| Q61P | 54 | TCEM | TCEM2 | DILDTAGPE | 2313 | 54 | 2575 | 0.99 | 1.00 | 1.00 |
| | | 1 | | | | DILD~TAGPEEYSAMR | | | | |
| Q61F | 55 | TCEM | GEM2 | ILDTAGPEE | 2314 | 55 | 2576 | 0.02 | 0.02 | 0.10 |
| | | 1 | | | | ILDT~AGPEEYSAMRD | | | | |
| Q61F | 56 | TCEM | TCEM2 | LDTAGPEEY | 2315 | 56 | 2577 | 0.08 | 0.00 | 0.00 |
| | | 1 | | | | LDTA~GPEEYSAMRDQ | | | | |
| Q61P | 57 | TCEM | TCEM2 | DTAGPEEYS | 2316 | 57 | 257 | 0.01 | 0.00 | 0.00 |
| | | 1 | | | | DTAG~PEEYSAMRDQY | | | | |
| Q61P | 58 | TCEM | GEM2 | TAGPEEYSA | 2317 | 58 | 2579 | 0.00 | 0.01 | 0.05 |
| | | 1 | | | | TAGP~EEYSAMRDQYM | | | | |
| Q61P | 59 | GEM1 | | AGPEEYSAM | 2318 | 59 | 2580 | 0.01 | 0.01 | 0.02 |
| | | | | | | AGPE~EYSAMRDQYMR | | | | |
| Q61F | 60 | GEM1 | | GPEEYSAMR | 2319 | 60 | 2581 | 0.12 | 0.00 | 0.99 |
| | | | | | | GPEE~YSAMRDQYMRT | | | | |
| Q61P | 61 | GEM1 | | PEEYSAMRD | 2320 | 61 | 2582 | 0.51 | 0.16 | 0.00 |
| | | | | | | PEEY~SAMRDQYMRTG | | | | |
| Q61P | 62 | | | EEYSAMRDQ | 2321 | 62 | 2583 | 0.05 | 0.10 | 0.00 |
| | | | | | | EEYS-AMRDQYMRTGE | | | | |
| Q61P | 63 | | | EYSAMRDQY | 2322 | 63 | 2584 | 0.02 | 0.01 | 0.00 |
| | | | | | | EYSA~MRDQYMRTGEG | | | | |
| Q61P | 64 | | | YSAMRDQYM | 2323 | 64 | 2585 | 0.00 | 0.01 | 0.00 |
| | | | | | | YSAM~RDQYMRTGEGF | | | | |
| Q61F | 47 | | | DGETCLLDI | 2324 | 47 | 2586 | 0.28 | 0.00 | 0.04 |
| | | | | | | DGET~CLLDILDTAGR | | | | |
| Q61R | 48 | | | GETCLLDIL | 2325 | 48 | 2587 | 0.00 | 0.06 | 0.00 |
| | | | | | | GETC~LLDILDTAGRE | | | | |
| Q61R | 49 | | | ETCLLDILD | 2326 | 49 | 2588 | 0.00 | 0.00 | 0.10 |
| | | | | | | ETCL~LDILDTAGREE | | | | |
| Q61F | 50 | | GEM2 | TCLLDILDT | 2327 | 50 | 2589 | 0.99 | 0.15 | 0.09 |
| | | | | | | TCLL~DILDTAGREEY | | | | |
| Q61F | 51 | | TCEM2 | CLLDILDTA | 2328 | 51 | 2590 | 0.79 | 0.78 | 0.37 |
| | | | | | | CLLD~ILDTAGREEYS | | | | |
| Q61F | 52 | | TCEM2 | LLDILDTAG | 2329 | 52 | 2591 | 0.00 | 0.00 | 0.98 |
| | | | | | | LLDI~LDTAGREEYSA | | | | |
| Q61R | 53 | GEM1 | GEM2 | LDILDTAGR | 2330 | 53 | 2592 | 0.96 | 0.95 | 0.27 |
| | | | | | | LDIL~DTAGREEYSAM | | | | |
| Q61F | 54 | TCEM | TCEM2 | DILDTAGRE | 2331 | 54 | 2593 | 1.00 | 0.90 | 1.00 |
| | | 1 | | | | DILD~TAGREEYSAMR | | | | |
| Q61R | 55 | TCEM | GEM2 | ILDTAGREE | 2332 | 55 | 2594 | 0.00 | 0.01 | 0.00 |
| | | 1 | | | | ILDT~AGREEYSAMRD | | | | |
| Q61R | 56 | TCEM | TCEM2 | LDTAGREEY | 2333 | 56 | 2595 | 0.01 | 0.01 | 0.00 |
| | | 1 | | | | LDTA~GREEYSAMRDQ | | | | |
| Q61R | 57 | TCEM | TCEM2 | DTAGREEYS | 2334 | 57 | 2596 | 0.01 | 0.33 | 0.00 |
| | | 1 | | | | DTAG~REEYSAMRDQY | | | | |
| Q61R | 58 | TCEM | GEM2 | TAGREEYSA | 2335 | 58 | 2597 | 0.00 | 0.00 | |
| | | 1 | | | | TAGR~EEYSAMRDQYM | | | | |
| Q61F | 59 | GEM1 | | AGREEYSAM | 2336 | 59 | 2598 | 0.00 | 0.00 | 0.01 |
| | | | | | | AGRE~EYSAMRDQYMR | | | | |
| Q61R | 60 | GEM1 | | GREEYSAMR | 2337 | 60 | 2599 | 0.01 | 0.00 | 0.86 |
| | | | | | | GREE~YSAMRDQYMRT | | | | |
| Q61R | 61 | GEM1 | | REEYSAMRD | 2338 | 61 | 2600 | 0.65 | 0.02 | 0.01 |
| | | | | | | REEY~SAMRDQYMRTG | | | | |
| Q61R | 62 | | | EEYSAMRDQ | 2339 | 62 | 2601 | 0.05 | 0.10 | 0.00 |
| | | | | | | EEYS~AMRDQYMRTGE | | | | |
| Q61R | 63 | | | EYSAMRDQY | 2340 | 63 | 2602 | 0.02 | 0.01 | 0.00 |
| | | | | | | EYSA~MRDQYMRTGEG | | | | |
| Q61R | 64 | | | YSAMRDQYM | 2341 | 64 | 2603 | 0.00 | 0.01 | 0.00 |
| | | | | | | YSAM~RDQYMRTGEGF | | | | |

Legend:
Pos = position of mutation; facet 1 and facet 2: show T cell exposed (TCEM) positions ys groove exposed (GEM) positions for MHC I and II respectively; 9 mer peptide shows sequential peptides spanning the mutant position; Peptide cut-site shows position to which cathepsin probability refers indicated by ~; CAT_S, CAT_L and CAT_B indicate predicted probability of cleavage at the dimer separated by ~ in cut-site column.

While the corresponding table is not provided for HRAS and NRAS, given the identity of sequences with KRAS in the first 86 positions, it will be understood that the predicted cleavage patterns are the same.

Given this high probability of cleavage it is unlikely that a mutant Ras is presented to a T cell as a neoantigen except under extremely rare conditions. The chance of invoking immune elimination of a Ras mutation in the hotspot positions G12/13 or Q61 is thus very low as long as cathepsins are present and in particular Cathepsin B. Expression of KRAS is associated with upregulated cathepsin expression. FIG. 8 also demonstrates RNA transcription upregulated across multiple different tumors analyzed. It follows that down-regulation or inhibition of cathepsin, and in preferred embodiments down regulate or inhibit cathepsin B, will enable the exposure of the mutant neoepitopes as T cell antigens and thereby facilitate an effective immune response to these neoepitopes. Thus, administration of a cathepsin inhibitor as an adjunct to vaccination with Ras neoepitope peptides is a preferred embodiment in vaccinating subjects affected by such Ras mutations. Similarly in other tumor antigens where a high probability of cathepsin cleavage is demonstrated, coadministration of a cathepsin inhibitor can enhance efficacy of a neoepitope vaccine; the present invention is not limited to the RAS mutation neoepitopes.

As noted above, a number of cathepsin inhibitors are known to the art. The following examples are not considered limiting and on-going active drug development may provide additional inhibitors of the cathepsins, and in particular cathepsin B which will be suitable for use in conjunction with a neoantigen vaccine.

These include, but are not limited to, inhibitors derived from the classes of compound comprising nitrile derivatives, ketone derivatives, acryl hydrazine, vinyl sulfonate derivatives, epoxy succinic acid, betalactams, surugamides, loxistatin derivatives, sulfonamide derivatives, and many other products, natural medicinal products such as caffeic acid and chlorogenic acid, and members of the cystatin family. Additional cathepsin inhibitors may include antibodies to cathepsins or molecules derived from such antibodies, whether administered alone, or as a targeted antibody fusion or antibody conjugate products. In yet other embodiments targeting to a tumor site of interest may be achieved by fusion of a cathepsin inhibitor to a specific receptor binding ligand, including but not limited to an antibody or scFV fusion directed to a tumor specific epitope or ligand.

Achieving an effective cytotoxic response when a neoepitope peptide is cleaved by cathepsin, or other peptidases, involves two steps. First a neoepitope vaccine comprising the putative neoantigen but successfully stimulate cognate T cell clones at the site of vaccination in antigen presenting cells. Secondly, the presentation of the neoepitope as an antigen in the tumor requires overcoming peptide cleavage in tumor cells or the immediate extracellular matrix. Addressing these two components may call for multiple strategies including either or both of systemic and local applications of cathepsin inhibitors. In some embodiments, therefore, a cathepsin inhibitor may be administered parenterally. In other embodiments the inhibitor may be administered intratumorally or topically, where the tumor is accessible on the skin, or applied to an affected mucosal surface. In some embodiments a cathepsin inhibitor maybe co-administered with the vaccine peptides or their encoding nucleic acid sequences. In other embodiments the administration of the vaccine and the inhibitor may be implemented independently and sequentially. In some particular embodiments a cystatin protein or a sub-component polypeptide may be encoded in a nucleic acid sequence for intratumoral expression.

While the application of cathepsin inhibitors and intratumoral cystatin is discussed here with reference to the Ras gene product mutations, KRAS HRAS and NRAS, it will be clear to those skilled in the art that where other mutations are observed to occur in peptides with a high predicted probability of cathepsin cleavage, or indeed cleavage by other peptidases, that the coadministration of a cathepsin inhibitor, cystatin or other peptidase inhibitor is a therapeutic intervention which will enhance the stimulation of an effective tumor specific cytotoxic response.

### Example 5: MHC Binding and Cross Presentation

Both CD8+ and CD4+ responses are needed for an optimal tumor targeting response and MHC I and MHC II allele binding peptides are commonly found in proximity in dominant epitopes in infectious organisms (25, 26, 27). Similarly for tumor epitopes for the establishment of a mature CD8+ response both MHC I and MHC II epitopes are needed (23, 24). CD4+ responses have been shown to be capable of bringing about tumor responses alone and when acting in concert with MHC I CD8+ (2). Methods for determining predicted MHC binding have been previously described. See, e.g., PCT APPLs. US/2011/029192 and US/2012/055038, each of which are incorporated herein by reference in their entirety.

In some instances the naturally occurring peptide sequence comprising the mutant amino acid or acids may bind to one or more MHC molecules of the subject's genotype. In other instances, while the natural binding affinity may allow sub-dominant presentation of the T cell neoepitope, the binding affinity can be enhanced in the vaccine to stimulate expansion of more T cell clones and/or more expansion of the reactive clones. Optimization of the binding affinity can be achieved by modification of the groove exposed motifs by amino acid substitution in these positions to optimize binding for particular HLA alleles of interest. See, e.g., PCT APPL. US/2020/037206, incorporated herein by reference in its entirety. Modification of the groove exposed motifs may also allow the design of a peptide with better properties for manufacturing and administration, for instance by substitution of a terminal cysteine residue to minimize cross linkages.

In an ideal situation the mutant amino acid is presented to T cell receptors by both T cell exposed motifs of MHC I and MHC II. That this occurs with an optimal binding affinity for both MHC types in the context of a particular subject's HLA alleles is a lower probability occurrence. In some cases such cross presentation can be enhanced, creating heteroclitic peptides. Choice of peptides for inclusion in a library for rapid response to common cancer mutations needs to include both peptides which will elicit a tumor specific CD4+ as well as a CD8+ response, whether through the inclusion of naturally binding peptides or heteroclitic peptides to optimize binding for a particular common allele.

### Example 6: Library for Common Mutations

Application of the criteria of down-selection by motif frequency and, cathepsin cleavage probability enables, in one embodiment, the establishment of a list of those T cell exposed motifs most likely to stimulate a T cell response if placed in the context of an optimally binding groove exposed motif. T cell exposed motifs may be excluded from the list when the corresponding pentamers have a zero or low frequency in the human proteome and a frequency of less than 5 in the gastrointestinal microbiome reference database. Motifs may also be excluded if their count in the human proteome exceeds 100 or in some embodiments 50 and in yet others 20 counts. For the selected T cell exposed motifs peptides, or the nucleic acids encoding them, are identified that bind to the most common HLA alleles. For instance peptides that are predicted to bind with an affinity of from about 200 nM to about 2000 nM are added to the library as the natural sequence. If the predicted binding affinity is between about greater than 2000 nM to about 10,000 nM, a heteroclitic peptide is designed to provide optimized binding for the allele of interest in the vaccine. The ranges of binding shown here are guidance and not considered limiting. If the predicted binding is less than 10,000 nM it is unlikely that presentation of that T cell motif would ever occur for that allele in vivo and the peptide is not added to the library.

In a first embodiment the library comprises peptides for the mutations shown in Table 2 for A0201, A0101, A0301, A2402, and A1101 and B0702, B0801, B4402, B1501, B5301 and B3501. In a second embodiment the library comprises peptides for the mutations shown in Table 3 for DRB1*0101, DRB1*0401, DRB1*0301, DRB1*1501 and DRB1*0701, In yet further embodiments peptides are added to the library for additional alleles and additional mutated gene products. In a yet further embodiment the library comprises the sequences shown in Table 14 as non-limiting examples.

TABLE 14
SEQ ID NO.:
1736
SEQ ID NO.:
1737
SEQ ID NO.:
1738
SEQ ID NO.:
1739
SEQ ID NO.:
1751
SEQ ID NO.:
1766
SEQ ID NO.:
1767
SEQ ID NO.:
1781
SEQ ID NO.:
1782
SEQ ID NO.:
1783
SEQ ID NO.:
1794
SEQ ID NO.:
1795
SEQ ID NO.:
1799
SEQ ID NO.:
1800
SEQ ID NO.:
1809
SEQ ID NO.:
1814
SEQ ID NO.:
1824
SEQ ID NO.:
1830
SEQ ID NO.:
1839
SEQ ID NO.:
1840
SEQ ID NO.:
1844
SEQ ID NO.:
1850
SEQ ID NO.:
1851
SEQ ID NO.:
1852
SEQ ID NO.:
1853
SEQ ID NO.:
1854
SEQ ID NO.:
1855
SEQ ID NO.:
1856
SEQ ID NO.:
1857
SEQ ID NO.:
1858
SEQ ID NO.:
1859
SEQ ID NO.:
1860
SEQ ID NO.:
1861
SEQ ID NO.:
1862
SEQ ID NO.:
1863
SEQ ID NO.:
1864
SEQ ID NO.:
1865
SEQ ID NO.:
1866
SEQ ID NO.:
1867
SEQ ID NO.:
1868
SEQ ID NO.:
1869
SEQ ID NO.:
1870
SEQ ID NO.:
1871
SEQ ID NO.:
1872
SEQ ID NO.:
1873
SEQ ID NO.:
1874
SEQ ID NO.:
1875
SEQ ID NO.:
1876
SEQ ID NO.:
1877
SEQ ID NO.:
1878
SEQ ID NO.:
1879
SEQ ID NO.:
1880
SEQ ID NO.:
1881
SEQ ID NO.:
1882
SEQ ID NO.:
1883
SEQ ID NO.:
1884
SEQ ID NO.:
1885
SEQ ID NO.:
1886
SEQ ID NO.:
1887
SEQ ID NO.:
1888
SEQ ID NO.:
1889
SEQ ID NO.:
1891
SEQ ID NO.:
1897
SEQ ID NO.:
1898
SEQ ID NO.:
1900
SEQ ID NO.:
1902
SEQ ID NO.:
1903
SEQ ID NO.:
1904
SEQ ID NO.:
1905
SEQ ID NO.:
1906
SEQ ID NO.:
1907
SEQ ID NO.:
1908
SEQ ID NO.:
1909
SEQ ID NO.:
1910
SEQ ID NO.:
1913
SEQ ID NO.:
1914
SEQ ID NO.:
1915
SEQ ID NO.:
1916
SEQ ID NO.:
1921
SEQ ID NO.:
1922
SEQ ID NO.:
1923
SEQ ID NO.:
1924
SEQ ID NO.:
1927
SEQ ID NO.:
1928
SEQ ID NO.:
1929
SEQ ID NO.:
1930
SEQ ID NO.:
1932
SEQ ID NO.:
1933
SEQ ID NO.:
1934
SEQ ID NO.:
1935
SEQ ID NO.:
1936
SEQ ID NO.:
1937
SEQ ID NO.:
1938
SEQ ID NO.:
1939
SEQ ID NO.:
1940
SEQ ID NO.:
1941
SEQ ID NO.:
1942
SEQ ID NO.:
1943
SEQ ID NO.:
1944
SEQ ID NO.:
1945
SEQ ID NO.:
1946
SEQ ID NO.:
1947
SEQ ID NO.:
1948
SEQ ID NO.:
1949
SEQ ID NO.:
1950
SEQ ID NO.:
1951

In one embodiment the library comprises an annotated list of peptide sequences; in a further embodiment the library comprises an annotated list of nucleic acid sequences encoding the peptides. In a yet further embodiment the peptides, nor encoding nucleic acids, are synthesized and stored in preparation for immediate use.

### Example 7: Selection of T Cell Clones

Further modes of immunotherapeutic intervention in cancer include autologous T cell transfer and the engineering of T cells for administration to affected subjects. Specificity of such T cells is of the highest importance to be sure only tumor cells and only their mutated proteins are targeted, leaving normal cells unharmed. Both, or either, CD8+ and CD4+ cells may be deployed.

The present invention provides a means of rapidly down-selecting the peptides which carry mutant amino acids and their T cell motifs to just those peptides which will engage specific T cells which have greatest probability of being represented in the precursor population, as well as peptides that also have a high probability of being presented by a subject's alleles. These are the T cells with highest probability of providing an effective CD8+ or CD4+ response. Given that tumors arise in the context of immune evasion, the most desirable T cells are typically rare clones in the overall T cell population. Furthermore, when T cells are harvested by collection of blood or a tumor biopsy, limited numbers of cells are typically available and it is essential to be able to expand the specific desired clonal populations over the competition of other clones present.

The methods provided here are of particular utility where the T cell exposed motifs that would expose the mutant amino acid is rare in both the human proteome and the exogenous proteins to which an individual subject may have been exposed in the establishment and maintenance of their T cell repertoire. In this situation smaller numbers of cognate T cells suitable for expansion are available. This includes many of the commonly occurring tumor mutations listed in Table 2.

In some instances, others have identified effective TCRs for rare T cell exposed motifs but this has been achieved by an empirical experimental approach which is laborious and costly. The present methods, by enabling rapid down selection to a few T cell exposed motifs of interest, provide a significant time savings over purely experimental approaches.

FIG. 5 and Tables 7 and 8 demonstrate for two cancer-affected individuals how peptides selected based on frequency as well as HLA binding can provide a more precise ability to select T cell clones which are suitable for expansion and administration to individuals with HLA shared with these subjects, and to avoid T cell exposed motifs and the peptides that encompass them which may not encounter a cognate T cell clone.

When peptides comprising neoepitopes are used as a means of capture of T cells of interest the binding affinity of the peptides that comprise these T cell motifs can be enhanced by providing a peptide comprising the T cell exposed motif of interest with modified groove exposed motifs to optimize binding for a particular HLA allele, as previously described (See, e.g., PCT APPL. US/2020/037206, incorporated herein by reference in its entirety). In those instances where a selected peptide comprising a mutated T cell exposed motif of interest is identified to bind to a set of one or more common HLA alleles, the opportunity exists to sequence the TCR and create a bank of T cells or engineered T cells for future use in individuals of such allele. In other instances, an autologous approach may be preferred due to a subject having less common alleles or mutations.

Methods of expansion of T cells and sequencing of the T cell receptors (TCR) therein are known to the art. Having identified the key T cell exposed motif based on the down-selection criteria and the peptide encompassing it, whether natural or heteroclitic, the peptide is presented by antigen presenting cells and then contacted with T cells to expand the specific clones. Methods of culturing of antigen presenting cells (APCs) to favor peptide presentation on MHC are well known to those skilled in the art (for example (102)). The APCs are then co-cultured in contact with the single selected peptide of interest comprising the T cell exposed motif. The T cell exposed motif may be a TCEM I (contiguous pentameric amino acid motifs designed to be presented on MHC I) in a 9mer or a TCEM II (comprising the T cell exposed as a discontinuous pentameric motif within preferably a 15mer, or a 11-22mer and designed to be presented by an MHC II. In alternative embodiments the peptide of interest is encoded in a nucleic acid sequence delivered to the APC by transfection or vector or virus or other gene transfer methods. In preferred embodiments the antigen presenting cells are antigen-naïve. The APCs presenting the T cell exposed motif of interest are then co-cultivated with T cells and the expanded T cell population harvested after an appropriate replication period. Confirmation that the expanded T cell clones are indeed specific to the selected T cell exposed motif of interest can be achieved by examining the response to the peptide comprising the mutated T cell exposed motif compared to the wild type of the same peptide and evaluating the cytokine profile of each or by evaluating the response to the mutated peptide is significantly increased compared to the wildtype peptide in an ELISPOT assay. These are methods well known to the art.

The expanded T cell population specific to the mutated T cell exposed motif and the MHC allele may be administered to the subject of origin as an autologous transfer, or frozen and stored for later administration. In yet other applications sequences of the alpha and beta chains of the TCR may be sequenced from one or more individual T cell cells or clones with specificity of the T cell exposed motif-allele combination, and the sequences engineered into other cells which may be cells in culture for research or assay purposes or naïve recipient T cells.

### Example 8: Mutations Other than Missense

Many types of genomic mutations occur in tumors. Those in coding regions are represented by mutated proteins. The mutations in the proteins comprise but are not limited to missense mutations, insertions and deletions, splice variants, and fusions that arise at both the DNA and RNA level. Any of these may produce a T cell exposed motif that is unique to the tumor and differentiates it from the wild-type of the same protein or proteins. The selection of neoepitope targets based on the frequency of their pentameric amino acids motifs in the human proteome and in reference databases representative of environmental exposure are equally relevant to mutations other than missense mutations. Table 15 illustrated the diverse pentamer frequency count in the human proteome and gastrointestinal microbiome of unique T cell exposed motif pentamers in two fusions of KIAA1549-BRAF that are found in gliomas (103). These are T cell exposed motifs that are unique to the tumor because they span the fusion bridge and are not found in either of the parent proteins. Hence in one embodiment the present invention provides a method for prioritizing neoepitopes in fusions proteins occurring in tumors, including but not limited to, those in KIAA1549-BRAF, EML4-ALK, BCR-ABL, DNAJB1-PRKCA, PTPRZ1-MET, FGFR3-TACC3, EWS-FLI and NTRK fusions. Exemplars from common fusion proteins and common HLA alleles are in a further embodiment added to the library of prepared neoantigen peptides or nucleic acids.

TABLE 15

| index | | | SEQ ID | | SEQ ID | hPPF | giPPF | hPPF | giPPF |
|---|---|---|---|---|---|---|---|---|---|
| position | curation | TCEM I | NO.: | TCEM IIa | NO.: | I | I | II | II |
| 1740 | KIAA1549_BRAF 16_9 | | | AN~P~SD | 1940 | 9 | 65 | 2 | 38 |
| 1741 | KIAA1549_BRAF 16_9 | | | NN~C~DL | 1941 | 3 | 2 | 2 | 4 |
| 1742 | KIAA1549_BRAF 16_9 | | | NP~S~LI | 1942 | 1 | 2 | 10 | 73 |
| 1743 | KIAA1549_BRAF 16_9 | ~~~NPCSD~ | 1932 | PC~D~IR | 1943 | 4 | 2 | 0 | 12 |
| 1744 | KIAA1549_BRAF 16_9 | ~~~PCSDL~ | 1933 | CS~L~RD | 1944 | 8 | 5 | 10 | 13 |
| 1745 | KIAA1549_BRAF 16_9 | ~~~CSDLI~ | 1934 | SD~I~DQ | 1945 | 0 | 6 | 2 | 64 |
| 1746 | KIAA1549_BRAF 16_9 | ~~~SDLIR~ | 1935 | | | 24 | 107 | 13 | 64 |
| 1634 | KIAA1549_BRAF 15_9 | | | AY~G~PD | 1946 | 2 | 46 | 2 | 38 |
| 1635 | KIAA1549_BRAF 15_9 | | | YI~C~DL | 1947 | 1 | 10 | 1 | 9 |
| 1636 | KIAA1549_BRAF 15_9 | | | IG~P~LI | 1948 | 1 | 6 | 2 | 117 |
| 1637 | KIAA1549_BRAF 15_9 | ~~~IGCPD~ | 1936 | GC~D~IR | 1949 | 1 | 8 | 1 | 21 |
| 1638 | KIAA1549_BRAF 15_9 | ~~~GCPDL~ | 1937 | CP~L~RD | 1950 | 5 | 28 | 5 | 6 |
| 1639 | KIAA1549_BRAF 15_9 | ~~~CPDLI~ | 1938 | PD~I~DQ | 1951 | 2 | 22 | 5 | 57 |
| 1640 | KIAA1549_BRAF 15_9 | ~~~PDLIR~ | 1939 | | | 4 | 83 | 13 | 64 |

### Example 9: Targeting B Cell Neoepitopes

Many tumor mutations occur in proteins which have a transmembrane domain and have sequences that are cell surface exposed. Among the most common ATM, EGFR, FGFR2, FGFR3 and PDGRFA are examples, but many passenger genes that are mutated have extracellular domains. About a quarter of missense mutations occur in or adjacent to probable B cell epitopes, thereby creating altered or novel B cell epitopes or create higher probability B cell epitopes.

Such novel B cell neoepitopes have the potential to stimulate antibodies with potential for antibody-dependent cell-mediated cytotoxicity (ADCC). When such epitopes are recognized in the course of tumor mutation analysis, the ability to rapidly synthesize peptides encompassing such novel B cell epitopes is advantageous and may be particularly relevant when administered in conjunction with CD4+ T helper cells. The peptide trimer building block approach described in Example 10 may thus be applied to the construction of longer peptides that span the novel B cell epitope. While the minimal size of a B cell linear may be 3 or 5 amino acids, by using trimer subunits such peptides may be constructed as any multiple of three amino acids, in some non-limiting examples up to 33 or 36 amino acids in length to encompass an MHC II binding peptide as a CD4+ helper.

### Example 10: Peptide Trimer Building Blocks

Neoepitope vaccines may be delivered to a subject in many ways, including as nucleic acids encoding peptides, or as peptides. In some preferred embodiments MHC I neoepitopes are delivered as a 9mer peptide and MHC II neoepitopes are delivered as a 15mer peptide. B cell epitopes may be delivered as longer peptides, in some cases spanning both linear B cell epitopes and a T helper MHC II epitope.

When neoepitopes are delivered to the subject as a peptide rapid assembly and streamlining of quality control is desirable to allow neoepitope vaccination to be initiated as swiftly as possible after diagnosis, biopsy and sequencing. While a 9 mer peptide has 20⁹ possible configurations and a 15 mer has 20¹⁵ potential configurations, a trimer has only 20³ possibilities or 8000 possible configurations. Thus a set of 3 or 5 trimers drawn from a set of 8000 trimers can be assembled to form any 9 mer or 15mer, or by assembly of more trimer units to make a longer peptide with length a multiple of three. In reality, some trimers are never or rarely encountered (for example CCC and WWW) or are found in peptides with less desirable epitope or manufacturing qualities, and hence a somewhat smaller library of trimers can address most needs. Methods of peptide synthesis are well known to those skilled in the art and are typically conducted by multiple sequential reactions to add of single amino acids with deprotection of the N or C end group, by removal of a Boc (tert-butoxycarbonyl) or Fmoc (9-fluorenylmethoxycarbonyl) protecting group to enable new bond formation as each new amino acid is added. This process is conducted stepwise for each amino acid addition. Significant savings in time and quality assurance steps can be achieved by building neoepitope vaccines from trimer subunit “building bricks” that are preassembled and stored for future use.

### REFERENCES

1. Tran E, Robbins P F, Rosenberg S A. ‘Final common pathway’ of human cancer immunotherapy: targeting random somatic mutations. Nat Immunol. 2017; 18(3):255-62.
2. Lang F, Schrors B, Lower M, Tureci O, Sahin U. Identification of neoantigens for individualized therapeutic cancer vaccines. Nature reviews Drug discovery. 2022; 21(4):261-82.
3. Wells D K, van Buuren M M, Dang K K, Hubbard-Lucey V M, Sheehan K C F, Campbell K M, et al. Key Parameters of Tumor Epitope Immunogenicity Revealed Through a Consortium Approach Improve Neoantigen Prediction. Cell. 2020; 183(3):818-34 e13.
4. Lefranc M P, Giudicelli V, Ginestoux C, Jabado-Michaloud J, Folch G, Bellahcene F, et al. IMGT, the international ImMunoGeneTics information system. Nucleic acids research. 2009; 37(Database issue):D1006-12.
5. Duan F, Duitama J, Al Seesi S, Ayres C M, Corcelli S A, Pawashe A P, et al. Genomic and bioinformatic profiling of mutational neoepitopes reveals new rules to predict anticancer immunogenicity. J Exp Med. 2014; 211(11):2231-48.
6. Schumacher T N, Schreiber R D. Neoantigens in cancer immunotherapy. Science. 2015; 348(6230):69-74.
7. Parkhurst M R, Robbins P F, Tran E, Prickett T D, Gartner J J, Jia L, et al. Unique Neoantigens Arise from Somatic Mutations in Patients with Gastrointestinal Cancers. Cancer Discov. 2019; 9(8):1022-35.
8. Garcia K C, Teyton L, Wilson I A. Structural basis of T cell recognition. Annu Rev Immunol. 1999; 17:369-97.
9. Rudolph M G, Stanfield R L, Wilson I A. How TCRs bind MHCs, peptides, and coreceptors. Annu Rev Immunol. 2006; 24:419-66.
10. Calis J J, de Boer R J, Kesmir C. Degenerate T-cell recognition of peptides on MHC molecules creates large holes in the T-cell repertoire. PLoS computational biology. 2012; 8(3):e1002412.
11. Naumov Y N, Hogan K T, Naumova E N, Pagel J T, Gorski J. A class I MHC-restricted recall response to a viral peptide is highly polyclonal despite stringent CDR3 selection: implications for establishing memory T cell repertoires in “real-world” conditions. J Immunol. 1998; 160(6):2842-52.
12. Falk K, Rotzschke O, Stevanovic S, Jung G, Rammensee H G. Allele-specific motifs revealed by sequencing of self-peptides eluted from MHC molecules. Nature. 1991; 351(6324):290-6.
13. Fritsch E F, Rajasagi M, Ott P A, Brusic V, Hacohen N, Wu C J. HLA-binding properties of tumor neoepitopes in humans. Cancer immunology research. 2014; 2(6):522-9.
14. Calis J J, Maybeno M, Greenbaum J A, Weiskopf D, De Silva A D, Sette A, et al. Properties of MHC class I presented peptides that enhance immunogenicity. PLoS computational biology. 2013; 9(10):e1003266.
15. Reddehase M J, Rothbard J B, Koszinowski U H. A pentapeptide as minimal antigenic determinant for MHC class I-restricted T lymphocytes. Nature. 1989; 337(6208):651-3.
16. Bremel R D, Homan E J. Frequency Patterns of T-Cell Exposed Amino Acid Motifs in Immunoglobulin Heavy Chain Peptides Presented by MHCs. Frontiers in immunology. 2014; 5:541.
17. Birnbaum M E, Mendoza J L, Sethi D K, Dong S, Glanville J, Dobbins J, et al. Deconstructing the Peptide-MHC Specificity of T Cell Recognition. Cell. 2014; 157(5):1073-87.
18. Wei P, Jordan K R, Buhrman J D, Lei J, Deng H, Marrack P, et al. Structures suggest an approach for converting weak self-peptide tumor antigens into superagonists for CD8 T cells in cancer. Proc Natl Acad Sci USA. 2021; 118(23).
19. Wang Y, Sosinowski T, Novikov A, Crawford F, Neau D B, Yang J, et al. C-terminal modification of the insulin B:11-23 peptide creates superagonists in mouse and human type 1 diabetes. Proc Natl Acad Sci USA. 2018; 115(1):162-7.
20. Nelson R W, Beisang D, Tubo N J, Dileepan T, Wiesner D L, Nielsen K, et al. T cell receptor cross-reactivity between similar foreign and self peptides influences naive cell population size and autoimmunity. Immunity. 2015; 42(1):95-107.
21. Rossjohn J, Gras S, Miles J J, Turner S J, Godfrey D I, McCluskey J. T cell antigen receptor recognition of antigen-presenting molecules. Annu Rev Immunol. 2015; 33:169-200.
22. Bremel R D, Homan J. Extensive T-cell epitope repertoire sharing among human proteome, gastrointestinal microbiome, and pathogenic bacteria: Implications for the definition of self. Frontiers in immunology. 2015; 6.
23. Alspach E, Lussier D M, Miceli A P, Kizhvatov I, DuPage M, Luoma A M, et al. MHC-II neoantigens shape tumour immunity and response to immunotherapy. Nature. 2019; 574(7780):696-701.
24. Zander R, Schauder D, Xin G, Nguyen C, Wu X, Zajac A, et al. CD4(+) T Cell Help Is Required for the Formation of a Cytolytic C D8(+) T Cell Subset that Protects against Chronic Infection and Cancer. Immunity. 2019; 51(6):1028-42 e4.
25. Sun J C, Bevan M J. Defective CD8 T cell memory following acute infection without CD4 T cell help. Science. 2003; 300(5617):339-42.
26. Janssen E M, Lemmens E E, Wolfe T, Christen U, von Herrath M G, Schoenberger S P. CD4+ T cells are required for secondary expansion and memory in CD8+T lymphocytes. Nature. 2003; 421(6925):852-6.
27. Bremel R D, Homan E J. Recognition of higher order patterns in proteins: immunologic kernels. PloS one. 2013; 8(7):e70115.
28. Kreiter S, Vormehr M, van de Roemer N, Diken M, Lower M, Diekmann J, et al. Mutant MHC class II epitopes drive therapeutic immune responses to cancer. Nature. 2015; 520(7549):692-6.
29. Klein L, Kyewski B, Allen P M, Hogquist K A. Positive and negative selection of the T cell repertoire: what thymocytes see (and don't see). Nature reviews Immunology. 2014; 14(6):377-91.
30. Takaba H, Takayanagi H. The Mechanisms of T Cell Selection in the Thymus. Trends in immunology. 2017; 38(11):805-16.
31. Fulton R B, Hamilton S E, Xing Y, Best J A, Goldrath A W, Hogquist K A, et al. The TCR's sensitivity to self peptide-MHC dictates the ability of naive CD8(+) T cells to respond to foreign antigens. Nat Immunol. 2015; 16(1):107-17.
32. Michelson D A, Hase K, Kaisho T, Benoist C, Mathis D. Thymic epithelial cells co-opt lineage-defining transcription factors to eliminate autoreactive T cells. Cell. 2022; 185(14):2542-58 e18.
33. Davis M M. Not-So-Negative Selection. Immunity. 2015; 43(5):833-5.
34. Koncz B, Balogh G M, Papp B T, Asztalos L, Kemeny L, Manczinger M. Self-mediated positive selection of T cells sets an obstacle to the recognition of nonself. Proc Natl Acad Sci USA. 2021; 118(37).
35. Hebbandi Nanjundappa R, Sokke Umeshappa C, Geuking M B. The impact of the gut microbiota on T cell ontogeny in the thymus. Cell Mol Life Sci. 2022; 79(4):221.
36. Zegarra-Ruiz D F, Kim D V, Norwood K, Kim M, Wu W H, Saldana-Morales F B, et al. Thymic development of gut-microbiota-specific T cells. Nature. 2021; 594(7863):413-7.
37. Ennamorati M, Vasudevan C, Clerkin K, Halvorsen S, Verma S, Ibrahim S, et al. Intestinal microbes influence development of thymic lymphocytes in early life. Proc Natl Acad Sci USA. 2020; 117(5):2570-8.
38. Hadeiba H, Lahl K, Edalati A, Oderup C, Habtezion A, Pachynski R, et al. Plasmacytoid dendritic cells transport peripheral antigens to the thymus to promote central tolerance. Immunity. 2012; 36(3):438-50.
39. Murray J M, Kaufmann G R, Hodgkin P D, Lewin S R, Kelleher A D, Davenport M P, et al. Naive T cells are maintained by thymic output in early ages but by proliferation without phenotypic change after age twenty. Immunology and cell biology. 2003; 81(6):487-95.
40. Palmer D B. The effect of age on thymic function. Frontiers in immunology. 2013; 4:316.
41. Palmer S, Albergante L, Blackburn C C, Newman T J. Thymic involution and rising disease incidence with age. Proc Natl Acad Sci USA. 2018; 115(8):1883-8.
42. Thyagarajan B, Faul J, Vivek S, Kim J K, Nikolich-Zugich J, Weir D, et al. Age-Related Differences in T-Cell Subsets in a Nationally Representative Sample of People Older Than Age 55: Findings From the Health and Retirement Study. J Gerontol A Biol Sci Med Sci. 2022; 77(5):927-33.
43. Qi Q, Liu Y, Cheng Y, Glanville J, Zhang D, Lee J Y, et al. Diversity and clonal selection in the human T-cell repertoire. Proc Natl Acad Sci USA. 2014; 111(36):13139-44.
44. Emerson R O, DeWitt W S, Vignali M, Gravley J, Hu J K, Osborne E J, et al. Immunosequencing identifies signatures of cytomegalovirus exposure history and HLA-mediated effects on the T cell repertoire. Nat Genet. 2017; 49(5):659-65.
45. Bessell C A, Isser A, Havel J J, Lee S, Bell D R, Hickey J W, et al. Commensal bacteria stimulate antitumor responses via T cell cross-reactivity. JCI Insight. 2020; 5(8).
46. Gopalakrishnan V, Spencer C N, Nezi L, Reuben A, Andrews M C, Karpinets T V, et al. Gut microbiome modulates response to anti-PD-1 immunotherapy in melanoma patients. Science. 2018; 359(6371):97-103.
47. Ott P A, Hu Z, Keskin D B, Shukla S A, Sun J, Bozym D J, et al. An immunogenic personal neoantigen vaccine for patients with melanoma. Nature. 2017; 547(7662):217-21.
48. Hilf N, Kuttruff-Coqui S, Frenzel K, Bukur V, Stevanovic S, Gouttefangeas C, et al. Actively personalized vaccination trial for newly diagnosed glioblastoma. Nature. 2019; 565(7738):240-5.
49. Li F, Chen C, Ju T, Gao J, Yan J, Wang P, et al. Rapid tumor regression in an Asian lung cancer patient following personalized neo-epitope peptide vaccination. Oncoimmunology. 2016; 5(12):e1238539.
50. Yarmarkovich M, Farrel A, Sison A, 3rd, di Marco M, Raman P, Parris J L, et al. Immunogenicity and Immune Silence in Human Cancer. Frontiers in immunology. 2020; 11:69.
51. McGranahan N, Rosenthal R, Hiley C T, Rowan A J, Watkins T B K, Wilson G A, et al. Allele-Specific HLA Loss and Immune Escape in Lung Cancer Evolution. Cell. 2017; 171(6):1259-71 ell.
52. Zhou C, Tuong Z K, Frazer I H. Papillomavirus Immune Evasion Strategies Target the Infected Cell and the Local Immune System. Frontiers in oncology. 2019; 9:682.
53. Oliveira G, Stromhaug K, Cieri N, Iorgulescu J B, Klaeger S, Wolff J O, et al. Landscape of helper and regulatory antitumour CD4(+) T cells in melanoma. Nature. 2022; 605(7910):532-8.
54. Chen D S, Mellman I. Elements of cancer immunity and the cancer-immune set point. Nature. 2017; 541(7637):321-30.
55. Philip M, Schietinger A. CD8(+) T cell differentiation and dysfunction in cancer. Nature reviews Immunology. 2022; 22(4):209-23.
56. McGranahan N, Swanton C. Cancer Evolution Constrained by the Immune Microenvironment. Cell. 2017; 170(5):825-7.
57. Joyce J A, Fearon D T. T cell exclusion, immune privilege, and the tumor microenvironment. Science. 2015; 348(6230):74-80.
58. Dunn G P, Bruce A T, Ikeda H, Old L J, Schreiber R D. Cancer immunoediting: from immunosurveillance to tumor escape. Nat Immunol. 2002; 3(11):991-8.
59. Schreiber R D, Old L J, Smyth M J. Cancer immunoediting: integrating immunity's roles in cancer suppression and promotion. Science. 2011; 331(6024):1565-70.
60. Matsushita H, Vesely M D, Koboldt D C, Rickert C G, Uppaluri R, Magrini V J, et al. Cancer exome analysis reveals a T-cell-dependent mechanism of cancer immunoediting. Nature. 2012; 482(7385):400-4.
61. Hobbs G A, Der C J, Rossman K L. RAS isoforms and mutations in cancer at a glance. J Cell Sci. 2016; 129(7):1287-92.
62. Tran E, Robbins P F, Lu Y C, Prickett T D, Gartner J J, Jia L, et al. T-Cell Transfer Therapy Targeting Mutant KRAS in Cancer. The New England journal of medicine. 2016; 375(23):2255-62.
63. Khleif S N, Abrams S I, Hamilton J M, Bergmann-Leitner E, Chen A, Bastian A, et al. A phase I vaccine trial with peptides reflecting ras oncogene mutations of solid tumors. J Immunother. 1999; 22(2):155-65.
64. Carbone D P, Ciernik I F, Kelley M J, Smith M C, Nadaf S, Kavanaugh D, et al. Immunization with mutant p53- and K-ras-derived peptides in cancer patients: immune response and clinical outcome. Journal of clinical oncology: official journal of the American Society of Clinical Oncology. 2005; 23(22):5099-107.
65. Poole A, Karuppiah V, Hartt A, Haidar J N, Moureau S, Dobrzycki T, et al. Therapeutic high affinity T cell receptor targeting a KRAS(G12D) cancer neoantigen. Nature communications. 2022; 13(1):5333.
66. Sorenson G D. Detection of mutated KRAS2 sequences as tumor markers in plasma/serum of patients with gastrointestinal cancer. Clin Cancer Res. 2000; 6(6):2129-37.
67. Vogelstein B, Papadopoulos N, Velculescu V E, Zhou S, Diaz L A, Jr., Kinzler K W. Cancer genome landscapes. Science. 2013; 339(6127):1546-58.
68. Grossman R L, Heath A P, Ferretti V, Varmus H E, Lowy D R, Kibbe W A, et al. Toward a Shared Vision for Cancer Genomic Data. The New England journal of medicine. 2016; 375(12):1109-12.
69. UniProt C. UniProt: the universal protein knowledgebase in 2021. Nucleic acids research. 2021; 49(D1):D480-D9.
70. Fonovic M, Turk B. Cysteine cathepsins and their potential in clinical therapy and biomarker discovery. Proteomics Clin Appl. 2014; 8(5-6):416-26.
71. Oldak L, Milewska P, Chludzinska-Kasperuk S, Grubczak K, Reszec J, Gorodkiewicz E. Cathepsin B, D and S as Potential Biomarkers of Brain Glioma Malignancy. J Clin Med. 2022; 11(22).
72. Aggarwal N, Sloane B F. Cathepsin B: multiple roles in cancer. Proteomics Clin Appl. 2014; 8(5-6):427-37.
73. Ferreira A, Pereira F, Reis C, Oliveira M J, Sousa M J, Preto A. Crucial Role of Oncogenic KRAS Mutations in Apoptosis and Autophagy Regulation: Therapeutic Implications. Cells. 2022; 11(14).
74. Gocheva V, Zeng W, Ke D, Klimstra D, Reinheckel T, Peters C, et al. Distinct roles for cysteine cathepsin genes in multistage tumorigenesis. Genes & development. 2006; 20(5):543-56.
75. Gopinathan A, Denicola G M, Frese K K, Cook N, Karreth F A, Mayerle J, et al. Cathepsin B promotes the progression of pancreatic ductal adenocarcinoma in mice. Gut. 2012; 61(6):877-84.
76. Zhang T, Maekawa Y, Hanba J, Dainichi T, Nashed B F, Hisaeda H, et al. Lysosomal cathepsin B plays an important role in antigen processing, while cathepsin D is involved in degradation of the invariant chain inovalbumin-immunized mice. Immunology. 2000; 100(1):13-20.
77. Gocheva V, Joyce J A. Cysteine cathepsins and the cutting edge of cancer invasion. Cell Cycle. 2007; 6(1):60-4.
78. Fonovic M, Turk B. Cysteine cathepsins and extracellular matrix degradation. Biochim Biophys Acta. 2014; 1840(8):2560-70.
79. Cavallo-Medved D, Dosescu J, Linebaugh B E, Sameni M, Rudy D, Sloane B F. Mutant K-ras regulates cathepsin B localization on the surface of human colorectal carcinoma cells. Neoplasia. 2003; 5(6):507-19.
80. Vasiljeva O, Korovin M, Gajda M, Brodoefel H, Bojic L, Kruger A, et al. Reduced tumour cell proliferation and delayed development of high-grade mammary carcinomas in cathepsin B-deficient mice. Oncogene. 2008; 27(30):4191-9.
81. Kramer L, Turk D, Turk B. The Future of Cysteine Cathepsins in Disease Management. Trends Pharmacol Sci. 2017; 38(10):873-98.
82. Senjor E, Kos J, Nanut M P. Cysteine Cathepsins as Therapeutic Targets in Immune Regulation and Immune Disorders. Biomedicines. 2023; 11(2).
83. Siklos M, BenAissa M, Thatcher G R. Cysteine proteases as therapeutic targets: does selectivity matter? A systematic review of calpain and cathepsin inhibitors. Acta Pharm Sin B. 2015; 5(6):506-19.
84. Li Y Y, Fang J, Ao G Z. Cathepsin B and L inhibitors: a patent review (2010-present). Expert Opin Ther Pat. 2017; 27(6):643-56.
85. Kuranaga T, Matsuda K, Sano A, Kobayashi M, Ninomiya A, Takada K, et al. Total Synthesis of the Nonribosomal Peptide Surugamide B and Identification of a New Offloading Cyclase Family. Angewandte Chemie. 2018; 57(30):9447-51.
86. Gornowicz A, Szymanowska A, Mojzych M, Czarnomysy R, Bielawski K, Bielawska A. The Anticancer Action of a Novel 1,2,4-Triazine Sulfonamide Derivative in Colon Cancer Cells. Molecules. 2021; 26(7).
87. Supuran C T, Casini A, Scozzafava A. Protease inhibitors of the sulfonamide type: anticancer, antiinflammatory, and antiviral agents. Med Res Rev. 2003; 23(5):535-58.
88. Hook G, Jacobsen J S, Grabstein K, Kindy M, Hook V. Cathepsin B is a New Drug Target for Traumatic Brain Injury Therapeutics: Evidence for E64d as a Promising Lead Drug Candidate. Front Neurol. 2015; 6:178.
89. Frlan R, Gobec S. Inhibitors of cathepsin B. Curr Med Chem. 2006; 13(19):2309-27.
90. Murata M, Miyashita S, Yokoo C, Tamai M, Hanada K, Hatayama K, et al. Novel epoxysuccinyl peptides. Selective inhibitors of cathepsin B, in vitro. FEBS Lett. 1991; 280(2):307-10.
91. Ulcakar L, Novinec M. Inhibition of Human Cathepsins B and L by Caffeic Acid and Its Derivatives. Biomolecules. 2020; 11(1).
92. Breznik B, Mitrovic A, T T L, Kos J. Cystatins in cancer progression: More than just cathepsin inhibitors. Biochimie. 2019; 166:233-50.
93. Bakshi M, Tuo W, Aroian R V, Zarlenga D. Immune reactivity and host modulatory roles of two novel Haemonchus contortus cathepsin B-like proteases. Parasites & vectors. 2021; 14(1):580.
94. Fusetani N, Fujita M, Nakao Y, Matsunaga S, Van Soest R W. Tokaramide A, a new cathepsin B inhibitor from the marine sponge Theonella aff, mirabilis. Bioorg Med Chem Lett. 1999; 9(24):3397-402.
95. Satoyoshi E. Therapeutic trials on progressive muscular dystrophy. Intern Med. 1992; 31(7):841-6.
96. Human Microbiome Project C. A framework for human microbiome research. Nature. 2012; 486(7402):215-21.
97. Honey K, Rudensky A Y. Lysosomal cysteine proteases regulate antigen presentation. Nature reviews Immunology. 2003; 3(6):472-82.
98. Hoglund R A, Torsetnes S B, Lossius A, Bogen B, Homan E J, Bremel R, et al. Human Cysteine Cathepsins Degrade Immunoglobulin G In Vitro in a Predictable Manner. Int J Mol Sci. 2019; 20(19).
99. Biniossek M L, Nagler D K, Becker-Pauly C, Schilling O. Proteomic identification of protease cleavage sites characterizes prime and non-prime specificity of cysteine cathepsins B, L, and S. JProteomeRes. 2011; 10(12):5363-73.
100. Tholen S, Biniossek M L, Gessler A L, Muller S, Weisser J, Kizhakkedathu J N, et al. Contribution of cathepsin L to secretome composition and cleavage pattern of mouse embryonic fibroblasts. BiolChem. 2011; 392(11):961-71.
101. de Condorcet M. Essay on the Application of Analysis to the Probability of Majority Decisions. L'Imprimerie Royale, France; 1785.
102. Kreher C R, Dittrich M T, Guerkov R, Boehm B O, Tary-Lehmann M. CD4+ and CD8+ cells in cryopreserved human PBMC maintain full functionality in cytokine ELISPOT assays. J Immunol Methods. 2003; 278(1-2):79-93.
103. Ryall S, Zapotocky M, Fukuoka K, Nobre L, Guerreiro Stucklin A, Bennett J, et al. Integrated Molecular and Clinical Analysis of 1,000 Pediatric Low-Grade Gliomas. Cancer Cell. 2020; 37(4):569-83 e5.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A method of selection of peptides for inclusion in a treatment for subjects having cancer, or at risk of having cancer, comprising:
Obtaining sequences of tumor proteins;
Identifying amino acid mutations in the tumor proteins as compared to corresponding wild-type sequences of the protein in the subject or a reference human subject;
Identifying pentamer amino acid motifs that correspond to T cell exposed motifs which comprise the identified amino acid mutations in the tumor proteins;
Determining the frequency of occurrence of the pentamer amino acid motifs in a reference database of reference proteins;
Selecting one or more T cell exposed motifs comprising the mutant amino acids based on the frequency of occurrence of the pentamer amino acid motifs in the reference database; and
Synthesizing one or more synthetic peptides, or nucleic acids encoding the one or more peptides, that comprise each of the one or more selected T cell exposed motifs.

2. The method of claim 1, further comprising the steps of:
Identifying peptides of from 8 to 18 amino acids in length which comprise the T cell exposed motifs comprising the mutant amino acid;
Determining the predicted probability of cleavage of the identified peptide by a peptidase;
Selecting or excluding one or more peptides comprising the T cell exposed motifs for synthesis, based on the probability of cleavage of that peptide by a peptidase; and
Synthesizing one or more synthetic peptides, or nucleic acids encoding the one or more peptides, that comprise each of the one or more selected T cell exposed motifs.

3. The method of any one of claims 1 to 2, further comprising incorporating the one or more synthetic peptide sequences, or the nucleic acid sequences encoding them, into a treatment formulation for administration to a subject.

4. The method of any one of claims 1 to 3, wherein the reference database of reference proteins comprises proteins of the human proteome.

5. The method of any one of claims 1 to 3, wherein the reference database of reference proteins comprises proteins of microorganisms.

6. The method of any one of claims 1 to 3, wherein the reference database of reference proteins comprises proteins of organisms of the gastrointestinal microbiome.

7. The method of any one of claims 1 to 3, wherein the reference database of reference proteins comprises proteins of the human immunoglobulinome.

8. The method of any of claims 1 to 7, wherein the reference database of reference proteins comprises of more than 1,000 proteins.

9. The method of any of claims 1 to 7, wherein the reference database of reference proteins comprises more than 10,000 proteins.

10. The method of any of claims 1 to 7, wherein the reference database of reference proteins comprises more than 20,000 proteins.

11. The method of any of claims 1 to 10, further comprising determining the frequency of the pentamer amino acid motifs in more than one reference database of reference proteins.

12. The method of any of claims 1 to 11, wherein the pentamer amino acid motifs that correspond to T cell exposed motifs in the tumor proteins are found at least once in the human proteome reference database.

13. The method of any of claims 1 to 11, wherein the pentamer amino acid motifs that correspond to T cell exposed motifs in the tumor proteins are found at least 5 times in the human proteome reference database.

14. The method of any of claims 1 to 11, wherein the pentamer amino acid motifs that correspond to T cell exposed motifs in the tumor proteins are found no more than 10 times in the human proteome reference database.

15. The method of any of claims 1 to 14, wherein the pentamer amino acid motifs that correspond to T cell exposed motifs in the tumor proteins are found at least once in the gastrointestinal reference database.

16. The method of any of claims 1 to 14, wherein the pentamer amino acid motifs that correspond to T cell exposed motifs in the tumor proteins occur at least 5 times in the gastrointestinal reference database.

17. The method of any of claims 1 to 14, wherein the pentamer amino acid motifs that correspond to T cell exposed motifs in the tumor proteins occur at least 20 times in a reference database of microbial proteins.

18. The method of any of claims 1 to 17, wherein the T cell exposed motif is a motif exposed to a T cell by a peptide bound to an MHC I molecule.

19. The method of any of claims 1 to 17, wherein the T cell exposed motif is a motif exposed to a T cell by a peptide bound to an MHC II molecule.

20. The method of any of claims 1 to 19, wherein the step of determining the frequency of occurrence of the pentamer amino acid motifs in a reference database of reference proteins informs a decision to exclude the peptide comprising a particular T cell exposed motif from a treatment formulation.

21. The method of claim 20, wherein the pentamer amino acid motif is absent from the human proteome database and the motif is excluded from a treatment formulation.

22. The method of claim 20, wherein the pentamer amino acid motif is present in less than 3 locations in the human proteome and the motif is excluded from a treatment formulation.

23. The method of claim 20, wherein the pentamer amino acid motif is present in more than 20 proteins in the human proteome and the motif is excluded from a treatment formulation.

24. The method of claim 20, wherein the pentamer amino acid motif is present in more than 50 proteins in the human proteome and the motif is excluded from a treatment formulation.

25. A method of selection of peptides for inclusion in a treatment for subjects having cancer, or at risk of having cancer, comprising:
Obtaining sequences of tumor proteins;
Identifying amino acid mutations in the tumor proteins as compared to corresponding wild-type sequences of the protein in the subject or a reference human subject;
Identifying pentamer amino acid motifs that correspond to T cell exposed motifs which comprise the identified amino acid mutations in the tumor proteins;
Identifying peptides of from 8 to 18 amino acids in length which comprise the T cell exposed motifs comprising the mutant amino acid;
Determining the predicted probability of cleavage by a peptidase of the identified peptide;
Selecting or excluding one or more peptides comprising the T cell exposed motifs for synthesis, based on the probability of cleavage of that peptide by a peptidase; and
Synthesizing one or more peptides, or nucleic acids encoding the peptides, that comprise each of the one or more selected T cell exposed motifs.

26. The method of any one of claims 2 to 25, wherein the peptidase is a cathepsin.

27. The method of claim 26 wherein the cathepsin is cathepsin B, L or S.

28. The method of any of claims 2 to 27, wherein peptides are selected that have a probability less than 0.5 of cleavage at any scissile bond position within 9 amino acids of the mutant amino acid.

29. The method of any of claims 2 to 27, wherein peptides are selected that have a probability less than 0.8 of cleavage at any scissile bond position within 9 amino acids of the mutant amino acid.

30. The method of any of claims 2 to 27, wherein the probability of cleavage at any scissile bond position within 9 amino acids of the mutant amino acid is greater than 0.7 and the peptide is excluded from a treatment formulation.

31. The method of claims 2 and 27 wherein the probability of cleavage at any scissile bond position within 9 amino acids of the mutant amino acid is greater than 0.9 and the peptide is excluded from a treatment formulation.

32. The method of any of claims 2 to 31 wherein the cleavage creates a novel T cell epitope and the novel T cell epitope is included in the treatment formulation.

33. The method of any of claims 2 to 32, wherein the peptides are selected that have a probability greater than 0.5 of cleavage at any scissile bond position within 9 amino acids of the mutant amino acid.

34. The method of any of claims 2 to 33, wherein the peptides are selected that have a probability greater than 0.8 of cleavage at any scissile bond position within 9 amino acids of the mutant amino acid.

35. The method of any of claims 2 to 34, wherein the peptides are selected that have a probability greater than 0.8 of cleavage at any scissile bond position within 9 amino acids of the mutant amino acid by more than one cathepsin.

36. The methods of any one of claims 28 to 35, wherein the cleavage occurs at a scissile bond position on the N terminal side of the mutation.

37. The method of any one of claims 2 to 36, wherein the selected peptides are peptides that occur in KRAS, NRAS or HRAS proteins.

38. The method of claim 37, wherein the selected peptides comprise any of the T cell exposed motifs of SEQ ID NOs. 546-635 or SEQ ID NOs. 1386-1475.

39. The method of any of claims 2 to 38, further comprising incorporating the selected peptide sequences, or the nucleic acids encoding them, into a treatment formulation for administration to a subject.

40. The method of any one of claims 2 to 39, wherein the treatment formulation is co-administered to the subject with a cathepsin inhibitor as a component of the treatment regimen.

41. The method of claim 40, wherein the co-administration is within the same treatment formulation.

42. The method of claim 40, wherein the co-administration is a sequential administration.

43. The method of any one of claims 40 to 42, wherein the cathepsin inhibitor is a synthetic organic molecule.

44. The method of any one of claims 40 to 43, wherein the cathepsin inhibitor is selected from the group consisting of nitrile derivatives, ketone derivatives, acryl hydrazine derivatives, vinyl sulfonate derivatives, epoxy succinic acids, surugamides, loxistatin derivatives, sulfonamide derivatives and betalactams.

45. The method of any one of claims 40 to 42, wherein the cathepsin inhibitor is a naturally occurring medicinal product.

46. The method of claim 45, wherein the cathepsin inhibitor is a cystatin protein or polypeptide derived therefrom.

47. The method of claim 46, wherein the cystatin protein or polypeptide derived therefrom is administered encoded in a nucleic acid.

48. The method of claims 40 to 47, wherein the cathepsin inhibitor is administered to the subject parenterally.

49. The method of any one of claims 40 to 47, wherein the cathepsin inhibitor is administered to the subject intratumorally, topically or to a mucosal surface.

50. The method of any one of claims 1 to 48, further comprising selecting peptides that have a predicted probability of binding to one or more MHC alleles with an affinity in the top 25% as compared to all peptides in the protein from which it is derived.

51. The method of claim 50, wherein the MHC is an MHC I.

52. The method of claim 50, wherein the MHC is an MHC II.

53. The method of any one of claims 50 to 52, wherein the binding is to at least three MHC alleles.

54. The method of any one of claims 1 to 53, further comprising for each selected T cell exposed motif, synthesizing a peptide, or the nucleic acids encoding such a peptide, of desired binding affinity for each of an array of MHCs of interest by selecting amino acids to comprise a groove exposed motif in the peptide, thereby synthesizing a peptide that is not naturally present in the tumor proteins of the subject or the reference human subject.

55. The method of claim 54, wherein the groove exposed motif amino acids are further selected based on a property or properties selected from one of more of solubility, stability, or reduced aggregation.

56. The method of any one of claims 1 to 55, wherein the tumor protein is an oncogene or tumor suppressor gene product.

57. The method of any one of claims 1 to 55, wherein the tumor protein comprises a passenger gene mutation.

58. The method of claim 57, wherein the tumor protein is selected from the group consisting of proteins corresponding to the gene identifiers listed in Table 1.

59. The method of any one of claims 56 to 58, wherein the mutated peptide in the tumor protein is selected from the group consisting of SEQ ID NOs: 1-420 and SEQ ID NOs: 841-1260.

60. The method of any one of claims 56 to 58, wherein the pentamer amino acid motif in the tumor protein the is mutated is selected from the group consisting of SEQ ID NO.: NOs: 421-840 and SEQ ID NOs: 1261-1680.

61. The method of any one of claims 1 to 60, wherein the one or more synthesized peptides, or nucleic acids encoding the peptides, have a maximum length of 60 amino acids or 180 nucleotides.

62. The method of any one of claims 1 to 60, wherein the one or more synthesized peptides, or nucleic acids encoding the peptides, have a maximum length of 48 amino acids or 144 nucleotides.

63. The method of any one of claims 1 to 60, wherein the one or more synthesized peptides, or nucleic acids encoding the peptides, have a maximum length of 36 amino acids or 108 nucleotides.

64. The method of any one of claims 1 to 60, wherein the one or more synthesized peptides, or nucleic acids encoding the peptides, have a length of from 9 amino acids or 27 nucleotides to 48 amino acids or 144 nucleotides.

65. The method of any one of claims 1 to 60, wherein the one or more synthesized peptides, or nucleic acids encoding the peptides, have a length of from 9 amino acids or 27 nucleotides to 36 amino acids or 108 nucleotides.

66. The method of any one of claims 1 to 60, wherein the one or more synthesized peptides, or nucleic acids encoding the peptides, have a length of from 9 amino acids or 17 nucleotides to 18 amino acids or 54 nucleotides.

67. The method of any one of claims 1 to 60, wherein the one or more synthesized peptides, or nucleic acids encoding the peptides, have a length of from 15 amino acids or 45 nucleotides to 48 amino acids or 144 nucleotides.

68. The method of any one of claims 1 to 60, wherein the one or more synthesized peptides, or nucleic acids encoding the peptides, have a length of from 15 amino acids or 45 nucleotides to 36 amino acids or 108 nucleotides.

69. The method of any one of claims 1 to 60, wherein the one or more synthesized peptides, or nucleic acids encoding the peptides, have a length of from 12 amino acids or 36 nucleotides to 38 amino acids or 114 nucleotides.

70. The method of any one of claims 1 to 69, wherein the one or more synthesized peptides, or nucleic acids encoding the peptides, are any multiple of 3 amino acids.

71. A method of assembling a library of peptides for treatment of one or more subjects having or at risk of having cancer comprising:
Selecting peptides by application of the method of any of claims 1 to 70; and
Synthesizing the peptides or the nucleic acids encoding the peptides and storing the peptides or nucleic acids.

72. The method of claim 71 wherein the library of peptides comprises pentamer amino acid motifs comprising mutations from at least 10 different tumor proteins or 10 different mutations in the same tumor protein.

73. The method of claim 71 wherein the library of peptides comprises pentamer amino acid motifs comprising mutations from at least 20 different tumor proteins or 20 different mutations in the same tumor protein.

74. The method of claim 71 wherein the library of peptides comprises pentamer amino acid motifs comprising mutations from at least 40 different tumor proteins or 40 different mutations in the same tumor protein.

75. The method of any one of claims 71 to 74, wherein the library of peptides comprises peptides selected to bind at least 3 MHC I alleles.

76. The method of any one of claims 71 to 74, wherein the library of peptides comprises peptides selected to bind at least 5 MHC I alleles.

77. The method of any one of claims 71 to 74, wherein the library of peptides comprises peptides selected to bind at least 3 MHC II alleles.

78. The method of any one of claims 71 to 74, wherein the library of peptides comprises peptides selected to bind at least 5 MHC II alleles.

79. The method of any one of claims 71 to 78, wherein the library of peptides comprises one or more heteroclitic peptides.

80. The method of any one of claims 71 to 79, wherein the library of peptides comprise five or more of the peptides selected from the group consisting of SEQ ID NO.: NOs: 1-420 and SEQ ID NOs:841-1260.

81. The method of any one of claims 71 to 79, wherein the library of peptides comprise five or more of the T cell exposed motifs selected from the group consisting of SEQ ID NO.: NOs: 421-840 and SEQ ID NOs: 1261-1680.

82. The method of any one of claims 71 to 79, wherein the library of peptides comprises five or more of the pentamer amino acid motifs selected from SEQ ID NOs: 421-840 and SEQ ID NOs: 1261-1680, with the proviso that the peptides are not one of sequences SEQ ID NOs: 1-420 or SEQ ID NOs: 841-1260.

83. The method of any one of claims 71 to 79, wherein the library of peptides comprise five or more of the pentamer amino acid motifs selected from the sequences listed in Table 14.

84. The method of any one of claims 71 to 83, wherein one or more peptides selected from the library are administered with additional peptides selected according to the method of claim 1 to target mutations unique to a particular subject's tumor.

85. A library of peptides, or nucleic acid sequences encoding the peptides, created by any of claims 71 to 84.

86. A library of peptides, or nucleic acids encoding the peptides, for use in a treatment for one or more subjects affected by cancer, or at risk of being affected by cancer, comprising five or more of the peptides selected from the group consisting of SEQ ID NOs: 1-420 and SEQ ID NOs: 841-1260.

87. A library of peptides, or nucleic acids encoding the peptides, for use in a treatment for one or more subjects affected by cancer, or at risk of being affected by cancer, comprising five or more of the T cell exposed motifs selected from the group consisting of SEQ ID NOs: 421-840 and SEQ ID NOs: 1261-1680.

88. A library of peptides, or nucleic acids encoding the peptides, for use in a treatment for one or more subjects affected by cancer, or at risk of being affected by cancer, comprising five or more of the T cell exposed motifs selected from the group consisting of SEQ ID NOs: 421-840 and SEQ ID NOs: 1261-1680, with the proviso that the peptides are not SEQ ID NOs: and SEQ ID NOs: 841-1260.

89. A library of peptides, or nucleic acids encoding the peptides, for use in a treatment for one or more subjects affected by cancer, or at risk of being affected by cancer, comprising five or more of the T cell exposed motifs selected from the group consisting of the sequences listed in Table 14.

90. The library of any one of claims 85 to 89, wherein the peptides are from 9 to 60 amino acids in length.

91. A vaccine for a subject affected by cancer or at risk of being affected by cancer comprising peptides or nucleic acids encoding the peptides selected by one or more of the methods of any one of claims 1 to 90 or identified in claims 1 to 90.

92. The vaccine of claim 91, wherein the vaccine comprises peptides.

93. The vaccine of claim 91, wherein the vaccine comprises nucleic acids encoding the selected peptides.

94. The vaccine of any one of claims 91 to 93, wherein the vaccine is formulated for parenteral delivery.

95. The vaccine of any one of claims 91 to 93, wherein the vaccine is formulated for non-parenteral delivery.

96. The vaccine of any one of claims 91 to 93, wherein the vaccine is formulated for oral delivery.

97. The vaccine of any one of claims 91 to 93, wherein the vaccine is formulated as a coated tablet.

98. The vaccine of any one of claims 91 to 93, wherein the vaccine is formulated for intradermal delivery.

99. The vaccine of any of the claims 91 to 98, wherein the vaccine is formulated as a lipid drug delivery system selected from the group consisting of lipid nanoparticles, emulsions, self-emulsifying drug delivery systems, nanocapsules and liposomes.

100. The vaccine of any one of claims 91 to 99, wherein the vaccine comprises an adjuvant.

101. The vaccine of any one of claims 91 to 100, wherein the vaccine comprises peptides or the nucleic acids encoding the peptides selected from a library of peptides designed prior to the diagnosis of cancer in a specific subject.

102. The vaccine of any one of claims 91 to 101, wherein the vaccine is administered in conjunction with a cathepsin inhibitor.

103. The vaccine of any one of claims 91 to 101, wherein the vaccine is formulated with a cathepsin inhibitor.

104. The vaccine of any one of claims 102 to 103, wherein said cathepsin inhibitor is selected from the group consisting of a synthetic organic molecule, a natural medicinal product, or a cystatin protein or polypeptide.

105. A treatment regimen comprising the vaccine of any of claims 91 to 101 and a cathepsin inhibitor.

106. A method of treating a subject in need thereof comprising administering a vaccine of any one of claims 91 to 101 to the subject.

107. The method of claim 106, further comprising administering a cathepsin inhibitor to the subject.

108. The method of any one of claims 106 to 107, wherein the subject has been diagnosed with cancer.

109. The method of any one of claims 106 to 107, wherein the subject is at risk of developing cancer.

110. A method comprising:
Contacting antigen presenting cells collected from the subject ex vivo with the vaccine of any one of claims 91 to 104, or peptides or nucleic acids encoding the peptides selected by one or more of the methods of any one of claims 1 to 90 or identified in claims 1 to 90; and
Administering the cells to the subject.

111. A method for selecting one or more T cell clones comprising:
Selecting and synthesizing a peptide, or a nucleic acid encoding the peptide, by a method according to any one of claims 1 to 90, or identified in claims 1 to 90;
Contacting the peptide in vitro with an antigen presenting cell harvested from a subject;
Collecting T cells from that subject;
Contacting the T cells with the antigen presenting cells thereby presenting the peptide of interest bound in the MHC of the antigen presenting cells to the T cells;
Selecting T cells stimulated by contact with the peptide of interest presented by the antigen presenting cells;
Culturing the T cells to provide expanded T cell clones; and
Harvesting the expanded T cell clones.

112. The method of claim 111, wherein the antigen presenting cell is a dendritic cell, a macrophage, or a B cell.

113. The method of any one of claims 111 to 112, wherein T cells derived from the expanded T cell clones are administered autologously to the subject.

114. The method of any one of claims 111 to 113, wherein T cells derived from the expanded T cell clones are administered to a non-autologous subject that shares one or more HLA alleles with the T cell source subject.

115. The method of any one of claims 111 to 114, wherein T cells derived from the expanded T cell clone population are preserved for future use.

116. The method of any one of claims 111 to 115, wherein the T cells from the subject are harvested from PBMCs.

117. The method of any one of claims 111 to 116, wherein the T cells from the subject are harvested from tumor infiltrating lymphocytes.

118. The method of any one of claims 111 to 117, further comprising nucleotide sequencing the T cell receptors of representative T cells drawn from the expanded T cell clones.

119. The method of claim 118, wherein the alpha and beta chains of the receptors of selected T cells are sequenced and/or cloned.

120. The method of any one of claims 118 and 119, further comprising inserting the alpha and/or beta chain nucleotide sequences of the T cell receptors into a recipient cell.

121. The method of claim 120, wherein the recipient cell is a cell maintained in culture.

122. The method of claim 121, wherein the cell in culture is a mammalian cell, a bacterial cell, or a yeast cell.

123. The method of claim 122, wherein the mammalian cell is a recipient T cell.

124. The method of claim 123, wherein the recipient T cell is an antigen naïve T cell.

125. The method of any one of claims 111 to 123, wherein the peptide comprises a T cell exposed motif selected from the group consisting of SEQ ID NOs: 421-840 and SEQ ID NOs: 1261-1680.

126. The method of any one of claims 111 to 123, wherein the peptide comprises a T cell exposed motif selected from the group consisting of the T cell exposed motif sequences listed in Table 14.

127. A T cell clone produced by a method according to any one of claims 110 to 126.

128. A T cell receptor sequence produced by the method according to any one of claim 110 to 126.

129. An engineered T cell comprising the T cell receptor sequence of claim 127.

130. The engineered T cell of claim 127, wherein the engineered T cell comprises a chimeric T cell receptor comprising the T cell receptor sequence of claim 128.

131. The method of claim 118 wherein the T cell receptor alpha and beta chain sequences are expressed in operable association with a second molecule

132. A method of assembling an array of peptides for inclusion in a treatment for one or more subjects affected by cancer, or at risk of being affected by cancer comprising:
Synthesizing an array of trimer peptides;
Selecting peptides for inclusion in a treatment regimen;
Assembling desired peptides from the trimer peptides; and
Administering the assembled peptides to the subject.

133. The method of claim 132, wherein the array comprises 8000 unique trimers.

134. The method of claim 132, wherein the array comprises at least 7000 unique trimers.

135. The method of claim 132 wherein, the array comprises at least 5000 unique trimers.

136. The method any one of claims 132 to 133, wherein the assembled peptides are 9 mers, or 15 mers.

137. The method of any one of claims 132 to 135, wherein the assembled peptides are 12 mers to 36 mers.

138. The method any one of claims 132 to 135, wherein the assembled peptides are any multiple of 3 amino acids.

139. The method of any one of claims 132 to 135, wherein the assembled peptides are selected according to any of the methods of claims 1-90 or identified in claims 1 to 90.

140. The method of any one of claims 132 to 139, wherein the peptide comprises a T cell epitope.

141. The method of any one of claims 132 to 139, wherein the peptide comprises a B cell epitope.

142. The method of any one of claims 132 to 139, further comprising administering the assembled peptides as a vaccine to a subject in need thereof.

143. A method of inhibiting a cathepsin comprising:
Identifying a linear B cell epitope in a cathepsin protein;
Immunizing a subject with a polypeptide encompassing the linear B cell epitope;
Selecting an antibody based on neutralization of cathepsin cleavage activity; and
Making a recombinant immunoglobulin comprising the variable region of the neutralizing antibody; and
Delivering the recombinant immunoglobulin to a desired cellular site

144. The method of claim 143, wherein said cathepsin is cathepsin B, L or S.

145. The method of claim 143, wherein said desired cellular site is an antigen presenting cell.

146. The method of claim 143, wherein said desired cellular site is a tumor cell.

147. The method of any one of claims 143 to 146, wherein said recombinant immunoglobulin is a tetrameric immunoglobulin molecule or a subcomponent of the immunoglobulin that comprises the variable region.

148. The method of any one of claims 143 to 146, wherein the immunoglobulin or subcomponent is encoded in a nucleic acid sequence.

149. The method of any one of claims 143 to 148, wherein said B cell epitopes comprise 5 or more sequential amino acids drawn from sequences in the group SEQ ID NOs: 2610-2629

150. The method of any one of claims 143 to 149, further comprising co-administering said immunoglobulin with a neoepitope peptide vaccine.

151. A method of inhibiting a cathepsin in a tumor cell comprising:
Identifying a protein that is upregulated in the tumor cell or in its extracellular matrix;
Identifying an antibody epitope binding site in that protein;
Expressing a recombinant immunoglobulin, or subcomponent thereof that has binding affinity for the epitope;
Providing a fusion or conjugate of said recombinant immunoglobulin, or subcomponent thereof with a recombinant cystatin or subcomponent thereof; and
Administering the fusion or conjugate to a subject affected by the tumor.

152. The method of claim 151, wherein the fusion or conjugate is encoded in a nucleic acid sequence.

153. The method of any one of claims 151 to 152, wherein said fusion or conjugate is co-administered with a neoepitope vaccine.
